SEQUENCE LISTING (176 sequences)

SEQ ID NO: 1 - DNA, 991 bp, Homo sapiens
  CDS (43)..(573)
gaattccgacaaggagcagcccgcaagcaccaagtgagaggcatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgtcctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtgaagtcatatagtccatgtctgtgatgtgagccaagtgatatcctgtagtacacattgtactgagtggtttttctgaataaattccatattttacctatgaaaaaaaaaaaaaaaaaaaaaaaccagctgagcgccggtcgctaccaaagccgaattc

SEQ ID NO: 2 - protein, 177 aa, Homo sapiens [sequence omitted]

SEQ ID NO: 3 - DNA, 531 bp, Homo sapiens
  CDS (1)..(531)
atgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgtcctcagct

SEQ ID NO: 4 - protein, 153 aa, Homo sapiens
LRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMSSA

SEQ ID NO: 5 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 178 aa, Artificial Sequence
  Description of Artificial Sequence Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLKCENKSKAVEQVKSDFNKLEDQGVYKAMNEFDIFINCIEAYMMIKMKS

SEQ ID NO: 7 - protein, 179 aa, Homo sapiens
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 8 - protein, 181 aa, Mus musculus
MSWGLQILPCLSLILLLWNQVPGLEGQEFRFGSCQVTGVVLPELWEAFWTVKNTVQTQDDITSIRLLKPQVLRNVSGAESCYLAHSLLKFYLNTVFKNYHSKIAKFKVLRSFSTLANNFIVIMSQLQPSKDNSMLPISESAHQRFLLFRRTFKQLDTEVALVKAFGEVDILLTWMQKFYHL

SEQ ID NO: 9 - protein, 179 aa, Homo sapiens
MQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 10 - DNA, 2920 bp, Homo sapiens
  CDS (143)..(2401)
cgtccgccacgcgtccggactagttctagatcgcgagcggccgcccttttttttttttttttggaagtcctaggactgatctccaggaccagcactcttctcccagcccttagggtcctgctcggccaaggccttccctgccatgcgacctgtcagtgtctggcagtggagcccctgggggctgctgctgtgcctgctgtgcagttcgtgcttggggtctccgtccccttccacgggccctgagaagaaggccgggagccaggggcttcggttccggctggctggcttccccaggaagccctacgagggccgcgtggagatacagcgagctggtgaatggggcaccatctgcgatgatgacttcacgctgcaggctgcccacatcctctgccgggagctgggcttcacagaggccacaggctggacccacagtgccaaatatggccctggaacaggccgcatctggctggacaacttgagctgcagtgggaccgagcagagtgtgactgaatgtgcctcccggggctgggggaacagtgactgtacgcacgatgaggatgctggggtcatctgcaaagaccagcgcctccctggcttctcggactccaatgtcattgaggtagagcatcacctgcaagtggaggaggtgcgaattcgacccgccgttgggtggggcagacgacccctgcccgtgacggaggggctggtggaagtcaggcttcctgacggctggtcgcaagtgtgcgacaaaggctggagcgcccacaacagccacgtggtctgcgggatgctgggcttccccagcgaaaagagggtcaacgcggccttctacaggctgctagcccaacggcagcaacactcctttggtctgcatggggtggcgtgcgtgggcacggaggcccacctctccctctgttccctggagttctatcgtgccaatgacaccgccaggtgccctggggggggccctgcagtggtgagctgtgtgccaggccctgtctacgcggcatccagtggccagaagaagcaacaacagtcgaagcctcagggggaggcccgtgtccgtctaaagggcggcgcccaccctggagagggccgggtagaagtcctgaaggccagcacatggggcacagtctgtgaccgcaagtgggacctgcatgcagccagcgtggtgtgtcgggagctgggcttcgggagtgctcgagaagctctgagtggcgctcgcatggggcagggcatgggtgctatccacctgagtgaagttcgctgctctggacaggagctctccctctggaagtgcccccacaagaacatcacagctgaggattgttcacatagccaggatgccggggtccggtgcaacctaccttacactggggcagagaccaggatccgactcagtgggggccgcagccaacatgaggggcgagtcgaggtgcaaatagggggacctgggccccttcgctggggcctcatctgtggggatgactgggggaccctggaggccatggtggcctgtaggcaactgggtctgggctacgccaaccacggcctgcaggagacctggtactgggactctgggaatataacagaggtggtgatgagtggagtgcgctgcacagggactgagctgtccctggatcagtgtgcccatcatggcacccacatcacctgcaagaggacagggacccgcttcactgctggagtcatctgttctgagactgcatcagatctgttgctgcactcagcactggtgcaggagaccgcctacatcgaagaccggcccctgcatatgttgtactgtgctgcggaagagaactgcctggccagctcagcccgctcagccaactggccctatggtcaccggcgtctgctccgattctcctcccagatccacaacctgggacgagctgacttcaggcccaaggctgggcgccactcctgggtgtggcacgagtgccatgggcattaccacagcatggacatcttcactcactatgatatcctcaccccaaatggcaccaaggtggctgagggccacaaagctagtttctgtctcgaagacactgagtgtcaggaggatgtctccaagcggtatgagtgtgccaactttggagagcaaggcatcactgtgggttgctgggatctctaccggcatgacattgactgtcagtggattgacatcacggatgtgaagccaggaaactacattctccaggttgtcatcaacccaaactttgaagtagcagagagtgactttaccaacaatgcaatgaaatgtaactgcaaatatgatggacatagaatctgggtgcacaactgccacattggtgatgccttcagtgaagaggccaacaggaggtttgaacgctaccctggccagaccagcaaccagattatctaagtgccactgccctctgcaaaccaccactggcccctaatggcaggggtctgaggctgccattacctcaggagcttaccaagaaacccatgtcagcaaccgcactcatcagaccatgcactatggatgtggaactgtcaagcagaagttttcaccctccttcagaggccagctgtcagtatctgtagccaagcatgggaatctttgctcccaggcccagcaccgagcagaacagaccagagcccaccacaccacaaagagcagcacctgactaactgcccacaaaagatggcagcagctcattttctttaataggaggtcaggatggtcagctccagtatctcccctaagtttagggggatacagctttacctctagccttttggtgggggaaaagatccagccctcccacctcattttttactataatatgttgctaggtataattttattttatataaaaagtgtttctgtgattcttcagaaaaaaaaaaaaaaaaaaaaaaaaaaaaaaaaaaa

SEQ ID NO: 11 - protein, 753 aa, Homo sapiens
MRPVSVWQWSPWGLLLCLLCSSCLGSPSPSTGPEKKAGSQGLRFRLAGFPRKPYEGRVEIQRAGEWGTICDDDFTLQAAHILCRELGFTEATGWTHSAKYGPGTGRIWLDNLSCSGTEQSVTECASRGWGNSDCTHDEDAGVICKDQRLPGFSDSNVIEVEHHLQVEEVRIRPAVGWGRRPLPVTEGLVEVRLPDGWSQVCDKGWSAHNSHVVCGMLGFPSEKRVNAAFYRLLAQRQQHSFGLHGVACVGTEAHLSLCSLEFYRANDTARCPGGGPAVVSCVPGPVYAASSGQKKQQQSKPQGEARVRLKGGAHPGEGRVEVLKASTWGTVCDRKWDLHAASVVCRELGFGSAREALSGARMGQGMGAIHLSEVRCSGQELSLWKCPHKNITAEDCSHSQDAGVRCNLPYTGAETRIRLSGGRSQHEGRVEVQIGGPGPLRWGLICGDDWGTLEAMVACRQLGLGYANHGLQETWYWDSGNITEVVMSGVRCTGTELSLDQCAHHGTHITCKRTGTRFTAGVICSETASDLLLHSALVQETAYIEDRPLHMLYCAAEENCLASSARSANWPYGHRRLLRFSSQIHNLGRADFRPKAGRHSWVWHECHGHYHSMDIFTHYDILTPNGTKVAEGHKASFCLEDTECQEDVSKRYECANFGEQGITVGCWDLYRHDIDCQWIDITDVKPGNYILQVVINPNFEVAESDFTNNAMKCNCKYDGHRIWVHNCHIGDAFSEEANRRFERYPGQTSNQII

SEQ ID NO: 12 - DNA, 2262 bp, Homo sapiens
  CDS (1)..(2259)
atgcgacctgtcagtgtctggcagtggagcccctgggggctgctgctgtgcctgctgtgcagttcgtgcttggggtctccgtccccttccacgggccctgagaagaaggccgggagccaggggcttcggttccggctggctggcttccccaggaagccctacgagggccgcgtggagatacagcgagctggtgaatggggcaccatctgcgatgatgacttcacgctgcaggctgcccacatcctctgccgggagctgggcttcacagaggccacaggctggacccacagtgccaaatatggccctggaacaggccgcatctggctggacaacttgagctgcagtgggaccgagcagagtgtgactgaatgtgcctcccggggctgggggaacagtgactgtacgcacgatgaggatgctggggtcatctgcaaagaccagcgcctccctggcttctcggactccaatgtcattgaggtagagcatcacctgcaagtggaggaggtgcgaattcgacccgccgttgggtggggcagacgacccctgcccgtgacggaggggctggtggaagtcaggcttcctgacggctggtcgcaagtgtgcgacaaaggctggagcgcccacaacagccacgtggtctgcgggatgctgggcttccccagcgaaaagagggtcaacgcggccttctacaggctgctagcccaacggcagcaacactcctttggtctgcatggggtggcgtgcgtgggcacggaggcccacctctccctctgttccctggagttctatcgtgccaatgacaccgccaggtgccctggggggggccctgcagtggtgagctgtgtgccaggccctgtctacgcggcatccagtggccagaagaagcaacaacagtcgaagcctcagggggaggcccgtgtccgtctaaagggcggcgcccaccctggagagggccgggtagaagtcctgaaggccagcacatggggcacagtctgtgaccgcaagtgggacctgcatgcagccagcgtggtgtgtcgggagctgggcttcgggagtgctcgagaagctctgagtggcgctcgcatggggcagggcatgggtgctatccacctgagtgaagttcgctgctctggacaggagctctccctctggaagtgcccccacaagaacatcacagctgaggattgttcacatagccaggatgccggggtccggtgcaacctaccttacactggggcagagaccaggatccgactcagtgggggccgcagccaacatgaggggcgagtcgaggtgcaaatagggggacctgggccccttcgctggggcctcatctgtggggatgactgggggaccctggaggccatggtggcctgtaggcaactgggtctgggctacgccaaccacggcctgcaggagacctggtactgggactctgggaatataacagaggtggtgatgagtggagtgcgctgcacagggactgagctgtccctggatcagtgtgcccatcatggcacccacatcacctgcaagaggacagggacccgcttcactgctggagtcatctgttctgagactgcatcagatctgttgctgcactcagcactggtgcaggagaccgcctacatcgaagaccggcccctgcatatgttgtactgtgctgcggaagagaactgcctggccagctcagcccgctcagccaactggccctatggtcaccggcgtctgctccgattctcctcccagatccacaacctgggacgagctgacttcaggcccaaggctgggcgccactcctgggtgtggcacgagtgccatgggcattaccacagcatggacatcttcactcactatgatatcctcaccccaaatggcaccaaggtggctgagggccacaaagctagtttctgtctcgaagacactgagtgtcaggaggatgtctccaagcggtatgagtgtgccaactttggagagcaaggcatcactgtgggttgctgggatctctaccggcatgacattgactgtcagtggattgacatcacggatgtgaagccaggaaactacattctccaggttgtcatcaacccaaactttgaagtagcagagagtgactttaccaacaatgcaatgaaatgtaactgcaaatatgatggacatagaatctgggtgcacaactgccacattggtgatgccttcagtgaagaggccaacaggaggtttgaacgctaccctggccagaccagcaaccagattatctaa

SEQ ID NO: 13 - protein, 38 aa, Artificial Sequence
  Description of Artificial Sequence (SRRD) consensus sequence
GXXXXXGXXEXXXXXXWGXXCXXXXXXXXXXXXCXXXG

SEQ ID NO: 14 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAGRTRTAGSSGVTAGRPRPTARHWFQAGYSTSRAREAGPSRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 15 - protein, 574 aa, Homo sapiens
MALARGSRQLGALVWGACLCVLVHGQQAQPGQGSDPARWRQLIQWENNGQVYSLLNSGSEYVPAGPQRSESSSRVLLAGAPQAQQRRSHGSPRRRQAPSLPLPGRVGSDTVRGQARHPFGFGQVPDNWREVAVGDSTGMALARTSVSQQRHGGSASSVSASAFASTYRQQPSYPQQFPYPQAPFVSQYENYDPASRTYDQGFVYYRPAGGGVGAGAAAVASAGVIYPYQPRARYEEYGGGEELPEYPPQGFYPAPERPYVPPPPPPPDGLDRRYSHSLYSEGTPGFEQAYPDPGPEAAQAHGGDPRLGWYPPYANPPPEAYGPPRALEPPYLPVRSSDTPPPGGERNGAQQGRLSVGSVYRPNQNGRGLPDLVPDPNYVQASTYVQRAHLYSLRCAAEEKCLASTAYAPEATDYDVRVLLRFPQRVKNQGTADFLPNRPRHTWEWHSCHQHYHSMDEFSHYDLLDAATGKKVAEGHKASFCLEDSTCDFGNLKRYACTSHTQGLSPGCYDTYNADIDCQWIDITDVQPGNYILKVHVNPKYIVLESDFTNNVVRCNIHYTGRYVSATNCKIVQS

SEQ ID NO: 16 - protein, 774 aa, Homo sapiens
MERPLCSHLCSCLAMLALLSPLSLAQYDSWPHYPEYFQQPAPEYHQPQAPANVAKIQLRLAGQKRKHSEGRVEVYYDGQWGTVCDDDFSIHAAHVVCRELGYVEAKSWTASSSYGKGEGPIWLDNLHCTGNEATLAACTSNGWGVTDCKHTEDVGVVCSDKRIPGFKFDNSLINQIENLNIQVEDIRIRAILSTYRKRTPVMEGYVEVKEGKTWKQICDKHWTAKNSRVVCGMFGFPGERTYNTKVYKMFASRRKQRYWPFSMDCTGTEAHISSCKLGPQVSLDPMKNVTCENGLPAVVSCVPGQVFSPDGPSRFRKAYKPEQPLVRLRGGAYIGEGRVEVLKNGEWGTVCDDKWDLVSASVVCRELGFGSAKEAVTGSRLGQGIGPIHLNEIQCTGNEKSIIDCKFNAESQGCNHEEDAGVRCNTPAMGLQKKLRLNGGRNPYEGRVEVLVERNGSLVWGMVCGQNWGIVEAMVVCRQLGLGFASNAFQETWYWHGDVNSNKVVMSGVKCSGTELSLAHCRHDGEDVACPQGGVQYGAGVACSETAPDLVLNAEMVQQTTYLEDRPMFMLQCAMEENCLSASAAQTDPTTGYRRLLRFSSQIHNNGQSDFRPKNGRHAWIWHDCHRHYHSMEVFTHYDLLNLNGTKVAEGHKASFCLEDTECEGDIQKNYECANFGDQGITMGCWDMYRHDIDCQWVDITDVPPGDYLFQVVINPNFEVAESDYSNNIMKCRSRYDGHRIWMYNCHIGGSFSEETEKKFEHFSGLLNNQLSPQ

SEQ ID NO: 17 - protein, 754 aa, Mus musculus
MRAVSVWYCCPWGLLLLHCLCSFSVGSPSPSISPEKKVGSQGLRFRLAGFPRKPYEGRVEIQRAGEWGTICDDDFTLQAAHVLCRELGFTEATGWTHSAKYGPGTGRIWLDNLSCRGTEGSVTECASRGWGNSDCTHDEDAGVICKDQRLPGFSDSNVIEVEHQLQVEEVRLRPAVEWGRRPLPVTEGLVEVRLPEGWSQVCDKGWSAHNSHVVCGMLGFPGEKRVNMAFYRMLAQKKQHSFGLHSVACVGTEAHLSLCSLEFYRANDTTRCSGGNPAVVSCVLGPLYATFTGQKKQQHSKPQGEARVRLKGGAHQGEGRVEVLKAGTWGTVCDRKWDLQAASVVCPELGFGTAREALSGARMGQGMGAIHLSEVRCSGQEPSLWRCPSKNITAEDCSHSQDAGVRCNLPYTGVETKIRLSGGRSRYEGRVEVQIGIPGHLRWGLICGDDWGTLEAMVACRQLGLGYANHGLQETWYWDSGNVTEVVMSGVRCTGSELSLNQCAHHSSHITCKKTGTRFTAGVICSETASDLLLHSALVQETAYIEDRPLHMLYCAAEENCLASSARSANWPYGHRRLLRFSSQIHNLGRADFRPKAGRHSWVWHECHGHYHSMDIFTHYDILTPNGTKVAEGHKASFCLEDTECQEDVSKRYECANFGEQGITVGCWDLYRHDIDCQWIDITDVKPGNYILQVVINPNFEVAESDFTNNAMKCNCKYDGHRIWVHNCHIGDAFSEEANRRFERYPGQTSNQIV

SEQ ID NO: 18 - protein, 11 aa, Artificial Sequence
  Description of Artificial Sequence Copper-binding talon consensus sequence
WEWHSCHQHYH

SEQ ID NO: 19 - protein, 451 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLTPFTKVRLVGGSGPHEGRVEILHSGQWGTICDDRWEVRVGQVVCRSLGYPGVQAVHKAAHFGQGTGPIWLNEVFCFGRESSIEECKIRQWGTRACSHSEDAGVTCTL

SEQ ID NO: 20 - DNA, 24 bp, Artificial Sequence
  Description of Artificial Sequence Primer
gcttaccaagaaacccatgtcagc

SEQ ID NO: 21 - DNA, 20 bp, Artificial Sequence
  Description of Artificial Sequence Primer
ggcagttagtcaggtgctgc

SEQ ID NO: 22 - DNA, 981 bp, Mus musculus
  CDS (107)..(748)
gaattcggcacgagggccggcacccccgcgccaccccagcctcaaactgcagtccggcgccgcggggcaggacaaggggaaggaataaacacgtttggtgagagccatggcactcaaggtcctacctctacacaggacggtgctcttcgctgccattctcttcctactccacctggcatgtaaagtgagttgcgaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtaccagcaaggtgaaccttgtgaagatctcctccaccgtctccagccctcgggacacggcgctggctgccgtcatctgcagtgctctggccacggtgctgctcgccctgctcatcctgtgtgtcatctactgcaagaggcagttcatggagaagaaacccagctgtaagctcccatccctctgtctcactgtgaagtgagcttgttagcattgtcacccaagagttctcaagacacctggctgagacctaagacctttagagcatcaacagctacttagaatacaagatgcaggaaaacgagcctcttcaggaatctcagggcctcctagggatgctggcaaggctgtgatgtctcaaggctaccaggaaaaaataaaagttgtctataccctaaaaaaaaaaaaaaaaaaaaaaaaacatgcggccgc

SEQ ID NO: 23 - protein, 214 aa, Mus musculus
MALKVLPLHRTVLFAAILFLLHLACKVSCETGDCRQQEFKDRSGNCVLCKQCGPGMELSKECGFGYGEDAQCVPCRPHRFKEDWGFQKCKPCADCALVNRFQRANCSHTSDAVCGDCLPGFYRKTKLVGFQDMECVPCGDPPPPYEPHCTSKVNLVKISSTVSSPRDTALAAVICSALATVLLALLILCVIYCKRQFMEKKPSCKLPSLCLTVK

SEQ ID NO: 24 - DNA, 642 bp, Mus musculus
  CDS (1)..(642)
atggcactcaaggtcctacctctacacaggacggtgctcttcgctgccattctcttcctactccacctggcatgtaaagtgagttgcgaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtaccagcaaggtgaaccttgtgaagatctcctccaccgtctccagccctcgggacacggcgctggctgccgtcatctgcagtgctctggccacggtgctgctcgccctgctcatcctgtgtgtcatctactgcaagaggcagttcatggagaagaaacccagctgtaagctcccatccctctgtctcactgtgaag

SEQ ID NO: 25 - DNA, 555 bp, Mus musculus
  CDS (1)..(555)
gaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtaccagcaaggtgaaccttgtgaagatctcctccaccgtctccagccctcgggacacggcgctggctgccgtcatctgcagtgctctggccacggtgctgctcgccctgctcatcctgtgtgtcatctactgcaagaggcagttcatggagaagaaacccagctgtaagctcccatccctctgtctcactgtgaag

SEQ ID NO: 26 - DNA, 655 bp, Mus musculus
  CDS (110)..(559)
gaattcggcacgagggcgtttggcgcggaagtgctaccaagctgcggaaagcgtgagtctggagcacagcactggcgagtagcaggaataaacacgtttggtgagagccatggcactcaaggtcctacctctacacaggacggtgctcttcgctgccattctcttcctactccacctggcatgtaaagtgagttgcgaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtgagtgatgtgccaagtggcagcagacctttaaaaaaaaaagaaaaaaaaacaaacaaaaacaaaaaaaaaaaaaaaaaaaaaaaaatttccgcggccgc

SEQ ID NO: 27 - protein, 150 aa, Mus musculus
MALKVLPLHRTVLFAAILFLLHLACKVSCETGDCRQQEFKDRSGNCVLCKQCGPGMELSKECGFGYGEDAQCVPCRPHRFKEDWGFQKCKPCADCALVNRFQRANCSHTSDAVCGDCLPGFYRKTKLVGFQDMECVPCGDPPPPYEPHCE

SEQ ID NO: 28 - DNA, 450 bp, Mus musculus
  CDS (1)..(450)
atggcactcaaggtcctacctctacacaggacggtgctcttcgctgccattctcttcctactccacctggcatgtaaagtgagttgcgaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtgag

SEQ ID NO: 29 - DNA, 363 bp, Mus musculus
  CDS (1)..(363)
gaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtgag

SEQ ID NO: 30 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 31 - DNA, 1344 bp, Homo sapiens
  CDS (273)..(1022)
gaattcggaacgaggggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcatcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgttcagacctggtcggggcccactggaagcatccagaacagcaccaccatctagcggcc

SEQ ID NO: 32 - protein, 250 aa, Homo sapiens [sequence omitted]

SEQ ID NO: 33 - DNA, 754 bp, Homo sapiens
  CDS (1)..(750)
atgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgat

SEQ ID NO: 34 - protein, 17 aa, Homo sapiens
VRIQDAGVYLLYSQVLF

SEQ ID NO: 35 - protein, 49 aa, Homo sapiens
MPASSPFLLAPKGPPGNMGGPVREPALSVALWLSWGAALGAVACAMALL

SEQ ID NO: 36 - DNA, 603 bp, Homo sapiens
  CDS (1)..(603)
acccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 37 - protein, 201 aa, Homo sapiens
TQQTELQSLRREVSRLQGTGGPSQNGEGYPWQSLPEQSSDALEAWENGERSRKRRAVLTQKQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 38 - DNA, 699 bp, Homo sapiens
  CDS (1)..(699)
atggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 39 - protein, 233 aa, Homo sapiens [sequence omitted]

SEQ ID NO: 40 - DNA, 615 bp, Homo sapiens
  CDS (1)..(615)
atggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactg

SEQ ID NO: 41 - protein, 205 aa, Homo sapiens [sequence omitted]

SEQ ID NO: 42 - protein, 17 aa, Artificial Sequence
  Description of Artificial Sequence Consensus sequence
VXIXXXGVYLLXXEVXF

SEQ ID NO: 43 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 44 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 45 - DNA, 1119 bp, Homo sapiens
  CDS (140)..(871)
gtcgacccacgcgtccggcaggatgtttgcagtgtcgcgcccagggctctgagactgagcctgccatccactcgcacgcctttctttcagggcttttcggctgttggctacactgatgtgacccccctccctttttggaatgatggggatctttttggtgtatgttggatttgttttcttttccgttttatatgtacaacaagggctttcttctcaagcaaaatttaccgagtttccgcggaacgtgacggcgaccgaggggcagaatgtggagatgtcctgcgccttccagagcggctccgcctcggtgtatctggagatccaatggtggttcctgcgggggccggaggacctggatcccggggccgagggggccggcgcgcaggtggagctcttgcccgacagagacccggacagcgacgggaccaagatcagcacagtgaaagtccaaggcaatgacatctcccacaagcttcagatttccaaagtgaggaaaaaggatgaaggcttatatgagtgcagggtgactgatgccaactacggggagcttcaggaacacaaggcccaggcctatctgaaagtcaatgccaacagccatgcccgcagaatgcaggccttcgaagcctcgcccatgtggctgcaggatatgaagccccgcaagaacgtctccgcagccatccccagcagcatccatggctctgccaaccaacgaacgcactccacctccagccctcaagtggtagccaaaatccccaaacaaagtccacaatcaggtatggaaacccatttcgagccttttattttaccactcacaaacgctccacagaaaggtcagtcgtatagagtagacagatttatgaatggtgatttttaaaatcggagacctagttcagtgcaagtgattatgagaggtgagcactgagcctgcaccaattcactcagagctcaaagcatgtgggtgcaccccgtcagtcccctagtggtgcttcatttccagggcatctgagagctggactctggtttttatcctttctgtatttacacattataagaacaataaatcatgtaatgttggttacattacaaaaaaaaaaaaaaaaaaaaaaaaagggcggccgc

SEQ ID NO: 46 - protein, 244 aa, Homo sapiens
MMGIFLVYVGFVFFSVLYVQQGLSSQAKFTEFPRNVTATEGQNVEMSCAFQSGSASVYLEIQWWFLRGPEDLDPGAEGAGAQVELLPDRDPDSDGTKISTVKVQGNDISHKLQISKVRKKDEGLYECRVTDANYGELQEHKAQAYLKVNANSHARRMQAFEASPMWLQDMKPRKNVSAAIPSSIHGSANQRTHSTSSPQVVAKIPKQSPQSGMETHFEPFILPLTNAPQKGQSYRVDRFMNGDF

SEQ ID NO: 47 - DNA, 735 bp, Homo sapiens
  CDS (1)..(732)
atgatggggatctttttggtgtatgttggatttgttttcttttccgttttatatgtacaacaagggctttcttctcaagcaaaatttaccgagtttccgcggaacgtgacggcgaccgaggggcagaatgtggagatgtcctgcgccttccagagcggctccgcctcggtgtatctggagatccaatggtggttcctgcgggggccggaggacctggatcccggggccgagggggccggcgcgcaggtggagctcttgcccgacagagacccggacagcgacgggaccaagatcagcacagtgaaagtccaaggcaatgacatctcccacaagcttcagatttccaaagtgaggaaaaaggatgaaggcttatatgagtgcagggtgactgatgccaactacggggagcttcaggaacacaaggcccaggcctatctgaaagtcaatgccaacagccatgcccgcagaatgcaggccttcgaagcctcgcccatgtggctgcaggatatgaagccccgcaagaacgtctccgcagccatccccagcagcatccatggctctgccaaccaacgaacgcactccacctccagccctcaagtggtagccaaaatccccaaacaaagtccacaatcaggtatggaaacccatttcgagccttttattttaccactcacaaacgctccacagaaaggtcagtcgtatagagtagacagatttatgaatggtgatttttaa

SEQ ID NO: 48 - DNA, 660 bp, Homo sapiens
  CDS (1)..(657)
caagcaaaatttaccgagtttccgcggaacgtgacggcgaccgaggggcagaatgtggagatgtcctgcgccttccagagcggctccgcctcggtgtatctggagatccaatggtggttcctgcgggggccggaggacctggatcccggggccgagggggccggcgcgcaggtggagctcttgcccgacagagacccggacagcgacgggaccaagatcagcacagtgaaagtccaaggcaatgacatctcccacaagcttcagatttccaaagtgaggaaaaaggatgaaggcttatatgagtgcagggtgactgatgccaactacggggagcttcaggaacacaaggcccaggcctatctgaaagtcaatgccaacagccatgcccgcagaatgcaggccttcgaagcctcgcccatgtggctgcaggatatgaagccccgcaagaacgtctccgcagccatccccagcagcatccatggctctgccaaccaacgaacgcactccacctccagccctcaagtggtagccaaaatccccaaacaaagtccacaatcaggtatggaaacccatttcgagccttttattttaccactcacaaacgctccacagaaaggtcagtcgtatagagtagacagatttatgaatggtgatttttaa

SEQ ID NO: 49 - protein, 219 aa, Homo sapiens
QAKFTEFPRNVTATEGQNVEMSCAFQSGSASVYLEIQWWFLRGPEDLDPGAEGAGAQVELLPDRDPDSDGTKISTVKVQGNDISHKLQISKVRKKDEGLYECRVTDANYGELQEHKAQAYLKVNANSHARRMQAFEASPMWLQDMKPRKNVSAAIPSSIHGSANQRTHSTSSPQVVAKIPKQSPQSGMETHFEPFILPLTNAPQKGQSYRVDRFMNGDF

SEQ ID NO: 50 - DNA, 3196 bp, Mus musculus
  CDS (268)..(1020)
cacgcgtccgcagccagccggcggcggagacacttcacggcgtggcaacccgggtctgtgccttgaagcctccggatcgcagccagctcggtccatccctcactagtcgcaatcccctgtgtccaagctactctttgctatgagcggcagcatgcgtgcagtatcgcgccccaggctctgagagcagcctgcggacacgcttgcctatctgtctttttaggttttggggctctgggctacacggatgtgccccactcccttggcatgatggggatctttttggcgtctgttggatttatgttcttttccgtgttatatgtacaacaagggctttcttctcaagcaaaatttaccgagttgccgagaaatgtgactgctaccgaagggcaaaatgtggagatgtcctgtgctttccaaagcggctctgcttcagtgtacctggagatccagtggtggttccttcgggggccagaggacctggagcaaggcacggaggctgcaggctcgcaggtggagctcttacccgacagagacccggacaacgatgggaccaagattagtacagtgaaagtccaaggcaatgatatctcccacaagcttcagatatccaaagtgagaaaaaaggatgaaggtttatacgagtgcagggtgactgacgctaactacggggagcttcaggaacacaaggcccaggcctatctgaaagtcaatgccaacagccatgctcggaggatgcaggcctttgaagcctcacctatgtggctgcaagacacgaagcctcgaaagaacgcatcatcggtggttcccagcagcgtccacaactctgccaaccaacgaatgcactccacctccagccctcaagcggtagccaaaatccccaagcaaagtccacaatcagcaaagagcaaatcgcctgtaaaatctacggagcggacagcaaagttgaccctatactccaagcaccattctgcacccctgtactctagttatctacacaaggagcatcagcttccggaagcataagtgaagacactgtcacacgctttattgataatattttctttgggaagttgctgatcttttatttcaagagaattaatgggaagagataggacattttccaattacaagaccaatttttttccttttatttcaacaaataaaacctgcatttcactgactgctcaggagttggcctgaatgacatcagtatactaaatatttccatggattccaccaatttcctaacgagggacacctaatcttcaagaagcaaacaaagatggaaaacctaagaaccacaaactgtctcatacagcaccccagctgaggaacaaaacaaatagctaaatgctgaccatggcaaatcaacatcagacaactttattttacatatggaataatcaaagaaagttttttttttacttcctttttgccccctggaatttatcttggagtttcccttttttccttgattgccgttttcgttcaatggtagcaagtgccaattatggccaatccttgtcaatcctggaaggtttatattcatatacattgagtgtggtatatatcaatgtattttaattcatttggcaatttctgtataggcaaacctggcaaattctgtaaattgcttatagtatgtgtgatatgacttcaaggtagataggctatgatgctcatgcaagctgactttcttcattctatatacaaatatattcatgagcatatattaggccaccaacttcttttcctaaagaattatttttcatttgtacctcatgtattttgtgaattttgtagtatatttctctgttccactagtttgaccgctacagtttgtctctgttgtcctctacttccttctggaaaaatttaaaattgtgtatgtctctgataaatgaattaattttgttgtgtgtatgctatgttggaatttgctgtgttcttttaaacatgtatttattaaggtttggggatcttgagttgagtctgaagaatgcacacctggtttttgacagagttcctcatgttaccaatattctatctcagagaaagaaagacaccaagtgggaaaactaagaagacattttgacttcccaagatcctggaagagcacttcacactctgactaaataatgttgctttttttgttcttcaagacttttttgtagctttgtctttctgttagttgctgctaattatattttaatgtctactaattaaaaattaaaatgtgattgttggctgaatacaatatgcaaatgactgcaaagcccatactgaagaaaatagatgtttaatcttcactcaataattataattttaaatagttcatcattattttttgaccttatgatattttgtttagacctgttctaattacatctttctctggcaaagaaagatagaacaatcaatacattccctcttacagtatggaatggttgtggcttaagaaagaatgcatccagatggtcttccagagagattattttattttcattataaaaccagaaaccatatatgtaggaatggttcattcctaatgtaaggccataaattgtagcttgaaggcaaggaatacatttgtttttttatggtaaaggactggcctctgacatgcacttataagcaatgtgaatattttcataatatgcttgacattctcctttaacaaatattgttttatggtaaatctttccttgccatttttcttctttcatttgattcattatttcattctaatgaagaaaataaaggtttaattatgatactttattaacatacaaatgtattttctttctaagttaaatatctgaaagttgtataaaatgatggtagagaaatattactcattcggtttctttgagctttaagaatcccatacattgcagtatatattagaatactgatttaacatcaaactgggggggaaaatcatgtattatacttttactcaatgtctaggtaatggattcagctaattttacagcaagccaaatgtgtacccgtatcagtaatgttcaccatgcttgtaataaaagggcatatgctagttttggaagaatgctcattagattcattgtatcagtgtccaaaataataaagacctgtttatcactgtgaaaaaaaaaaaaaaaaaaaaaaaaaaaaaaaaaaaaaaaaaaaaaaaaaaaaaaaaaagggcggccgc

SEQ ID NO: 51 - protein, 251 aa, Mus musculus
MGIFLASVGFMFFSVLYVQQGLSSQAKFTELPRNVTATEGQNVEMSCAFQSGSASVYLEIQWWFLRGPEDLEQGTEAAGSQVELLPDRDPDNDGTKISTVKVQGNDISHKLQISKVRKKDEGLYECRVTDANYGELQEHKAQAYLKVNANSHARRMQAFEASPMWLQDTKPRKNASSVVPSSVHNSANQRMHSTSSPQAVAKIPKQSPQSAKSKSPVKSTERTAKLTLYSKHHSAPLYSSYLHKEHQLPEA

SEQ ID NO: 52 - DNA, 756 bp, Mus musculus
  CDS (1)..(753)
atggggatctttttggcgtctgttggatttatgttcttttccgtgttatatgtacaacaagggctttcttctcaagcaaaatttaccgagttgccgagaaatgtgactgctaccgaagggcaaaatgtggagatgtcctgtgctttccaaagcggctctgcttcagtgtacctggagatccagtggtggttccttcgggggccagaggacctggagcaaggcacggaggctgcaggctcgcaggtggagctcttacccgacagagacccggacaacgatgggaccaagattagtacagtgaaagtccaaggcaatgatatctcccacaagcttcagatatccaaagtgagaaaaaaggatgaaggtttatacgagtgcagggtgactgacgctaactacggggagcttcaggaacacaaggcccaggcctatctgaaagtcaatgccaacagccatgctcggaggatgcaggcctttgaagcctcacctatgtggctgcaagacacgaagcctcgaaagaacgcatcatcggtggttcccagcagcgtccacaactctgccaaccaacgaatgcactccacctccagccctcaagcggtagccaaaatccccaagcaaagtccacaatcagcaaagagcaaatcgcctgtaaaatctacggagcggacagcaaagttgaccctatactccaagcaccattctgcacccctgtactctagttatctacacaaggagcatcagcttccggaagcataa

SEQ ID NO: 53 - DNA, 684 bp, Mus musculus
  CDS (1)..(681)
caagcaaaatttaccgagttgccgagaaatgtgactgctaccgaagggcaaaatgtggagatgtcctgtgctttccaaagcggctctgcttcagtgtacctggagatccagtggtggttccttcgggggccagaggacctggagcaaggcacggaggctgcaggctcgcaggtggagctcttacccgacagagacccggacaacgatgggaccaagattagtacagtgaaagtccaaggcaatgatatctcccacaagcttcagatatccaaagtgagaaaaaaggatgaaggtttatacgagtgcagggtgactgacgctaactacggggagcttcaggaacacaaggcccaggcctatctgaaagtcaatgccaacagccatgctcggaggatgcaggcctttgaagcctcacctatgtggctgcaagacacgaagcctcgaaagaacgcatcatcggtggttcccagcagcgtccacaactctgccaaccaacgaatgcactccacctccagccctcaagcggtagccaaaatccccaagcaaagtccacaatcagcaaagagcaaatcgcctgtaaaatctacggagcggacagcaaagttgaccctatactccaagcaccattctgcacccctgtactctagttatctacacaaggagcatcagcttccggaagcataa

SEQ ID NO: 54 - protein, 227 aa, Mus musculus
QAKFTELPRNVTATEGQNVEMSCAFQSGSASVYLEIQWWFLRGPEDLEQGTEAAGSQVELLPDRDPDNDGTKISTVKVQGNDISHKLQISKVRKKDEGLYECRVTDANYGELQEHKAQAYLKVNANSHARRMQAFEASPMWLQDTKPRKNASSVVPSSVHNSANQRMHSTSSPQAVAKIPKQSPQSAKSKSPVKSTERTAKLTLYSKHHSAPLYSSYLHKEHQLPEA

SEQ ID NO: 55 - protein, 89 aa, Homo sapiens
GQNVEMSCAFQSGSASVYLEIQWWFLRGPEDLDPGAEGAGAQVELLPDRDPDSDGTKISTVKVQGNDISHKLQISKVRKKDEGLYECRV

SEQ ID NO: 56 - protein, 89 aa, Mus musculus
GQNVEMSCAFQSGSASVYLEIQWWFLRGPEDLEQGTEAAGSQVELLPDRDPDNDGTKISTVKVQGNDISHKLQISKVRKKDEGLYECRV

SEQ ID NO: 57 - protein, 47 aa, Artificial Sequence
  Description of Artificial Sequence Consensus sequence
GQSVTLTCMVSFHPPDYTIWWYRNGQPITLTINSWQYEDSETYWCMV

SEQ ID NO: 58 - DNA, 2852 bp, Homo sapiens
  CDS (160)..(2178)
gtcgacccacgcgtccggagcccggggcgggtggacgcggactcgaacgcagttgcttcgggacccaggaccccctcgggcccgacccgccaggaaagactgaggccgcggcctgccccgcccggctccctgcgccgccgccgcctcccgggacagaagatgtgctccagggtccctctgctgctgccgctgctcctgctactggccctggggcctggggtgcagggctgcccatccggctgccagtgcagccagccacagacagtcttctgcactgcccgccaggggaccacggtgccccgagacgtgccacccgacacggtggggctgtacgtctttgagaacggcatcaccatgctcgacgcaggcagctttgccggcctgccgggcctgcagctcctggacctgtcacagaaccagatcgccagcctgcccagcggggtcttccagccactcgccaacctcagcaacctggacctgacggccaacaggctgcatgaaatcaccaatgagaccttccgtggcctgcggcgcctcgagcgcctctacctgggcaagaaccgcatccgccacatccagcctggtgccttcgacacgctcgaccgcctcctggagctcaagctgcaggacaacgagctgcgggcactgcccccgctgcgcctgccccgcctgctgctgctggacctcagccacaacagcctcctggccctggagcccggcatcctggacactgccaacgtggaggcgctgcggctggctggtctggggctgcagcagctggacgaggggctcttcagccgcttgcgcaacctccacgacctggatgtgtccgacaaccagctggagcgagtgccacctgtgatccgaggcctccggggcctgacgcgcctgcggctggccggcaacacccgcattgcccagctgcggcccgaggacctggccggcctggctgccctgcaggagctggatgtgagcaacctaagcctgcaggccctgcctggcgacctctcgggcctcttcccccgcctgcggctgctggcagctgcccgcaaccccttcaactgcgtgtgccccctgagctggtttggcccctgggtgcgcgagagccacgtcacactggccagccctgaggagacgcgctgccacttcccgcccaagaacgctggccggctgctcctggagcttgactacgccgactttggctgcccagccaccaccaccacagccacagtgcccaccacgaggcccgtggtgcgggagcccacagccttgtcttctagcttggctcctacctggcttagccccacagcgccggccactgaggcccccagcccgccctccactgccccaccgactgtagggcctgtcccccagccccaggactgcccaccgtccacctgcctcaatgggggcacatgccacctggggacacggcaccacctggcgtgcttgtgccccgaaggcttcacgggcctgtactgtgagagccagatggggcaggggacacggcccagccctacaccagtcacgccgaggccaccacggtccctgaccctgggcatcgagccggtgagccccacctccctgcgcgtggggctgcagcgctacctccaggggagctccgtgcagctcaggagcctccgtctcacctatcgcaacctatcgggccctgataagcggctggtgacgctgcgactgcctgcctcgctcgctgagtacacggtcacccagctgcggcccaacgccacttactccgtctgtgtcatgcctttggggcccgggcgggtgccggagggcgaggaggcctgcggggaggcccatacacccccagccgtccactccaaccacgccccagtcacccaggcccgcgagggcaacctgccgctcctcattgcgcccgccctggccgcggtgctcctggccgcgctggctgcggtgggggcagcctactgtgtgcggcgggggcgggccatggcagcagcggctcaggacaaagggcaggtggggccaggggctgggcccctggaactggagggagtgaaggtccccttggagccaggcccgaaggcaacagagggcggtggagaggccctgcccagcgggtctgagtgtgaggtgccactcatgggcttcccagggcctggcctccagtcacccctccacgcaaagccctacatctaagccagagagagacagggcagctggggccgggctctcagccagtgagatggccagccccctcctgctgccacaccacgtaagttctcagtcccaacctcggggatgtgtgcagacagggctgtgtgaccacagctgggccctgttccctctggacctcggtctcctcatctgtgagatgctgtggcccagctgacgagccctaacgtccccagaaccgagtgcctatgaggacagtgtccgccctgccctccgcaacgtgcagtccctgggcacggcgggccctgccatgtgctggtaacgcatgcctgggccctgctgggctctcccactccaggcggaccctgggggccagtgaaggaagctcccggaaagagcagagggagagcgggtaggcggctgtgtgactctagtcttggccccaggaagcgaaggaacaaaagaaactggaaaggaagatgctttaggaacatgttttgcttttttaaaatatatatatatttataagagatcctttcccatttattctgggaagatgtttttcaaactcagagacaaggactttggtttttgtaagacaaacgatgatatgaaggccttttgtaagaaaaaataaaagatgaagtgtgaaaaaaaaaaaaaaaaaaaaaaaaaaaaaaaaaaagggcggccgc

SEQ ID NO: 59 - protein, 673 aa, Homo sapiens
MCSRVPLLLPLLLLLALGPGVQGCPSGCQCSQPQTVFCTARQGTTVPRDVPPDTVGLYVFENGITMLDAGSFAGLPGLQLLDLSQNQIASLPSGVFQPLANLSNLDLTANRLHEITNETFRGLRRLERLYLGKNRIRHIQPGAFDTLDRLLELKLQDNELRALPPLRLPRLLLLDLSHNSLLALEPGILDTANVEALRLAGLGLQQLDEGLFSRLRNLHDLDVSDNQLERVPPVIRGLRGLTRLRLAGNTRIAQLRPEDLAGLAALQELDVSNLSLQALPGDLSGLFPRLRLLAAARNPFNCVCPLSWFGPWVRESHVTLASPEETRCHFPPKNAGRLLLELDYADFGCPATTTTATVPTTRPVVREPTALSSSLAPTWLSPTAPATEAPSPPSTAPPTVGPVPQPQDCPPSTCLNGGTCHLGTRHHLACLCPEGFTGLYCESQMGQGTRPSPTPVTPRPPRSLTLGIEPVSPTSLRVGLQRYLQGSSVQLRSLRLTYRNLSGPDKRLVTLRLPASLAEYTVTQLRPNATYSVCVMPLGPGRVPEGEEACGEAHTPPAVHSNHAPVTQAREGNLPLLIAPALAAVLLAALAAVGAAYCVRRGRAMAAAAQDKGQVGPGAGPLELEGVKVPLEPGPKATEGGGEALPSGSECEVPLMGFPGPGLQSPLHAKPYI

SEQ ID NO: 60 - DNA, 2019 bp, Homo sapiens
  CDS (1)..(2019)
atgtgctccagggtccctctgctgctgccgctgctcctgctactggccctggggcctggggtgcagggctgcccatccggctgccagtgcagccagccacagacagtcttctgcactgcccgccaggggaccacggtgccccgagacgtgccacccgacacggtggggctgtacgtctttgagaacggcatcaccatgctcgacgcaggcagctttgccggcctgccgggcctgcagctcctggacctgtcacagaaccagatcgccagcctgcccagcggggtcttccagccactcgccaacctcagcaacctggacctgacggccaacaggctgcatgaaatcaccaatgagaccttccgtggcctgcggcgcctcgagcgcctctacctgggcaagaaccgcatccgccacatccagcctggtgccttcgacacgctcgaccgcctcctggagctcaagctgcaggacaacgagctgcgggcactgcccccgctgcgcctgccccgcctgctgctgctggacctcagccacaacagcctcctggccctggagcccggcatcctggacactgccaacgtggaggcgctgcggctggctggtctggggctgcagcagctggacgaggggctcttcagccgcttgcgcaacctccacgacctggatgtgtccgacaaccagctggagcgagtgccacctgtgatccgaggcctccggggcctgacgcgcctgcggctggccggcaacacccgcattgcccagctgcggcccgaggacctggccggcctggctgccctgcaggagctggatgtgagcaacctaagcctgcaggccctgcctggcgacctctcgggcctcttcccccgcctgcggctgctggcagctgcccgcaaccccttcaactgcgtgtgccccctgagctggtttggcccctgggtgcgcgagagccacgtcacactggccagccctgaggagacgcgctgccacttcccgcccaagaacgctggccggctgctcctggagcttgactacgccgactttggctgcccagccaccaccaccacagccacagtgcccaccacgaggcccgtggtgcgggagcccacagccttgtcttctagcttggctcctacctggcttagccccacagcgccggccactgaggcccccagcccgccctccactgccccaccgactgtagggcctgtcccccagccccaggactgcccaccgtccacctgcctcaatgggggcacatgccacctggggacacggcaccacctggcgtgcttgtgccccgaaggcttcacgggcctgtactgtgagagccagatggggcaggggacacggcccagccctacaccagtcacgccgaggccaccacggtccctgaccctgggcatcgagccggtgagccccacctccctgcgcgtggggctgcagcgctacctccaggggagctccgtgcagctcaggagcctccgtctcacctatcgcaacctatcgggccctgataagcggctggtgacgctgcgactgcctgcctcgctcgctgagtacacggtcacccagctgcggcccaacgccacttactccgtctgtgtcatgcctttggggcccgggcgggtgccggagggcgaggaggcctgcggggaggcccatacacccccagccgtccactccaaccacgccccagtcacccaggcccgcgagggcaacctgccgctcctcattgcgcccgccctggccgcggtgctcctggccgcgctggctgcggtgggggcagcctactgtgtgcggcgggggcgggccatggcagcagcggctcaggacaaagggcaggtggggccaggggctgggcccctggaactggagggagtgaaggtccccttggagccaggcccgaaggcaacagagggcggtggagaggccctgcccagcgggtctgagtgtgaggtgccactcatgggcttcccagggcctggcctccagtcacccctccacgcaaagccctacatc

SEQ ID NO: 61 - protein, 560 aa, Homo sapiens
MLRGTLLCAVLGLLRAQPFPCPPACKCVFRDAAQCSGGDVARISALGLPTNLTHILLFGMGRGVLQSQSFSGMTVLQRLMISDSHISAVAPGTFSDLIKLKTLRLSRNKITHLPGALLDKMVLLEQLFLDHNALRGIDQNMFQKLVNLQELALNQNQLDFLPASLFTNLENLKLLDLSGNNLTHLPKGLLGAQAKLERLLLHSNRLVSLDSGLLNSLGALTELQFHRNHIRSIAPGAFDRLPNLSSLTLSRNHLAFLPSALFLHSHNLTLLTLFENPLAELPGVLFGEMGGLQELWLNRTQLRTLPAAAFRNLSRLRYLGVTLSPRLSALPQGAFQGLGELQVLALHSNGLTALPDGLLRGLGKLRQVSLRRNRLRALPRALFRNLSSLESVQLDHNQLETLPGDVFGALPRLTEVLLGHNSWRCDCGLGPFLGWLRQHLGLVGGEEPPRCAGPGAHAGLPLWALPGGDAECPGPRGPPPRPAADSSSEAPVHPALAPNSSEPWVWAQPVTTGKGQDHSPFWGFYFLLLAVQAMITVIIVFAMIKIGQLFRKLIRERALG

SEQ ID NO: 62 - protein, 605 aa, Papio hamadryas
MALRKGGLALALLLLSWVALGPRSLEGAEPGTPGEAEGPACPATCACSYDDEVNELSVFCSSRNLTRLPDGIPGGTQALWLDSNNLSSIPPAAFRNLSSLAFLNLQGGQLGSLEPQALLGLENLCHLHLERNQLRSLAVGTFAYTPALALLGLSNNRLSRLEDGLFEGLGNLWDLNLGWNSLAVLPDAAFRGLGGLRELVLAGNRLAYLQPALFSGLAELRELDLSRNALRAIKANVFAQLPRLQKLYLDRNLIAAVAPGAFLGLKALRWLDLSHNRVAGLLEDTFPGLLGLRVLRLSHNAIASLRPRTFEDLHFLEELQLGHNRIRQLAERSFEGLGQLEVLTLDHNQLQEVKVGAFLGLTNVAVMNLSGNCLRNLPEQVFRGLGKLHSLHLEGSCLGRIRPHTFAGLSGLRRLFLKDNGLVGIEEQSLWGLAELLELDLTSNQLTHLPHQLFQGLGKLEYLLLSHNRLAELPADALGPLQRAFWLDVSHNRLEALPGSLLASLGRLRYLNLRNNSLRTFTPQPPGLERLWLEGNPWDCSCPLKALRDFALQNPSAVPRFVQAICEGDDCQPPVYTYNNITCASPPEVAGLDLRDLGEAHFAPC

SEQ ID NO: 63 - protein, 22 aa, Artificial Sequence
  Xaas at positions 1, 3-4, 6, 8-9, 11, 14, 15 may be any amino acid
XXXXXXXXXXXXXXXXXXXXXX

SEQ ID NO: 64 - protein, 46 aa, Artificial Sequence
  Xaas at positions 2-7, 9-15, 17-28 and 32-45, if present, may be any amino acid
CXXXXXXCXXXXXXXCXXXXXXXXXXXXCXCXXXXXXXXXXXXXXC

SEQ ID NO: 65 - protein, 38 aa, Artificial Sequence
  Xaas at positions 2-5, 7-11, 13-18, 24 and 26-33 may be any amino acid
CXXXXCXXXXXCXXXXXXXXXXCXCXXXXXXXXXXXXC

SEQ ID NO: 66 - protein, 73 aa, Artificial Sequence
  Description of Artificial SequenceConsensus sequence
PSPPRNLRVTDITPTSITVSWTPPEGNGPITGYRIQYRWPVNDNEWNEFNVPRTTNSYTITNLRPGTEYEFRV

SEQ ID NO: 67 - DNA, 2815 bp, Mus musculus
  CDS (197)..(2215)
gtcgacccacgcgtccgggagccggagccagagaccggggctgggaaaccccagcccgggacgggacgcagcagcctctggatcccgggaccccggacctctcaggaccggccagaggtgaaggactgaggccccactgaggccttggaccgcaccgcctggctccttcagccgcagtcgtctcctgggacagaagatgcactccaggagctgcctgccacctctcctgttgttgcttctggtgctcctggggtctggagtacagggttgcccatcaggctgccagtgcaaccagccacagacagtcttctgcactgcccgtcagggaaccacagtgccccgagacgtgccacctgacacagtgggcctgtacatctttgagaacggcatcacgacacttgatgtgggctgttttgctggccttccgggcctgcagcttctggacttgtcacagaaccagatcactagcctgcccgggggcatctttcagccacttgttaacctcagtaacctggacctgactgccaacaaactgcacgagatctccaacgagaccttccgtggcctgcggcgcctggagcgcctctacctgggcaagaaccgaattcgccacatccaaccgggtgccttcgacgcgcttgatcgcctcctggagctcaagctgccagacaatgagcttcgggtgttgcccccattgcacttgccccgcctgctgctgcttgacctcagccacaacagcatcccagccctggaagccggaatactggataccgccaatgtagaggcattgaggttggctggcctagggctgcggcagctggatgaggggctttttggccgccttctcaacctccatgacttggatgtttctgacaaccagttggagcatatgccatctgtgattcaaggcctgcgtggcctgacacgcctgcggctggctggcaacacccgtattgcccagatacggcccgaggacctcgctggtctgactgccctacaggaattggatgtgagcaacctaagcctgcaggccctgcccagtgacctctcgagtctctttccccgcctgcgcctcttagcagctgccaggaaccccttcaactgcttgtgccccttgagctggtttggtccttgggtgcgtgagaaccatgttgtgttggccagccctgaggagacgcgttgtcactttccacccaagaatgctggccgactgctcctggatctggattatgcagattttggctgcccagtcaccactaccacggccacagtacctactataaggtctactatcagggaacccacactttcaacttctagccaagctcccacctggcccagcctcacagagccaactacccaggcctccaccgtactatcgactgccccaccaaccatgaggccagctcctcagccccaggactgtccagcatccatctgcctgaatggtggtagctgccgtttgggagcaagacaccactgggagtgcctatgccctgagggcttcattggcctgtactgtgagagtccagtggagcaagggatgaagcccagctccataccagacactccaaggccccctccactgctgcctctcagcattgagccggtgagccccacctccttgcgtgtgaagctgcagcgctacttgcagggtaacactgtgcagctacggagcctccggctcacctatcgcaacctgtctggccctgacaaacgactggtgacattacggctgcctgcttcacttgcagagtatacagtcacccagctgcgacccaatgccacctattctatctgtgtcacacccttgggagctggacggacacctgaaggtgaggaggcctgtggggaggccaacacttcccaggcagtccgctctaaccatgccccagttacccaggcccgtgagggcaacctgccactcctcattgcgcctgccctggctgctgtacttctggctgtgttagccgctgcaggggcagcctactgtgtgcggcgggcacgggcaacttctacagctcaggacaaagggcaggtggggccagggactggacccctggaactagagggggtgaaagcccctttggagccaggctccaaggcaacagagggaggtggggaggctttgtcaggtggtcctgaatgtgaggtgcctcttatgggctacccagggcccagccttcagggggtcctccctgctaagcactacatttagactggtgagaaagagcagccagggggtcaggctttcagtcaccaccctcctgctgccacagaaggaagttctcagtatacaccacagtgcacgtgcatgatggagctgtgggaccctctctgggctgggtctcatctgtaagctgctacagcccagatgaactctgccagccgccagtgcatccagtacagcgcctgccatcttgtgcaatgtgcaaccctgggatgtgagccctgccatgtgctggtaacatggctaggcatgttgggcttcccaaaccatggagtctggtaaccagtgaaggaagcccccagaaataatgagtggggaaggtactagggcactggccttggcctcaaaagtgcaggcacacttgaaactggaaaggaaggtgctctgggcacatgtggatttgcttctattgttttgttttgttttttctaatgtatttataaaagatcttttcccatttatgctgggaaagtgtttttcaaactcagtgacaaggactttggtttttgtaagactgttgatgatatgaaggccttttgtaagaaaataaaaaataaagtaaaaaaaaaaaaaaagggcggccgc

SEQ ID NO: 68 - protein, 673 aa, Mus musculus
MHSRSCLPPLLLLLLVLLGSGVQGCPSGCQCNQPQTVFCTARQGTTVPRDVPPDTVGLYIFENGITTLDVGCFAGLPGLQLLDLSQNQITSLPGGIFQPLVNLSNLDLTANKLHEISNETFRGLRRLERLYLGKNRIRHIQPGAFDALDRLLELKLPDNELRVLPPLHLPRLLLLDLSHNSIPALEAGILDTANVEALRLAGLGLRQLDEGLFGRLLNLHDLDVSDNQLEHMPSVIQGLRGLTRLRLAGNTRIAQIRPEDLAGLTALQELDVSNLSLQALPSDLSSLFPRLRLLAAARNPFNCLCPLSWFGPWVRENHVVLASPEETRCHFPPKNAGRLLLDLDYADFGCPVTTTTATVPTIRSTIREPTLSTSSQAPTWPSLTEPTTQASTVLSTAPPTMRPAPQPQDCPASICLNGGSCRLGARHHWECLCPEGFIGLYCESPVEQGMKPSSIPDTPRPPPLLPLSIEPVSPTSLRVKLQRYLQGNTVQLRSLRLTYRNLSGPDKRLVTLRLPASLAEYTVTQLRPNATYSICVTPLGAGRTPEGEEACGEANTSQAVRSNHAPVTQAREGNLPLLIAPALAAVLLAVLAAAGAAYCVRRARATSTAQDKGQVGPGTGPLELEGVKAPLEPGSKATEGGGEALSGGPECEVPLMGYPGPSLQGVLPAKHYI

SEQ ID NO: 69 - DNA, 2022 bp, Mus musculus
  CDS (1)..(2019)
atgcactccaggagctgcctgccacctctcctgttgttgcttctggtgctcctggggtctggagtacagggttgcccatcaggctgccagtgcaaccagccacagacagtcttctgcactgcccgtcagggaaccacagtgccccgagacgtgccacctgacacagtgggcctgtacatctttgagaacggcatcacgacacttgatgtgggctgttttgctggccttccgggcctgcagcttctggacttgtcacagaaccagatcactagcctgcccgggggcatctttcagccacttgttaacctcagtaacctggacctgactgccaacaaactgcacgagatctccaacgagaccttccgtggcctgcggcgcctggagcgcctctacctgggcaagaaccgaattcgccacatccaaccgggtgccttcgacgcgcttgatcgcctcctggagctcaagctgccagacaatgagcttcgggtgttgcccccattgcacttgccccgcctgctgctgcttgacctcagccacaacagcatcccagccctggaagccggaatactggataccgccaatgtagaggcattgaggttggctggcctagggctgcggcagctggatgaggggctttttggccgccttctcaacctccatgacttggatgtttctgacaaccagttggagcatatgccatctgtgattcaaggcctgcgtggcctgacacgcctgcggctggctggcaacacccgtattgcccagatacggcccgaggacctcgctggtctgactgccctacaggaattggatgtgagcaacctaagcctgcaggccctgcccagtgacctctcgagtctctttccccgcctgcgcctcttagcagctgccaggaaccccttcaactgcttgtgccccttgagctggtttggtccttgggtgcgtgagaaccatgttgtgttggccagccctgaggagacgcgttgtcactttccacccaagaatgctggccgactgctcctggatctggattatgcagattttggctgcccagtcaccactaccacggccacagtacctactataaggtctactatcagggaacccacactttcaacttctagccaagctcccacctggcccagcctcacagagccaactacccaggcctccaccgtactatcgactgccccaccaaccatgaggccagctcctcagccccaggactgtccagcatccatctgcctgaatggtggtagctgccgtttgggagcaagacaccactgggagtgcctatgccctgagggcttcattggcctgtactgtgagagtccagtggagcaagggatgaagcccagctccataccagacactccaaggccccctccactgctgcctctcagcattgagccggtgagccccacctccttgcgtgtgaagctgcagcgctacttgcagggtaacactgtgcagctacggagcctccggctcacctatcgcaacctgtctggccctgacaaacgactggtgacattacggctgcctgcttcacttgcagagtatacagtcacccagctgcgacccaatgccacctattctatctgtgtcacacccttgggagctggacggacacctgaaggtgaggaggcctgtggggaggccaacacttcccaggcagtccgctctaaccatgccccagttacccaggcccgtgagggcaacctgccactcctcattgcgcctgccctggctgctgtacttctggctgtgttagccgctgcaggggcagcctactgtgtgcggcgggcacgggcaacttctacagctcaggacaaagggcaggtggggccagggactggacccctggaactagagggggtgaaagcccctttggagccaggctccaaggcaacagagggaggtggggaggctttgtcaggtggtcctgaatgtgaggtgcctcttatgggctacccagggcccagccttcagggggtcctccctgctaagcactacatttag

SEQ ID NO: 70 - DNA, 2915 bp, Homo sapiens
  CDS (404)..(1294)
gaattcccgggtcgacccacgcgtccgcccacgcgtccgcccacgcgtccggctgagcgaagcgcggcggcggcggcggcgcctaggggagggaggggcggcggggccgagcccacctagcggagcgcgccggccgccggtggccgccgccagcatgccccggcccgcgggccgctccgccgccagccacccccgcggccctcggcggcctgcgctcggcccgggggcgcgggaaccgcagccggagccggaggcgggagcagcgagccggagccccgggcgctcgaatgcaggatgctcgtggtccccagcatccttgagccaccaggagtgagggctgctgctccctgagacctggctccaaggaggatgccacagccgcctgccagctccggtctgcaccatgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggactctacgcccatccccgaaaggtctgcagtgagacagggagaggactggggcaaagaccagcctgaggggtttcatccaagcagcaggcaagactgccttccctgagccattgcaggacatgaggacatgagctccagaatggtgccaggccgagccctgtgcccacaggtggtgagcttcagcagcctgcgggccgacgcctcagcgccctggatggcactctgcgtgctgtggtgctccgtggcccaggccctgcttccgacctgtcctgcctcagtttccccatctgtgatgagcaggtgaccacgttaactctcagggctgtttgaagtctcttggttgtaggccctcgccactgagtggcccaggtgtgagaggtagtctagagccctctgcggcctttgtggaggtccgttctcagcatgtggcctggtgtggctccctaggcctgaggcctcccactctcagtgccccctgcccccttgggaacccacactccaccccagctaagcacaacactgtggaccaccaccaatggcacctgagccaccttgaccaccattagtgtccccaccctcattactctgccgtcgttgcctgtccccaccacatcatctccctccatggctcccaccatgacgctggcaggtgcaagcggagagcacgagggcagcagggcctggccttccgtgtccctgcgtcatccccagcctcactccccacccgtgcaggcctggccaggaagggatcctggcaggggcttccaggttctcagctcaaggcctggtcccgggcaggcgtccaacccctgggagcaatgtatttctttgccttccatcctgggcagaccccttacaggcctgggcattgccatgggccctgggtcttcccaggctaaggagaaccaggaacagctataaccttgagctagtgaaatagggtagatgagaaggctgtctcctccagacccctacccctacacagtggccccacaatatgaagacctggggtaattccaaggtgagcatagagcctgcctgtgcccagttccttctggccctcaggtggccaagcccatctcttcatccttcagatagggtcccactcccagaagaagctgctggggtgggggtgggaggctgcctaagcctgtctgtgcttcagaggcccctccagtccctggctgtggggtaactgggggtatgagctgtggccacaggtgagcaaggcagggaactgcaatccagccctggccgcgggaggggccatctctggccaatgctgctgtgccttcaaggactgacaagttacgtaggggcagaggtcgccagctagccagtgtctcctccatctggggggcgtctgtccacttgtcaccttaggttttcactcatttgtcaccttggggttttgctctgtgtgtttcatatccaacggcaatacttgcagggggacagagtcctctaaatactccaatcctgcggtttttacaaacataaagggggagaccccaagtggaggaccctgggcctggagctccctcccaaactttgtccagcatccagcctgttccctgggctcactggggagggagttgtcttcatagcacacccagagccagggatccctttgtagttttttgacaacggagcatttctcttctgtacaggacccaataaaaacttccttatgatttgcaaaaaaaaaaaaaaagggcggccgc

SEQ ID NO: 71 - protein, 297 aa, Homo sapiens
MSDERRLPGSAVGWLVCGGLSLLANAWGILSVGAKQKKWKPLEFLLCTLAATHMLNVAVPIATYSVVQLRRQRPDFEWNEGLCKVFVSTFYTLTLATCFSVTSLSYHRMWMVCWPVNYRLSNAKKQAVHTVMGIWMVSFILSALPAVGWHDTSERFYTHGCRFIVAEIGLGFGVCFLLLVGGSVAMGVICTAIALFQTLAVQVGRQADHRAFTVPTIVVEDAQGKRRSSIDGSEPAKTSLQTTGLVTTIVFIYDCLMGFPVLDSTPIPERSAVRQGEDWGKDQPEGFHPSSRQDCLP

SEQ ID NO: 72 - DNA, 891 bp, Homo sapiens
  CDS (1)..(891)
atgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggactctacgcccatccccgaaaggtctgcagtgagacagggagaggactggggcaaagaccagcctgaggggtttcatccaagcagcaggcaagactgccttccc

SEQ ID NO: 73 - DNA, 4166 bp, Homo sapiens
  CDS (334)..(2160)
gaattcccgggtcgacccacgcgtccggcggcggggccgagcccacctagcggagcgcgccggccgccggtggccgccgccagcatgccccggcccgcgggccgctccgccgccagccacccccgcggccctcggcggcctgcgctcggcccgggggcgcgggaaccgcagccggagccggaggcgggagcagcgagccggagccccgggcgctcgaatgcaggatgctcgtggtccccagcatccttgagccaccaggagtgagggctgctgctccctgagacctggctccaaggaggatgccacagccgcctgccagctccggtctgcaccatgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggtggtgagcttcagcagcctgcgggccgacgcctcagcgccctggatggcactctgcgtgctgtggtgctccgtggcccaggccctgctgctgcctgtgttcctctgggcctgcgaccgctaccgggctgacctcaaagctgtccgggagaagtgcatggccctcatggccaacgacgaggagtcagacgatgagaccagcctggaaggtggcatctccccggacctggtgttggagcgctccctggactatggctatggaggtgattttgtggccctagataggatggccaagtatgagatctccgccctggaggggggcctgccccagctctacccactgcggcccttgcaggaggacaagatgcaatacctgcaggtcccgcccacgcggcgcttctcccacgacgatgcggacgtgtgggccgccgtcccgctgcccgccttcctgccgcgctggggctccggcgaggacctggccgccctggcgcacctggtgctgcctgccgggcccgagcggcgccgcgccagcctcctggccttcgcggaggacgcaccaccgtcccgcgcgcgccgccgctcggccgagagcctgctgtcgctgcggacctcggccctggatagcggcccgcggggagcccgcgactcgccccccggcagcccgcgccgccgccccgggcccggcccccgctccgcctcggcctcgctgctgcccgacgccttcgccctgaccgccttcgagtgcgagccacaggccctgcgccgcccgcccgggcccttccccgctgcgcccgccgcccccgacggcgcagatcccggagaggccccgacgcccccaagcagcgcccagcggagcccagggccacgcccctctgcgcactcgcacgccggctctctgcgccccggcctgagcgcgtcgtggggcgagcccggggggctgcgcgcggcgggcggcggcggcagcaccagcagcttcctgagttccccctccgagtcctcgggctacgccacgctgcactcggactcgctgggctccgcgtcctaggaccgccggcgcctccccacggacgccaggcaggccaggccgctctccggggccgcagcaccaaagacgcccgcctcccccccgcgcgcagacatgcgccacccctcccaggggtgagggggcgttggcctcagcgtttgtcttccggctcctcccagctggccttgtcccaggggcgacggctgccccggacgactgcgctgggcaccgcatgtcccgggccgagtgaggtcgggcctggggagctgagtgacatcccaagcttgggcttgggtagtgagtgacatgtgcacacgtccagctgcgccatcaccagccctgcgcaacaggacgtcgggagcagggaacctgagacagggccactgcgggatcggacaaagccccgctttggagaggctgagctggagccattggcctccccaggggctttccacccacactgcaccataaccgccacacccttcggggggggggagggtacagagggtctctaagcacaggggtgttcagagcccgaacaagctttgatcaggtttccctgcttccgacctgtcctgcctcagtttccccatctgtgatgagcaggtgaccacgttaactctcagggctgtttgaagtctcttggttgtaggccctcgccactgagtggcccaggtgtgagaggtagtctagagccctctgcggcctttgtggaggtccgttctcagcatgtggcctggtgtggctccctaggcctgaggcctcccactctcagtgccccctgcccccttgggaacccacactccaccccagctaagcacaacactgtggaccaccaccaatggcacctgagccaccttgaccaccattagtgtccccaccctcattactctgccgtcgttgcctgtccccaccacatcatctccctccatggctcccaccatgacgctggcaggtgcaagcggagagcacgagggcagcagggcctggccttccgtgtccctgcgtcatccccagcctcactccccacccgtgcaggcctggccaggaagggatcctggcaggggcttccaggttctcagctcaaggcctggtcccgggcaggcgtccaacccctgggagcaatgtatttctttgccttccatcctgggcagaccccttacaggcctgggcattgccatgggccctgggtcttcccaggctaaggagaaccaggaacagctataaccttgagctagtgaaatagggtagatgagaaggctgtctcctccagacccctacccctacacagtggccccacaatatgaagacctggggtaattccaaggtgagcatagagcctgcctgtgcccagttccttctggccctcaggtggccaagcccatctcttcatccttcagatagggtcccactcccagaagaagctgctggggtgggggtgggaggctgcctaagcctgtctgtgcttcagaggcccctccagtccctggctgtggggtaactgggggtatgagctgtggccacaggtgagcaaggcagggaactgcaatccagccctggccgcgggaggggccatctctggccaatgctgctgtgccttcaaggactgacaagttacgtaggggcagaggtcgccagctagccagtgtctcctccatctggggggcgtctgtccacttgtcaccttaggttttcactcatttgtcaccttggggttttgctctgtgtgtttcatatccaacggcaatacttgcagggggacagagtcctctaaatactccaatcctgcggtttttacaaacataaagggggagaccccaagtggaggaccctgggcctggagctccctcccaaactttgtccagcatccagcctgttccctgggctcactggggagggagttgtcttcatagcacacccagagccagggatccctttgtagttttttgacaacggagcatttctcttctgtacaggacccaataaaaacttccttatgaaaaaaaaaaaaaaagggcggccgc

SEQ ID NO: 74 - protein, 609 aa, Homo sapiens
MSDERRLPGSAVGWLVCGGLSLLANAWGILSVGAKQKKWKPLEFLLCTLAATHMLNVAVPIATYSVVQLRRQRPDFEWNEGLCKVFVSTFYTLTLATCFSVTSLSYHRMWMVCWPVNYRLSNAKKQAVHTVMGIWMVSFILSALPAVGWHDTSERFYTHGCRFIVAEIGLGFGVCFLLLVGGSVAMGVICTAIALFQTLAVQVGRQADHRAFTVPTIVVEDAQGKRRSSIDGSEPAKTSLQTTGLVTTIVFIYDCLMGFPVLVVSFSSLRADASAPWMALCVLWCSVAQALLLPVFLWACDRYRADLKAVREKCMALMANDEESDDETSLEGGISPDLVLERSLDYGYGGDFVALDRMAKYEISALEGGLPQLYPLRPLQEDKMQYLQVPPTRRFSHDDADVWAAVPLPAFLPRWGSGEDLAALAHLVLPAGPERRRASLLAFAEDAPPSRARRRSAESLLSLRTSALDSGPRGARDSPPGSPRRRPGPGPRSASASLLPDAFALTAFECEPQALRRPPGPFPAAPAAPDGADPGEAPTPPSSAQRSPGPRPSAHSHAGSLRPGLSASWGEPGGLRAAGGGGSTSSFLSSPSESSGYATLHSDSLGSAS

SEQ ID NO: 75 - DNA, 1827 bp, Homo sapiens
  CDS (1)..(1827)
atgagtgatgagcggcggctgcctggcagtgcagtgggctggctggtatgtgggggcctctccctgctggccaatgcctggggcatcctcagcgttggcgccaagcagaagaagtggaagcccttggagttcctgctgtgtacgctcgcggccacccacatgctaaatgtggccgtgcccatcgccacctactccgtggtgcagctgcggcggcagcgccccgacttcgagtggaatgagggtctctgcaaggtcttcgtgtccaccttctacaccctcaccctggccacctgtttctctgtcacctccctctcctaccaccgcatgtggatggtctgctggcctgtcaactaccggctgagcaatgccaagaagcaggcggtgcacacagtcatgggtatctggatggtgtccttcatcctgtcggccctgcctgccgttggctggcacgacaccagcgagcgcttctacacccatggctgccgcttcatcgtggctgagatcggcctgggctttggcgtctgcttcctgctgctggtgggcggcagcgtggccatgggcgtgatctgcacagccatcgccctcttccagacgctggccgtgcaggtggggcgccaggccgaccaccgcgccttcaccgtgcccaccatcgtggtggaggacgcgcagggcaagcggcgctcctccatcgatggctcggagcccgccaaaacctctctgcagaccacgggcctcgtgaccaccatagtcttcatctacgactgcctcatgggcttccctgtgctggtggtgagcttcagcagcctgcgggccgacgcctcagcgccctggatggcactctgcgtgctgtggtgctccgtggcccaggccctgctgctgcctgtgttcctctgggcctgcgaccgctaccgggctgacctcaaagctgtccgggagaagtgcatggccctcatggccaacgacgaggagtcagacgatgagaccagcctggaaggtggcatctccccggacctggtgttggagcgctccctggactatggctatggaggtgattttgtggccctagataggatggccaagtatgagatctccgccctggaggggggcctgccccagctctacccactgcggcccttgcaggaggacaagatgcaatacctgcaggtcccgcccacgcggcgcttctcccacgacgatgcggacgtgtgggccgccgtcccgctgcccgccttcctgccgcgctggggctccggcgaggacctggccgccctggcgcacctggtgctgcctgccgggcccgagcggcgccgcgccagcctcctggccttcgcggaggacgcaccaccgtcccgcgcgcgccgccgctcggccgagagcctgctgtcgctgcggacctcggccctggatagcggcccgcggggagcccgcgactcgccccccggcagcccgcgccgccgccccgggcccggcccccgctccgcctcggcctcgctgctgcccgacgccttcgccctgaccgccttcgagtgcgagccacaggccctgcgccgcccgcccgggcccttccccgctgcgcccgccgcccccgacggcgcagatcccggagaggccccgacgcccccaagcagcgcccagcggagcccagggccacgcccctctgcgcactcgcacgccggctctctgcgccccggcctgagcgcgtcgtggggcgagcccggggggctgcgcgcggcgggcggcggcggcagcaccagcagcttcctgagttccccctccgagtcctcgggctacgccacgctgcactcggactcgctgggctccgcgtcc

SEQ ID NO: 76 - protein, 177 aa, Homo sapiens
  Xaas at positions 38, 55 and 56 may be any amino acid
GNILVIWVICRYRRMRTPMNYFIVNLAVADLLFSLFTXMPFWMVYYVMQGRWPFXXGDFMCRIWMYFDYMNMYASIFFLTCISIDRYLWAICHPMRYMRWMTPRHRAWVMIIIIWVMSFLISMPPFLMFRWSTYRDENEWNMTWCMIYDWPEWMWRWYVILMTIIMGFYIPMIIMLF

SEQ ID NO: 77 - protein, 107 aa, Homo sapiens
  Xaa at position 84 may be any amino acid
IQERMNELNDRWERLKELMEQRRQMLEDSMRLQQFFRDMDEEESWINEKEQILNSDDYGKDLTSVQNLLKKHQAFEADIAAHEXDRIQALNEFAQQLIQENHYASEE

SEQ ID NO: 78 - protein, 588 aa, Homo sapiens
MARGGAGAEEASLRSNALSWLACGLLALLANAWIILSISAKQQKHKPLELLLCFLAGTHILMAAVPLTTFAVVQLRRQASSDYDWNESICKVFVSTYYTLALATCFTVASLSYHRMWMVRWPVNYRLSNAKKQALHAVMGIWMVSFILSTLPSIGWHNNGERYYARGCQFIVSKIGLGFGVCFSLLLLGGIVMGLVCVAITFYQTLWARPRRARQARRVGGGGGTKAGGPGALGTRPAFEVPAIVVEDARGKRRSSLDGSESAKTSLQVTNLVSAIVFLYDSLTGVPILVVSFFSLKSDSAPPWMVLAVLWCSMAQTLLLPSFIWSCERYRADVRTVWEQCVAIMSEEDGDDDGGCDDYAEGRVCKVRFDANGATGPGSRDPAQVKLLPGRHMLFPPLERVHYLQVPLSRRLSHDETNIFSTPREPGSFLHKWSSSDDIRVLPAQSRALGGPPEYLGQRHRLEDEEDEEEAEGGGLASLRQFLESGVLGSGGGPPRGPGFFREEITTFIDETPLPSPTASPGHSPRRPRPLGLSPRRLSLGSPESRAVGLPLGLSAGRRCSLTGGEESARAWGGSWGPGNPIFPQLTL

SEQ ID NO: 79 - protein, 227 aa, Homo sapiens
ITFYQTLWARPRRARQARRVGGGGGTKAGGPGALGTRPAFEVPAIVVEDARGKRRSSLDGSESAKTSLQVTNLVVSFFSLKSDSAPPWMVLAVLWCSMAQTLLLPSFIWSCERYRADVRTVWEQCVAIMSEEDGDDDGGCDDYAEGRVCKVRFDANGATGPGSRDPAQVKLLPGRHMLFPPLERVHYLQLKKLDLAAAAAHTFFVANPMHLQMREDMAKYRRMSGVR

SEQ ID NO: 80 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLVLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISSCIDPLIYYYASSECQRYVYSILCCKESSDPSSYNSSGQLMASKMDTCSSNLNNSIYKKLLT

SEQ ID NO: 81 - protein, 348 aa, Homo sapiens [sequence omitted]

SEQ ID NO: 82 - protein, 460 aa, Rattus Norvegicus
MNTSVPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPESPPGRCCRCCRAPRLLQAYSWKEEEEEDEGSMESLTSSEGEEPGSEVVIKMPMVDSEAQAPTKQPPKSSPNTVKRPTKKGRDRGGKGQKPRGKEQLAKRKTFSLVKEKKAARTLSAILLAFILTWTPYNIMVLVSTFCKDCVPETLWELGYWLCYVNSTVNPMCYALCNKAFRDTFRLLLLCRWDKRRWRKIPKRPGSVHRTPSRQC

SEQ ID NO: 83 - protein, 350 aa, Homo sapiens [sequence omitted]

SEQ ID NO: 84 - protein, 601 aa, Drosophila melanogaster
MPSADQILFVNVTTTVAAAALTAAAAVSTTKSGNGNAARGYTDSDDDAGMGTEAVANISGSLVEGLTTVTAALSTAQADKDSAGECEGAVEELHASILGLQLAVPEWEALLTALVLSVIIVLTIIGNILVILSVFTYKPLRIVQNFFIVSLAVADLTVALLVLPFNVAYSILGRWEFGIHLCKLWLTCDVLCCTSSILNLCAIALDRYWAITDPINYAQKRTVGRVLLLISGVWLLSLLISSPPLIGWNDWPDEFTSATPCELTSQRGYVIYSSLGSFFIPLAIMTIVYIEIFVATRRRLRERARANKLNTIALKSTELEPMANSSPVAASNSGSKSRLLASWLCCGRDRAQFATPMIQNDQESISSETHQPQDSSKAGPHGNSDPQQQHVVVLVKKSRRAKTKDSIKHGKTRGGRKSQSSSTCEPHGEQQLLPAGGDGGSCQPGGGHSGGGKSDAEISTESGSDPKGCIQVCVTQADEQTSLKLTPPQSSTGVAAVSVTPLQKKTSGVNQFIEEKQKISLSKERRAARTLGIIMGVFVICWLPFFLMYVILPFCQTCCPTNKFKNFITWLGYINSGLNPVIYTIFNLDYRRAFKRLLGLN

SEQ ID NO: 85 - DNA, 19 bp, Artificial Sequence
  Description of Artificial Sequence Primer
agatgccaccttccaggct

SEQ ID NO: 86 - DNA, 19 bp, Artificial Sequence
  Description of Artificial Sequence Primer
ggagaagtgcatggccctc

SEQ ID NO: 87 - DNA, 27 bp, Artificial Sequence
  Description of Artificial Sequence Probe
tctcatcgtctgactcctcgtcgttgg

SEQ ID NO: 88 - DNA, 18 bp, Artificial Sequence
  Description of Artificial Sequence Primer
ggcggtgcacacagttat

SEQ ID NO: 89 - DNA, 21 bp, Artificial Sequence
  Description of Artificial Sequence Primer
agagagcgctccaaataccat

SEQ ID NO: 90 - protein, 4 aa, Homo sapiens
KRRS

SEQ ID NO: 91 - protein, 4 aa, Homo sapiens
SSID

SEQ ID NO: 92 - protein, 4 aa, Homo sapiens
SRQD

SEQ ID NO: 93 - protein, 6 aa, Homo sapiens
GSAVGW

SEQ ID NO: 94 - protein, 6 aa, Homo sapiens
GLGFGV

SEQ ID NO: 95 - protein, 6 aa, Homo sapiens
GGSVAM

SEQ ID NO: 96 - protein, 6 aa, Homo sapiens
GVICTA

SEQ ID NO: 97 - protein, 6 aa, Homo sapiens
GSEPAK

SEQ ID NO: 98 - protein, 6 aa, Homo sapiens
GLVTTI

SEQ ID NO: 99 - protein, 4 aa, Homo sapiens
QGKR

SEQ ID NO: 100 - protein, 4 aa, Homo sapiens
KRRS

SEQ ID NO: 101 - protein, 4 aa, Homo sapiens
RRFS

SEQ ID NO: 102 - protein, 4 aa, Homo sapiens
RRAS

SEQ ID NO: 103 - protein, 4 aa, Homo sapiens
RRRS

SEQ ID NO: 104 - protein, 4 aa, Homo sapiens
SSID

SEQ ID NO: 105 - protein, 4 aa, Homo sapiens
SSDE

SEQ ID NO: 106 - protein, 4 aa, Homo sapiens
TSLE

SEQ ID NO: 107 - protein, 4 aa, Homo sapiens
SALE

SEQ ID NO: 108 - protein, 4 aa, Homo sapiens
SHDD

SEQ ID NO: 109 - protein, 4 aa, Homo sapiens
SGED

SEQ ID NO: 110 - protein, 4 aa, Homo sapiens
SALD

SEQ ID NO: 111 - protein, 4 aa, Homo sapiens
TAFE

SEQ ID NO: 112 - protein, 4 aa, Homo sapiens
SWGE

SEQ ID NO: 113 - protein, 4 aa, Homo sapiens
SPSE

SEQ ID NO: 114 - protein, 7 aa, Homo sapiens
RSLDYGY

SEQ ID NO: 115 - protein, 6 aa, Homo sapiens
GSAVGW

SEQ ID NO: 116 - protein, 6 aa, Homo sapiens
GLGFGV

SEQ ID NO: 117 - protein, 6 aa, Homo sapiens
GGSVAM

SEQ ID NO: 118 - protein, 6 aa, Homo sapiens
GVICTA

SEQ ID NO: 119 - protein, 6 aa, Homo sapiens
GSEPAK

SEQ ID NO: 120 - protein, 6 aa, Homo sapiens
GLVTTI

SEQ ID NO: 121 - protein, 6 aa, Homo sapiens
GADPGE

SEQ ID NO: 122 - protein, 6 aa, Homo sapiens
GLSASW

SEQ ID NO: 123 - protein, 6 aa, Homo sapiens
GGLRAA

SEQ ID NO: 124 - protein, 6 aa, Homo sapiens
GGGGST

SEQ ID NO: 125 - protein, 4 aa, Homo sapiens
QGKR

SEQ ID NO: 126 - protein, 458 aa, Mus Musculus
MTKEMTENQRLCPHEREDADCSSESVKFDARSMTASLPHSTKNGPSVQEKLKSFKAALIALYLLVFAVLIPVVGIVTAQLLNWEMKNCLVCSRNTSDTSQGPMEKENTSNVEMRFTIIMAHMKDMEERIQSISNSKADLIDTGRFQNFSMATDQRLNDILLQLNSLILSVQEHGNSLDAISKSLQSLNMTLLDVQLHTETLHVRVRESTAKQQEDISKLEERVYKVSAEVQSVKEEQAHVEQEVKQEVRVLNNITNDLRLKDWEHSQTLKNITFIQGPPGPQGEKGDRGLTGQTGPPGAPGIRGIPGVKGDRGQIGFPGGRGNPGAPGKPGRSGSPGPKGQKGEKGSVGGSTPLKTVRLVGGSGAHEGRVEIFHQGQWGTICDDRWDIRAGQVVCRSLGYQEVLAVHKRAHFGQGTGPIWLNEVMCFGRESSIENCKINQWGVLSCSHSEDAGVTCTS

SEQ ID NO: 127 - protein, 4 aa, Artificial Sequence
  Xaa at position 2 and 4 may be any amino acid except Pro
NXXX

SEQ ID NO: 128 - protein, 11 aa, Artificial Sequence
  Description of Artificial Sequence Consensus sequence
CXCXCXCXCXC

SEQ ID NO: 129 - protein, 22 aa, Artificial Sequence
  Description of Artificial Sequence Consensus sequence
CXFYHXCXCXCXDNEQSKPXXC

SEQ ID NO: 130 - protein, 39 aa, Mus musculus
CRQQEFKDRSGNCVLCKQCGPGMELSKECGFGYGEDAQC

SEQ ID NO: 131 - protein, 40 aa, Mus musculus
CRPHRFKEDWGFQKCKPCADCALVNRFQRANCSHTSDAVC

SEQ ID NO: 132 - protein, 39 aa, Mus musculus; identical to SEQ ID NO: 130

SEQ ID NO: 133 - protein, 40 aa, Mus musculus; identical to SEQ ID NO: 131

SEQ ID NO: 134 - protein, 29 aa, Mus musculus
MALKVLPLHRTVLFAAILFLLHLACKVSC

SEQ ID NO: 135 - protein, 29 aa, Mus musculus
MALKVLPLHRTVLFAAILFLLHLACKVSC

SEQ ID NO: 136 - protein, 25 aa, Mus musculus
ALAAVICSALATVLLALLILCVIYC

SEQ ID NO: 137 - protein, 31 aa, Homo sapiens
TLFVPSVYTGVFVVSLPLNIMAIVVFILKMK

SEQ ID NO: 138 - protein, 31 aa, Homo sapiens
FSMLAAYMFLLIVLGFPINFLTLYVTVQHKK

SEQ ID NO: 139 - protein, 31 aa, Rattus Norvegicus
VAFIGITTGLLSLATVTGNLLVLISFKVNTE

SEQ ID NO: 140 - protein, 31 aa, Homo sapiens
KYVVIIAYALVFLLSLLGNSLVMLVILYSRV

SEQ ID NO: 141 - protein, 31 aa, Drosophila melanogaster
ALLTALVLSVIIVLTIIGNILVILSVFTYKP

SEQ ID NO: 142 - protein, 28 aa, Homo sapiens
VVYMLHLATADVLFVSVLPFKISYYFSG

SEQ ID NO: 143 - protein, 28 aa, Homo sapiens
NYILLNLAVADLFMVLGGFTSTLYTSLH

SEQ ID NO: 144 - protein, 28 aa, Rattus Norvegicus
NYFLLSLACADLIIGTFSMNLYTTYLLM

SEQ ID NO: 145 - protein, 28 aa, Homo sapiens
DVYLLNLALADLLFALTLPIWAASKVNG

SEQ ID NO: 146 - protein, 28 aa, Drosophila melanogaster
NFFIVSLAVADLTVALLVLPFNVAYSIL

SEQ ID NO: 147 - protein, 25 aa, Homo sapiens
RFVTAAFYCNMYASILLMTVISIDR

SEQ ID NO: 148 - protein, 25 aa, Homo sapiens
NLEGFFATLGGEIALWSLVVLAIER

SEQ ID NO: 149 - protein, 25 aa, Rattus Norvegicus
DLWLALDYVASNASVMNLLLISFDR

SEQ ID NO: 150 - protein, 25 aa, Homo sapiens
KVVSLLKEVNFYSGILLLACISVDR

SEQ ID NO: 151 - protein, 25 aa, Drosophila melanogaster
KLWLTCDVLCCTSSILNLCAIALDR

SEQ ID NO: 152 - protein, 27 aa, Homo sapiens
TLGRASFTCLAIWALAIAGVVPLVLKE

SEQ ID NO: 153 - protein, 27 aa, Homo sapiens
GENHAIMGVAFTWVMALACAAPPLAGW

SEQ ID NO: 154 - protein, 27 aa, Rattus Norvegicus
TPRRAALMIGLAWLVSFVLWAPAILFW

SEQ ID NO: 155 - protein, 27 aa, Homo sapiens
KRHLVKFVCLGCWGLSMNLSLPFFLFR

SEQ ID NO: 156 - protein, 27 aa, Drosophila melanogaster
TVGRVLLLISGVWLLSLLISSPPLIGW

SEQ ID NO: 157 - protein, 29 aa, Homo sapiens
AYYFSAFSAVFFFVPLIISTVCYVSIIRC

SEQ ID NO: 158 - protein, 29 aa, Homo sapiens
ESFVIYMFVVHFTIPMIIIFFCYGQLVFT

SEQ ID NO: 159 - protein, 29 aa, Rattus Norvegicus
PIITFGTAMAAFYLPVTVMCTLYWRIYRE

SEQ ID NO: 160 - protein, 29 aa, Homo sapiens
MVLRILPHTFGFIVPLFVMLFCYGFTLRT

SEQ ID NO: 161 - protein, 29 aa, Drosophila melanogaster
RGYVIYSSLGSFFIPLAIMTIVYIEIFVA

SEQ ID NO: 162 - protein, 28 aa, Homo sapiens
FLSAAVFCIFIICFGPTNVLLIAHYSFL

SEQ ID NO: 163 - protein, 28 aa, Homo sapiens
RMVIIMVIAFLICWVPYASVAFYIFTHQ

SEQ ID NO: 164 - protein, 28 aa, Rattus Norvegicus
RTLSAILLAFILTWTPYNIMVLVSTFCK

SEQ ID NO: 165 - protein, 28 aa, Homo sapiens
RVIFAVVLIFLLCWLPYNLVLLADTLMR

SEQ ID NO: 166 - protein, 28 aa, Drosophila melanogaster
RTLGIIMGVFVICWLPFFLMYVILPFCQ

SEQ ID NO: 167 - protein, 33 aa, Homo sapiens
EAAYFAYLLCVCVSSISSCIDPLIYYYASSECQ

SEQ ID NO: 168 - protein, 33 aa, Homo sapiens
NFGPIFMTIPAFFAKSAAIYNPVIYIMMNKQFR

SEQ ID NO: 169 - protein, 33 aa, Rattus Norvegicus
CVPETLWELGYWLCYVNSTVNPMCYALCNKAFR

SEQ ID NO: 170 - protein, 33 aa, Homo sapiens
NNIGRALDATEILGFLHSCLNPIIYAFIGQNFR

SEQ ID NO: 171 - protein, 33 aa, Drosophila melanogaster
CPTNKFKNFITWLGYINSGLNPVIYTIFNLDYR

SEQ ID NO: 172 - protein, 174 aa, Artificial Sequence
  Description of Artificial Sequence 7 transmembrane receptor consensus sequence
GNILVIWVICRYRRMRTPMNYFIVNLAVADLLFSLFTMPFWMVYYVMQGRWPFGDFMCRIWMYFDYMNMYASIFFLTCISIDRYLWAICHPMRYMRWMTPRHRAWVMIIIIWVMSFLISMPPFLMFRWSTYRDENEWNMTWCMIYDWPEWMWRWYVILMTIIMGFYIPMIIMLF

SEQ ID NO: 173 - protein, 168 aa, Homo sapiens
ANAWGILSVGAKQKKWKPLEFLLCTLAATHMLNVAVPIATYSVVQLRRQRPDFEWNEGLCKVFVSTFYTLTLATCFSVTSLSYHRMWMVCWPVNYRLSNAKKQAVHTVMGIWMVSFILSALPAVGWHDTSERFYTHGCRFIVAEIGLGFGVCFLLLVGGSVAMGVICT

SEQ ID NO: 174 - protein, 106 aa, Artificial Sequence
  Description of Artificial Sequence Consensus sequence
IQERMNELNDRWERLKELMEQRRQMLEDSMRLQQFFRDMDEEESWINEKEQILNSDDYGKDLTSVQNLLKKHQAFEADIAAHEDRIQALNEFAQQLIQENHYASEE

SEQ ID NO: 175 - protein, 107 aa, Homo sapiens
FSSLRADASAPWMALCVLWCSVAQALLLPVFLWACDRYRADLKAVREKCMALMANDEESDDETSLEGGISPDLVLERSLDYGYGGDFVALDRMAKYEISALEGGLPQ

SEQ ID NO: 176 - protein, 6 aa, Artificial Sequence
  Xaa at position 2 may be any amino acide except Glu, Asp, Arg, Lys, His, Phe, Pro, Tyr, or Trp
GXXXXX

(19) United States
(12) Patent Application Publication
(10) Pub. No.: US 2011/0020798 A1
(43) Pub. Date: Jan. 27, 2011

(54) Novel italy, LOR-2, strife, trash, BDSF, LRSG, and STMST protein and nucleic acid molecules and uses therefor

(75) Inventors: Douglas A. Holtzman, Jamaica Plain, MA (US); Sean A. McCarthy, San Diego, CA (US); Kyle J. MacBeth, Boston, MA (US); Samantha J. Busfield, Maddington (AU); Yang Pan, Bellevue, WA (US); David White, Braintree, MA (US); Mehran M. Khodadoust, Brookline, MA (US); Wei Gu, Brookline, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 12/653,955

(22) Filed: Dec. 18, 2009

Related U.S. Application Data
(63) Continuation of application No. 12/001,395, filed on Dec. 11, 2007, now abandoned.
Application No. 12/001,395 is a continuation of application No. 10/806,018, filed on Mar. 22, 2004, now abandoned.
Application No. 10/806,018 is a continuation of application No. 09/782,980, filed on Feb. 13, 2001, now abandoned.
Application No. 09/782,980 is a continuation-in-part of application No. PCT/US00/02125, filed on Jan. 27, 2000.
Application No. PCT/US00/02125 is a continuation-in-part of application No. 09/448,076, filed on Nov. 23, 1999, now Pat. No. 6,300,092.
Application No. 09/448,076 is a continuation-in-part of application No. 09/276,400, filed on Mar. 25, 1999, now Pat. No. 6,140,056.
Application No. 09/782,980 is a continuation-in-part of application No. 09/014,195, filed on Jan. 27, 1998, now abandoned.
Application No. 09/014,195 is a continuation-in-part of application No. 09/014,348, filed on Jan. 27, 1998, now abandoned.
Application No. 09/014,348 is a continuation-in-part of application No. 09/086,892, filed on May 29, 1998, now abandoned.
Application No. 09/086,892 is a continuation-in-part of application No. 09/296,208, filed on Apr. 21, 1999, now abandoned.
Application No. 09/296,208 is a continuation-in-part of application No. 09/063,950, filed on Apr. 21, 1998, now Pat. No. 6,225,085.
Application No. 09/782,980 is a continuation-in-part of application No. 09/561,381, filed on Apr. 28, 2000, now abandoned.
Application No. 09/561,381 is a continuation-in-part of application No. 09/561,810, filed on Apr. 28, 2000, now abandoned.
Application No. 09/561,810 is a continuation-in-part of application No. 09/087,121, filed on May 29, 1998, now abandoned.
Application No. 09/782,980 is a continuation-in-part of application No. 09/672,721, filed on Sep. 28, 2000, now abandoned.
Application No. 09/672,721 is a continuation of application No. 09/049,799, filed on Mar. 27, 1998, now abandoned.
(60) Provisional application No. 60/117,580, filed on Jan. 27, 1999.

(51) Int. Cl. C12Q 1/68; G01N 33/566; C12N 5/10; C12P 21/02; C07K 16/18; C07H 21/00; C07K 14/47; C12N 15/63

(52) U.S. Cl. 435/6; 435/7.21; 435/325; 435/69.1; 530/387.9; 536/23.5; 530/350; 435/320.1; 436/501

(57) ABSTRACT

Novel ITALY, LOR-2, STRIFE, TRASH, BDSF, LRSG, and STMST polypeptides, proteins, and nucleic acid molecules are disclosed. In addition to isolated, full-length ITALY, LOR-2, STRIFE, TRASH, BDSF, LRSG, and STMST proteins, the invention further provides isolated ITALY, LOR-2, STRIFE, TRASH, BDSF, LRSG, and STMST fusion proteins, antigenic peptides and anti-ITALY, LOR-2, STRIFE, TRASH, BDSF, LRSG, and STMST antibodies. The invention also provides ITALY, LOR-2, STRIFE, TRASH, BDSF, LRSG, and STMST nucleic acid molecules, recombinant expression vectors containing a nucleic acid molecule of the invention, host cells into which the expression vectors have been introduced and non-human transgenic animals in which an ITALY, LOR-2, STRIFE, TRASH, BDSF, LRSG, and STMST gene has been introduced or disrupted. Diagnostic, screening and therapeutic methods utilizing compositions of the invention are also provided.

### RELATED APPLICATIONS

This application is a continuation of U.S. application Ser. No. 12/001,395, filed Dec. 11, 2007 (pending), which is a continuation of U.S. application Ser. No. 10/806,018, filed Mar. 22, 2004 (now abandoned), which is a continuation of U.S. application Ser. No. 09/782,980, filed on Feb. 13, 2001 (now abandoned).

U.S. application Ser. No. 09/782,980 is a continuation-in-part of PCT Application Serial No. PCT/US00/02125, filed Jan. 27, 2000, now abandoned, which is a continuation-in-part of U.S. patent application Ser. No. 09/448,076, filed Nov. 23, 1999, now U.S. Pat. No. 6,300,092, which is a continuation-in-part of U.S. patent application Ser. No. 09/276,400, filed Mar. 25, 1999, now U.S. Pat. No. 6,140,056, which claims the benefit of U.S. Provisional Application Ser. No. 60/117,580, filed Jan. 27, 1999, now abandoned. U.S. application Ser. No. 09/782,980 is also related to U.S. patent application Ser. No. 09/702,572, filed Oct. 31, 2000, now U.S. Pat. No. 6,391,602.

U.S. application Ser. No. 09/782,980 is also a continuation-in-part of U.S. patent application Ser. No. 09/014,195, filed Jan. 27, 1998, now abandoned.

U.S. application Ser. No. 09/782,980 is also a continuation-in-part of U.S. patent application Ser. No. 09/014,348, filed Jan. 27, 1998, now abandoned.

U.S. application Ser. No. 09/782,980 is also a continuation-in-part of U.S. patent application Ser. No. 09/086,892, filed May 29, 1998, now abandoned.

U.S. application Ser. No. 09/782,980 is also a continuation-in-part of U.S. patent application Ser. No. 09/296,208, filed Apr. 21, 1999, now abandoned, which is a continuation-in-part of U.S. patent application Ser. No. 09/063,950, filed Apr. 21, 1998, now U.S. Pat. No. 6,225,085.

U.S. application Ser. No. 09/782,980 is also a continuation-in-part of U.S. patent application Ser. No. 09/561,381, filed Apr. 28, 2000, now abandoned, and of U.S. patent application Ser. No. 09/561,810, filed Apr. 28, 2000, now abandoned, both of which are continuations-in-part of U.S. patent application Ser. No. 09/087,121, filed May 29, 1998, now abandoned.

U.S. application Ser. No. 09/782,980 is also a continuation-in-part of U.S. patent application Ser. No. 09/672,721, filed Sep. 28, 2000, now abandoned, which is a continuation of U.S. patent application Ser. No. 09/049,799, filed Mar. 27, 1998, now abandoned.

The entire content of each of the above-referenced patent applications is incorporated herein by this reference.

### BACKGROUND OF THE INVENTION

The immune system, which includes B cells, T cells, antigen presenting cells, and the biochemical mediators which help communicate and relay signals between them, is one of many systems involved in inflammation, host defense, and other viral processes. This involvement may include direct cellular contact, chemical mediators or both. One such chemical mediator is Interleukin-10 (IL-10). Human IL-10 is a 160 amino acid protein which contains four disulfide bond forming cysteine residues. Reduction of these disulfide bonds results in loss of helical structure with concomitant loss of biologic activity (Windsor W. T. et al. (1993) Biochemistry 32:8807-15). The tertiary structure of IL-10 is that of a V-shaped homodimer. Each half of the V-shaped structure has six helices. Four of these helices form an “up-up-down-down” bundle, a structure present in all helical cytokines.

IL-10 acts on a variety of cells within the immune system. Its effects are both complex and varied, and include the inhibition of antigen stimulated T cell proliferation (both directly and indirectly). More specifically, IL-10 has been shown to decrease surface expression of MHC class II molecules on a variety of antigen presenting cells (APC), including dendritic cells, monocytes, and Langerhans' cells (Bejarano M. T. et al. (1992) Int. Immunol. 4:1389-97 and Caux C. et al. (1994) Int. Immunol. 6:1177-85). IL-10 also down regulates co-stimulatory pathways such as APC surface expression of intracellular adhesion molecules (ICAM), CD80 (B7-1), and CD86 (B7-2) (Williams F. et al. (1994) J. Immunol. 24:1007-09 and Kubin M. et al. (1994) J. Exp. Med. 180:211-22). IL-10 is also capable of directly inhibiting T cell proliferation, independent of its actions on APCs. IL-10 inhibits anti-CD3 mAb stimulated T cell proliferation via inhibition of IL-2 production by T cells (deWaal M. R. et al. (1993) J. Immunol. 150:4754-65), and CD80-86/CD28 dependent IL-5 secretion by T cells (Schwartz R. H. et al. (1996) J. Exp. Med. 184:1-8).

IL-10 can also enhance immune activity by, for example, stimulating proliferation, activation and chemotaxis of CD28+ T cells (Chen W. F. et al. (1991) J. Immunol. 147:528-34). IL-10 has also been shown up regulate Fc receptors on monocytes, thus enhancing antibody dependent cytotoxicity (Velde T. et al. (1992) J. Immunol. 142:4048-52). IL-10 has further been demonstrated to increase B cell proliferation, differentiation, and antibody production (Defrance T. et al. (1992) J. Exp. Med. 175:671-82).

Given the above described functions, IL-10 plays an important role in immune and inflammation disorders such as Rheumatoid Arthritis (Bucht A. et al. (1996) Clin. Exp. Immunol. 103:357-67 and Walmsley M. et al. (1996) Arthritis and Rheumatism 39:495-503), Systemic Lupus Erythematosus (Houssian F. A. et al. (1995) Lupus 4:393-5), Myasthenia Gravis (Zhang G. X. et al. (1997) Muscle Nerve 20:543-51), Grave's Disease (Kallmann B. A. et al. 1997) IDDM 46:237-43), Sjogren Syndrome (Ohyama Y. et al. (1996) Arthritis Rheumatism 39:1376-84), Polymyositis and Dermatomyositis (Hagiwara E. et al. (1996) Clin. Exp. Rheum. 14:485-91), Psoriasis (Michel G. et al. (1997) Inflammation Res. 46:32-4), Pemphigus Vulgaris (Wucherpfenig K. W. et al. (1995) Proc. Natl. Acad. Sci. 92:11935-9), Bullous Pemphigoid (Schmidt E. et al. (1996) Arch. Dermatol. Res. 288:353-7), Inflammatory Bowel Disease (Berg D. J. et al. (1996) J. Clin. Invest. 98:1010-44), Kawasaki Disease (Hira J. et al. (1997) Int. Arch. Allergy Immunol. 112:152-6), Asthma, (Kawano Y. et al. (1995) Clin. Exp. Immunol. 102:389-94), and Graft v. Host Disease.

Another molecule sharing structural homology to IL-10 is MDA-7. The MDA-7 gene was cloned from a human melanoma library and encodes a 206 amino acid protein (Jiang H. et al. (1993) Mol. Cell. Differ. 1:285-299). MDA-7 is induced as a function of growth arrest and induction of terminal differentiation in human melanoma cells (Jiang H. et al. (1994) Mol. Cell. Differ. 2:221-239). MDA-7 expression also inversely correlates with melanoma progression. Moreover, MDA-7 is growth inhibitory toward human melanoma cells in transient transfection assays and in stable transformed cells containing a dexamethasone inducible MDA-7 gene. In addition to its role in melanoma, MDA-7 has been shown to be a potent growth suppressing factor in cancer cells of diverse origin including breast, central nervous system, cervix, colon, prostate, and connective tissue. As a result of the above described functions, MDA-7 plays an important role in the modulation of growth in a variety of proliferative disorders, e.g., cancer such as melanoma, prostate cancer, cervical cancer, breast cancer, colon cancer, and sarcoma.

Lysyl oxidase (“LOX”) is an extracellular copper enzyme that initiates the crosslinking of collagens and elastin by catalyzing oxidative deamination of the ε-amino group in certain lysine and hydroxylysine residues of collagens and lysine residues of elastin (Smith-Mungo and Kagan (1998) Matrix Biol. 16:387-398 and Kaman in Biology of Extracellular Matrix, ed. Mecham (1986) Academic Press pp. 321-389). Lysyl oxidase has been shown to be important in a variety of cellular and physiologic processes including biogenesis of connective tissue matrices and bone resorption. A deficiency in lysyl oxidase activity is found in two X-linked, recessively inherited connective tissue disorders, the type IX variant of the Ehlers-Danlos syndrome and the Menkes syndrome, and in the X-linked, recessively inherited mottled series of allelic mutant mice (all characterized by abnormalities in copper metabolism). (Byers et al. (1980) New Engl. J. Med. 303:61-65; Royce et al. (1980) Biochemistry J. 192:579-586; Kuivaniemi et al. (1982) J. Clin. Invest. 69:730-733; Kuivaniemi et al. (1985) Amer. J. Human. Genet. 37:798-808; Peltonen et al. (1983) Biochemistry 22:6156-6163; Rowe et al. (1977) J. Biol. Chem. 252:939-942; Starcher et al. (1977) Biochem. Biophys. Res. Commun. 78:706-712; Danks in The Metabolic Basis of Inherited Disease”, eds. Stanbury et al. (1983), McGraw-Hill pp. 1251-1268). Increased lysyl oxidase activity has been associated with fibrotic disorders such as atherosclerosis, hypertension, and liver and pulmonary fibrosis. (Kagan, supra).

More recently there have been identified proteins having structural and/or functional similarities to lysyl oxidase. For example, a lysyl oxidase-like protein, referred to herein as “LOL”, was identified from a human skin fibroblast cDNA library that contains extensive homology to several coding domains within the human lysyl oxidase mRNA which is believed to be involved in collagen maturation. (Kenyon et al. (1993) J. Biol. Chem. 268:18435-18437 and Kim et al. (1995) J. Biol. Chem. 270:7176-7182). Recent cloning and analysis of the mouse LOL gene (Kim et al. (1999) J. Cell Biochem. 72:181-188) demonstrated that steady state levels of LOL mRNA and type III procollagen mRNA increased coincidentally early in the development of liver fibrosis. In contrast, steady state levels of lysyl oxidase mRNA increased throughout the onset of hepatic fibrosis and appeared in parallel with the increased steady state levels of pro-alpha (I) collagen mRNA, suggesting that the LOL protein is involved in the development of lysine-derived cross-links in collagenous substrates. Moreover, the substrate specificity of the LOL protein may be different to that of lysyl oxidase and this difference may be collagen-type specific.

Likewise, a protein referred to herein as lysyl-oxidase related protein (“Lor”) has been identified which inhibits many of the structural features of lysyl oxidase and is overexpressed in senescent fibroblasts and is believed to play a role in age-associated changes in extracellular proteins. (Saito et al. (1997) J. Biol. Chem. 272:8157-8160). Lor contains four domains referred to herein as scavenger receptor cysteine-rich domains (“SRCR domains”) which are believed to be involved in binding to other cell surface proteins or extracellular molecules. The SRCR domain joins a long list of other widely distributed cysteine-containing domains found in extracellular portions of membrane proteins and in secreted proteins (Doolittle (1985) Trends Biochem. Sci. 10:233-237; Krieger in Molecular Structures of Receptors, eds. Rossow et al. (1986) Horwood, Chichester, U.K. pp. 210-231). Examples include the EGF-like domain, immunoglobulin superfamily domains, the LDL receptor/complement. C9 domain, clotting factor Kringle domains, and fibronectin domains. These disulfide cross-linked domains appear to provide stable core structures that (i) are able to withstand the rigors of the extracellular environment; (ii) are well suited for a variety of biochemical tasks, often involving binding; and (iii) are readily juxtaposed to other types of domains to permit the construction of complex mosaic proteins. (Doolittle supra; Sudhof et al. (1985) Science 228:815-822). Lastly, a mouse cDNA encoding a putative protein having sequence homology to lysyl oxidase has recently been identified having the Accession No. AF053368, referred to herein as “Lor-2”.

Lysyl oxidases (“LOXs”) have been immunolocalized to the extracellular matrix regions of stroma surrounding early breast cancers (Decitre et al. (1998) Lab Invest. 78:143-151), with decreased expression observed in the stroma surrounding invasive breast cancers (Peyrol et al. (1997) Am. J. Pathol. 150:497-507). A progressive loss of LOX expression has also been observed during prostrate cancer progression in mice (Ren et al. (1998) Cancer Res. 58:1285-1290). These observations suggest that lysyl oxidases may function as tumor suppressors.

It has further been shown that human Lor is highly expressed in all adherent tumor cell lines examined, but not in cell lines that grow in suspension (Saito et al., supra), suggesting that LOXs can increase the adhesion properties of tumor cells. Lor expression was demonstrated to be concomitant with upregulation of type I procollagen. As adhesion properties contribute to the ability of tumor cells to colonize new sites, a tumor-promoting role for LOXs is also probable.

A greater understanding of the role which lysyl oxidase-like as well as SCRC domain containing proteins play in various disorders would lead to the determination of highly specific drug targets which would work to treat these disorders, e.g., cardiovascular disorders, a disorder arising from altered lysyl oxidase-like activity or a disorder arising from improperly regulated SRCR-domain containing protein activity giving rise to improperly regulated cellular processes.

The tumor necrosis factor receptor (TNFR) superfamily of proteins encompasses over a dozen members, most of which are type I transmembrane proteins, related by the presence of conserved cysteine-rich repeats (CRRs) in their N-terminal cysteine-rich domains (CRDs). Members of the TNFR superfamily include TNFR1 (p55), TNFR2 (p75), TNFR3 (TNF-RP), Fas (also known as CD95 and Apo1), OX-40, 41-BB, CD40, CD30, CD27, OPG, and p75 NGFR. (Smith et al. (1993) Cell 76:959-962; Armitage, R. J. (1994) Curr. Opin. Immunol. 6:407-413; Gruss et al. (1995) Blood 85, 3378-3404; Baker et al. (1996) Oncogene 12:1-9; and Simonet et al. (1997) Cell 89:309-319.) A TNFR superfamily member is typically a membrane-bound, trimeric or multimeric complex which is stabilized via intracysteine disulfide bonds that are formed between the cysteine-rich domains of individual subunit members (Banner et al. (1993) Cell 73:431-445). The proteins themselves do not have intrinsic catalytic activity, rather they function via association with other proteins to transduce cellular signals.

A functional TNFR superfamily protein can also exist in a soluble form. Soluble versions of the superfamily bind cognate ligands and influence bioavailability. For instance, the osteoprotegerin protein family exists as a soluble protein. (Simonet et al. (1997) Cell 89:309-319.) Many soluble forms of the TNFR have been identified. Certain soluble TNFRs are elevated in disease states such as lupus and rheumatoid arthritis. (Gabay et al. (1997) J. Rheumatol. 24(2):303-308. The soluble superfamily members lack the transmembrane domain characteristic of the majority of superfamily members due to either proteolytic cleavage or, at least in one instance, to alternative splicing (Gruss et al. (1995) Blood 85, 3378-3404.)

Cytokines are small peptide molecules produced by a variety of cells that mediate a wide range of biological activities. Arai, K.-I. et al. (1990) Annu. Rev. Biochem. 59:783 and Paul, W. E. and R. A. Seder (1994) Cell 76:241. Through a complex network, cytokines regulate functions including cellular growth, inflammation, immunity, differentiation and repair. Mosmann, T. R. (1991) Curr. Opin. Immunol. 3:311. One family of cytokines, includes the tumor necrosis factor ligand (TNFL) superfamily of proteins which includes two structurally and functionally related proteins, TNF-α and TNF-β. TNF-α (also known as cachectin) is synthesized as a type II membrane protein which then undergoes post-translational cleavage liberating the extracellular domain. TNF-α possesses a wide variety of functions including the ability to induce cytolysis of certain tumor cell lines and the induction of cachexia. TNF-α is a potent pyrogen, causing fever by direct action or by stimulation of interleukin-1 secretion. In addition, TNF-α can stimulate cell proliferation and induce cell differentiation under certain conditions. TNF-α is characteristically produced at the sites of inflammation by infiltrating mononuclear cells. TNF-α plays a beneficial role as an immunostimulant and an important mediator of host resistance to many infectious agents. Overproduction of TNF-α can lead to severe systemic toxicity and even death. TNF-α has also been implicated in the pathogenesis of some autoimmune disorders.

CD27L, CD30L, CD40L, FASL, Lt-β, and 4-1BBL also appear to be type II membrane proteins. All of these cytokines appear to form homotrimeric complexes that are recognized by their specific receptors.

Given members of the TNFL superfamily of proteins are involved in the activation of a large array of cellular genes and of multiple signal transduction pathways, kinases and transcription factors, there exists a need for the identification of novel TNFL-like molecules, as well as, for modulators of such molecules for use in regulating a variety of cellular responses.

Signaling factors play an important role in the development and functioning of different cell types by allowing for communication between interacting cells. Such factors provide a signal between cells which can cause cells which recognize the signal to perform specialized tasks, such as cell growth, differentiation and/or proliferation.

For example, cells of the immune system characteristically express a variety of signaling proteins which are crucial to proper functioning of the immune system. Such proteins include secreted immunoglobulins and non-immunoglobulin molecules which interact with cellular adhesion molecules, as well as other selected target molecules. Many of these proteins are members of the immunoglobulin (Ig) superfamily of proteins, characterized by the existence of at least one immunoglobulin (Ig)-like domain. Such proteins function in an variety of immune cell functions ranging from immune cell development and differentiation, antigen recognition, antibody production, cellular signal transduction, and cellular homing of immune responsive cells from the circulation to sites of increased antigen concentration.

Given the importance of such signaling proteins in the proper functioning of the immune system, there exists a need to identify novel signaling factors which function to regulate the immune response and whose aberrant function can result in immune response disorders such as congenital or acquired immunodeficiency, and inflammatory disorders such as arthritis.

Leucine-rich repeats (“LRRs”) were first discovered in leucine-rich α2-glycoprotein, a protein of unknown function from human serum (Takashashi, et al. (1985) Proc. Natl. Acad. Sci. USA 82:1906-1910). LRR-containing proteins now represent a diverse group of molecules with differing functions and cellular locations in a variety of organisms (for review see Kobe and Deisenhofer (1994) Trends Biochem. Sci. 19:415-421). In particular, LRR-containing proteins are known to be involved in a wide range of functions including protein-protein interactions and signal transduction. For example, adhesive proteins represent the largest group in the LRR superfamily. One family of adhesive LRR-containing proteins includes the small proteoglycans: biglycan, fibromodulin, decorin, lumican, proteoglycan-Lb and osteoinductive factor (OIF, also called osteoglycan). Small proteoglycans bind various components of the extracellular matrix and growth factors. Decorin and fibromodulin regulate collagen-fibril formation; and OIF, in conjunction with the transforming growth factors TGF-β and TGF-β2, induces bone formation.

Another exemplary family of adhesive proteins comprises the proteins of the Ib-V-IX system of platelet glycoproteins. This complex constitutes the receptor for von Willebrand factor and mediates the adhesion of platelets to injured vascular surfaces. The LRR superfamily further contains several families of signal-transducing receptors (e.g., CD14 and the proto-oncogene trk).

As the name implies, LRRs are distinguished by a consensus sequence consisting predominantly of leucines. The consensus sequence compiled from known LRR containing proteins contains leucines or other aliphatic residues at positions 2, 5, 7, 12, 16, 21 and 24, and asparagine, cysteine or threonine at position 10. Most proteins contain exclusively asparagine at position 10.

Given the wide range of important functions of LRR containing proteins, such as protein:protein interactions, matrix association and signal transduction, there exists a need for identifying novel LRR containing proteins as well as for modulators of such molecules for use in regulating a variety of cellular responses.

Molecular cloning studies have shown that G protein-coupled receptors (“GPCRs”) form one of the largest protein superfamilies found in nature, and it is estimated that greater than 1000 different such receptors exist in mammals. Upon binding of extracellular ligands, GPCRs interact with a specific subset of heterotrimeric G-proteins that can then, in their activated forms, inhibit or activate various effector enzymes and/or ion channels. The ligands for many of these receptors are known although there exists an ever-increasing number of GPCRs which have been identified in the sequencing of the human genome which for have no ligands have yet been identified. This latter subfamily of GPCRs is called the orphan family of GPCRs. In addition to both GPCRs with known ligands, as well as orphan GPCRs, there exist a family of GPCR-like molecules which share significant homology as well as many of the structural properties of the GPCR superfamily. For example, a family of GPCR-like proteins which arises from three alternatively-spliced forms of a gene occurring between the CD4 and triosephosphate isomerase genes at human chromosome 12p13, has been recently identified (including protein A-1, A-2, and A-3). Ansari-Lari et al. (1996) Genome Res. 6(4):314-326. Comparative sequence analysis of the syntenic region in mouse chromosome 6 has further revealed a murine homologue of at least the A-2 splice product. Ansari-Lari et al. (1998) Genome Res. 8(1):29-40.

The fundamental knowledge that GPCRs play a role in regulating that activity of virtually every cell in the human body has fostered an extensive search for modulators of such receptors for use as human therapeutics. In fact, the superfamily of GPCRs has proven to be among the most successful drug targets. Consequently, it has been recognized that the newly isolated orphan GPCRs, as well as the GPCR-like proteins, have great potential for drug discovery.

With the identification of each new GPCR, orphan GPCR, and GPCR-like protein, there exists a need for identifying the surrogate ligands for such molecules as well as for modulators of such molecules for use in regulating a variety of cellular responses.

### SUMMARY OF THE INVENTION

The present invention is based, at least in part, on the discovery of novel molecules, referred to herein as “Interleukin Ten Associated Locus Yang” (“ITALY”, also referred to herein as “TANGO 116”), “Lysyl Oxidase Related-2” (“Lor-2”, also referred to herein as Myocardium Secreted Protein-18 or “MSP-18”), “STRIFE” (e.g., “STRIFE1”, also referred to herein as “Tango127a” or “T127a”, and “STRIFE2”, also referred to herein as “Tango127b” or “T127b”), “TRASH”, also referred to as “TANGO 118”, “Brain-Derived Signaling Factor” (“BDSF”, e.g., human “BDSF-1”, also referred to herein as “TANGO 122” or “hT122”, and mouse “BDSF-1”, also referred to as “mT122”), “Leucine-rich Surface Glycoprotein” (“LRSG”, e.g., “LRSG-1”, also referred to herein as human “TANGO 124”), and “Seven Transmembrane Signal Transducer” (“STMST”, e.g., STMST-1 and STMST-2, also referred to herein as “TANGO123a” and “TANGO123c”, respectively) nucleic acid and protein molecules. As used herein, the terms “nucleic acid molecules of the invention” and “polypeptides or proteins of the invention” refer to the ITALY, Lor-2, STRIFE, TRASH, BDSF, LRSG, and STMST nucleic acid and polypeptide or protein molecules of the invention, as described herein.

The nucleic acid and polypeptide molecules of the present invention are useful as modulating agents in regulating a variety of cellular processes. Accordingly, in one aspect, this invention provides isolated nucleic acid molecules encoding ITALY, Lor-2, STRIFE, TRASH, BDSF, LRSG, and STMST proteins or biologically active portions thereof, as well as nucleic acid fragments suitable as primers or hybridization probes for the detection of ITALY, Lor-2, STRIFE, TRASH, BDSF, LRSG, and STMST-encoding nucleic acids.

In one embodiment, a nucleic acid molecule of the invention is at least 58%, 60%, 65%, 70%, 75%, 80%, 85%, 90%, 95% or more homologous to the nucleotide sequence (e.g., to the entire length of the nucleotide sequence) shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, or a complement thereof.

In a preferred embodiment, an isolated ITALY nucleic acid molecule has the nucleotide sequence shown SEQ ID NO:3, or a complement thereof. In another embodiment, the ITALY nucleic acid molecule further comprises nucleotides 1-42 of SEQ ID NO:1. In another embodiment, the ITALY nucleic acid molecule further comprises nucleotides 574-991 of SEQ ID NO:1. In another preferred embodiment, the ITALY nucleic acid molecule has the nucleotide sequence shown in SEQ ID NO:1. In another preferred embodiment, the ITALY nucleic acid molecule comprises a fragment of at least 588 nucleotides of the nucleotide sequence of SEQ ID NO:1, SEQ ID NO:3, or a complement thereof.

In another preferred embodiment, an isolated Lor-2 nucleic acid molecule includes the nucleotide sequence shown in SEQ ID NO:10, or a complement thereof. In another embodiment, the Lor-2 nucleic acid molecule includes nucleotides 143-2401 shown in SEQ ID NO:10. In another preferred embodiment, the Lor-2 nucleic acid molecule has the nucleotide sequence shown in SEQ ID NO:10. In another preferred embodiment, the Lor-2 nucleic acid molecule comprises a fragment of at least 50 contiguous nucleotides of the nucleotide sequence shown in SEQ ID NO:10, or a complement thereof.

In another preferred embodiment, an isolated STRIFE1 nucleic acid molecule has the nucleotide sequence shown SEQ ID NO:24, or a complement thereof. In another embodiment, a STRIFE nucleic acid molecule further comprises nucleotides 1-106 of SEQ ID NO:22. In yet another preferred embodiment, a STRIFE1 nucleic acid molecule further comprises nucleotides 751-981 of SEQ ID NO:22. In another preferred embodiment, an isolated STRIFE1 nucleic acid molecule has the nucleotide sequence shown in SEQ ID NO:22.

In another preferred embodiment, an isolated STRIFE2 nucleic acid molecule has the nucleotide sequence shown SEQ ID NO:28, or a complement thereof. In another embodiment, a STRIFE2 nucleic acid molecule further comprises nucleotides 1-109 of SEQ ID NO:26. In yet another preferred embodiment, a STRIFE2 nucleic acid molecule further comprises nucleotides 562-655 of SEQ ID NO:26. In another preferred embodiment, an isolated STRIFE2 nucleic acid molecule has the nucleotide sequence shown in SEQ ID NO:26.

In another preferred embodiment, an isolated TRASH nucleic acid molecule has the nucleotide sequence of SEQ ID NO:33 or a complement thereof. In another embodiment, a TRASH nucleic acid molecule further comprises nucleotides 1-272 of SEQ ID NO:31. In yet another preferred embodiment, a TRASH nucleic acid molecule further comprises nucleotides 1026-1346 of SEQ ID NO:31.

In another preferred embodiment, an isolated BDSF nucleic acid molecule has the nucleotide sequence of SEQ ID NO:47 or a complement thereof. In another embodiment, an isolated BDSF nucleic acid molecule further comprises nucleotides 244-701 of SEQ ID NO:45. In another embodiment, the BDSF nucleic acid molecule further comprises nucleotides 31-487 of SEQ ID NO:48.

In another preferred embodiment, an isolated LSRG nucleic acid molecule has the nucleotide sequence shown SEQ ID NO:60, or a complement thereof. In another embodiment, the LSRG nucleic acid molecule further comprises nucleotides 1-159 of SEQ ID NO:58. In another embodiment, the LSRG nucleic acid molecule further comprises nucleotides 2179-2852 of SEQ ID NO:58. In another preferred embodiment, an isolated LSRG nucleic acid molecule has the nucleotide sequence shown in SEQ ID NO:58. In yet another preferred embodiment, an isolated LSRG nucleic acid molecule has the nucleotide sequence of the DNA insert of the plasmid deposited with ATCC as Accession Number 98960, 98756, or 98695, or a complement thereof.

In another preferred embodiment, an isolated STMST nucleic acid molecule has the nucleotide sequence shown SEQ ID NO:72, or a complement thereof. In another embodiment, an STMST nucleic acid molecule further comprises nucleotides 1-403 of SEQ ID NO:70. In another embodiment, an STMST nucleic acid molecule further comprises nucleotides 1295-2915 of SEQ ID NO:70. In another preferred embodiment, an isolated STMST nucleic acid molecule has the nucleotide sequence shown in SEQ ID NO:70.

In another preferred embodiment, an isolated STMST nucleic acid molecule has the nucleotide sequence shown SEQ ID NO:75, or a complement thereof. In another embodiment, an STMST nucleic acid molecule further comprises nucleotides 1-333 of SEQ ID NO:73. In another embodiment, an STMST nucleic acid molecule further comprises nucleotides 2161-4166 of SEQ ID NO:73. In another preferred embodiment, an isolated STMST nucleic acid molecule has the nucleotide sequence shown in SEQ ID NO:73.

In another embodiment, a nucleic acid molecule of the invention includes a nucleotide sequence encoding a protein having an amino acid sequence sufficiently homologous to the amino acid sequence of SEQ ID NO:2, 4, 11, 23, 27, 32, 37, 39, 41, 46, 49, 51, 54, 59, 68, 71, or 74 or an amino acid sequence encoded by the DNA insert of the plasmid deposited with ATCC as Accession Number 98960, 98756, or 98695. As used herein, the term “sufficiently homologous” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least 50% homology, preferably 60% homology, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 50%, preferably 60%, more preferably 70-80%, or 90-95% homology and share a common functional activity are defined herein as sufficiently homologous.

In a preferred embodiment, a nucleic acid molecule of the invention includes a nucleotide sequence encoding a protein having an amino acid sequence at least 32%, 35%, 40%, 45%, 50%, 55%, 60%, 65%, 70%, 75%, 80%, 85%, 90%, 95% or more homologous to the amino acid sequence of SEQ ID NO:2, 4, 11, 23, 27, 32, 37, 39, 41, 46, 49, 51, 54, 59, 68, 71, or 74 or the amino acid sequence encoded by the DNA insert of the plasmid deposited with ATCC as Accession Number 98960, 98756, or 98695. In another preferred embodiment, an isolated nucleic acid molecule encodes the amino acid sequence of one of the polypeptides of the invention. In yet another preferred embodiment, the nucleic acid molecule includes a nucleotide sequence encoding a protein having the amino acid sequence of SEQ ID NO:2, 4, 11, 23, 27, 32, 37, 39, 41, 46, 49, 51, 54, 59, 68, 71, or 74 or the amino acid sequence encoded by the DNA insert of the plasmid deposited with ATCC as Accession Number 98960, 98756, or 98695.

In another embodiment, an isolated nucleic acid molecule of the present invention encodes a protein, preferably an ITALY protein, which includes a disulfide bond forming cysteine. In another embodiment, an isolated nucleic acid molecule of the present invention encodes a protein, preferably an ITALY protein, which includes an α-helical structure. In another embodiment, an isolated nucleic acid molecule of the present invention encodes a protein, preferably an ITALY protein, which includes a disulfide bond forming cysteine and an α-helical structure. In another embodiment, an isolated nucleic acid molecule of the present invention encodes a protein, preferably an ITALY protein, which includes a disulfide bond forming cysteine and an α-helical structure and, preferably, is secreted. In yet another embodiment, an ITALY nucleic acid molecule encodes an ITALY protein and is a naturally occurring nucleotide sequence.

In another embodiment, an isolated nucleic acid molecule of the present invention encodes a protein, preferably a Lor-2 protein, which includes one or more of the following: a signal sequence, a LOX domain and at least one SCRC domain. In another embodiment, the isolated nucleic acid molecule encodes a protein, preferably a Lor-2 protein, which includes a signal sequence, a LOX domain and at least two, three, or four SCRC domains. In yet another embodiment, the isolated nucleic acid molecule encodes a protein, preferably a Lor-2 protein, which includes a signal sequence, a LOX domain, at least one SCRC domain and is expressed and/or functions in cells of the cardiovascular system.

In another embodiment, an isolated nucleic acid molecule of the present invention encodes a STRIFE1 protein which includes a cysteine-rich domain, optionally a signal sequence, and is membrane bound. In another embodiment, an isolated nucleic acid molecule of the present invention encodes a STRIFE1 protein which includes a signal sequence and a cysteine-rich domain, wherein the cysteine-rich domain comprises at least one module, and is membrane bound. In yet another embodiment, a STRIFE1 nucleic acid molecule encodes a STRIFE1 protein and is a naturally occurring nucleotide sequence.

In another embodiment, an isolated nucleic acid molecule of the present invention encodes a STRIFE2 protein which includes a cysteine-rich domain, optionally a signal sequence, and is secreted. In another embodiment, an isolated nucleic acid molecule of the present invention encodes a STRIFE2 protein which includes a signal sequence and a cysteine-rich domain, wherein the cysteine-rich domain comprises at least one module, and is secreted. In yet another embodiment, a STRIFE2 nucleic acid molecule encodes a STRIFE2 protein and is a naturally occurring nucleotide sequence.

In another embodiment, an isolated nucleic acid molecule of the present invention encodes a TRASH protein which includes a TNF signature motif. In another embodiment, an isolated nucleic acid molecule of the present invention encodes a TRASH protein which includes a TNF signature motif and a TNF-like N-terminal transmembrane anchor for a type II membrane protein. In another embodiment, the TRASH nucleic acid molecule encodes a TRASH protein and is a naturally occurring nucleotide sequence. In yet another embodiment, an isolated nucleic acid molecule of the present invention encodes a TRASH protein and comprises a nucleotide sequence which hybridizes under stringent hybridization conditions to a nucleic acid molecule having the nucleotide sequence of SEQ ID NO:31, SEQ ID NO:33, SEQ ID NO:36, SEQ ID NO:38, or SEQ ID NO:40.

In another embodiment, an isolated BSDF nucleic acid molecule of the present invention encodes a protein, preferably a BDSF protein, which includes an immunoglobulin-like domain. In another embodiment, an isolated nucleic acid molecule of the present invention encodes a protein, preferably a BDSF protein, which includes a signal sequence, an immunoglobulin-like domain, and, preferably, is secreted. In yet another embodiment, a BDSF nucleic acid molecule encodes a BDSF protein and is a naturally occurring nucleotide sequence.

In another embodiment, an isolated LSRG nucleic acid molecule of the present invention encodes a protein, preferably a LRSG protein, which includes a leucine-rich repeat region. In another embodiment, an isolated nucleic acid molecule of the present invention encodes a protein, preferably a LRSG protein, which includes an EGF-like domain. In another embodiment, an isolated nucleic acid molecule of the present invention encodes a protein, preferably a LRSG protein, which includes a fibronectin type III-like (Fn type III) domain. In another embodiment, an isolated nucleic acid molecule of the present invention encodes a protein, preferably a LRSG protein, which includes a leucine-rich repeat region, an EGF-like domain and a FN type III-like domain. In another embodiment, an isolated nucleic acid molecule of the present invention encodes a protein, preferably a LRSG protein, which includes a signal sequence, a leucine-rich repeat region, an EGF-like domain and a FN type III-like domain, and, preferably, is membrane bound. In yet another embodiment, a LRSG nucleic acid molecule encodes a LRSG protein and is a naturally occurring nucleotide sequence.

In another embodiment, an isolated STMST nucleic acid molecule of the present invention encodes an STMST protein which includes at least one transmembrane domain. In another embodiment, an isolated nucleic acid molecule of the present invention encodes a protein which includes a 7 transmembrane receptor profile. In another embodiment, an isolated nucleic acid molecule of the present invention encodes a protein which includes a spectrin α-chain motif. In yet another embodiment, an STMST nucleic acid molecule encodes an STMST protein and is a naturally occurring nucleotide sequence.

Another embodiment of the invention features nucleic acid molecules, preferably ITALY, Lor-2, STRIFE, TRASH, BDSF, LRSG, or STMST nucleic acid molecules, which specifically detect ITALY, Lor-2, STRIFE, TRASH, BDSF, LRSG, or STMST nucleic acid molecules relative to nucleic acid molecules encoding other ITALY, Lor-2, STRIFE, TRASH, BDSF, LRSG, or STMST family members. For example, in one embodiment, an ITALY nucleic acid molecule is at least 588, 600-650, 651-700, 701-750, or 751-800 nucleotides in length and hybridizes under stringent conditions to a nucleic acid molecule comprising the nucleotide sequence shown in SEQ ID NO:1, or the nucleotide sequence of the DNA insert of the plasmid deposited with ATCC as Accession Number 98960, or a complement thereof. In preferred embodiments, the nucleic acid molecules are at least 15 (e.g., contiguous) nucleotides in length and hybridize under stringent conditions to nucleotides 1-332 or 936-991 of SEQ ID NO:1. In other preferred embodiments, the nucleic acid molecules comprise nucleotides 1-332 or 936-991 of SEQ ID NO:1. In another embodiment, a Lor-2 nucleic acid molecule is at least 300, 400, 500, 600, 650, 700, 750, or 753 nucleotides in length and hybridizes under stringent conditions to a nucleic acid molecule comprising the nucleotide sequence shown in SEQ ID NO:10, or a complement thereof. In a particularly preferred embodiment, the nucleic acid molecule comprises a fragment of at least 50 contiguous nucleotides of the nucleotide sequence of SEQ ID NO:10 or 12, or a complement thereof. In another preferred embodiment, the nucleic acid molecules are at least 25, 50, 75, 100, 150, 200, 250 or more nucleotides (e.g., contiguous) in length and hybridize under stringent conditions to SEQ ID NO:10. In another embodiment, a STRIFE1 or STRIFE2 nucleic acid molecule hybridizes under stringent conditions to a nucleic acid molecule comprising the nucleotides 107-751, 1-16, 413-602, or 711-981 of the nucleotide sequence shown in SEQ ID NO:22, or to nucleotides 110-562, 1-16, 416-489, or 519-655 of nucleotide sequence shown in SEQ ID NO:26, respectively. In another embodiment, the STRIFE1 or STRIFE2 nucleic acid molecule is at least 450 nucleotides in length and hybridizes under stringent conditions to a nucleic acid molecule comprising the nucleotide sequence shown in SEQ ID NO:22 or 26, respectively, or a complement thereof. In another embodiment, a TRASH nucleic acid molecule hybridizes under stringent conditions to a nucleic acid molecule comprising nucleotides 273-1025 of SEQ ID NO:31. In another embodiment, the TRASH nucleic acid molecule is at least 500 nucleotides in length and hybridizes under stringent conditions to a nucleic acid molecule comprising the nucleotide sequence shown in SEQ ID NO:31, 33, 36, 38, 40, or a complement thereof. In another embodiment, a BDSF nucleic acid molecule is at least 450, preferably 500-700, more preferably 700-900, more preferably 900-1100, and even more preferably 1100-1120 nucleotides in length and hybridizes under stringent conditions to a nucleic acid molecule comprising the nucleotide sequence shown in SEQ ID NO:45, or 50, or the nucleotide sequence of the DNA insert of the plasmid deposited with ATCC as Accession Number 98756, or a complement thereof. In another embodiment, an LRSG nucleic acid molecule is at least 1000, preferably 1000-1250, more preferably 1250-1500, more preferably 1500-1750, and even more preferably 1750-2000 nucleotides in length and hybridizes under stringent conditions to a nucleic acid molecule comprising the nucleotide sequence shown in SEQ ID NO:58 or 67, or the nucleotide sequence of the DNA insert of the plasmid deposited with ATCC as Accession Number 98695, or a complement thereof. In another embodiment, an STMST nucleic acid molecule is at least 350 nucleotides in length and hybridizes under stringent conditions to a nucleic acid molecule comprising the nucleotide sequence shown in SEQ ID NO:70 or 73, or a complement thereof.

In another embodiment, the invention provides an nucleic acid molecule which comprises at least 15 consecutive (e.g., at least 15 contiguous) nucleotides of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75.

In other preferred embodiments, the nucleic acid molecule encodes a naturally occurring allelic variant of a polypeptide comprising the amino acid sequence of SEQ ID NO:2, 4, 11, 23, 27, 32, 37, 39, 41, 46, 49, 51, 54, 59, 68, 71, or 74 or an amino acid sequence encoded by the DNA insert of the plasmid deposited with ATCC as Accession Number 98960, 98756, or 98695, wherein the nucleic acid molecule hybridizes to a nucleic acid molecule comprising SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75 under stringent conditions.

Another embodiment of the invention provides an isolated nucleic acid molecule which is antisense to a nucleic acid molecule of the invention, e.g., the coding strand of an ITALY, Lor-2, STRIFE, TRASH, BDSF, LRSG, or STMST nucleic acid molecule.

Another aspect of the invention provides a vector comprising a nucleic acid molecule of the invention. In certain embodiments, the vector is a recombinant expression vector. In another embodiment, the invention provides a host cell containing a vector of the invention. The invention also provides a method for producing a protein, preferably a protein of the invention, by culturing in a suitable medium, a host cell, e.g., a mammalian host cell such as a non-human mammalian cell, of the invention containing a recombinant expression vector, such that the protein is produced.

Another aspect of this invention features isolated or recombinant proteins and polypeptides.

In one embodiment, the isolated protein, preferably an ITALY protein, includes a disulfide bond forming cysteine. In another embodiment, the isolated protein, preferably an ITALY protein, includes an α-helical structure. In another embodiment, the isolated protein, preferably an ITALY protein, includes a disulfide bond forming cysteine and an α-helical structure and is, preferably, secreted. In yet another embodiment, the isolated protein, preferably an ITALY protein, has an amino acid sequence sufficiently homologous, as defined herein, to the amino acid sequence of SEQ ID NO:2.

In another embodiment, invention features isolated or recombinant Lor-2 proteins and polypeptides. In one embodiment, the isolated polypeptide includes one or more of the following: a signal sequence, a LOX domain and at least one SCRC domain. In another embodiment, the isolated polypeptide includes a signal sequence, a LOX domain and at least two, three, or four SCRC domains. In another embodiment, the isolated protein preferably includes a signal sequence, a LOX domain, at least one SCRC domain and has an amino acid sequence which is sufficiently homologous, as defined herein, to the amino acid sequence shown in SEQ ID NO:11. In yet another embodiment, the isolated protein, preferably a Lor-2 protein, includes a signal sequence, a LOX domain, at least one SCRC domain and is expressed and/or functions in cells of the cardiovascular system.

In another embodiment, an isolated STRIFE1 protein has a cysteine-rich domain, optionally a signal sequence, and is membrane bound. In another embodiment, an isolated STRIFE2 protein has a cysteine-rich domain, optionally a signal sequence, and is secreted. In yet another embodiment, an isolated STRIFE1 or STRIFE2 protein has an amino acid sequence sufficiently homologous, as defined herein, to the amino acid sequence of SEQ ID NO:23, or SEQ ID NO:27, respectively.

In another embodiment, an isolated TRASH protein has a TNF signature motif. In another embodiment, an isolated TRASH protein has a TNF signature motif and a TNF-like N-terminal signal transmembrane anchor for a type II membrane protein. In a preferred embodiment, an isolated TRASH protein further comprises two cysteine residues that may be disulfide linked. In yet another preferred embodiment, an isolated TRASH protein further comprises two putative N-linked glycosylation sites. In another embodiment, an isolated TRASH protein has an amino acid sequence sufficiently homologous, as defined herein, to the amino acid sequence of SEQ ID NO:32, 39, or 40.

In another embodiment, an isolated protein, preferably a BDSF protein, includes an immunoglobulin-like domain. In another embodiment, an isolated protein, preferably a BDSF protein, includes a signal sequence, an immunoglobulin-like domain, and is, preferably, secreted. In another embodiment, an isolated protein, preferably a BDSF protein, has an amino acid sequence sufficiently homologous, as defined herein, to the amino acid sequence of SEQ ID NO:46 or SEQ ID NO:49.

In another embodiment, an isolated protein, preferably a LRSG protein, includes a leucine-rich repeat region. In another embodiment, an isolated protein, preferably a LRSG protein, includes an EGF-like domain. In another embodiment, an isolated protein, preferably a LRSG protein, includes a Fn type III-like domain. In another embodiment, an isolated protein, preferably a LRSG protein, includes a leucine-rich repeat region, an EGF-like domain and a FN type III-like domain. In another embodiment, an isolated protein, preferably a LRSG protein, includes a signal sequence, a leucine-rich repeat region, an EGF-like domain and a FN type III-like domain and is, preferably, membrane bound. In another embodiment, an isolated protein, preferably a LRSG protein, has an amino acid sequence sufficiently homologous, as defined herein, to the amino acid sequence of SEQ ID NO:59 or SEQ ID NO:68.

In another embodiment, an isolated STMST protein includes at least one transmembrane domain. In another embodiment, an isolated STMST protein includes at least six transmembrane domains. In another embodiment, an isolated STMST protein includes seven transmembrane domains. In another embodiment, an isolated STMST protein includes a 7 transmembrane receptor profile. In another embodiment, an isolated STMST protein includes a spectrin α-chain profile. In another embodiment, an isolated STMST protein has an amino acid sequence sufficiently homologous, as defined herein, to the amino acid sequence of SEQ ID NO:68, 71, or 74.

In a preferred embodiment, the protein, preferably an ITALY, Lor-2, STRIFE, TRASH, BDSF, LRSG, or STMST protein, has an amino acid sequence at least about 32%, 35%, 40%, 45%, 50%, 55%, 60%, 65%, 70%, 75%, 80%, 85%, 90%, 95% homologous to the amino acid sequence of SEQ ID NO:2, 4, 11, 23, 27, 32, 37, 39, 41, 46, 49, 51, 54, 59, 68, 71, or 74 or the amino acid sequence encoded by the DNA insert of the plasmid deposited with ATCC as Accession Number 98960, 98756, or 98695. In another embodiment, the invention features fragments of the proteins having the amino acid sequence of SEQ ID NO:2, 4, 11, 23, 27, 32, 37, 39, 41, 46, 49, 51, 54, 59, 68, 71, or 74, wherein the fragment comprises at least 15 amino acids (e.g., contiguous amino acids) of the amino acid sequence of SEQ ID NO:2, or an amino acid sequence encoded by the DNA insert of the plasmid deposited with the ATCC as Accession Number 98960, 98756, or 98695. In another embodiment, the protein has the amino acid sequence of SEQ ID NO:2, 4, 11, 23, 27, 32, 37, 39, 41, 46, 49, 51, 54, 59, 68, 71, or 74.

Another embodiment of the invention features an isolated protein which is encoded by a nucleic acid molecule having a nucleotide sequence at least about 30%, 35%, 40%, 45%, 50%, 55%, 60%, 65%, 70%, 75%, 80%, 85%, 90%, 95% or more homologous to a nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or a complement thereof. This invention further features an isolated protein which is encoded by a nucleic acid molecule having a nucleotide sequence which hybridizes under stringent hybridization conditions to a nucleic acid molecule comprising the nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or a complement thereof.

In another embodiment, the invention provides an isolated protein which comprises at least 10 consecutive (e.g., at least 10 contiguous) amino acid residues of SEQ ID NO:2, 4, 11, 23, 27, 32, 37, 39, 41, 46, 49, 51, 54, 59, 68, 71, or 74. In another embodiment, the invention provides an isolated protein which corresponds to at least half of a full-length amino acid sequence of SEQ ID NO:2, 4, 11, 23, 27, 32, 37, 39, 41, 46, 49, 51, 54, 59, 68, 71, or 74.

The proteins of the present invention or biologically active portions thereof, can be operatively linked to a non-ITALY, Lor-2, STRIFE, TRASH, BDSF, LRSG, or STMST polypeptide (e.g., heterologous amino acid sequences) to form fusion proteins. The invention further features antibodies, such as monoclonal or polyclonal antibodies, that specifically bind proteins of the invention, preferably proteins of the invention. In addition, the proteins of the invention or biologically active portions thereof can be incorporated into pharmaceutical compositions, which optionally include pharmaceutically acceptable carriers.

In another aspect, the present invention provides a method for detecting the presence of a nucleic acid molecule, protein or polypeptide of the invention in a biological sample by contacting the biological sample with an agent capable of detecting a nucleic acid molecule, protein or polypeptide of the invention such that the presence of a nucleic acid molecule, protein or polypeptide of the invention is detected in the biological sample (e.g., a breast tissue sample or tumor sample).

In another aspect, the present invention provides a method for detecting the presence of activity of a polypeptide of the invention in a biological sample by contacting the biological sample with an agent capable of detecting an indicator of ITALY, Lor-2, STRIFE, TRASH, BDSF, LRSG, or STMST activity such that the presence of ITALY, Lor-2, STRIFE, TRASH, BDSF, LRSG, or STMST activity is detected in the biological sample (e.g., a breast tissue sample or tumor sample).

In another aspect, the invention provides a method for modulating the activity of a polypeptide of the invention comprising contacting a cell capable of expressing a polypeptide of the invention with an agent that modulates the activity of the polypeptide, such that the activity of the polypeptide in the cell is modulated. In one embodiment, the agent inhibits the activity of the polypeptide. In another embodiment, the agent stimulates the activity of the polypeptide. In one embodiment, the agent is an antibody that specifically binds to a protein of the invention. In another embodiment, the agent modulates expression of a polypeptide of the invention by modulating transcription of an ITALY, Lor-2, STRIFE, TRASH, BDSF, LRSG, or STMST gene or translation of an ITALY, Lor-2, STRIFE, TRASH, BDSF, LRSG, or STMST mRNA. In yet another embodiment, the agent is a nucleic acid molecule having a nucleotide sequence that is antisense to the coding strand of an ITALY, Lor-2, STRIFE, TRASH, BDSF, LRSG, or STMST mRNA or an ITALY, Lor-2, STRIFE, TRASH, BDSF, LRSG, or STMST gene.

In one embodiment, the methods of the present invention are used to treat a subject having a disorder characterized by aberrant ITALY, Lor-2, STRIFE, TRASH, BDSF, LRSG, or STMST protein or nucleic acid expression or activity by administering an agent which is a modulator of a polypeptide of the invention to the subject. In one embodiment, the ITALY, Lor-2, STRIFE, TRASH, BDSF, LRSG, or STMST modulator is a protein of the invention. In another embodiment the ITALY, Lor-2, STRIFE, TRASH, BDSF, LRSG, or STMST modulator is a nucleic acid molecule of the invention. In yet another embodiment, the ITALY, Lor-2, STRIFE, TRASH, BDSF, LRSG, or STMST modulator is a peptide, peptidomimetic, or other small molecule. In a preferred embodiment, the disorder characterized by aberrant ITALY, Lor-2, STRIFE, TRASH, BDSF, LRSG, or STMST protein or nucleic acid expression is an immune or inflammatory disorder, a developmental disorder, a differentiative disorder, a respiratory disorder (e.g., asthma), a cell death disorder, a bone disorder, an angiogenesis disorder, a vascular disorder, a hypothalamic disorder, or a proliferative disorder.

The present invention also provides a diagnostic assay for identifying the presence or absence of a genetic alteration characterized by at least one of (i) aberrant modification or mutation of a gene encoding a protein of the invention; (ii) mis-regulation of the gene; and (iii) aberrant post-translational modification of a protein of the invention, wherein a wild-type form of the gene encodes an protein with an ITALY, Lor-2, STRIFE, TRASH, BDSF, LRSG, or STMST activity.

In another aspect the invention provides a method for identifying a compound that binds to or modulates the activity of a protein of the invention, by providing a indicator composition comprising a protein of the invention having ITALY, Lor-2, STRIFE, TRASH, BDSF, LRSG, or STMST activity, contacting the indicator composition with a test compound, and determining the effect of the test compound on ITALY, Lor-2, STRIFE, TRASH, BDSF, LRSG, or STMST activity in the indicator composition to identify a compound that modulates the activity of an ITALY, Lor-2, STRIFE, TRASH, BDSF, LRSG, or STMST protein.

Other features and advantages of the invention will be apparent from the following detailed description and claims.

### BRIEF DESCRIPTION OF THE DRAWINGS

FIG. 1 depicts the cDNA sequence and predicted amino acid sequence of human ITALY. The nucleotide sequence corresponds to nucleic acids 1 to 991 of SEQ ID NO:1. The amino acid sequence corresponds to amino acids 1 to 177 of SEQ ID NO:2. The coding region without the 5′ and 3′ untranslated regions of the human ITALY gene is shown in SEQ ID NO:3 and is depicted as triplets underneath the corresponding encoded amino acid residues. The mature protein without the signal peptide sequence is shown in SEQ ID NO:4.

FIG. 2 depicts an alignment of the amino acid sequence of human ITALY with the amino acid sequences of the following proteins: human IL-10 (SEQ ID NO:5), mouse IL-10 (SEQ ID NO:6), viral IL-10 (SEQ ID NO:7), mouse MDA-7 (SEQ ID NO:8), and human MDA-7 (SEQ ID NO:9). The location of a conserved cysteine residue is indicated by a box.

FIGS. 3A-3C depict the cDNA sequence of human Lor-2. The nucleotide sequence corresponds to nucleic acids 1-2920 of SEQ ID NO:10.

FIGS. 4A-4C depict the amino acid sequence of human Lor-2. The amino acid sequence corresponds to amino acids 1-753 of SEQ ID NO:11.

FIGS. 5A-5B depict the coding sequence of human Lor-2. The nucleotide sequence corresponds to nucleic acids 1-2259 of SEQ ID NO:12.

FIG. 6 shows a protein analysis of the Lor-2 amino acid sequence depicted in SEQ ID NO:11. Shown are regions identified with the following algorithms: alpha, beta turn and coil regions, Garnier-Robson algorithm (Garnier et al. (1978) J. Mol. Biol. 120:97); alpha, beta, and turn regions, Chou-Fasman algorithm (Chou and Fasman (1978) Adv. Enzymol. Mol. 47:45-148); hydrophilicity and hydrophobicity plots, Kyte-Doolittle algorithm (Kyte and Doolittle (1982) J. Mol. Biol. 157:105-132); alpha amphipathic and beta amphipathic regions, Eisenberg algorithm (Eisenberg et al. (1982) Nature 299:371-374); flexible regions, Karplus-Schulz algorithm (Karplus and Schulz (1985) Naturwissens-Chafen 72:212-213); antigenic index, Jameson-Wolf algorithm (Jameson and Wolf (1988) CABIOS 4:121-136); surface probability plot, Emini algorithm (Emini et al. (1985) J. Virol. 55:836-839).

FIGS. 7A-7B depict a multiple sequence alignment of the amino acid sequence of human lysyl oxidase, LOX (Accession Number 2144342) (SEQ ID NO:14), human lysyl oxidase-like protein, LOL (Accession Number L21186) (SEQ ID NO:15), human lysyl oxidase-related protein, Lor (Accession Number U89942) (SEQ ID NO:16), murine lysyl oxidase-related protein 2, Lor-2 (Accession No. AF053368, SEQ ID NO:17), and the amino acid sequence of human Lor-2 (corresponding amino acids 1 to 753 of SEQ ID NO:11). The alignment was generated using the Clustal algorithm which is part of the MegAlign™ software package. The multiple alignment parameters are as follows: Gap Penalty=10; Gap Length Penalty=10. The pairwise alignment parameters are as follows: K-tuple=1; Gap Penalty=3; Window=5; Diagonals Saved=5; Weight Residue Table=PAM250. The SCRC domains are indicated in italics. The lysyl oxidase domain is indicated in bold. The copper binding site is overlined. The lysyl oxidase-related region is underlined. (For the huLO sequence depicted, the amino acid residues corresponding to the processed enzyme are underlined.)

FIG. 8 depicts the results of radiation hybrid mapping of the gene encoding human Lor-2 (i.e., clone Fbh21967). The location of clone Fbh21967, relative to various markers, is depicted as are the relative distances between markers.

FIG. 9 is a graphic depiction of the relative levels of Lor-2 expression in various normal tissue samples.

FIG. 10 is a graphic depiction of the relative levels of Lor-2 expression in additional tissue and cell samples.

FIG. 11 is a graphic depiction of the relative levels of Lor-2 expression in various normal versus tumor tissue samples.

FIGS. 12A-12B depict the cDNA sequence and predicted amino acid sequence of murine STRIFE1. The nucleotide sequence corresponds to nucleic acids 1 to 981 of SEQ ID NO:22. The amino acid sequence corresponds to amino acids 1 to 214 of SEQ ID NO:23. The coding region without the 5′ and 3′ untranslated regions of the murine STRIFE1 gene is shown in SEQ ID NO:24 and is depicted as triplets underneath the corresponding encoded amino acid residues.

FIG. 13 depicts the cDNA sequence and predicted amino acid sequence of murine STRIFE2. The nucleotide sequence corresponds to nucleic acids 1 to 655 of SEQ ID NO:26. The amino acid sequence corresponds to amino acids 1 to 150 of SEQ ID NO:27. The coding region without the 5′ and 3′ untranslated regions of the murine STRIFE2 gene is shown in SEQ ID NO:28 and is depicted as triplets underneath the corresponding encoded amino acid residues.

FIGS. 14A-14B depict an alignment of the amino acid sequences of murine STRIFE1 (also referred to herein as “Tango127a” or “T127a”), STRIFE2 (also referred to herein as “Tango127b” or “T127b”), and murine OX40 (Accession Number P47741). Amino acid residues which are conserved between murine STRIFE1 and STRIFE2 family members are boxed.

FIGS. 15A-15B depict an alignment to a hit resulting from a FASTA search using the amino acid sequence of STRIFE1 as a query. Identical amino acid residues are shown with colons (:). Similar amino acid residues are shown with periods (.).

FIG. 16A-16I depict an alignment to a hit resulting from a FASTA search using the nucleotide sequence of STRIFE1 as a query. Identical amino acid residues are shown with colons (:). Similar amino acid residues are shown with periods (.).

FIG. 17 depicts the cDNA sequence and predicted amino acid sequence of a human TRASH. The nucleotide sequence corresponds to nucleic acids 1 to 1346 of SEQ ID NO:31. The amino acid sequence corresponds to amino acids 1 to 250 of SEQ ID NO:32. The coding region without the 5′ and 3′ untranslated regions of the human TRASH gene is shown in SEQ ID NO:33 and is depicted as triplets underneath the corresponding encoded amino acid residues.

FIG. 18 depicts an alignment of the amino acid sequences of human TRASH I (corresponding to amino acids 1 to 250 of SEQ ID NO:32), human TNF-α (Swiss-Prot™ Accession No. P01375; SEQ ID NO:43), and human Tweak (Swiss-Prot™ Accession No. AF030099; SEQ ID NO:44).

FIGS. 19A-19B depict the cDNA sequence and predicted amino acid sequence of human BDSF-1. The nucleotide sequence corresponds to nucleic acids 1 to 1119 of SEQ ID NO:45. The amino acid sequence corresponds to amino acids 1 to 244 of SEQ ID NO:46. The coding region without the 5′ and 3′ untranslated regions of the human BDSF-1 gene is shown in SEQ ID NO:47 and is depicted as triplets above the corresponding encoded amino acid residues.

FIGS. 20A-20C depicts the cDNA sequence and predicted amino acid sequence of murine BDSF-1. The nucleotide sequence corresponds to nucleic acids 1 to 3196 of SEQ ID NO:50. The amino acid sequence corresponds to amino acids 1 to 251 of SEQ ID NO:51. The coding region without the 5′ and 3′ untranslated regions of the murine BDSF-1 gene is shown in SEQ ID NO:52 and is depicted as triplets above the corresponding encoded amino acid residues.

FIG. 21 depicts an alignment of the amino acid sequence of human BDSF-1, corresponding to SEQ ID NO:46, with the amino acid sequence of murine BDSF-1, corresponding to SEQ ID NO:51. The immunoglobulin-like domains are underlined. The conserved cysteine residues of the immunoglobulin-like domain of human and murine BDSF are indicated with an asterisk. Identical amino acid residues are shown with colons (:). Similar amino acid residues are shown with periods (.). The alignment was performed using the ALIGN program with a PAM120 weight residue table, a gap length penalty of 12, and a gap penalty of 4.

FIGS. 22A-22E depict the cDNA sequence and predicted amino acid sequence of human LRSG-1. The nucleotide sequence corresponds to nucleic acids 1 to 2852 of SEQ ID NO:58 The amino acid sequence corresponds to amino acids 1 to 673 of SEQ ID NO:59. The coding region without the 5′ and 3′ untranslated regions of the human LRSG-1 gene is shown in SEQ ID NO:60 and is depicted as triplets below the corresponding encoded amino acid residues.

FIGS. 23A-23B depict an alignment of the amino acid sequence of human LRSG-1 (corresponding to SEQ ID NO:59° with the amino acid sequences of platelet glycoprotein V precursor (GPV) (SwisProt Accession No. P40197), corresponding to SEQ ID NO:61 and insulin-like growth factor binding protein complex acid labile chain precursor (ALS) (SwisProt Accession No. 002833), corresponding to SEQ ID NO:62. The leucine-rich repeat regions are indicated in italics. The EGF-like domain of LRSG-1 is underlined. The Fn type III-like domain of LRSG-1 is indicated in bold. The conserved cysteine residues of the EGF-like domain of LRSG-1 are indicated with an asterisk.

FIGS. 24A-24E depicts the cDNA sequence and predicted amino acid sequence of murine LRSG-1. The nucleotide sequence corresponds to nucleic acids 1 to 2815 of SEQ ID NO:67. The amino acid sequence corresponds to amino acids 1 to 673 of SEQ ID NO:68. The coding region without the 5′ and 3′ untranslated regions of the murine LRSG-1 gene is shown in SEQ ID NO:69 and is depicted as triplets below the corresponding encoded amino acid residues.

FIGS. 25A-25C depict an alignment of the amino acid sequence of human LRSG-1 (corresponding to SEQ ID NO:59) with the amino acid sequences of murine LRSG-1 (corresponding to SEQ ID NO:68). Identical amino acid residues are shown with colons (:). Similar amino acid residues are shown with periods (.). The alignment was generated using the ALIGN algorithm, version 2, which is part the GCG software package. The alignment was generated using a PAM120 scoring matrix and gap penalties of −12/−4.

FIGS. 26A-26C depict an alignment of the amino acid sequence of human LRSG-1 (corresponding to SEQ ID NO:59) with the amino acid sequences of murine LRSG-1 (corresponding to SEQ ID NO:68), platelet glycoprotein V precursor (GPV) (SwisProt Accession No. P40197), corresponding to SEQ ID NO:61, and insulin-like growth factor binding protein complex acid labile chain precursor (ALS) (SwisProt Accession No. 002833), corresponding to SEQ ID NO:62. The leucine-rich repeat regions are indicated in italics. The EGF-like domain of LRSG-1 is underlined. The Fn type III-like domain of LRSG-1 is indicated in bold. The conserved cysteine residues of the EGF-like domain of LRSG-1 are indicated with an asterisk.

FIGS. 27A-27B depict the cDNA sequence and predicted amino acid sequence of human STMST-1. The nucleotide sequence corresponds to nucleic acids 1 to 2915 of SEQ ID NO:70. The amino acid sequence corresponds to amino acids 1 to 297 of SEQ ID NO:71. The coding region without the 5′ and 3′ untranslated regions of the human STMST-1 gene is shown in SEQ ID NO:72 and is depicted as triplets below the corresponding encoded amino acid residues.

FIGS. 28A-28C depict the cDNA sequence and predicted amino acid sequence of human STMST-2. The nucleotide sequence corresponds to nucleic acids 1 to 4166 of SEQ ID NO:73. The amino acid sequence corresponds to amino acids 1 to 609 of SEQ ID NO:74. The coding region without the 5′ and 3′ untranslated regions of the human STMST-2 gene is shown in SEQ ID NO:75 and is depicted as triplets below the corresponding encoded amino acid residues.

FIGS. 29A-29B depict an alignment of the amino acid sequences of human STMST-1 (SEQ ID NO:71), human STMST-2 (SEQ ID NO:74), human protein A-2 (Accession No. U47928, SEQ ID NO:78), and human protein A-3 (Accession No. U47929, SEQ ID NO:79). The 7 transmembrane receptor profile is indicated in italics. The transmembrane domains are underlined. The spectrin α-chain profile is indicated in bold.

FIGS. 30A-30B are graphic representations of relative STMST expression levels as determined by TaqMan™ RT-PCR of mRNA samples from various cells including osteoblast cells lines and primary osteoblasts treated as indicated. FIG. 30C is a graphic representation of STMST expression levels as determined by transcription profiling analysis using a cDNA array.

FIG. 31 depicts relative mRNA expression levels of GPCR 13467 as determined by microarray hybridization. Human umbilical vein endothelial cells (HUVECs) were grown under a variety of conditions. Lanes 2-4: HUVECs cultured on tissue culture plastic; lane 2: without added growth factors; lane 3: with IL-1β; lane 4: with VEGF. Lanes 5-7: HUVECs cultured on Matrigel, which induces vascular-like tube formation; lane 5: 2 hours after plating on Matrigel—early stage of active tube formation; lane 6: 6 hours after plating on Matrigel—active tube formation; lane 7: 16 hours after plating on Matrigel—late stage of active tube formation.

### DETAILED DESCRIPTION OF THE INVENTION

The present invention is based, at least in part, on the discovery of a variety of cDNA molecules which encode proteins which are herein designated “Interleukin Ten Associated Locus Yang” or “ITALY”, “Lysyl Oxidase Related-2” or “Lor-2”, “STRIFE” (e.g., “STRIFE1” and “STRIFE2”), “TRASH”, “Brain-Derived Signaling Factor” or “BDSF” (e.g., “BDSF-1”), “Leucine-rich Surface Glycoprotein” or “LRSG” (e.g., “LRSG-1”), and “Seven Transmembrane Signal Transducer” or “STMST”. These proteins exhibit a variety of physiological activities, and are included in a single application for the sake of convenience. It is understood that the allowability or non-allowability of claims directed to one of these proteins has no bearing on the allowability of claims directed to the others. The characteristics of each of these proteins and the cDNAs encoding them are described separately in the ensuing sections. In addition to the full length mature and immature human proteins described in the following sections, the invention includes fragments, derivatives, and variants of these proteins, as described herein. These proteins, fragments, derivatives, and variants are collectively referred to herein as polypeptides of the invention or proteins of the invention.

The term “family” when referring to the protein and nucleic acid molecules of the invention is intended to mean two or more proteins or nucleic acid molecules having a common structural domain or motif and having sufficient amino acid or nucleotide sequence homology as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics.

One embodiment of the invention features ITALY, Lor-2, STRIFE, TRASH, BDSF, LRSG, and STMST nucleic acid and protein molecules, preferably human ITALY, Lor-2, STRIFE, TRASH, BDSF, LRSG, and STMST nucleic acid and protein molecules. The ITALY, Lor-2, STRIFE, TRASH, BDSF, LRSG, and STMST nucleic acid and protein molecules of the invention are described in further detail in the following subsections.

A. ITALY Nucleic Acid and Protein Molecules

The human ITALY cDNA, which is approximately 991 nucleotides in length (shown in SEQ ID NO:1), encodes a protein which is approximately 177 amino acid residues in length (shown in SEQ ID NO:2). The human ITALY protein has at least a disulfide forming cysteine residue. A disulfide forming cysteine residue, for example, is found at amino acids 5, 75, 120, and 127 of SEQ ID NO:2. The human ITALY protein further has at least an α-helical structure. The human ITALY protein is predicted to be a secreted protein which further contains a signal sequence at about amino acids 1-24 of SEQ ID NO:2. The prediction of such a signal peptide can be made, for example, utilizing the computer algorithm SIGNALP (Henrik, et al. (1997) Protein Eng. 10:1-6).

In one embodiment, the isolated proteins of the present invention, preferably ITALY proteins, are proteins having an amino acid sequence of about 130-210, preferably about 140-200, more preferably about 150-190, more preferably about 160-180, and even more preferably about 170-180 amino acid residues in length, of which at least about 4-10, preferably 4-8, and more preferably 4-6 amino acids are cysteine residues, which are preferably conserved between family members and are capable of forming disulfide bonds. For example, Cys5, Cys75, Cys120, and Cys127 of SEQ ID NO:2 are capable of forming disulfide bonds. Such residues are highly conserved among IL-10 family members.

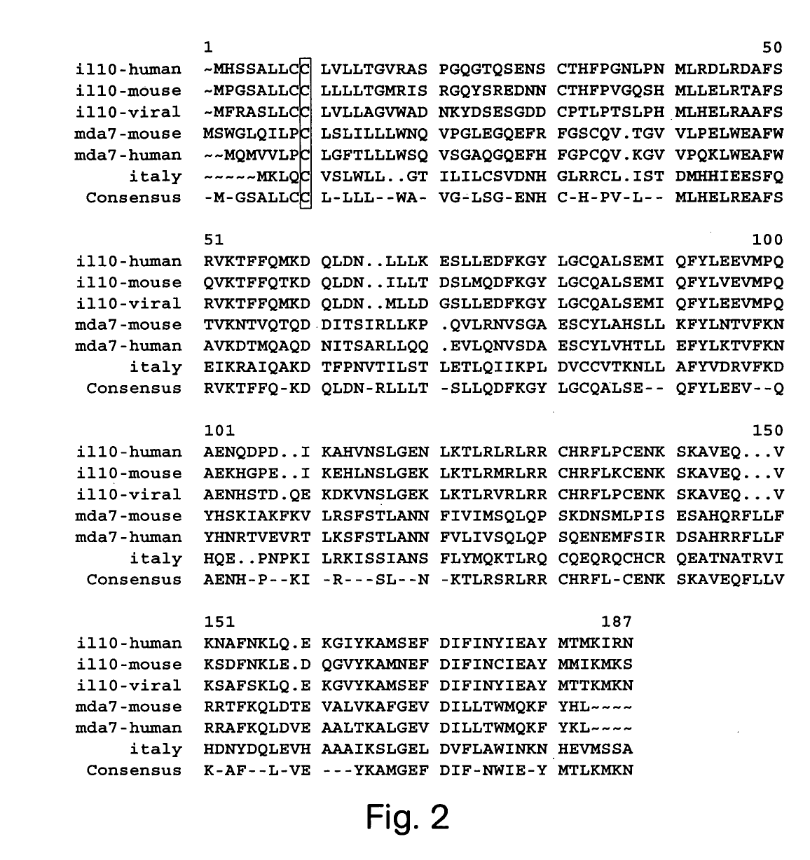

In one embodiment, the isolated proteins of the present invention, preferably ITALY proteins, are identified based on the presence of at least two, three or four cysteine residues in the protein or corresponding nucleic acid molecule. Preferably, the IL-10 family member has cysteine residues which are located in the same or similar positions as cysteine residues in an IL-10 protein family member. For example, when a human ITALY protein of the invention is aligned with the human IL-10, the mouse IL-10, the viral IL-10, the mouse MDA-7, and the human MDA-7 for purposes of comparison (see, e.g., FIG. 2) preferred cysteine residues of the invention are those in which cysteine residues in the amino acid sequence of ITALY are located in the same or similar position as the cysteine residues in other IL-10 family members. As an illustrative embodiment, FIG. 2 shows cysteine residues located in the same or similar positions of the human ITALY protein (corresponding to SEQ ID NO:2) and human IL-10 (GenBank Accession No. NP_{—}000563; SEQ ID NO:5), mouse IL-10 (GenBank Accession No. AAA39275; SEQ ID NO:6), viral IL-10 (corresponding to SEQ ID NO:7), mouse MDA-7 (corresponding to SEQ ID NO:8), and human MDA-7 (corresponding to SEQ ID NO:9) in the following locations: amino acid number 5 of human ITALY and the corresponding amino acid in the other proteins; amino acid number 75 of human ITALY and the corresponding amino acid in the other proteins; amino acid number 120 of human ITALY and the corresponding amino acid in the other proteins; and amino acid number 127 of human ITALY and the corresponding amino acid in the other proteins.

In another embodiment, an ITALY family member is identified based on the presence of an “α-helical structure” in the protein molecule. As used herein, the term “α-helical structure” refers to the secondary structure of a protein. An α-helical structure is generated when a single polypeptide chain turns regularly about itself to make a rigid cylinder in which each peptide bond is regularly hydrogen bonded to other peptide bonds elsewhere in the chain. The side chains of each amino acid protrude radially along the outside of the helix. Accordingly, in one embodiment, an ITALY protein is human ITALY having an α-helical structure.

In another embodiment, an ITALY family member is identified based on the presence of a “four-α-helical bundle”. As used herein, the term “four-α-helical bundle” refers to the tertiary structure formed when four α-helices fold in an antiparallel fashion to form a bundle. Hydrophilic loops usually separate the α-helices forming the four-α-helical bundle. The sequences of four-α-helical bundle proteins are characterized by a pattern of hydrophilic and hydrophobic amino acids which is repeated every seven residues. Accordingly, in one embodiment, an ITALY protein is human ITALY having four-α-helical bundle. These α-helical structures and four-α-helical bundles are described in, for example, Paliakasis, C. D. et al. (1992) Protein Eng. 5(8):739-48, and Manavalan, P. et al. (1992) J. Prot. Chem. 11 (3): 321-31, the contents of which are incorporated herein by reference.

In another embodiment of the invention, an ITALY protein has at least one disulfide bond forming cysteine residue, and/or at least one α-helical structure, and, preferably, a signal sequence. In another embodiment, an ITALY protein has at least one and preferably 4-6 disulfide bond forming cysteine residues and an α-helical structure, and, preferably a signal sequence. As used herein, a “signal sequence” refers to a peptide of about 20-30 amino acid residues in length which occurs at the N-terminus of secretory and integral membrane proteins and which contains a majority of hydrophobic amino acid residues. For example, a signal sequence contains at least about 15-45 amino acid residues, preferably about 20-40 amino acid residues, more preferably about 25-35 amino acid residues, and more preferably about 28-32 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, an ITALY protein contains a signal sequence of about amino acids 1-24 of SEQ ID NO:2. The “signal sequence” is cleaved during processing of the mature protein. The mature ITALY protein corresponds to amino acids 25 to 177 of SEQ ID NO:3, shown separately as SEQ ID NO:4.

Isolated proteins of the present invention, preferably ITALY proteins, have an amino acid sequence sufficiently homologous, as defined herein, to the amino acid sequence of SEQ ID NO:2 or are encoded by a nucleotide sequence sufficiently homologous to SEQ ID NO:1 or SEQ ID NO:3.

As described in the Examples section the human ITALY protein having a nucleotide and amino acid sequence set forth in SEQ ID NO:1 and SEQ ID NO:2, respectively, is homologous to IL-10 family members such as human, mouse, and viral IL-10 and human and mouse MDA-7. IL-10 is a cytokine which acts on a variety of cells within the immune system. Its functions include the inhibition of surface expression of MHC class II molecules on a variety of antigen presenting cells (APC), the regulation of co-stimulatory pathways such as APC surface expression of intracellular adhesion molecules, and the inhibition of T cell proliferation via inhibition of IL-2 production as well as IL-5 secretion by T cells (e.g., CD80-86/CD28 dependent IL-5 secretion). IL-10 also enhances immune activity by, for example, stimulating proliferation, activation, and chemotaxis of CD28+ T cells, upregulation of Fc receptors on monocytes, thus enhancing antibody dependent cytotoxicity, and increasing B cell proliferation, differentiation, and antibody production. As a result of the above described functions, IL-10 is involved in immune and inflammation disorders such as rheumatoid arthritis, systemic lupus erythematosus, myasthenia gravis, Grave's disease, Sjogren's Syndrome, polymyositis and dermatomyositis, psoriasis, pemphigus vulgaris, bullous pemphigoid, inflammatory bowel disease, Kawasaki disease, asthma, and graft versus host disease.

The MDA-7 gene was cloned from a human melanoma library and encodes a 206 amino acid protein. MDA-7 is induced as a function of growth arrest and induction of terminal differentiation in human melanoma cells (as described in, for example, Jiang H. et al. (1994) Mol. Cell. Differ. 2:221-239). MDA-7 expression also inversely correlates with melanoma progression. Moreover, MDA-7 is growth inhibitory toward human melanoma cells. In addition to its role in melanoma, MDA-7 has been shown to be a potent growth suppressing factor in cancer cells of diverse origin including breast, central nervous system, cervix, colon, prostate, and connective tissue. As a result of the above described functions, MDA-7 is involved in the modulation of cell growth in a variety of proliferative disorders, e.g., cancer such as melanoma, prostate cancer, cervical cancer, breast cancer, colon cancer, or sarcoma.

As used interchangeably herein an “ITALY activity”, “biological activity of ITALY” or “functional activity of ITALY”, refer to an activity exerted by an ITALY protein, polypeptide or nucleic acid molecule on an ITALY responsive cell as determined in vivo, or in vitro, according to standard techniques. In one embodiment, an ITALY activity is a direct activity, such as an association with a cell-surface protein (e.g., an ITALY receptor). In another embodiment, an ITALY activity is an indirect activity, such as the induction of synthesis of a second protein (e.g., a cellular cytokine) mediated by interaction of the ITALY protein with a cell surface protein.

Accordingly, another embodiment of the invention features isolated ITALY proteins and polypeptides having an ITALY activity. Preferred proteins are ITALY proteins having at least one disulfide forming cysteine residue and, preferably, an ITALY activity. Additional preferred proteins are ITALY proteins having at least an α-helical structure and, preferably, an ITALY activity. In another preferred embodiment, the isolated protein further comprises a signal sequence. In still another preferred embodiment, the isolated protein is an ITALY protein having at least one disulfide forming cysteine residue, an α-helical structure, an ITALY activity, and, preferably, an amino acid sequence sufficiently homologous, as defined herein, to an amino acid sequence of SEQ ID NO:2, and, optionally, a signal sequence.

Accordingly, another embodiment of the invention features isolated ITALY proteins and polypeptides having an ITALY activity. Preferred proteins are ITALY proteins having at least one disulfide forming cysteine residue and, preferably, an ITALY activity. Additional preferred proteins are ITALY proteins having at least an α-helical structure and, preferably, an ITALY activity. In another preferred embodiment, the isolated protein further comprises a signal sequence. In still another preferred embodiment, the isolated protein is an ITALY protein having at least one disulfide forming cysteine residue, an α-helical structure, an ITALY activity, and, preferably, an amino acid sequence sufficiently homologous, as defined herein, to an amino acid sequence of SEQ ID NO:2, and, optionally, a signal sequence.

Accordingly, as members of the IL-10 family of molecules, the ITALY protein and nucleic acid molecules of the invention have, preferably, at least one or more of the following activities: (i) interaction of an ITALY protein in the extracellular milieu with a non-ITALY protein molecule on the surface of the same cell which secreted the ITALY protein molecule; (ii) interaction of an ITALY protein in the extracellular milieu with a non-ITALY protein molecule on the surface of a different cell from that which secreted the ITALY protein molecule; (iii) complex formation between an ITALY protein and an ITALY receptor; (iv) complex formation between an ITALY protein and non-ITALY receptor; and (v) interaction of an ITALY protein with a second protein in the extracellular milieu. In yet another preferred embodiment, an ITALY activity is at least one or more of the following activities: (i) activation of an ITALY-dependent signal transduction pathway; (ii) modulation of secretion of a proinflammatory cytokine e.g., IL-1α, IL-1β, IL-6, or TNF-α; (iii) modulation of secretion of a chemokine, e.g., IL-8 or macrophage inflammatory protein; (iv) modulation of surface expression of MHC molecules, e.g., MHC class II molecules, or cellular adhesion molecules, e.g., ICAM1; (v) modulation of inflammatory activity of a cell, e.g., a T cell; (vi) modulation of immune activity of a cell, e.g., a T cell (e.g., T cell proliferation, IL-2 secretion, or IL-5 secretion) or a B cell (e.g., B cell proliferation, differentiation, or antibody production); and (vii) modulation of proliferation and/or differentiation of a cell, e.g., a B cell, a T cell, a central nervous system cell, a prostate cell, a cervical cell, a breast cell, a colon cell, or an epithelial cell.

B. Lor-2 Nucleic Acid and Protein Molecules

The human Lor-2 cDNA (SEQ ID NO:10), which is approximately 2920 nucleotides in length, encodes a protein having a molecular weight of approximately 83.166 kD (with signal sequence) and 80.404 kD (without signal sequence) and which is approximately 753 (with signal sequence) (SEQ ID NO:11) and 728 amino acid residues (without signal sequence) in length. Lor-2 nucleic acid and polypeptide molecules play a role in or function in a variety of cellular processes in the cardiovascular system, e.g., cardiac cell function. One embodiment of the invention features Lor-2 nucleic acid molecules, preferably human Lor-2 molecules, which were identified from a cDNA library made from the heart of a patient with congestive heart failure (CHF).

In another embodiment, the isolated proteins of the present invention, preferably Lor-2 proteins, can be identified based on the presence at least one SRCR domain and/or a lysyl oxidase domain and/or and a signal sequence.

In a preferred embodiment, a Lor-2 family member includes at least 1 or more scavenger receptor cysteine-rich (“SRCR”) domains. Scavenger receptors are proteins which have been implicated in the development of atherosclerosis and other macrophage-associated functions. For example, the type I mammalian macrophage scavenger receptors are membrane glycoproteins implicated in the pathologic deposition of cholesterol in arterial walls during atherogenesis (Freeman et al. (1990) Proc. Natl. Acad. Sci. U.S.A. 87:8810-8814). Scavenger receptors are characterized by the presence of a cysteine-rich domain, which is proposed to be involved in binding of physiological ligands (e.g., cell-surface proteins). This cysteine rich domain is referred to herein and in the art as a scavenger receptor cysteine-rich (“SRCR”) domains. Intra- or intercellular binding of ligand to the SRCR domain is believed to play a role in signaling or adhesion.

As defined herein, a SRCR domain includes a protein domain which is about 88-112 amino acid residues in length and has about 16-60% identity with a SRCR of type I human macrophage scavenger receptor (e.g., amino acid residues 353-450 of SEQ ID NO:19). In another embodiment, a SRCR is abuse 90-110, 92-108, 94-106, or 95, 96, 97, 98, 99, 100, 101, 102, 103, 104, 105, or 106 amino acid residues in length and has about 22-54%, 26-50%, 28-48%, or 29%, 30%, 31%, 32%, 33%, 34%, 35%, 36%, 37%, 38%, 39%, 40%, 41%, 42%, 43%, 44%, 45%, 46%, or 47% identity with a SRCR of type I human macrophage scavenger receptor (e.g., amino acid residues 353-450 of SEQ ID NO:19). For example, a SRCR domain can be found in murine type I scavenger receptor (Accession No. 1709140; SEQ ID NO:126) from about amino acid residues 360-457. SRCR domains also have been found in diverse secreted and other cell-surface proteins from humans (e.g., CD5 and complement factor I), mice (Ly-1), and sea urchins (speract receptor). Moreover, many proteins include more than one SRCR domain (e.g., Ly-1 includes 3 SRCR domains and the speract receptor includes 4 SRCR domains). Likewise, human Lor-2 includes 4 SRCR domains, as set forth below.

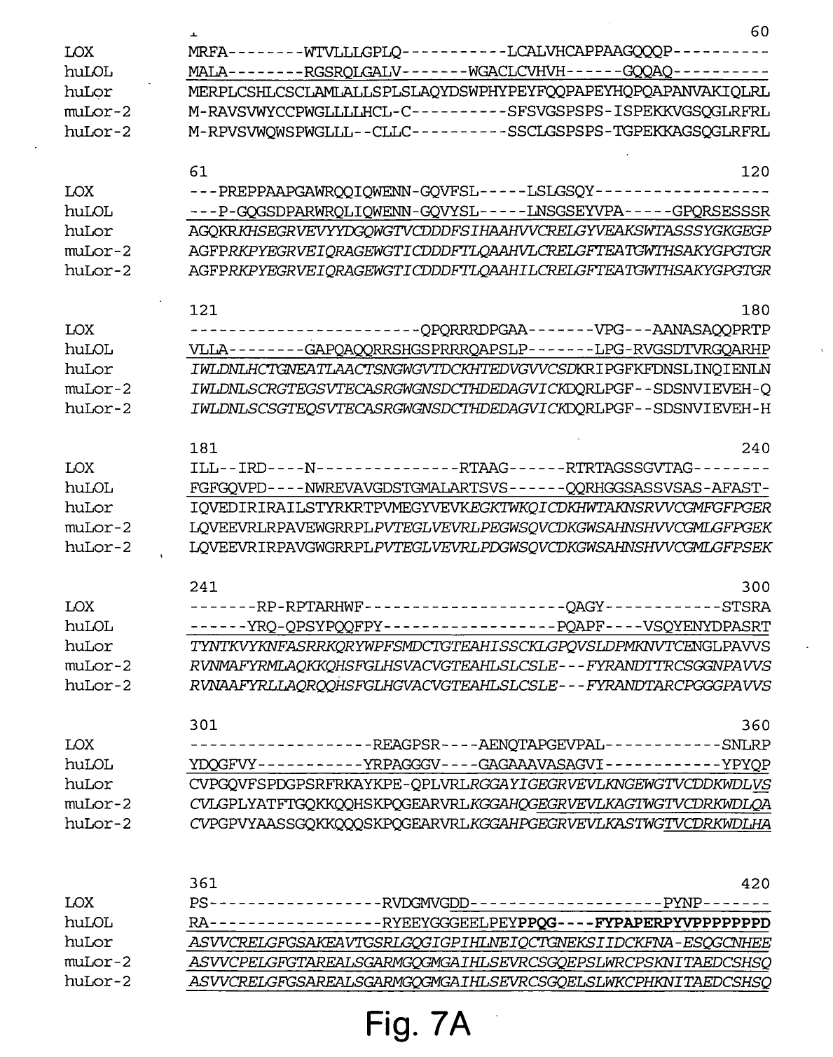

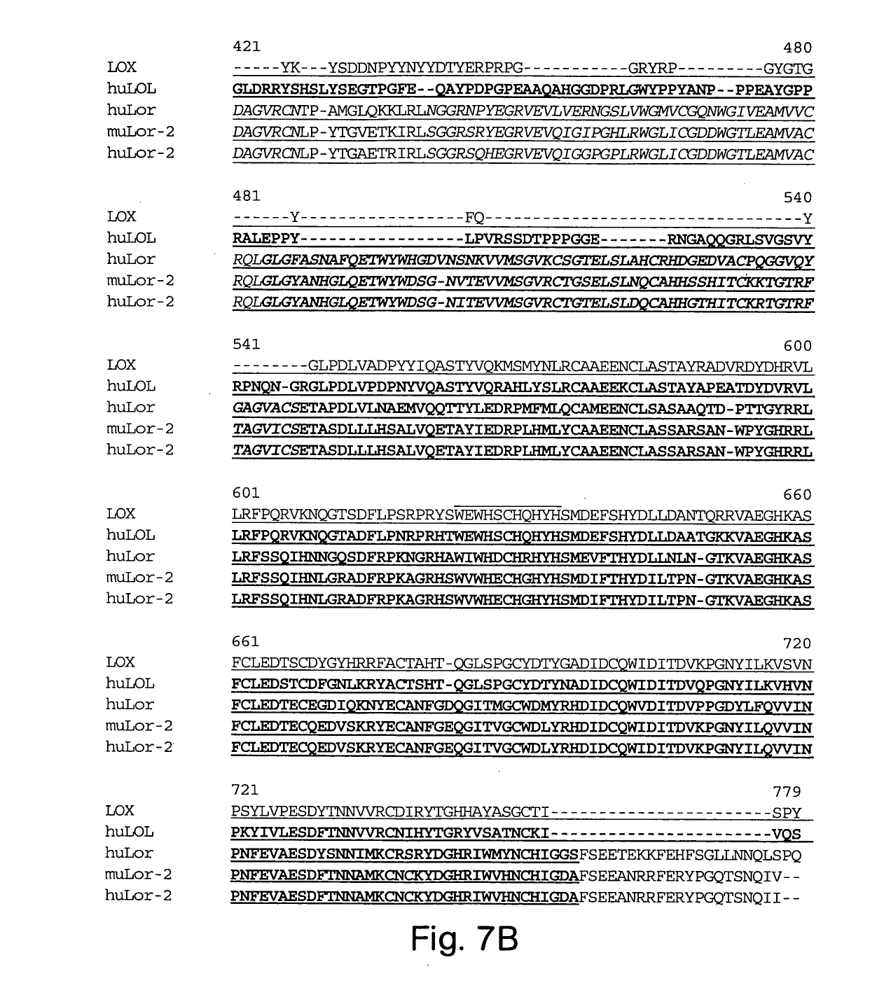

To identify the presence of an SRCR in a Lor-2 family member, the amino acid sequence of the protein family member can be searched against a database of HMMs (e.g., the Pfam database, release 3.3) e.g., using the default parameters. For example, the search can be performed using the hmmsf program (family specific) and threshold score of 15 for determining a hit. hmmsf is available as part of the HMMER package of search programs (HMMER 2.1.1, December 1998) which is freely distributed by the Washington University school of medicine. In one embodiment, a hit to a SRCR HMM having a score of at least 30-40, preferably at least 50-60, more preferably at least 70-80, and more preferably at least 90 or more is determinative of the presence of a SRCR domain within a query protein. A search using the amino acid sequence of SEQ ID NO:11 was performed against the HMM database resulting in the identification of 4 SRCR domains in the amino acid sequence of SEQ ID NO:11. Accordingly, in one embodiment of the invention, a Lor-2 protein has an SRCR domain at about amino acids 51-145 of SEQ ID NO:11. (Score of 91.4 against the SRCR domain profile HMM Accession No. PF00530). In another embodiment, a Lor-2 protein has an SRCR domain at about amino acids 183-282 of SEQ ID NO:11. (Score of 35.8). In another embodiment, a Lor-2 protein has an SRCR domain at about amino acids 310-407 of SEQ ID NO:11. (Score of 128.9). In another embodiment, a Lor-2 protein has an SRCR domain at about amino acids 420-525 of SEQ ID NO:11. (Score of 55.2). The SRCRs of Lor-2, as well as those of huLor and muLor-2 are indicated by italics in FIGS. 7A-7B.

Lor-2 family members can further include at least one or more speract receptor repeated domain (“SRRD”) signatures. The speract receptor is a transmembrane glycoprotein of 500 amino acid residues (Dangott et al. (1989) Proc. Natl. Acad. Sci. U.S.A. 86:2128-2132) which consists of a large extracellular domain of 450 which contains four repeats of a ˜115 amino acids termed more speract receptor repeated domain or “SRRDs”. Multiple sequence alignment of the four repeats reveals at least 17 perfectly conserved residues (including six cysteines, six glycines, and three glutamates). A SRRD signature has been generated from an alignment of the four SRRDs and has the consensus sequence:

(SEQ ID NO: 13)

G-x(5)-G-x(2)-E-x(6)-W-G-x(2)-C-x(3)-[FYW]-x(8)-
C-x(3)-G

wherein [FYW] indicates any one of phenylalanine, tyrosine, or tryptophan, and wherein x(5) indicates any 5 successive amino acid residues, and wherein single amino acid codes are the standard IUPAC one-letter codes for the amino acids. The SRRD signature is further described in PROSITE Document Accession No. PDOC00348 and as PROSITE Accession No. PS00420. In one embodiment, a SRRD signature is included within a SRCR. For example, a SRRD can be found in a SRCR of the C-terminal section of the mammalian macrophage scavenger receptor type I (Freeman et al. (1990) Proc. Natl. Acad. Sci. U.S.A. 87:8810-8814). Likewise, a SRRD signature can be found within the SRCR domain of human Lor-2 from about amino acids 312-349 of SEQ ID NO:11.

The consensus sequences herein are described according to standard Prosite Signature designation (e.g., all amino acids are indicated according to their universal single letter designation; X designates any amino acid; X(n) designates any n amino acids, e.g., X (2) designates any 2 amino acids; [FYW] indicates any one of the amino acids appearing within the brackets, e.g., any one of F, Y, or W, in the alternative, any one of Phe, Tyr, or Trp; and {×} indicates any amino but the amino acid included within the brackets.)

Lor-2 family members can further include at least one domain characteristic of lysyl oxidase, referred to herein as a lysyl oxidase domain or “LOX domain”. Lysyl oxidase is an extracellular copper-dependent enzyme that catalyzes the oxidative deamination of peptidyl lysine residues in precursors of various collagens and elastins. The deaminated lysines are then able to form aldehyde cross-links. (Krebs et al. (1993) Biochem. Biophys. Acta. 1202:7-12). The amino acid sequence of lysyl oxidase includes a signal sequence (e.g., amino acids 1 to 21 of human lysyl oxidase set forth as SEQ ID NO:14, a pro-peptide region (e.g., amino acids 22 to 168 of SEQ ID NO:14), and a region corresponding to the active, processed protein (e.g., amino acids 169-417 of SEQ ID NO:14), which is responsible for the enzymatic function of the molecule. Lysyl oxidase can be further characterized by the presence of a copper-binding site (Krebs et al. (1993) Biochem. Biophys. Acta. 12-2:7-12) having four conserved histidine residues that presumably supply the nitrogen ligands for copper coordination, and a quinone cofactor binding site (Wang et al. (1996) Science 273:1078-1084) (e.g., his289, his292, h is 294, and his296 of SEQ ID NO:14), also referred to as a “copper talon”. The copper binding site of human Lor-2 can be found, for example, at about amino acids 286-296 of SEQ ID NO:14.

Accordingly, as used herein, the term “LOX domain” includes a protein domain which is about 245-275 amino acid residues in length, and has about 38-64% identity with the amino acid sequence of processed lysyl oxidase (e.g., amino acid residues 169-417 of SEQ ID NO:14). Preferably, a LOX domain is about 225-300, more preferably about 230-290 amino acid residues in length, and more preferably about 235-285, or 240-280 amino acid residues in length, and has about 34-65% identity, preferably about 42-62%, and more preferably about 46-56% or 50-52% identity with the amino acid sequence of processed lysyl oxidase (e.g., amino acid residues 169-417 of SEQ ID NO:14). For example, a LOX domain can be found in huLOL (SEQ ID NO:15) from about amino acids 310-574; in huLor (SEQ ID NO:16) from about amino acids 481-751; in mu Lor-2 (SEQ ID NO:17) from about amino acids 464-733; and in huLor-2 (SEQ ID NO:11) from about amino acids 463-732. The LOX domains of huLOL, huLor, muLor-2, and huLor-2 are indicated in bold in FIGS. 7A-7B.

In another embodiment, a LOX domain is involved in a lysyl oxidase or lysyl oxidase-like function. Lysyl oxidase or lysyl oxidase-like functions include, for example, aminotransferase activity, peptidyl lysine oxidation, oxidative deamination of lysine, cross-linking of extracellular matrix components, copper binding, and/or copper metabolism. Lysyl oxidase or lysyl oxidase-like functions are described in detail, for example, in Kagan et al. in Catalytic Properties and structural components of lysyl oxidase, John Wiley & Sons (1995) pp. 100-121, the contents of which are incorporated herein by reference. In yet another embodiment, a LOX domain has at least one, preferably two, and more preferably three or four histidine residues corresponding to the conserved histidine residues of lysyl oxidase which are involved in copper binding. For example, a LOX domain of a human Lor-2 sequence set forth in SEQ ID NO:11 (e.g., amino acid residues 330-732 in SEQ ID NO:11) has four histidine residues (e.g., his604, his607, his609, and his611 of SEQ ID NO:11) which correspond to those of human lysyl oxidase set forth as SEQ ID NO:14.

A LOX domain in a protein can further be included within a lysyl oxidase-related region (“LOX-related region”). A LOX-related region within a protein (e.g., within a Lor-2 family member) includes a protein region which is about 380-580, preferably about 390-550, more preferably about 400, 420, 450 or 500 amino acid residues in length and has at least 30-35%, 40-45%, 50-55%, 60-65%, 70-75%, 80-85%, or 90-95% homology with, for example, the amino acid sequence of human LOX. To identify the presence of a LOX-related region in a Lor-2 family member, the amino acid sequence of the protein family member can be searched against the HMM database, as described previously. In one embodiment, a hit to a LOX HMM having a score of at least 100-110, preferably at least 120-130, more preferably at least 140-150, and more preferably at least 160 or more is determinative of the presence of a LOX-related region within a query protein. A search using the amino acid sequence of SEQ ID NO:2 was performed against the HMM database resulting a hit to a LOX HMM from about amino acids 330-732 of SEQ ID NO:11. (Score of 166.6 against the LOX domain profile HMM Accession No. PF01186). Similar LOX-related regions were identified in muLor-2 from about amino acids 318-733 of SEQ ID NO:17 (Score of 162.8), in huLOL from about amino acids 1-574 of SEQ ID NO:15 (Score of 382.2) and in huLor from about amino acids 358-751 of SEQ ID NO:16 (Score of 146.8). In yet another embodiment, a lysyl oxidase-related region has at least 40-45%, 50-55%, 60-65%, 70-75%, 80-85%, or 90-95% homology with the amino acid sequence of a LOX domain of a human Lor-2 sequence set forth in SEQ ID NO:11 (e.g., amino acid residues 330-732 in SEQ ID NO:11). The lysyl oxidase-related regions of huLOL, huLor, muLor-2 and huLor-2 are underlined in FIGS. 7A-7B, as are the amino acids corresponding to processed lysyl oxidase (e.g., amino acids 169-417 of SEQ ID NO:14).

Another embodiment of the invention features a protein of the invention, preferably a Lor-2 protein, which contains a signal sequence, as defined herein. For example, in one embodiment, a Lor-2 protein contains a signal sequence containing about amino acids 1-25 of SEQ ID NO:11.

In yet another embodiment, a protein of the invention, preferably a Lor-2 protein, encodes a mature protein. As used herein, the term “mature protein” refers to a protein of the invention, preferably a Lor-2 protein, from which the signal peptide has been cleaved. In an exemplary embodiment, a mature Lor-2 protein contains amino acid residues 26 to 753 of SEQ ID NO:11.

In yet another embodiment, Lor-2 family members include at least 1 or more N-glycosylation sites. Predicted N-glycosylation sites are found, for example, from about amino acid 111-114, 266-269, 390-393, 481-484, and 625-628 of SEQ ID NO:11. Lor-2 family members can further include at least 1 or more or more Protein kinase C (“PKC”) phosphorylation sites. Predicted PKC phosphorylation sites are found, for example, from about amino acid 97-99, 104-106, 221-223, 268-270, 352-354, 510-512, 564-566, and 649-651 of SEQ ID NO:11. Lor-2 family members can further include at least 1 or more Casein kinase II phosphorylation sites. Predicted casein kinase II phosphorylation sites are found, for example, from about amino acid 31-34, 68-71, 115-118, 120-123, 135-138, 330-333, 352-355, 377-380, 392-395, 411-414, 424-427, 493-496, 527-530, and 617-620 of SEQ ID NO:11. Lor-2 family members can further include at least 1 or more N-myristoylation sites. Predicted N-myristoylation sites are found, for example, from about amino acids 13-18, 116-121, 130-135, 273-278, 312-317, 359-364, 378-383, 403-408, 443-448, 451-456, 463-468, 470-475, 489-494, 506-511, 515-520, 521-526, 626-631, 661-666, and 746-751 of SEQ ID NO:11.

Lor-2 family members can further include at least one or more amidation sites. A predicted amidation site is found, for example, from amino acid 117-180 of SEQ ID NO:11. As used herein, the site(s) have a consensus sequence selected from:

| | |
|---|---|
| N-{P}-[ST]-{P} | (SEQ ID NO: 127) |

where N is a glycosylation site (see PROSITE document PS00001);

[ST]-X-[RK]

where S or T is a phosphorylation site (see PROSITE document PS00005);

[ST]-X (2)-[DE]

where S or T is a phosphorylation site (see PROSITE document PS00006);

| | |
|---|---|
| G-{EDRKHPFYW}-X(2)-[STAGCNH]-{P} | (SEQ ID NO: 176) |

where G is an N-myristoylation site (see PROSITE Accession No. PS00008); and

X-G-[RK]-[RK]

where X is an amidation site (see PROSITE document PS00009). These sites are further described in PROSITE documents PDOC00005, PDOC00006, PDOC00008, and PS00009, respectively.

Isolated proteins of the present invention, preferably Lor-2 proteins, have an amino acid sequence sufficiently homologous, as defined herein, to the amino acid sequence of SEQ ID NO:11 or are encoded by a nucleotide sequence which includes a nucleotide sequence sufficiently homologous to SEQ ID NO:10.

Accordingly, another embodiment of the invention features isolated Lor-2 proteins and polypeptides having a Lor-2 activity. Preferred proteins are Lor-2 proteins having at least a signal sequence, a LOX domain, and at least one SRRD signature. Other preferred proteins are Lor-2 proteins having at least two, three, or four SRRD signatures. Other preferred proteins are Lor-2 proteins having at least a signal sequence, a LOX domain, and a SRCR domain. Other preferred proteins are Lor-2 proteins having at least a signal sequence, a LOX domain, and at least two SCRC domains. Other preferred proteins are Lor-2 proteins having at least a signal sequence, a LOX domain, and at least three SCRC domains. Other preferred proteins are Lor-2 proteins having at least a signal sequence, a LOX domain, and at least four SCRC domains.

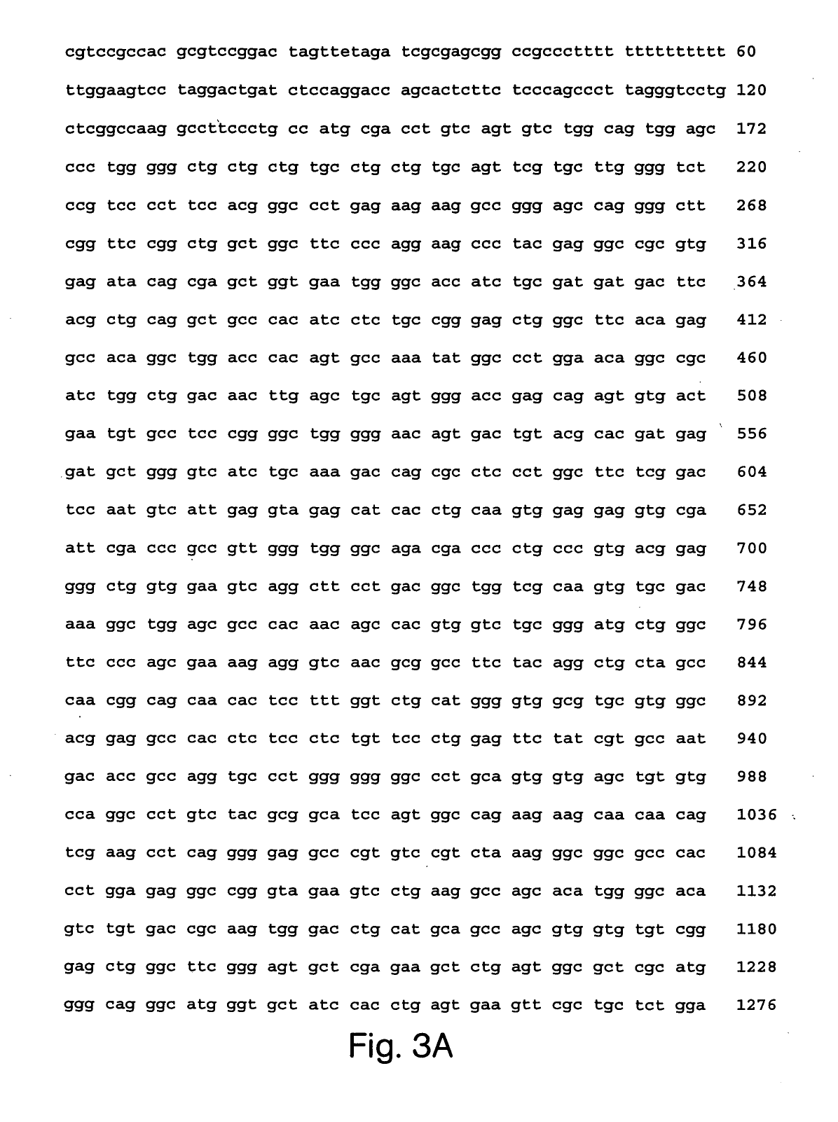

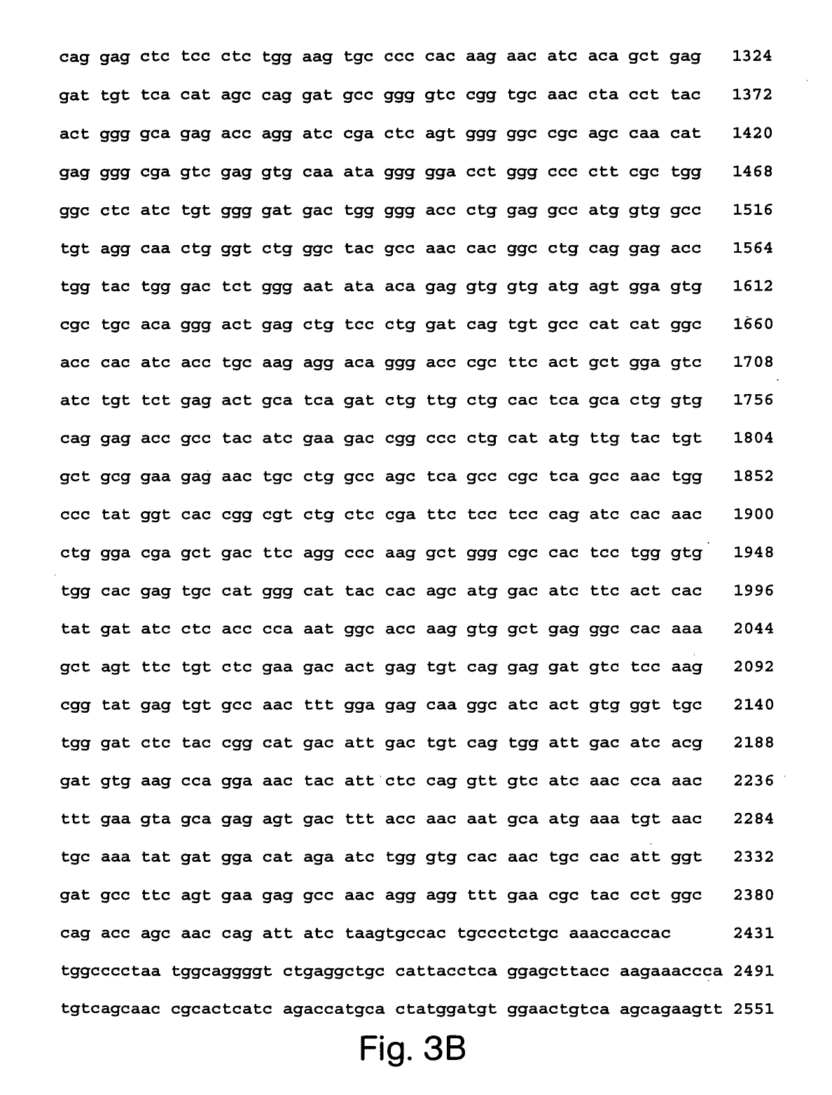

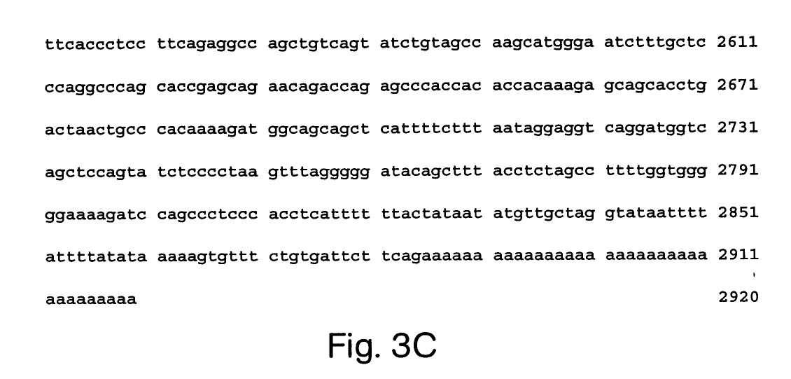

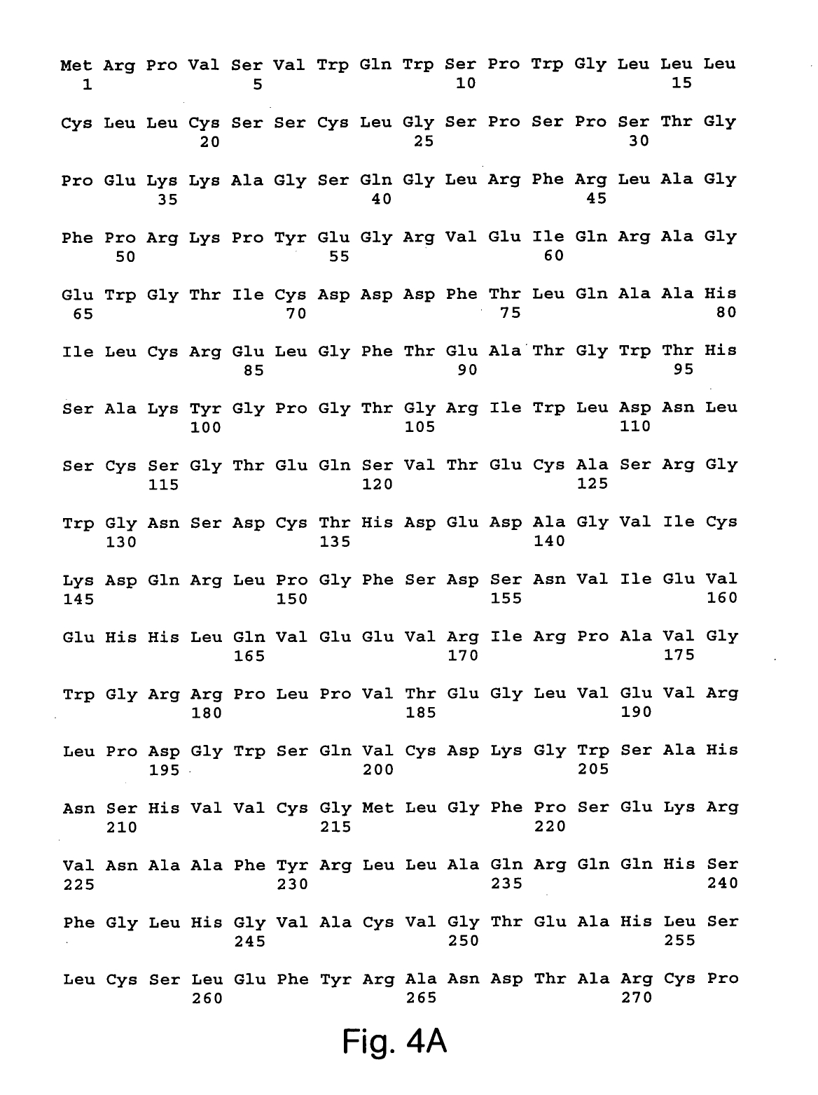

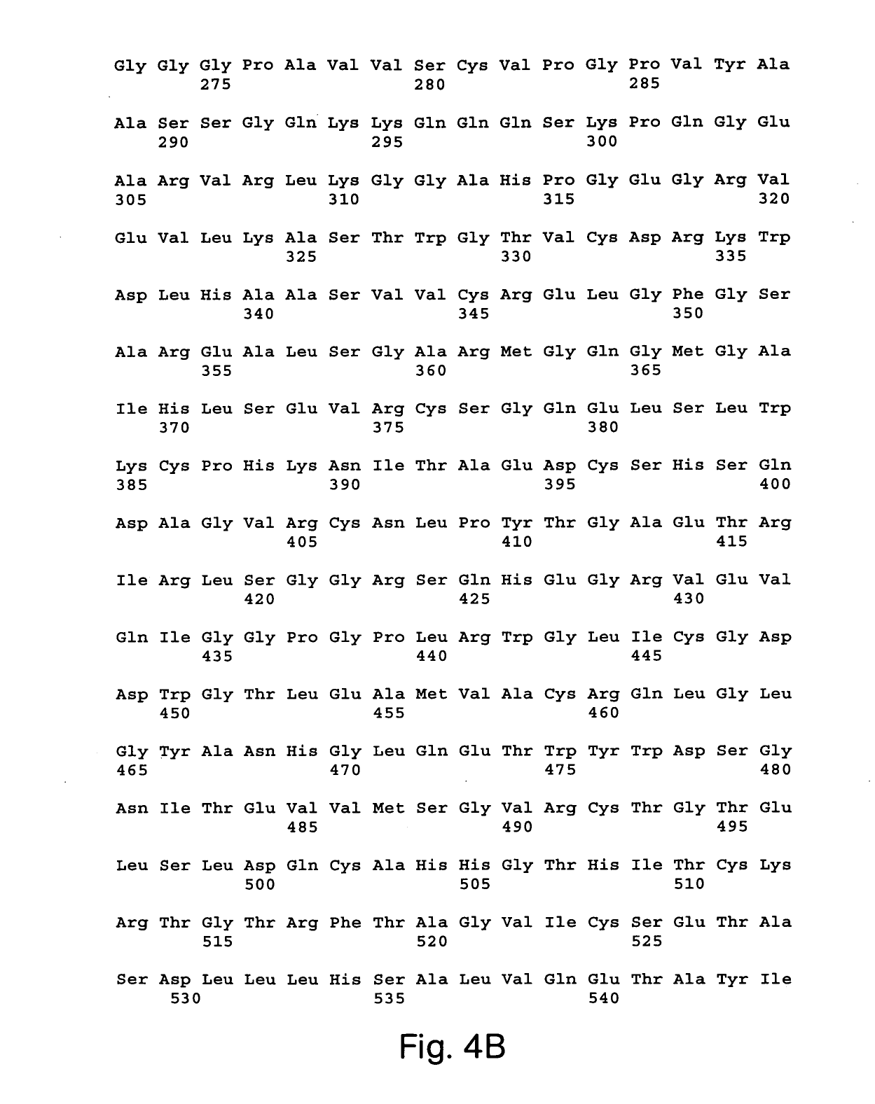

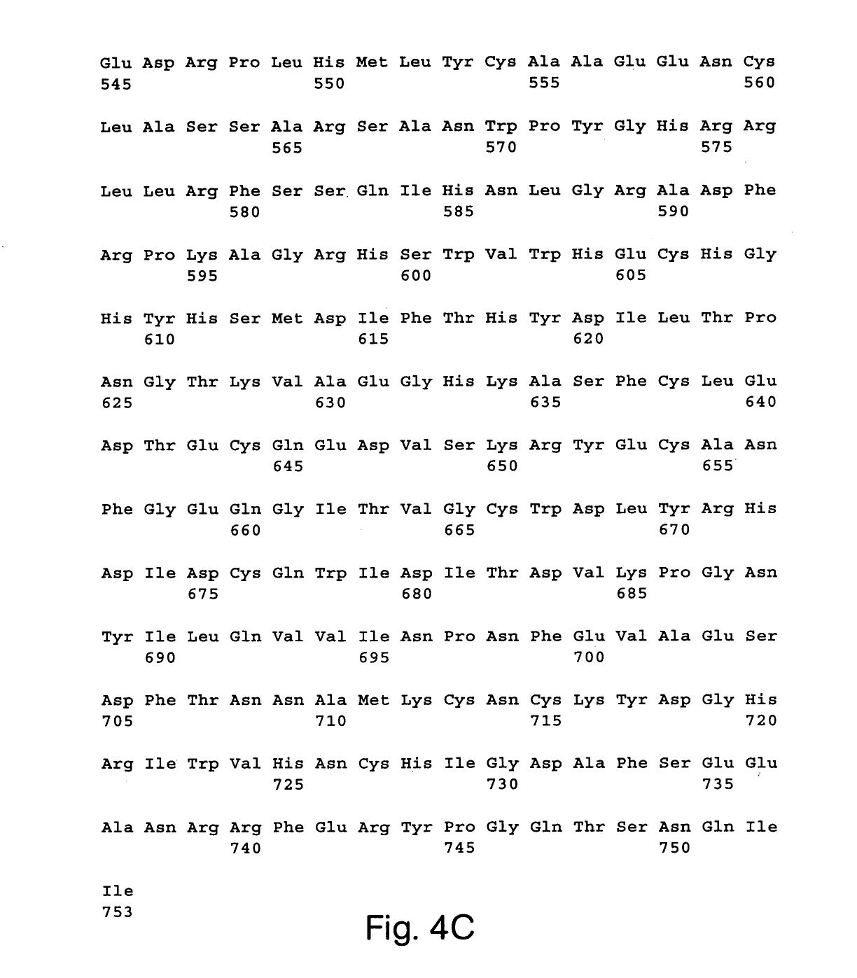

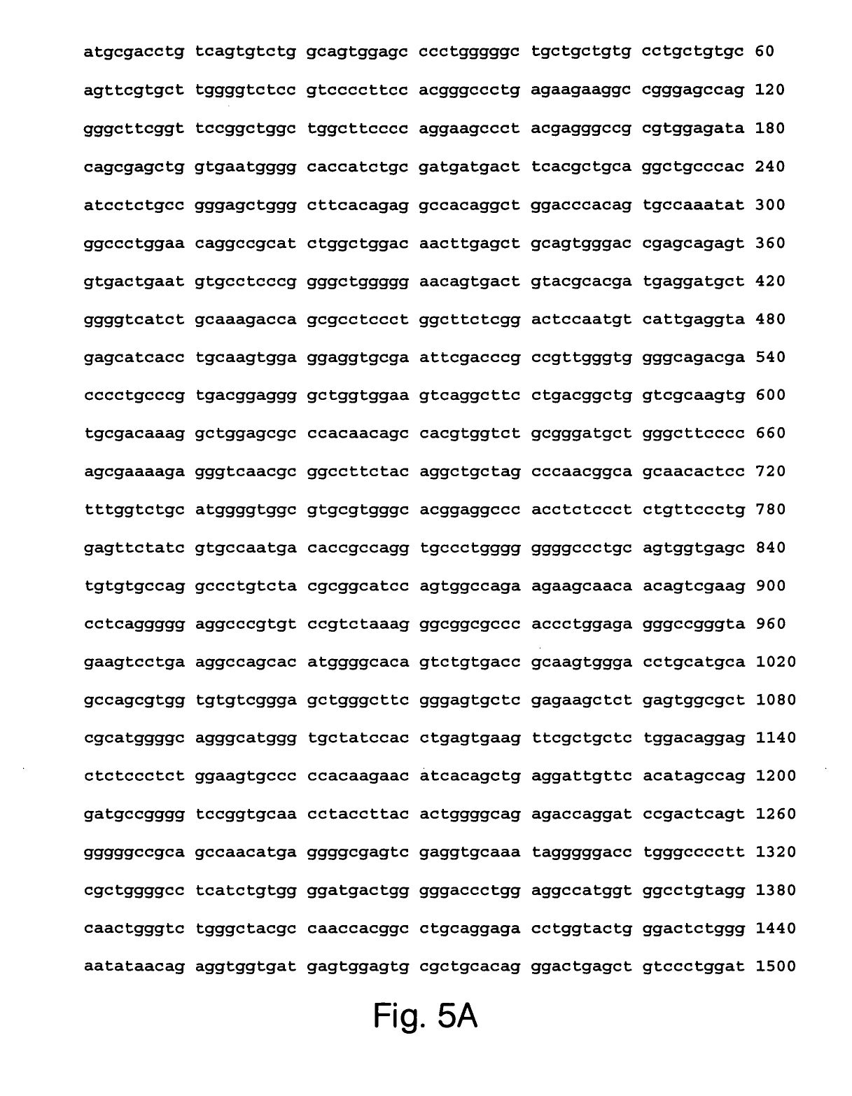

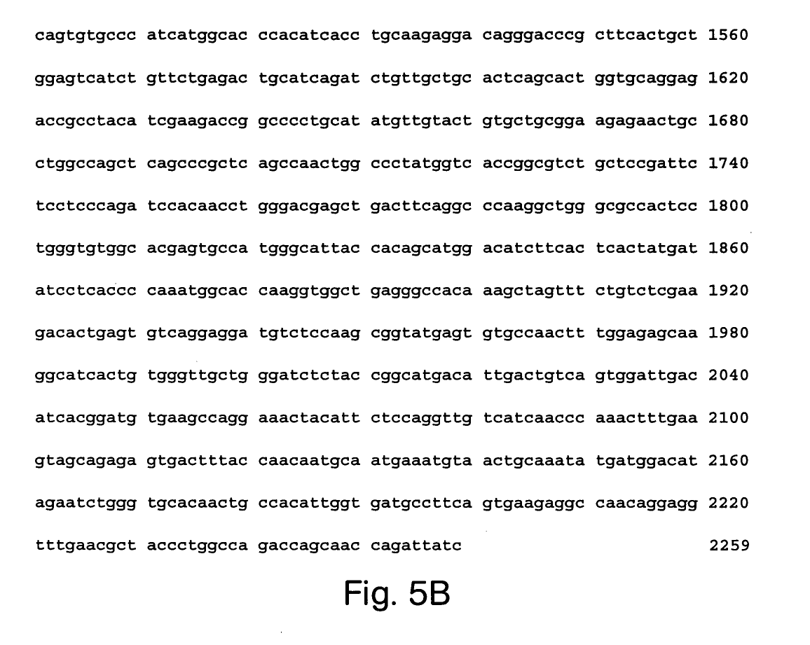

The nucleotide sequence of the isolated human Lor-2 cDNA and the predicted amino acid sequence of the human Lor-2 polypeptide are shown in FIGS. 3A-3C and 4A-4C (SEQ ID NOs:10 and 11), respectively.

An ˜3.0 kb Lor-2 message was found to be expressed most tissues tested but was most highly expressed in heart and placenta (at least heart, brain, placenta, lung, liver, skeletal muscle, kidney, and pancreas tissues were tested). High expression of Lor-2 was also observed in the G361 melanoma cell line and in the SW480 adenocarcinoma colon cell line (at least G361, SW480, HL60, Hela 53, K562, Molty, Raji, and A549 cell lines were tested).

In a preferred embodiment, Lor-2 proteins of the invention have an amino acid sequence of at least 600-900, preferably about 650-850, more preferably about 700-800, and even more preferably about 720-760, 728 or 753 amino acid residues in length.

As used interchangeably herein, a “Lor-2 activity”, “biological activity of Lor-2” or “functional activity of Lor-2”, include an activity exerted by a Lor-2 protein, polypeptide or nucleic acid molecule as determined in vivo, in vitro, or in situ, according to standard techniques. In one embodiment, a Lor-2 activity is a direct activity, such as an association with a Lor-2-target molecule. As used herein, a “target molecule” is a molecule with which a Lor-2 protein binds or interacts in nature, such that Lor-2-mediated function is achieved. A Lor-2 target molecule can be a Lor-2 protein or polypeptide of the present invention or a non-Lor-2 molecule. For example, a Lor-2 target molecule can be a non-Lor-2 protein molecule. Alternatively, a Lor-2 activity is an indirect activity, such as an activity mediated by interaction of the Lor-2 protein with a Lor-2 target molecule such that the target molecule modulates a downstream cellular activity (e.g., interaction of a Lor-2 molecule with a Lor-2 target molecule can modulate the activity of that target molecule on a cardiac cell).

In a preferred embodiment, a Lor-2 activity is at least one or more of the following activities: (i) interaction of a Lor-2 protein with a Lor-2 target molecule; (ii) interaction of a Lor-2 protein with a Lor-2 target molecule, wherein the Lor-2 target is a ligand; (iii) interaction of a Lor-2 protein with a Lor-2 target molecule, wherein the Lor-2 target is an extracellular matrix component (e.g., collagen or elastin); and (iv) modification of a Lor-2 target molecule (e.g., posttranslational modification).

In yet another preferred embodiment, a Lor-2 activity is at least one or more of the following activities: (1) crosslinking an extracellular matrix component; (2) regulating bone resorption and/or metabolism; (3) regulating copper metabolism; (4) modulating maturation, stabilization and/or degradation of extracellular matrix components; (5) regulating cellular signaling; and (6) regulating cellular adhesion (e.g. adhesion of a tumor cell).

In another embodiment of the invention, a Lor-2 molecule or preferably, a Lor-2 modulator, is useful for regulating, preventing and/or treating at least one or more of the following diseases or disorders: (1) diseases or disorders involving impaired copper metabolism (e.g., type IX of the Ehlers-Danlos syndrome and the Menkes syndrome); (2) bone disorders (e.g., osteoporosis or osteoarthritis); (3) fibrotic disorders (e.g., atherosclerosis, tissue and/or organ fibrosis); (4) proliferative disorders (e.g., cancer, for example, prostate cancer, breast cancer, lung cancer and the like); (5) vascular disorders (e.g., ischemia, ischemic-reperfusion injury); and (6) cardiac trauma (e.g., iatrogenic, accidental).

In yet another embodiment of the invention, a Lor-2 molecule or preferably, a Lor-2 modulator, is useful for regulating, preventing and/or treating at least one or more of the following diseases or disorders: (1) cardiac hypertrophy and cardiomyopathy; (2) cardiac pathologies; (3) myocardial hypertrophy and cardiovascular lesions; (4) myocardial aneurysms; (5) atherosclerotic cardiovascular disease; (6) fibrotic disease; (7) osteoporosis; (8) metastasis/prostate cancer; (9) cellular senescence/tumor suppression; (10) tumor progression; (11) liver fibrosis; (12) wound healing; (13) hypertension; (14) diabetes; (15) arthritis; and (16) bone disease (e.g., osteoporosis or osteoarthritis).

In yet another embodiment, a Lor-2 modulator is useful for regulating (e.g., inhibiting) tumor progression. For example, Lor-2 may be secreted by a tumor cell facilitating adhesion (e.g., enhancing the adhesive properties) of the cell. Accordingly, Lor-2 modulators can be used to affect the adhesive properties of tumor cells (e.g., to surrounding tissues).

In yet another embodiment, a Lor-2 modulator, is useful for regulating or preventing immunosuppression by tumor cells. For example, Lor-2 may be secreted by a tumor cell, conferring on that cell a growth advantage (e.g., maintaining the growth, differentiation, and transformed phenotype of the tumor cell). In such a situation, secreted Lor-2 can inhibit cytotoxicity (e.g., lymphocytotoxicity, for example, IL-2-induced lymphocytotoxicity). Accordingly, Lor-2 may function to suppress the generation and/or proliferation of lymphocytic cells (e.g., lymphocyte-activated killer cells).

As used herein, the term “cardiovascular disorder” includes a disease, disorder, or state involving the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. Examples of such disorders include hypertension, atherosclerosis, coronary artery spasm, coronary artery disease, valvular disease, arrhythmias, and cardiomyopathies.

As used herein, the term “congestive heart failure” includes a condition characterized by a diminished capacity of the heart to supply the oxygen demands of the body. Symptoms and signs of congestive heart failure include diminished blood flow to the various tissues of the body, accumulation of excess blood in the various organs, e.g., when the heart is unable to pump out the blood returned to it by the great veins, exertional dyspnea, fatigue, and/or peripheral edema, e.g., peripheral edema resulting from left ventricular dysfunction. Congestive heart failure may be acute or chronic. The manifestation of congestive heart failure usually occurs secondary to a variety of cardiac or systemic disorders that share a temporal or permanent loss of cardiac function. Examples of such disorders include hypertension, coronary artery disease, valvular disease, and cardiomyopathies, e.g., hypertrophic, dilative, or restrictive cardiomyopathies. Congestive heart failure is described in, for example, Cohn J. N. et al. (1998) American Family Physician 57:1901-04, the contents of which are incorporated herein by reference.

As used herein, the term “cardiac cellular processes” includes intra-cellular or inter-cellular processes involved in the functioning of the heart. Cellular processes involved in the nutrition and maintenance of the heart, the development of the heart, or the ability of the heart to pump blood to the rest of the body are intended to be covered by this term. Such processes include, for example, cardiac muscle contraction, distribution and transmission of electrical impulses, and cellular processes involved in the opening and closing of the cardiac valves. The term “cardiac cellular processes” further includes processes such as the transcription, translation and post-translational modification of proteins involved in the functioning of the heart, e.g., myofilament specific proteins, such as troponin I, troponin T, myosin light chain 1 (MLC1), and α-actinin. In another embodiment, the Lor-2 molecules of the present invention modulate the activity of one or more proteins involved in a cardiovascular disorder, e.g., congestive heart failure, ischemia, cardiac hypertrophy, ischemic-reperfusion injury.

C. STRIFE Nucleic Acid and Protein Molecules

The murine STRIFE1 cDNA, which is approximately 981 nucleotides in length, encodes a protein which is approximately 214 amino acid residues in length. The murine STRIFE2 cDNA, which is approximately 655 nucleotides in length, encodes a protein which is approximately 150 amino acid residues in length. STRIFE1 and STRIFE2 are alternately spliced forms of STRIFE; STRIFE1 is membrane-bound, while STRIFE2 is secreted. TNF receptors are typically membrane-bound, trimeric or multimeric complexes which are stabilized via intracysteine disulfide bonds that are formed between the cysteine-rich domains of individual subunit members (Banner et al. (1993) Cell 73:431-445). Functional TNF receptors can also exist in a soluble form. Soluble members of the superfamily bind cognate ligands and influence bioavailability. The soluble superfamily members lack the transmembrane domain characteristic of the majority of superfamily members due to either proteolytic cleavage or, at least in one instance, to alternative splicing (Gruss et al. (1995) Blood 85, 3378-3404).

TNF receptors are the sole mediators of Tumor Necrosis Factor (TNF) signaling. TNF is a cytokine that is capable of acting independently or in conjunction with other factors to affect various different body functions. In vitro, TNF has diverse biological effects, including killing of transformed cells, stimulation of granulocytes and fibroblasts, damage to endothelial cells, and anti-parasitic effects. In vivo, TNF plays a key role as an endogenous mediator of inflammatory, immune, and host defense functions. In addition, TNF plays a role in various neoplastic disease states.

The STRIFE1 and STRIFE2 molecules of the present invention having homology to the TNF receptors may also be TNF receptors involved in TNF signaling. Thus, the STRIFE1 and STRIFE2 molecules of the present invention may play a role in mediating inflammatory, immune, and host defense functions. In addition, the STRIFE1 and STRIFE2 molecules of the present invention may play a role in various neoplastic disease states. Thus, the STRIFE1 and STRIFE2 molecules may be useful as targets for developing novel diagnostic and therapeutic agents to treat TNF-associated disorders and TNF receptor-associated disorders, as described herein.

In one embodiment, a STRIFE1 and a STRIFE2 family member is identified based on the presence of at least one “cysteine-rich domain” in the protein or corresponding nucleic acid molecule. As used herein, the term “cysteine-rich domain” refers to a protein domain of about 110-160 amino acid residues in length, preferably about 100-150 amino acid residues in length, more preferably about 90-140 amino acid residues in length, and even more preferably at least about 80-130 amino acid residues in length, of which at least about 10-30, preferably about 10-20, and more preferably about 12, 13, 14, or 15 amino acid residues are cysteine residues. In a preferred embodiment, a cysteine-rich domain is located in the N-terminal region of a STRIFE1 and STRIFE2 protein and includes about amino acid residues 34 through 114 of SEQ ID NO:23 and SEQ ID NO:27, respectively. Preferred cysteine rich domains contain at least about two, three, or four modules or motifs, wherein each module is a region of about 20-60 amino acid residues in length, preferably 30-50 amino acid residues in length, more preferably 40 amino acid residues in length and includes about 3-10 cysteines, preferably 5-7 cysteines, and more preferably 6 cysteines. In one embodiment, the module has the following motif:

(SEQ ID NO: 128)
C-Xaa1(4-14)-C-Xaa2(0-2)-C-Xaa3(2-4)-C-Xaa4(6-12)-
C-Xaa5(6-10)-C,

wherein “C” is the amino acid cysteine and “Xaa1-Xaa5” can be any amino acid residue. In a preferred embodiment, Xaa1 is between 4-6, 6-8, 8-10, 10-12, or 12-14 amino acid residues; Xaa4 is between 6-8, 8-10, or 10-12 amino acid residues; and Xaa5 is between 6-8 or 8-10 amino acid residues. In another preferred embodiment, Xaa1 is 4-6 amino acid residues, of which at least one is the amino acid phenylalanine, at least one is the amino acid tyrosine, and/or at least one is the amino acid histidine. In yet another preferred embodiment, Xaa5 is 6-10 amino acid residues, of which at least one is the amino acid aspartic acid, at least one is the amino acid asparagine, at least one is the amino acid glutamic acid, at least one is the amino acid glutamine, at least one is the amino acid serine, at least one is the amino acid lysine, and/or at least one is the amino acid proline. In another embodiment, the module has the following motif:

(SEQ ID NO: 129)

C-Xaa1(4,6)-FYH-Xaa2(5,10)-C-Xaa3(0,2)-C-
Xaa4(2,3)-C-Xaa5(7,11)-C-Xaa6(4,6)-DNEQSKP-
Xaa7(2)-C.

For example, in one embodiment, a STRIFE1 protein contains a cysteine-rich domain including a first module containing about amino acids 34-72 of SEQ ID NO:23 (shown separately as SEQ ID NO:130) having 6 cysteine residues at positions indicated by the aforementioned motifs, and a second module containing about amino acids 75-114 of SEQ ID NO:23 (shown separately as SEQ ID NO:131) having 6 cysteine residues at positions indicated by the aforementioned motifs. In another embodiment, a STRIFE2 protein contains a cysteine rich domain including a first module containing about amino acids 34-72 of SEQ ID NO:27 (shown separately as SEQ ID NO:132) having 6 cysteine residues at positions indicated by the aforementioned motifs, and a second module containing about amino acids 75-114 of SEQ ID NO:27 (shown separately as SEQ ID NO:133) having 6 cysteine residues at positions indicated by the aforementioned motifs.

In another embodiment of the invention, a STRIFE1 and STRIFE2 protein has at least one cysteine-rich domain and a signal sequence, as defined herein. For example, in one embodiment, a STRIFE1 protein contains a signal sequence of about amino acids 1-29 of SEQ ID NO:23 (shown separately as SEQ ID NO:134). In another embodiment, a STRIFE2 protein contains a signal sequence of about amino acids 1-29 of SEQ ID NO:27 (shown separately as SEQ ID NO:135).

Accordingly, one embodiment of the invention features a STRIFE1 or a STRIFE2 protein having at least one cysteine-rich domain. Another embodiment features a STRIFE1 or a STRIFE2 protein having at least one cysteine-rich domain, wherein the cysteine-rich domain includes at least one module having the predicted motif of SEQ ID NO:129. Another embodiment features a STRIFE1 or a STRIFE2 protein having at least one cysteine-rich domain, wherein the cysteine-rich domain includes at least two modules. Another embodiment features a protein having 20, 30, 40, 50, 60, 70, 80, 90, 95, or 99% homology to a cysteine-rich domain of a STRIFE1 or a STRIFE2 protein of the invention.

Yet another embodiment of the invention features a STRIFE1 or a STRIFE2 protein having at least one cysteine-rich domain and a signal peptide. Another embodiment features a STRIFE1 or a STRIFE2 protein having at least one cysteine-rich domain and a signal peptide, wherein the cysteine-rich domain includes at least one module having the predicted motif of SEQ ID NO:129.

In yet another embodiment of the invention, a STRIFE1 protein has a transmembrane domain. As used herein, the term “transmembrane domain” refers to an amino acid sequence having at least about 10, preferably about 13, preferably about 16, more preferably about 19, and even more preferably about 21, 23, 25, 30, 35 or 40 amino acid residues, of which at least about 60-70%, preferably about 80% and more preferably about 90% of the amino acid residues contain non-polar side chains, for example, alanine, valine, leucine, isoleucine, proline, phenylalanine, tryptophan, and methionine. A transmembrane domain is lipophilic in nature. For example, a STRIFE1 protein contains a transmembrane domain containing amino acids 169-193 of SEQ ID NO:23 (shown separately as SEQ ID NO:136).

Preferred STRIFE1 or STRIFE2 molecules of the present invention have an amino acid sequence sufficiently homologous, as defined herein, to the amino acid sequence of SEQ ID NO:23, or SEQ ID NO:27, respectively.

As used interchangeably herein, a “STRIFE1 and a STRIFE2 activity”, “biological activity of STRIFE1 and STRIFE2” or “functional activity of STRIFE1 and STRIFE2”, refer to an activity exerted by a STRIFE1 and a STRIFE2 protein, polypeptide or nucleic acid molecule on a STRIFE1 or a STRIFE2 responsive cell as determined in vivo, or in vitro, according to standard techniques. In one embodiment, a STRIFE1 and a STRIFE2 activity is a direct activity, such as an association with a STRIFE1 or a STRIFE2-target molecule. As used herein, a “target molecule” is a molecule with which a STRIFE1 and a STRIFE2 protein binds or interacts in nature, such that STRIFE1 or STRIFE2-mediated function is achieved. A STRIFE1 or a STRIFE2 target molecule can be a non-STRIFE1 and a non-STRIFE2 molecule or a STRIFE1 or STRIFE2 protein or polypeptide of the present invention. In an exemplary embodiment, a STRIFE2 target molecule is a membrane-bound protein (e.g., a “STRIFE2 receptor”) or a modified form of such a protein which has been altered such that the protein is soluble (e.g., recombinantly produced such that the protein does not express a membrane-binding domain). In another embodiment, a STRIFE 1 or a STRIFE2 target is a second soluble protein molecule (e.g., a “STRIFE1 or a STRIFE2 binding partner” or a “STRIFE1 and STRIFE2 substrate”). In such an exemplary embodiment, a STRIFE1 and a STRIFE2 binding partner can be a soluble non-STRIFE1 and non-STRIFE2 protein or a second STRIFE1 and a STRIFE2 protein molecule of the present invention. Alternatively, a STRIFE1 and a STRIFE2 activity is an indirect activity, such as a cellular signaling activity mediated by interaction of the STRIFE1 and the STRIFE2 protein with a second protein (e.g., a STRIFE1 ligand or a STRIFE2 receptor).

In a preferred embodiment, a STRIFE1 activity is at least one or more of the following activities: (i) interaction of a STRIFE1 protein on the cell surface with a second non-STRIFE1 protein molecule on the surface of the same cell; (ii) interaction of a STRIFE1 protein on the cell surface with a second non-STRIFE1 protein molecule on the surface of a different cell; (iii) complex formation between a membrane-bound STRIFE1 protein and a cytokine, e.g., TNF; (iv) interaction of a STRIFE1 protein with an intracellular protein including SH2 domain-containing proteins or cytoskeletal proteins; (v) formation of a homogeneous multimeric signaling complex with STRIFE1-like proteins; and (vi) formation of a heterogeneous multimeric signaling complex with other TNFR superfamily proteins.

In another preferred embodiment, a STRIFE2 activity is at least one or more of the following activities: (i) interaction of a STRIFE2 protein with a membrane-bound STRIFE2 receptor; (ii) interaction of a STRIFE2 protein with a soluble form of a STRIFE2 receptor; (iii) interaction of a STRIFE2 protein with an intracellular protein via a membrane-bound STRIFE2 receptor; (iv) complex formation between a soluble STRIFE2 protein and a second soluble STRIFE2 binding partner; (v) complex formation between a soluble STRIFE2 protein and a second soluble STRIFE2 binding partner, wherein the STRIFE2 binding partner is a non-STRIFE2 protein molecule; and (vi) complex formation between a soluble STRIFE2 protein and a second soluble STRIFE2 binding partner, wherein the STRIFE2 binding partner is a second STRIFE2 protein molecule.

In yet another preferred embodiment, a STRIFE1 or a STRIFE2 activity is at least one or more of the following activities: (i) modulation of cellular signal transduction, either in vitro or in vivo; (ii) regulation of gene transcription in a cell involved in development or differentiation, either in vitro or in vivo; (iii) modulation of cellular signal transduction; (iv) regulation of cellular proliferation; (v) regulation of cellular differentiation; and (vi) regulation of cell survival.

Accordingly, another embodiment of the invention features isolated STRIFE1 and STRIFE2 proteins and polypeptides having a STRIFE1 and/or STRIFE2 activity, respectively. Preferred STRIFE1 and STRIFE2 proteins have at least one cysteine-rich domain and a STRIFE1 and/or a STRIFE2 activity. In another preferred embodiment, the STRIFE1 and STRIFE2 protein has at least one cysteine-rich domain, wherein the cysteine-rich domain comprises at least one module, and a STRIFE1 and STRIFE2 activity, respectively. In another preferred embodiment, the STRIFE1 and STRIFE2 protein has at least one cysteine-rich domain, wherein the cysteine-rich domain comprises at least two modules, and a STRIFE1 and STRIFE2 activity, respectively. In yet another preferred embodiment, a STRIFE1 and a STRIFE2 protein further comprises a signal sequence. In still another preferred embodiment, a STRIFE1 and a STRIFE2 protein has a cysteine-rich domain, a STRIFE1 and a STRIFE2 activity, and an amino acid sequence sufficiently homologous, as defined herein, to an amino acid sequence of SEQ ID NO:23, or SEQ ID NO:27, respectively.

The murine STRIFE1 protein contains an N-terminal signal sequence and a cysteine-rich domain comprising two modules. A STRIFE1 cysteine-rich domain can be found at least, for example, from about amino acids 34-114 of SEQ ID NO:23. The STRIFE1 cysteine-rich domain comprises a first module from about amino acids 34-72 of SEQ ID NO:23 (shown separately as SEQ ID NO:130) and a second module from about amino acids 75-114 of SEQ ID NO:23 (shown separately as SEQ ID NO:131). The murine STRIFE1 protein is a membrane bound protein which contains a transmembrane domain at about amino acids 169-193 of SEQ ID NO:23 (shown separately as SEQ ID NO:136) and a signal sequence at about amino acids 1-29 of SEQ ID NO:23 (shown separately as SEQ ID NO:134). The prediction of such a signal peptide can be made, for example, utilizing the computer algorithm SIGNALP (Henrik, et al. (1997) Protein Eng. 10:1-6).

The murine STRIFE2 protein contains an N-terminal signal sequence and a cysteine-rich domain comprising two modules. A STRIFE2 cysteine-rich domain can be found at least, for example, from about amino acids 34-114 of SEQ ID NO:27. The STRIFE2 cysteine-rich domain comprises a first module from about amino acids 34-72 of SEQ ID NO:27 (shown separately as SEQ ID NO:132) and a second module from about amino acids 75-114 of SEQ ID NO:27 (shown separately as SEQ ID NO:133). The murine STRIFE2 protein is a secreted protein which further contains a signal sequence at about amino acids 1-29 of SEQ ID NO:27 (shown separately as SEQ ID NO:135).

As used herein, the terms “TNF-associated disorder” and “TNF receptor-associated disorder” include any disorder, disease, or condition which is associated with an abnormal or undesired TNF or TNF receptor function or an abnormal or undesired TNF or TNF receptor level, e.g., plasma, tissue, or cellular levels or concentration. Examples of TNF-associated and TNF receptor-associated disorders include, but are not limited to, sepsis syndrome, including cachexia; circulatory collapse and shock resulting from acute or chronic bacterial infection; acute and chronic parasitic or infectious processes, including bacterial, viral and fungal infections; acute and chronic immune and autoimmune pathologies, such as systemic lupus erythematosus and rheumatoid arthritis; alcohol-induced hepatitis; chronic inflammatory pathologies such as sarcoidosis and Crohn's pathology; vascular inflammatory pathologies such as disseminated intravascular coagulation; graft-versus-host pathology; Rawasaki's pathology; malignant pathologies involving TNF-secreting tumors; cerebral malaria; and multiple sclerosis.

D. TRASH Nucleic Acid and Protein Molecules

The human TRASH I cDNA set forth in SEQ ID NO:31, is approximately 1346 nucleotides in length and encodes a protein which is approximately 250 amino acid residues in length (SEQ ID NO:32). In a preferred embodiment, the human TRASH protein contains a TNF signature motif, a TNF-like N-terminal signal transmembrane anchor for a type II membrane protein, two cysteine residues that may be disulfide linked, and two putative N-linked glycosylation sites. A TRASH TNF signature motif can be found at least, for example, from about amino acids 155-171 of SEQ ID NO:32. A TRASH TNF-like N-terminal signal transmembrane anchor for a type II membrane protein can be found at least, for example, from about amino acids 1-44 of SEQ ID NO:32. Two cysteine residues that may be disulfide linked can be found at least, for example, at about amino acids 196 and 211 of SEQ ID NO:32. Two putative N-linked glycosylation sites can be found at least, for example, at about amino acids 124 and 237 of SEQ ID NO:32.

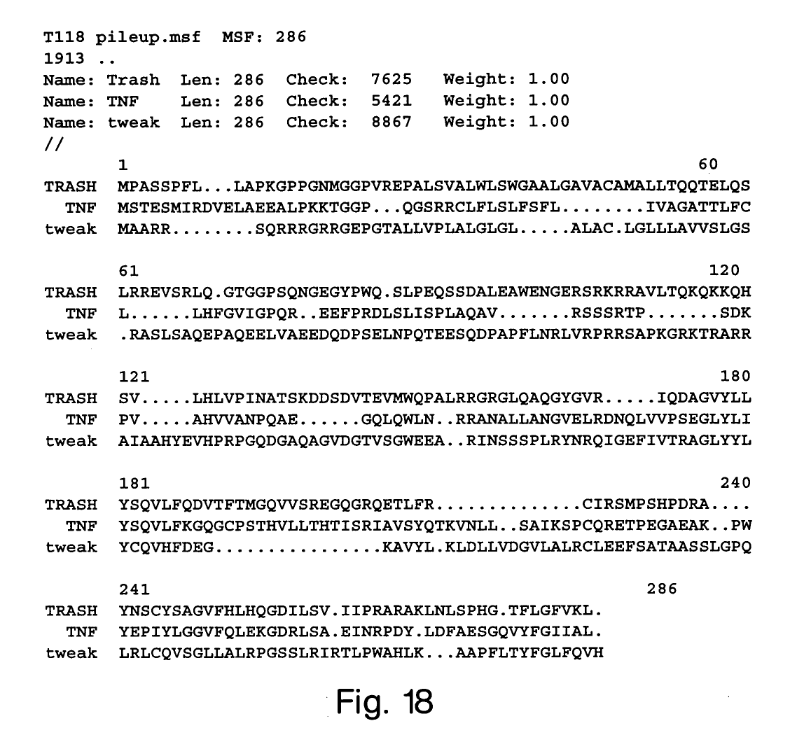

An alignment of the amino acid sequences of human TRASH (corresponding to amino acids 1-250 of SEQ ID NO:32), TNF-α (Swiss-Prot™ Accession No. P01375; SEQ ID NO:43), and human Tweak (Swiss-Prot™ Accession No. AF030099; SEQ ID NO:44) is shown in FIG. 18. (The alignment was generated using MegAlign™ sequence alignment software. The initial pairwise alignment step was performed using a Wilbur-Lipmann algorithm with a K-tuple of 1, a GAP penalty of 3, a window of 5, and diagonals saved set to =5. The multiple alignment step was performed using the Clustal algorithm with a PAM 250 residue weight Table, a GAP penalty of 10, and a GAP length penalty of 10.)

In another embodiment of the invention, a TRASH family member is identified based on the presence of a “TNF signature motif” in the protein or corresponding nucleic acid molecule. As used herein, the term “TNF signature motif” refers to a protein domain which is a signature for the TNF family of proteins and contains the consensus sequence

V-Xaa₁-I-Xaa₂₍₁₋₃₎-GVYLL-Xaa₃₍₁₋₂₎-Q-V-Xaa₄-F

wherein V is the amino acid valine, Xaa₁ represents any amino acid, I is isoleucine, Xaa₂₍₁₋₃₎ represents 1 to 3 amino acids which can be the same or different, G is glycine, V is valine, Y is tyrosine, L is leucine, Xaa₃₍₁₋₂₎ represents 1 to 2 amino acids which can be the same or different, Q is glutamine, V is valine, Xaa₄ represents any amino acid, and F is phenylalanine (as represented in SEQ ID NO:42). In a preferred embodiment, the TRASH protein comprises a TNF signature motif which is at least about 10-20 amino acid residues in length, preferably at least about 12-18 amino acid residues in length, and more preferably at least about 15-17 amino acid residues in length. In another embodiment, the TRASH protein comprises a TNF signature motif which is at least about 55%, preferably at least about 65%, more preferably at least about 75-85% homologous, and even more preferably at least about 90-95% homologous to the TNF signature motif of SEQ ID NO:32 (about amino acids 155-171 or SEQ ID NO:34). In a preferred embodiment, the TRASH protein comprises the TNF signature motif of SEQ ID NO:32 (about amino acids 155-171 or SEQ ID NO:34).

In another embodiment, an TRASH family member is identified based on the presence of a TNF-like N-terminal signal transmembrane anchor for a type II membrane protein in the protein or corresponding nucleic acid molecule. As used herein, the term “TNF-like N-terminal signal transmembrane anchor for a type II membrane protein” refers to a protein domain located at the extreme N-terminal end of secretory and integral membrane proteins, contains a number of hydrophobic residues, and which serves to direct a protein containing such a sequence to a lipid bilayer. In a preferred embodiment, a TRASH protein comprises a TNF-like N-terminal signal transmembrane anchor for a type II membrane protein and contains at least 30-60, preferably about 35-55 amino acid residues, more preferably about 40-50 amino acid residues, still more preferably about 42-48 amino acid residues, and most preferably about 44-46 amino acid residues, of which at least about 40-70%, preferably about 50-65%, and more preferably about 55-60% are hydrophobic amino acid residues (e.g., Alanine, Valine, Leucine, Isoleucine, Phenylalanine, Tyrosine, Tryptophan, or Proline). In another preferred embodiment, a TRASH protein comprises a TNF-like N-terminal signal transmembrane anchor for a type II membrane protein which is at least about 55-60%, preferably at least about 65-70%, more preferably at least about 75-80%, still more preferably at least about 85-90%, and yet more preferably 5-98% homologous to the TNF-like N-terminal signal transmembrane anchor for a type H membrane protein shown in SEQ ID NO:32 (about amino acids 1-44 or SEQ ID NO:35). In still another preferred embodiment, a TRASH protein comprises a TNF-like N-terminal signal transmembrane anchor for a type II membrane protein of SEQ ID NO:32 (about amino acids 1-44 or SEQ ID NO:35).

In a preferred embodiment, a TRASH protein contains both a TNF signature motif and a TNF-like N-terminal signal transmembrane anchor for a type II membrane protein. In another preferred embodiment, a TRASH protein further contains two cysteine residues that may be disulfide linked. In another preferred embodiment, a TRASH protein further contains two putative N-linked glycosylation sites. In one exemplary embodiment, a TRASH protein contains a TNF signature motif including about amino acids 155-171 of SEQ ID NO:32. In another exemplary embodiment, a TRASH protein contains a TNF-like N-terminal signal transmembrane anchor for a type II membrane protein including about amino acids 1-44 of SEQ ID NO:32. In yet another exemplary embodiment, a TRASH protein further includes two cysteine residues that may be disulfide linked at amino acid 196 and 211 of SEQ ID NO:32. In still yet another exemplary embodiment, a TRASH protein further includes two putative N-linked glycosylation sites at about amino acid 124 and 237 of SEQ ID NO:32.

Preferred TRASH molecules of the present invention have an amino acid sequence sufficiently homologous, as defined herein, to the amino acid sequence of SEQ ID NO:32, SEQ ID NO:37, SEQ ID NO:39, or SEQ ID NO:41.

As used interchangeably herein an “TRASH activity”, “biological activity of TRASH” or “functional activity of TRASH”, refer to an activity exerted by a TRASH protein, polypeptide or nucleic acid molecule on a TRASH responsive cell as determined in vivo, or in vitro, according to standard techniques. In one embodiment, a TRASH activity is a direct activity, such as an association with a cell-surface protein (e.g., a TRASH receptor). In another embodiment, a TRASH activity is an indirect activity, such as the induction of synthesis of a second protein (e.g. a cellular cytokine) mediated by interaction of the TRASH protein with a cell surface protein. In a preferred embodiment, a TRASH activity is at least one or more of the following activities: (i) interaction of a TRASH protein in the extracellular milieu with a non-TRASH protein molecule on the surface of the same cell which secreted the TRASH protein molecule; (ii) interaction of a TRASH protein in the extracellular milieu with a non-TRASH protein molecule on the surface of a different cell from that which secreted the TRASH protein molecule; (iii) complex formation between a TRASH protein and a TRASH receptor; (iv) complex formation between a TRASH protein and non-TRASH receptor; and (v) interaction of a TRASH protein with a second protein in the extracellular milieu. In yet another preferred embodiment, a TRASH activity is at least one or more of the following activities: (i) activation of a TRASH-dependent signal transduction pathway; (ii) cytolysis of certain tumor cell lines; (iii) modulation of secretion of inflammatory mediators/cytokines; (iv) modulation of the development or differentiation of a TRASH-expressing cell; (v) modulation of the development or differentiation of a non-TRASH-expressing cell; (vi) modulation of host resistance to infectious agents.

Accordingly, another embodiment of the invention features isolated TRASH proteins and polypeptides having a TRASH activity. Preferred TRASH proteins have a TNF signature motif and a TRASH activity. In another embodiment, the TRASH protein has a TNF-like N-terminal signal transmembrane anchor for a type II membrane protein and a TRASH activity. In another embodiment of the invention, the TRASH protein has a TNF signature motif, a TNF-like N-terminal signal transmembrane anchor for a type II membrane protein, and a TRASH activity. In another preferred embodiment, the TRASH protein has a TNF signature motif, a TNF-like N-terminal signal transmembrane anchor for a type II membrane protein, a TRASH activity, and an amino acid sequence sufficiently homologous, as defined herein, to an amino acid sequence of SEQ ID NO:32, SEQ ID NO:37, SEQ ID NO:39, or SEQ ID NO:41. In still another preferred embodiment, the TRASH protein further comprises two cysteine residues that may be disulfide linked. In yet another preferred embodiment, the TRASH protein further comprises two putative N-linked glycosylation sites.

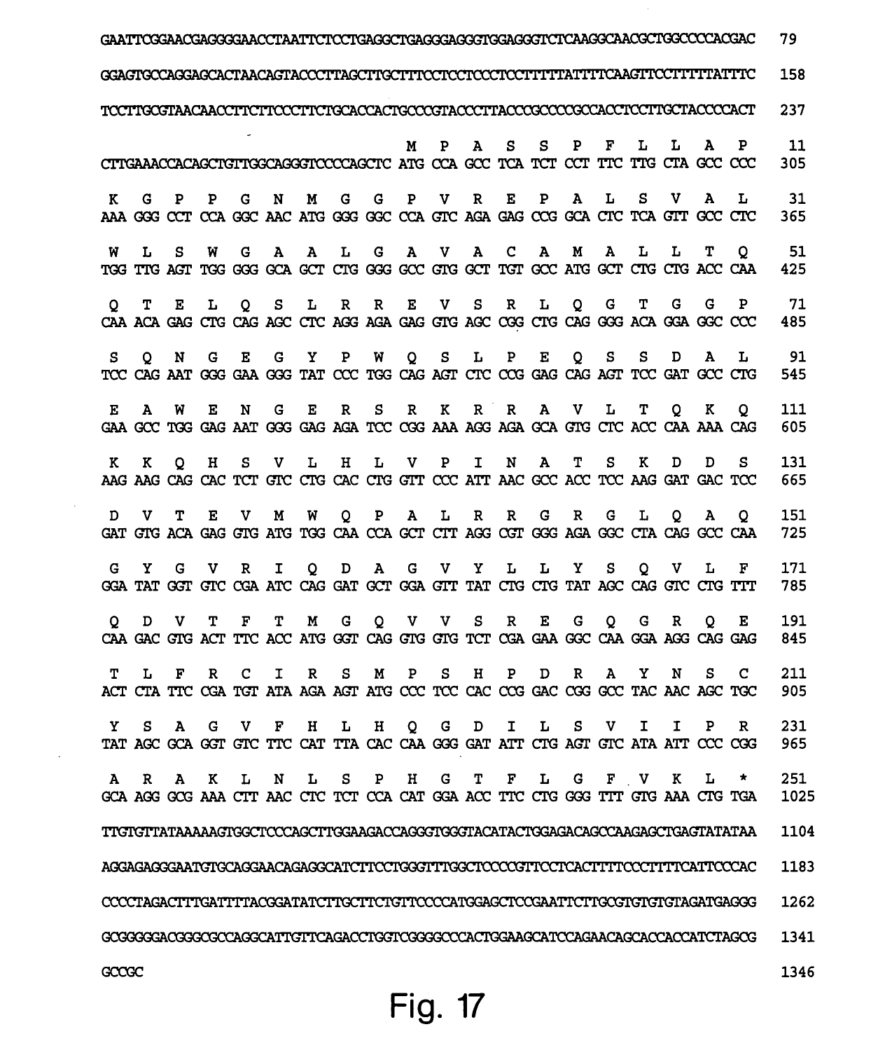

In a particularly preferred embodiment, the TRASH protein and nucleic acid molecules of the present invention are human TRASH molecules. A nucleotide sequence of an isolated human TRASH cDNA and the predicted amino acid sequence of the human TRASH protein are shown in FIG. 17 and in SEQ ID NOs:31 and 32, respectively. In addition, the nucleotide sequence corresponding to the coding region of the human TRASH cDNA is represented as SEQ ID NO:33.

TRASH mRNA transcripts of approximately 1.5 kb were predominantly expressed in peripheral blood leukocytes. A transcript of approximately 1.5 kb is seen in the spleen and lymph nodes and lung. A transcript of approximately 1.7 kb is also observed in the colon, kidney, and spleen (See Example 7).

E. BDSF Nucleic Acid and Protein Molecules

The human BDSF-1 cDNA, which is approximately 1119 nucleotides in length, encodes a protein which is approximately 244 amino acid residues in length. The human BDSF-1 protein has an Ig-like domain. An Ig-like domain includes, for example, about amino acids 41-129 of SEQ ID NO:46. The Ig-like domain further contains at least about two conserved cysteine residues. Cysteine residues can be found at least about at amino acids 48 and 127 of SEQ ID NO:46. The human BDSF-1 protein is predicted to be a secreted protein which contains a signal sequence at about amino acids 1-25 of SEQ ID NO:46. The prediction of such a signal peptide can be made, for example, utilizing the computer algorithm SIGNALP (Henrik, et al. (1997) Protein Eng. 10:1-6).

The murine BDSF-1 cDNA, which is approximately 3196 nucleotides in length, encodes a protein which is approximately 251 amino acid residues in length. The murine BDSF-1 protein has an Ig-like domain. An Ig-like domain includes, for example, about amino acids 40-128 of SEQ ID NO:51. The Ig-like domain further contains at least about two conserved cysteine residues. Cysteine residues can be found at least about at amino acids 47 and 126 of SEQ ID NO:51. The murine BDSF-1 protein is predicted to be a secreted protein which contains a signal sequence at about amino acids 1-24 of SEQ ID NO:51.

In one embodiment, the isolated proteins of the present invention, preferably BDSF proteins, are proteins having an amino acid sequence of about 150-340 amino acid residues in length, preferably about 170-320, more preferably about 190-300, more preferably about 210-280, or about 230-260 amino acid residues in length. In one embodiment, an isolated protein of the present invention, preferably a BDSF protein, includes an immunoglobulin (Ig)-like domain. As used herein, the term an “immunoglobulin-like domain” includes an amino acid sequence having about 65-115, preferably about 70-110, more preferably about 80-100 amino acid residues, and even more preferably at least about 85-95 amino acids in length and having a bit score for the alignment of the sequence to the Ig family Hidden Markov Model (HMM) of at least 10, preferably 10-15, more preferably 15-20, more preferably 20-25, even more preferably 25-35, 35-55, 55-100 or greater. The Ig family HMM has been assigned the PFAM Accession PF00047 (see PFAM website, available through Washington University in Saint Louis).

To identify the presence of an Ig-like domain in a BDSF family member, the amino acid sequence of the family member is searched against a database of HMMs (e.g., the Pfam database, release 2.1) using the default parameters. For example, the hmmsf program, which is available as part of the HMMER package of search programs, is a family specific default program for PF00047 having a score of 15 as the default threshold score for determining a hit. For example, a search using the amino acid sequence of SEQ ID NO:46 was performed against the HMM database resulting in the identification of an Ig-like domain in the amino acid sequence of SEQ ID NO:46 and a score of 22.43 against the Ig family HMM Accession PF00047. The results of the search are set forth in Example 9.

In another example, a search was performed using the amino acid sequence of SEQ ID NO:51 against the HMM database resulting in the identification of an Ig-like domain in the amino acid sequence of SEQ ID NO:51 and a score of 22.43 against the Ig family HMM Accession PF00047. The results of the search are set forth in Example 9.

Accordingly, in one embodiment of the invention, a BDSF protein is a human BDSF-1 protein having an Ig-like domain at about amino acids 41-129 of SEQ ID NO:46. Such an Ig-like domain has the amino acid sequence of SEQ ID NO:55.

Accordingly, BDSF family members having at least 50-60% homology, preferably about 60-70%, more preferably about 70-80%, or about 80-90% homology with the Ig-like domain of human BDSF-1 (e.g., SEQ ID NO:55) are within the scope of the invention.

In yet another embodiment of the invention, a BDSF protein is a murine BDSF-1 protein having an Ig-like domain at about amino acids 40-128 of SEQ ID NO:51. Such an Ig-like domain has the amino acid sequence of SEQ ID NO:56.

Accordingly, a BDSF family member having at least 50-60% homology, preferably about 60-70%, more preferably about 70-80%, or about 80-90% homology with the Ig-like domain of murine BDSF-1 (e.g., SEQ ID NO:56) is within the scope of the invention. Description of the Pfam database can be found in Sonhammer et al. (1997) Proteins 28(3)405-420, and description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated by reference.

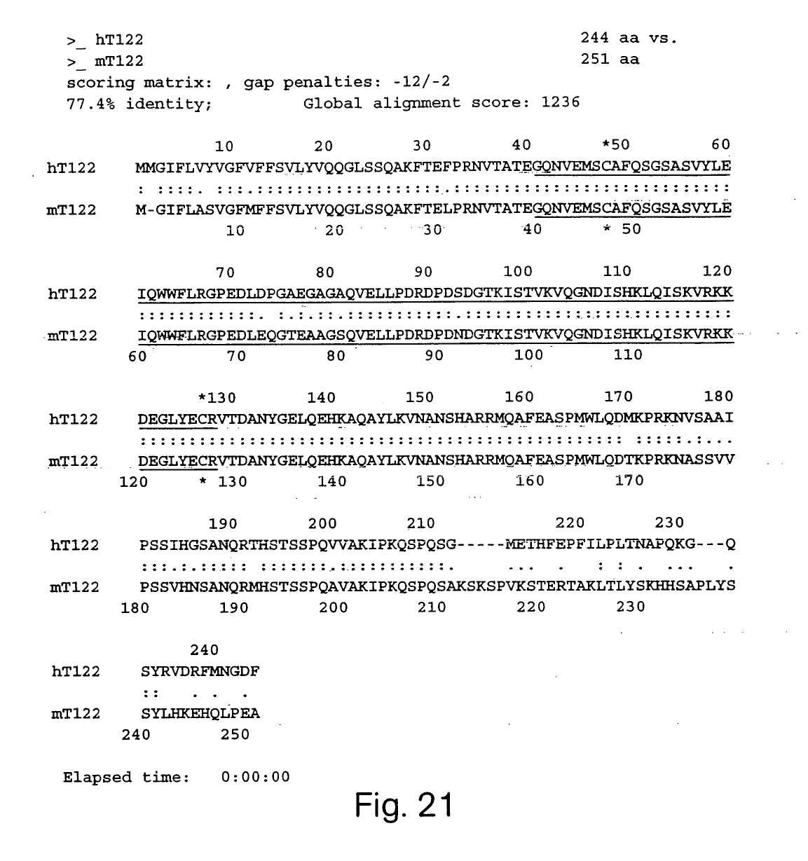

An Ig-like domain further contains at least one, preferably two, cysteine residues which are conserved between BDSF molecules. Preferably, the Ig-like domain of a protein, preferably a BDSF protein, has cysteine residues which are located in the same or similar positions as cysteine residues in other BDSF protein family members. For example, when a BDSF protein of the invention is aligned with a BDSF family member for purposes of comparison (see e.g., FIG. 21) preferred cysteine residues of the invention are those in which cysteine residues in the amino acid sequence of BDSF are located in the same or similar position as the cysteine residues in other BDSF family members. As an illustrative embodiment, FIG. 21 shows cysteine residues located in the same or similar positions of the human BDSF protein (corresponding to SEQ ID NO:46) and murine BDSF protein (corresponding to SEQ ID NO:51) in the following locations: amino acid number 48 of human BDSF and amino acid number 47 of murine BDSF; and amino acid number 127 of human BDSF and amino acid number 126 of murine BDSF.

In another embodiment of the invention, a BDSF protein has an Ig-like domain and a signal sequence, as defined herein. For example, in one embodiment, a BDSF protein contains a signal sequence of about amino acids 1-25 of SEQ ID NO:46, or a signal sequence of about amino acids 1-24 of SEQ ID NO:51.

Isolated proteins of the present invention, preferably BDSF proteins, have an amino acid sequence sufficiently homologous, as defined herein, to the amino acid sequence of SEQ ID NO:46, SEQ ID NO:49, SEQ ID NO:51 or SEQ ID NO:54 or are encoded by a nucleotide sequence sufficiently homologous to SEQ ID NO:45, SEQ ID NO:48, SEQ ID NO:50 or SEQ ID NO:53.

As used interchangeably herein, a “BDSF activity”, “biological activity of BDSF” or “functional activity of BDSF”, refer to an activity exerted by a BDSF protein, polypeptide or nucleic acid molecule as determined in vivo, or in vitro, according to standard techniques. In one embodiment, a BDSF activity is a direct activity, such as an association with a BDSF-target molecule. As used herein, a “target molecule” is a molecule with which a BDSF protein binds or interacts in nature (e.g., a BDSF receptor), such that BDSF-mediated function is achieved. A BDSF target molecule can be a BDSF protein or polypeptide of the present invention or a non-BDSF molecule. Alternatively, a BDSF activity is an indirect activity, such as an activity mediated by interaction of the BDSF protein with a BDSF target molecule such that the target molecule modulates a downstream cellular activity (e.g., interaction of an BDSF molecule with a BDSF target molecule can modulate the activity of that target molecule on an intracellular signaling pathway). In a preferred embodiment, a BDSF activity is at least one or more of the following activities: (i) interaction of a BDSF protein in the extracellular milieu with a non-BDSF protein molecule on the surface of the same cell which secreted the BDSF protein molecule; (ii) interaction of a BDSF protein in the extracellular milieu with a non-BDSF protein molecule on the surface of a different cell from that which secreted the BDSF protein molecule; (iii) complex formation between a BDSF protein and a BDSF receptor; (iv) complex formation between a BDSF protein and non-BDSF receptor; and (v) interaction of a BDSF protein with a second protein in the extracellular milieu. In yet another preferred embodiment, a BDSF activity is at least one or more of the following activities: (1) modulation of cellular signal transduction, either in vitro or in vivo; (2) modulation of protein:protein interactions, either in vitro or in vivo; (3) regulation of cellular proliferation; or (4) regulation of cellular differentiation.

Accordingly, another embodiment of the invention features isolated BDSF proteins and polypeptides having a BDSF activity. Preferred proteins are BDSF proteins having an Ig-like domain, and, preferably, a BDSF activity. In another preferred embodiment, the isolated protein further comprises a signal sequence. In still another preferred embodiment, the isolated protein is a BDSF protein having an Ig-like domain, a BDSF activity, preferably an amino acid sequence sufficiently homologous, as defined herein, to an amino acid sequence of SEQ ID NO:46 or SEQ ID NO:51, and optionally a signal sequence and/or propeptide.

F. LRSG Nucleic Acid and Protein Molecules

The human LRSG-1 cDNA, which is approximately 2852 nucleotides in length, encodes a protein which is approximately 673 amino acid residues in length. The human LRSG-1 protein has at least one leucine rich region. A leucine-rich region includes, for example, about amino acids 77-309 of SEQ ID NO:59. The leucine rich region further contains at least about 7 leucine-rich repeats. Leucine-rich repeats can be found at least about at amino acids 77-99, 101-123, 125-147, 149-171, 217-238, 240-263, and 289-309 of SEQ ID NO:59. The human LRSG-1 protein further has at least an EGF-like domain. An EGF-like domain includes, for example, about amino acids 409-441 of SEQ ID NO:59. The human LRSG-1 protein further has at least an Fn type III-like domain. A Fn type III-like domain includes, for example, about amino acids 460-535 of SEQ ID NO:59. The human LRSG-1 protein is predicted to be a membrane bound protein which further contains a signal sequence at about amino acids 1-23 of SEQ ID NO:59. The prediction of such a signal peptide can be made, for example, utilizing the computer algorithm SIGNALP (Henrik, et al. (1997) Protein Eng. 10:1-6). Furthermore, the human LRSG-1 protein is predicted to contain a transmembrane domain at about amino acids 576-599 of SEQ ID NO:59.

The murine LRSG-1 cDNA, which is approximately 2815 nucleotides in length, encodes a protein which is approximately 673 amino acid residues in length. The murine LRSG-1 protein has at least one leucine rich region. A leucine-rich region includes, for example, about amino acids 78-310 of SEQ ID NO:68. The leucine rich region further contains at least about 7 leucine-rich repeats. Leucine-rich repeats can be found at least about at amino acids 78-100, 102-124, 126-148, 150-172, 218-239, 241-264, and 290-310 of SEQ ID NO:68. The murine LRSG-1 protein further has at least an EGF-like domain. An EGF-like domain includes, for example, about amino acids 410-442 of SEQ ID NO:68. The murine LRSG-1 protein further has at least an Fn type III-like domain. An Fn type III-like domain includes, for example, about amino acids 461-536 of SEQ ID NO:68. The murine LRSG-1 protein is predicted to be a membrane bound protein which further contains a signal sequence at about amino acids 1-24 of SEQ ID NO:68. The prediction of such a signal peptide can be made, for example, utilizing the computer algorithm SIGNALP (Henrik, et al. (1997) Protein Eng. 10:1-6). Furthermore, the murine LRSG-1 protein is predicted to contain a transmembrane domain at about amino acids 577-600 of SEQ ID NO:68.

In one embodiment, the isolated proteins of the present invention, preferably LRSG proteins, are proteins having an amino acid sequence of about 450-900 amino acid residues in length, preferably about 500-850, more preferably about 550-800, more preferably about 600-750, and even more preferably about 650-700 amino acid residues in length. In one embodiment, an isolated protein of the present invention, preferably an LRSG protein, includes at least one leucine-rich repeat region. As used herein, a leucine-rich repeat (LRR) region is a region of a protein having an amino acid sequence of about 100-600 amino acid residues in length, preferably about 150-550, more preferably about 200-500, more preferably about 300-450 or about 350-400 amino acid residues in length, of which at least about 30-140, preferably about 40-130, more preferably about 50-120, more preferably about 60-90 or about 70-80 amino acid residues are leucine residues. In another embodiment, an LRR region has at least about 10-15% leucine residues, preferably about 15-20% leucine residues, more preferably about 20-25% or about 25-30% leucine resides. Accordingly, in one embodiment, an LRSG protein is human LRSG-1 having an LRR region of about amino acid residues 77-309 of SEQ ID NO:59. In another embodiment, an LRSG protein is murine LRSG-1 having an LRR region of about amino acid residues 78-310 of SEQ ID NO:68.

In a preferred embodiment, a leucine-rich repeat region includes about 4-28, preferably about 8-24, more preferably about 10-20, more preferably about 12-18 or about 14-16 leucine-rich repeats. As used herein, a “leucine-rich repeat” (“LRR”) is an amino acid motif having an amino acid sequence of about 15-30, preferably about 17-25, and more preferably 19-22 amino acid residues in length, of which about 2-12, preferably 3-10, more preferably 4-9, and more preferably 5-7 amino acid residues are leucine residues. Preferably, an LRR has the following consensus sequence:

(SEQ ID NO: 63)
X-[LIVMAFY]-X(2)-[LIVMAFY]-X-[LIVMAFY]-X(2)-[NCT]-
X(1,2)-[LIVMAFY]-X(2,3)-[LIVMAFY]-X(0-4)-
[LIVMAFY].

Accordingly, in one embodiment, an LRSG protein is human LRSG-1 having an LRR region of about amino acid residues 77-309 of SEQ ID NO:59, including about 7 LRRs. LRR 1 is about amino acid residues 77-99 of SEQ ID NO:59. LRR 2 is about amino acid residues 101-123 of SEQ ID NO:59. LRR 3 is about amino acid residues 125-147 of SEQ ID NO:59. LRR 4 is about amino acid residues 149-171 of SEQ ID NO:59. LRR 5 is about amino acid residues 217-238 of SEQ ID NO:59. LRR 6 is about amino acid residues 240-263 of SEQ ID NO:59. LRR 7 is about amino acid residues 289-309 of SEQ ID NO:59.

In another embodiment, an LRSG protein is murine LRSG-1 having an LRR region of about amino acid residues 78-310 of SEQ ID NO:68, including about 7 LRRs. LRR 1 is about amino acid residues 78-100 of SEQ ID NO:68. LRR 2 is about amino acid residues 102-124 of SEQ ID NO:68. LRR 3 is about amino acid residues 126-148 of SEQ ID NO:68. LRR 4 is about amino acid residues 150-172 of SEQ ID NO:68. LRR 5 is about amino acid residues 218-239 of SEQ ID NO:68. LRR 6 is about amino acid residues 241-264 of SEQ ID NO:68. LRR 7 is about amino acid residues 290-310 of SEQ ID NO:68.

In another embodiment, an LRSG family member is identified based on the presence of at least one “EGF-like domain” in the protein or corresponding nucleic acid molecule. As used herein, the term “EGF-like domain” refers to an amino acid sequence of at least about 15-55 amino acids in length, preferably about 20-50, more preferably about 25-45, and more preferably 30-40 amino acid residues in length, of which about 3-9, preferably 4-8, more preferably 5-7, and more preferably 6 amino acids are cysteine residues. Preferably, one or more cysteine residues in the EGF-like domain are conserved among LRSG family members or other proteins containing EGF-like domains (i.e., located in the same or similar position as the cysteine residues in other LRSG family members or other proteins containing EGF-like domains). In a preferred embodiment, an “EGF-like domain” has the following consensus sequence:

(SEQ ID NO: 64)

C-X(0-6)-C-X(0-7)-C-X(0-12)-C-X-C-X(0-14)-C.

In another referred embodiment, an “EGF-like domain” has the following consensus sequence:

(SEQ ID NO: 65)

C-X(4)-C-X(5)-C-X(6-10)-C-X-C-X(8-12)-C.

Accordingly, in one embodiment, an LRSG protein is human LRSG-1 having an EGF-like domain containing about amino acid residues 409-441 of SEQ ID NO:59. In another embodiment, an LRSG protein is murine LRSG-1 having an EGF-like domain containing about amino acid residues 410-442 of SEQ ID NO:68. The EGF-like domain is further described in PROSITE Document, Accession No. PDOC00021 and as PROSITE Accession No. PS0022.

In another embodiment, an LRSG family member is identified based on the presence of at least one “fibronectin type III-like domain “(“Fn type III-like domain”) in the protein or corresponding nucleic acid molecule. As used herein, the term “Fn type III-like domain” refers to an amino acid sequence of at least about 50-100, preferably about 60-90, more preferably about 70-80, and more preferably at least about 75-76 amino acid residues in length, of which at least about 50-80%, preferably 60-70%, more preferably 65% of the amino acid residues are identical or similar amino acids to the Fn type III consensus domain (SEQ ID NO:66) as shown in Example 10.

In a preferred embodiment, the Fn type III-like domain has at least about 60%, preferably at least about 70-80%, 90-95%, 96%, 97%, 98%, or 99% homology to the an Fn type III-like domain of human LRSG-1 having about amino acid residues 460-535 of SEQ ID NO:59. In another embodiment, the Fn type III-like domain is about amino acid residues 460-535 of SEQ ID NO:59. In another embodiment, the Fn type III-like domain is about amino acid residues 461-536 of SEQ ID NO:68. Accordingly, a preferred LRSG protein is a human LRSG-1 having an Fn type III-like domain containing about amino acid residues 460-535 of SEQ ID NO:59. The Fn type III domain is further described in Skorstengaard et al. (1986) Eur. J. Biochem. 161:441-453.

The domains described herein are described according to standard Prosite Signature designation (e.g., all amino acids are indicated according to their universal single letter designation; X designates any amino acid; X(n) designates any n amino acids, e.g., X (2) designates any 2 amino acids; and [LIVM] indicates any one of the amino acids appearing within the brackets, e.g., any one of L, I, V, or M, in the alternative, any one of Leu, Ile, Val, or Met.)

In another embodiment of the invention, an LRSG protein has at least one LRR region, and/or an EGF-like domain, and/or at least one Fn type III-like domain, and, preferably, a signal sequence. In another embodiment, an LRSG has an LRR region, an EGF-like domain, an Fn type III-like domain, and, preferably a signal sequence, as defined herein. For example, in one embodiment, an LRSG-1 protein contains a signal sequence of about amino acids 1-23 of SEQ ID NO:59. In another embodiment, an LRSG-1 protein contains a signal sequence of about amino acids 1-24 of SEQ ID NO:68.

In another embodiment of the invention, an LRSG protein has at least one LRR region and/or at least one EGF-like domain, and/or at least one Fn type III-like domain, and a transmembrane domain, as defined herein. For example, a transmembrane domain can be found at about amino acids 576-599 of SEQ ID NO:59. A transmembrane domain can also be found at about amino acids 577-600 of SEQ ID NO:68.

Accordingly, one embodiment of the invention features an LRSG protein having an LRR region and/or at least an Fn type III-like domain and a transmembrane domain. Another embodiment features an LRSG protein having an LRR region, and/or at least EGF-like domain, an Fn type III-like domain, and a transmembrane domain. Another embodiment features an LRSG protein having at least a leucine-rich region, an EGF-like domain, an Fn type III-like domain, and a transmembrane domain.

Isolated proteins of the present invention, preferably LRSG proteins, have an amino acid sequence sufficiently homologous, as defined herein, to the amino acid sequence of SEQ ID NO:59 or SEQ ID NO:68 or are encoded by a nucleotide sequence sufficiently homologous to SEQ ID NO:58, SEQ ID NO:60, SEQ ID NO:167 or SEQ ID NO:69.

As used interchangeably herein, a “LRSG activity”, “biological activity of LRSG” or “functional activity of LRSG”, refer to an activity exerted by an LRSG protein, polypeptide or nucleic acid molecule as determined in vivo, or in vitro, according to standard techniques. In one embodiment, an LRSG activity is a direct activity, such as an association with an LRSG-target molecule. As used herein, a “target molecule” is a molecule with which an LRSG protein binds or interacts in nature, such that LRSG-mediated function is achieved. An LRSG target molecule can be an LRSG protein or polypeptide of the present invention or a non-LRSG molecule. For example, an LRSG target molecule can be a non-LRSG protein molecule. Alternatively, an LRSG activity is an indirect activity, such as an activity mediated by interaction of the LRSG protein with an LRSG target molecule such that the target molecule modulates a downstream cellular activity (e.g., interaction of an LRSG molecule with an LRSG target molecule can modulate the activity of that target molecule on an intracellular signaling pathway).

In a preferred embodiment, an LRSG activity is at least one or more of the following activities: (i) interaction of an LRSG protein with an LRSG target molecule; (ii) interaction of an LRSG protein with an LRSG target molecule, wherein the LRSG target is an extracellular matrix protein; (iii) interaction of an LRSG protein with an LRSG target molecule, wherein the LRSG target is an intracellular signaling molecule; and (iv) interaction of an LRSG protein with an LRSG target molecule, wherein the LRSG target is a second molecule on the cell surface which interacts with an intracellular signaling molecule.

In yet another preferred embodiment, an LRSG activity is at least one or more of the following activities: (1) modulation of cellular signal transduction, either in vitro or in vivo; (2) modulation of protein:protein interactions, either in vitro or in vivo; (3) regulation of cellular proliferation; or (4) regulation of cellular differentiation.

Accordingly, another embodiment of the invention features isolated LRSG proteins and polypeptides having an LRSG activity. Preferred proteins are LRSG proteins having an LRR region and/or at least an Fn type III-like domain and, preferably, an LRSG activity. Additional preferred proteins are LRSG proteins having an LRR region and/or at least an EGF-like domain, an Fn type III-like domain and, preferably, an LRSG activity. In another preferred embodiment, the isolated protein further comprises a signal sequence. In still another preferred embodiment, the isolated protein is an LRSG protein having an LRR region, an Fn type III-like domain, an EGF-like domain, an LRSG activity, preferably an amino acid sequence sufficiently homologous, as defined herein, to an amino acid sequence of SEQ ID NO:59 or SEQ ID NO:68, and optionally a signal sequence and/or propeptide.

G. STMST Nucleic Acid and Protein Molecules

The human STMST-1 cDNA, which is approximately 2915 nucleotides in length, encodes a protein which is approximately 297 amino acid residues in length. The human STMST-1 protein contains 6 transmembrane domains at about amino acids 11-34, 44-67, 85-106, 127-149, 172-196, and 244-262 of SEQ ID NO:71 The human STMST-1 protein further contains a 7 transmembrane receptor profile. The 7 transmembrane receptor profile can be found at least, for example, from about amino acids 24-191 of SEQ ID NO:71.

The human STMST-2 cDNA, which is approximately 4166 nucleotides in length, encodes approximately 609 amino acid residues of the human STMST-1 protein. The human STMST-2 protein contains 7 transmembrane domains at about amino acids 11-34, 44-67, 85-106, 127-149, 172-196, 245-269, and 277-300 of SEQ ID NO:74. The human STMST-2 protein further contains a 7 transmembrane receptor profile. The 7 transmembrane receptor profile can be found at least, for example, from about amino acids 24-191 of SEQ ID NO:74. Moreover, the human STMST protein contains a spectrin α-chain profile from about amino acids 266-372 of SEQ ID NO:74. In another embodiment, the family of G protein-coupled receptors (GPCRs), to which the STMST proteins of the present invention bear significant homology, comprise an N-terminal extracellular domain, seven transmembrane domains (also referred to as membrane-spanning domains), three extracellular domains (also referred to as extracellular loops), three cytoplasmic domains (also referred to as cytoplasmic loops), and a C-terminal cytoplasmic domain (also referred to as a cytoplasmic tail). Members of the GPCR family also share certain conserved amino acid residues, some of which have been determined to be critical to receptor function and/or G protein signaling. For example, GPCRs contain the following features: a conserved asparagine residue in the first transmembrane domain; a cysteine residue in the first extracellular loop which is believed to form a disulfide bond with a conserved cysteine residue in the second extracellular loop; a conserved leucine and aspartate residue in the second transmembrane domain; an aspartate-arginine-tyrosine motif (DRY motif) at the interface of the third transmembrane domain and the second cytoplasmic loop of which the arginine residue is almost invariant (members of the rhodopsin subfamily of GPCRs comprise a histidine-arginine-methionine motif (HRM motif) as compared to a DRY motif); a conserved tryptophan and proline residue in the fourth transmembrane domain; a conserved phenylalanine residue which is commonly found as part of the motif FXXCXXP; and a conserved leucine residue in the seventh transmembrane domain which is commonly found as part of the motif DPXXY or NPXXY. Example 13 depicts an alignment of the transmembrane domain of 5 GPCRs.

The amino acid sequences of thrombin (Accession No. P25116), rhodopsin (Accession No. P08100), m1ACh (Accession No. P08482), IL-8RA (Accession No. P25024), octopamine (Accession No. P22270), can be found as SEQ ID NO:80, SEQ ID NO:81, SEQ ID NO:82, SEQ ID NO:83, and SEQ ID NO:84, respectively. Accordingly, GPCR-like proteins such as the STMST proteins of the present invention contain a significant number of structural characteristics of the GPCR family. For instance, the STMSTs of the present invention contain conserved cysteines found in the first 2 extracellular loops (prior to the third and fifth transmembrane domains) of most GPCRs (cys 83 and cys 161 of SEQ ID NO:71 or SEQ ID NO:74). A highly conserved asparagine residue in the first transmembrane domain is present (asn25 in SEQ ID NO:71 or SEQ ID NO:74). Transmembrane domain two of the STMST proteins contains a highly conserved leucine (leu49 of SEQ ID NO:71 or SEQ ID NO:74). The two cysteine residues are believed to form a disulfide bond that stabilizes the functional protein structure. A highly conserved tryptophan and proline in the fourth transmembrane domain of the STMST proteins is present (trp135 and pro 145 of SEQ ID NO:71 or SEQ ID NO:74). The third cytoplasmic loop contains 49 amino acid residues and is thus the longest cytoplasmic loop of the three, characteristic of G protein coupled receptors. Moreover, a highly conserved proline in the sixth transmembrane domain is present (pro260 of SEQ ID NO:71 and SEQ ID NO:74). The proline residues in the fourth, fifth, sixth, and seventh transmembrane domains are thought to introduce kinks in the alpha-helices and may be important in the formation of the ligand binding pocket. Furthermore, the conserved (in the second cytoplasmic loop) HRM motif found in almost all Rhodopsin family GPCRs is present in the STMST proteins of the instant invention (his 107, arg108, met109 of SEQ ID NO:71 or SEQ ID NO:74). (The arginine of the HRM sequence is thought to be the most important amino acid in GPCRs and is invariant.) Moreover, an almost invariant proline is present in the seventh transmembrane domain of STMST-2 (pro294 of SEQ ID NO:74).

In one embodiment, the STMST proteins of the present invention are proteins having an amino acid sequence of about 150-450, preferably about 200-400, more preferably about 225-375, more preferably about 250-350, or about 275-325 amino acids in length. In another embodiment, the STMST proteins of the present invention are proteins having an amino acid sequence of about 450-750, preferably about 500-700, more preferably about 525-675, even more preferably about 550-650, and even more preferably about 575-625 amino acid residues in length. In one embodiment, the STMST proteins of the present invention contain at least one transmembrane domain, as defined herein. For example, a transmembrane domain can be found at about amino acids 11-34 of SEQ ID NO:71 or SEQ ID NO:74. In a preferred embodiment, an STMST protein of the present invention has more than one transmembrane domain, preferably 2, 3, 4, 5, 6, or 7 transmembrane domains. For example, transmembrane domains can be found at about amino acids 11-34, 44-67, 85-106, 127-149, 172-196, and 244-262 of SEQ ID NO:71 as well as at 11-34, 44-67, 85-106, 127-149, 172-196, 245-269, and 277-300 of SEQ ID NO:74. In a particularly preferred embodiment, an STMST protein of the present invention has 7 transmembrane domains.

In another embodiment, an STMST family member is identified based on the presence of at least one cytoplasmic loop, also referred to herein as a cytoplasmic domain. In another embodiment, an STMST family member is identified based on the presence of at least one extracellular loop. As defined herein, the term “loop” includes an amino acid sequence having a length of at least about 4, preferably about 5-10, preferably about 10-20, and more preferably about 20-30, 30-40, 40-50, 50-60, 60-70, 70-80, 80-90, 90-100, or 100-150 amino acid residues, and has an amino acid sequence that connects two transmembrane domains within a protein or polypeptide. Accordingly, the N-terminal amino acid of a loop is adjacent to a C-terminal amino acid of a transmembrane domain in a naturally-occurring GPCR or GPCR-like molecule, and the C-terminal amino acid of a loop is adjacent to an N-terminal amino acid of a transmembrane domain in a naturally-occurring GPCR or GPCR-like molecule.

As used herein, a “cytoplasmic loop” includes an amino acid sequence located within a cell or within the cytoplasm of a cell. For example, a cytoplasmic loop is found at about amino acids 35-43, 107-126, and 197-243 of SEQ ID NO:71, or alternatively, at about amino acid residues 35-43, 107-126, and 197-244 of SEQ ID NO:74. Also as used herein, an “extracellular loop” includes an amino acid sequence located outside of a cell, or extracellularly. For example, an extracellular loop can be found at about amino acid residues 68-84 and 150-171 of SEQ ID NO:71, or alternatively, at about amino acid residues 86-84, 150-171, or 270-276 of SEQ ID NO:74.

In another embodiment of the invention, an STMST family member is identified based on the presence of a “C-terminal cytoplasmic domain”, also referred to herein as a C-terminal cytoplasmic tail, in the sequence of the protein. As used herein, a “C-terminal cytoplasmic domain” includes an amino acid sequence having a length of at least about 10, preferably about 10-25, more preferably about 25-50, more preferably about 50-75, even more preferably about 75-100, 100-150, 150-200, 200-250, 250-300, 300-400, 400-500, or 500-600 amino acid residues and is located within a cell or within the cytoplasm of a cell. Accordingly, the N-terminal amino acid residue of a “C-terminal cytoplasmic domain” is adjacent to a C-terminal amino acid residue of a transmembrane domain in a naturally-occurring GPCR or GPCR-like protein. For example, a C-terminal cytoplasmic domain is found at about amino acid residues 301-609 of SEQ ID NO:74.

In another embodiment, an STMST family member is identified based on the presence of an “N-terminal extracellular domain”, also referred to herein as an N-terminal extracellular loop in the amino acid sequence of the protein. As used herein, an “N-terminal extracellular domain” includes an amino acid sequence having about 1-500, preferably about 1-400, more preferably about 1-300, more preferably about 1-200, even more preferably about 1-100, and even more preferably about 1-50, 1-25, or 1-10 amino acid residues in length and is located outside of a cell or extracellularly. The C-terminal amino acid residue of a “N-terminal extracellular domain” is adjacent to an N-terminal amino acid residue of a transmembrane domain in a naturally-occurring GPCR or GPCR-like protein. For example, an N-terminal cytoplasmic domain is found at about amino acid residues 1-10 of SEQ ID NO:71 or SEQ ID NO:74.

Accordingly in one embodiment of the invention, an STMST family member includes at least one, preferably 6 or 7, transmembrane domains and/or at least one cytoplasmic loop, and/or at least one extracellular loop. In another embodiment, the STMST family member further includes an N-terminal extracellular domain and/or a C-terminal cytoplasmic domain. In another embodiment, the STMST family member can include six transmembrane domains, three cytoplasmic loops, and two extracellular loops, or can include six transmembrane domains, three extracellular loops, and 2 cytoplasmic loops. The former embodiment can further include an N-terminal extracellular domain. The latter embodiment can further include a C-terminal cytoplasmic domain. In another embodiment, the STMST family member can include seven transmembrane domains, three cytoplasmic loops, and three extracellular loops and can further include an N-terminal extracellular domain or a C-terminal cytoplasmic domain.

In another embodiment, an STMST family member is identified based on the presence of at least one “7 transmembrane receptor profile”, also referred to as a “Rhodopsin family sequence profile”, in the protein or corresponding nucleic acid molecule. As used herein, the term “7 transmembrane receptor profile” includes an amino acid sequence having at least about 100-400, preferably about 150-350, more preferably about 200-300 amino acid residues, or at least about 250-275 amino acids in length and having a bit score for the alignment of the sequence to the 7tm_{—}1 family Hidden Markov Model (HMM) of at least 20, preferably 20-30, more preferably 30-40, more preferably 40-50, 50-75, 75-100, 100-200 or greater. The 7tm_{—}1 family HMM has been assigned the PFAM Accession PF00001 (see the PFAM website, available through Washington University in Saint Louis).

To identify the presence of a 7 transmembrane receptor profile in an STMST family member, the amino acid sequence of the protein family member is searched against a database of HMMs (e.g., the Pfam database, release 2.1) using the default parameters. For example, the hmmsf program, which is available as part of the HMMER package of search programs, is a family specific default program for PF00001 and score of 15 is the default threshold score for determining a hit. For example, a search using the amino acid sequence of SEQ ID NO:71 was performed against the HMM database resulting in the identification of a 7 TM receptor profile in the amino acid sequence of SEQ ID NO:71. The results of the search are set forth in Example 13.

Likewise, a search using the amino acid sequence of SEQ ID NO:74 results in an identical hit with a score of 44.14 against the 7tm_{—}1 family HMM Accession PF00001. Accordingly, in one embodiment of the invention, an STMST protein is a human STMST-1 or a human STMST-2 protein having a 7 transmembrane receptor profile at about amino acids 24-191 of SEQ ID NO:71 or SEQ ID NO:74, respectively. Such a 7 transmembrane receptor profile has the amino acid sequence of SEQ ID NO:173).

Accordingly, STMST family members having at least 50-60% homology, preferably about 60-70%, more preferably about 70-80%, or about 80-90% homology with the 7 transmembrane receptor profile of human STMST-1 or STMST-2 (e.g., SEQ ID NO:173) are within the scope of the invention.

In another embodiment, an STMST family member is identified based on the presence of a “spectrin α-chain profile” in the protein or corresponding nucleic acid molecule. As used herein, the term “spectrin α-chain profile” includes a protein domain having an amino acid sequence of about 50-250, preferably about 75-225, more preferably about 100-200 amino acid residues, or about 125-175 amino acids and having a bit score for the alignment of the sequence to the spectrin family (HMM) of at least 7, preferably 8-10, more preferably 10-30, more preferably 30-50, even more preferably 50-75, 75-100, 100-200 or greater. The spectrin family HMM has been assigned the PFAM Accession PF00435 (see the PFAM website, available through Washington University in Saint Louis).

To identify the presence of a spectrin alpha chain profile in a STMST family member, make the determination that a protein of interest has a particular profile, the amino acid sequence of the protein is searched against a database of HMMs (e.g., the Pfam database, release 2.1) using the default parameters. For example, the hmmsf program, which is available as part of the HMMER package of search programs, is a family specific default program for PF00435 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3)405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. A search was performed against the HMM database resulting in the identification of a spectrin alpha chain profile in the amino acid sequence of SEQ ID NO:71. The results of the search are set forth in Example 13.

Accordingly, in one embodiment, an STMST protein is human STMST-2 protein which includes a spectrin α-chain profile at about amino acids 266-372 of SEQ ID NO:74. Such a spectrin α-chain profile has the amino acid sequence of SEQ ID NO:175).

Accordingly, STMST family members having at least 50-60% homology, preferably about 60-70%, more preferably about 70-80%, or about 80-90% homology with a spectrin α-chain profile of human STMST-2 (e.g., SEQ ID NO:175) are within the scope of the invention.

In another embodiment, an STMST protein includes at least a spectrin α-chain profile. In another embodiment, an STMST protein includes a spectrin α-chain profile and a 7 transmembrane receptor profile. In another embodiment, an STMST protein is human STMST-2 which includes a spectrin α-chain profile having about amino acids 266-372 of SEQ ID NO:74. In yet another embodiment, an STMST protein is human STMST-2 which includes a 7 transmembrane receptor profile having about amino acids 24-191 of SEQ ID NO:74 and a spectrin α-chain profile having about amino acids 266-372 of SEQ ID NO:74.

Preferred STMST molecules of the present invention have an amino acid sequence sufficiently homologous, as defined herein, to the amino acid sequence of SEQ ID NO:71 or SEQ ID NO:74.

As used interchangeably herein, an “STMST activity”, “biological activity of STMST” or “functional activity of STMST”, refer to an activity exerted by an STMST protein, polypeptide or nucleic acid molecule on an STMST responsive cell as determined in vivo, or in vitro, according to standard techniques. In one embodiment, an STMST activity is a direct activity, such as an association with an STMST-target molecule. As used herein, a “target molecule” or “binding partner” is a molecule with which an STMST protein binds or interacts in nature, such that STMST-mediated function is achieved. An STMST target molecule can be a non-STMST molecule or an STMST protein or polypeptide of the present invention. In an exemplary embodiment, an STMST target molecule is an STMST ligand. Alternatively, an STMST activity is an indirect activity, such as a cellular signaling activity mediated by interaction of the STMST protein with an STMST ligand.

In a preferred embodiment, an STMST activity is at least one or more of the following activities: (i) interaction of an STMST protein with soluble STMST ligand; (ii) interaction of an STMST protein with a membrane-bound non-STMST protein; (iii) interaction of an STMST protein with an intracellular protein (e.g., an intracellular enzyme or signal transduction molecule); and (iv) indirect interaction of an STMST protein with an intracellular protein (e.g., a downstream signal transduction molecule).

STMST is a GPCR-like protein having significant homology to at least the A-1 family of GPCR-like proteins. STMST has been determined by Northern blot analysis to be expressed in many tissues, including but not limited to heart, brain (including fetal brain), placenta, lung, liver, skeletal muscle and kidney (fetal). Moreover, detailed analysis of expression by TaqMan™ RT-PCR analysis indicates that STMST is highly expressed in osteoblasts (e.g., osteoblastic cell lines as well as primary osteoblasts). Expression in primary osteoblasts is further inducible by treatment of cells with parathyroid hormone (PTH) suggesting that STMST and/or STMST agonism may mimic PTH anabolic effects on bone. Expression is also inducible by dexamethasone treatment which stimulates primary osteoblasts to differentiate in vitro. Northern blot analysis confirms expression of STMST in primary osteoblasts. STMST has further been determined by in situ analysis to be expressed in osteoblasts of human fetal bone. STMST is also expressed within the arcuate nucleus and the ventromedial nucleus of the hypothalamus, both of which are implicated in control of feeding behavior. STMST is further expressed in endothelial cells and is downregulated during tube formation induced by plating on Matrigel.

Accordingly, in one embodiment, an STMST modulator, is useful for (i) modulating osteogenic cell function (e.g., osteoblast function); (ii) modulating bone homeostasis; (iii) modulation of bone resorption; and (iv) modulation of bone formation (e.g., stimulation of bone mass and/or inhibition of bone loss). In another embodiment, an STMST modulator is useful for (1) regulating, preventing and/or treating bone-related disorders including, but not limited to osteoporosis, Paget's disease, osteoarthritis, degenerative arthritis, osteogenesis imperfecta, fibrous dysplasia, hypophosphatasia, bone sarcoma, myeloma bone disorder (e.g., osteolytic bone lesions) and hypercalcemia; (2) management of bone fragility (e.g., decrease bone fragility); and (3) prevention and/or treatment of bone pain, bone deformities, and/or bone fractures.

In another embodiment, an STMST activity is at least one or more of the following activities: (1) modulation of cellular signal transduction, either in vitro or in vivo; (2) regulation of gene transcription in a cell expressing an STMST protein; (3) regulation of gene transcription in a cell expressing an STMST protein, wherein said cell is involved inflammation; (4) regulation of cellular proliferation; (5) regulation of cellular differentiation; (6) regulation of development; (7) regulation of cell death; (8) regulation of regulation of inflammation; (9) regulation of respiratory cell function (e.g., asthma); (10) regulation of actin binding; (11) regulation of cytoskeletal attachment; and (12) regulation of chemotaxis, trafficking and/or migration.

Yet another embodiment of the invention features isolated STMST proteins and polypeptides having an STMST activity. Preferred STMST proteins have at least one transmembrane domain and an STMST activity. In a preferred embodiment, an STMST protein has a 7 transmembrane receptor profile and an STMST activity. In another preferred embodiment, an STMST protein has a spectrin α-chain profile and an STMST activity. In still another preferred embodiment, an STMST protein has a 7 transmembrane receptor profile, a spectrin α-chain profile, and STMST activity. In still another preferred embodiment, an STMST protein has a 7 transmembrane receptor profile, a spectrin α-chain profile, an STMST activity, and an amino acid sequence sufficiently homologous, as defined herein, to an amino acid sequence of SEQ ID NO:71, SEQ ID NO:74, or SEQ ID NO:77.

For convenience, the following table lists SEQ ID NOs for the nucleic acid and polypeptide sequences of the invention:

| SEQ ID NO: | Description of Sequence | Figure No. |
|---|---|---|
| 1 | human ITALY nucleic acid sequence | 1 |
| 2 | human ITALY amino acid sequence | 1 |
| 3 | human ITALY coding sequence | 1 |
| 4 | human ITALY mature polypeptide sequence | |
| | without the signal sequence | |
| 10 | human Lor-2 nucleic acid sequence | 3A-3C |
| 11 | human Lor-2 amino acid sequence | 4A-4C |
| 12 | human Lor-2 coding sequence | 5A-5B |
| 22 | mouse STRIFE1 nucleic acid sequence | 12A-12B |
| 23 | mouse STRIFE1 amino acid sequence | 12A-12B |
| 24 | mouse STRIFE1 coding sequence | 12A-12B |
| 25 | mouse STRIFE1 nucleic acid sequence | |
| 26 | mouse STRIFE2 nucleic acid sequence | 13 |
| 27 | mouse STRIFE2 amino acid sequence | 13 |
| 28 | mouse STRIFE2 coding sequence | 13 |
| 29 | mouse STRIFE2 nucleic acid sequence | |
| 31 | human TRASH nucleic acid sequence | 17 |
| 32 | human TRASH amino acid sequence | 17 |
| 33 | human TRASH coding sequence | 17 |
| 36 | human TRASH nucleic acid sequence | |
| 37 | human TRASH amino acid sequence | |
| 38 | human TRASH coding sequence - alternate | |
| | translation start site | |
| 39 | human TRASH amino acid sequence - alternate | |
| | translation start site | |
| 40 | human TRASH coding sequence - alternate | |
| | translation start site | |
| 41 | human TRASH amino acid sequence - alternate | |
| | translation start site | |
| 45 | human BDSF-1 nucleic acid sequence | 19A-19B |
| 46 | human BDSF-1 amino acid sequence | 19A-19B |
| 47 | human BDSF-1 coding sequence | 19A-19B |
| 48 | human BDSF-1 nucleic acid sequence | |
| 49 | human BDSF-1 amino acid sequence | |
| 50 | mouse BDSF-1 nucleic acid sequence | 20A-20C |
| 51 | mouse BDSF-1 amino acid sequence | 20A-20C |
| 52 | mouse BDSF-1 coding sequence | 20A-20C |
| 53 | mouse BDSF-1 nucleic acid sequence | |
| 54 | mouse BDSF-1 amino acid sequence | |
| 58 | human LRSG-1 nucleic acid sequence | 22A-22E |
| 59 | human LRSG-1 amino acid sequence | 22A-22E |
| 60 | human LRSG-1 coding sequence | 22A-22E |
| 67 | mouse LRSG-1 nucleic acid sequence | 24A-24E |
| 68 | mouse LRSG-1 amino acid sequence | 24A-24E |
| 69 | mouse LRSG-1 coding sequence | 24A-24E |
| 70 | human STMST-1 nucleic acid sequence | 27A-27B |
| 71 | human STMST-1 amino acid sequence | 27A-27B |
| 72 | human STMST-1 coding sequence | 27A-27B |
| 73 | human STMST-2 nucleic acid sequence | 28A-28C |
| 74 | human STMST-2 amino acid sequence | 28A-28C |
| 75 | human STMST-2 coding sequence | 28A-28C |

Various aspects of the invention are described in further detail in the following subsections:

### I. Isolated Nucleic Acid Molecules

One aspect of the invention pertains to isolated nucleic acid molecules that encode proteins of the invention or biologically active portions thereof, as well as nucleic acid fragments sufficient for use as hybridization probes to identify ITALY, Lor-2, STRIFE, TRASH, BDSF, LRSG, and STMST-encoding nucleic acids (e.g., ITALY, Lor-2, STRIFE, TRASH, BDSF, LRSG, and STMST mRNA) and fragments for use as PCR primers for the amplification or mutation of the nucleic acid molecules of the invention. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule of the invention can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, or a portion thereof, can be isolated using standard molecular biology techniques and the sequence information provided herein. Using all or portion of the nucleic acid sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, as a hybridization probe, the nucleic acid molecules of the invention can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695 can be isolated by the polymerase chain reaction (PCR) using synthetic oligonucleotide primers designed based upon the sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to nucleotide sequences of the invention can be prepared by standard synthetic techniques, e.g., using an automated DNA synthesizer.

In a preferred embodiment, an isolated nucleic acid molecule of the invention comprises the nucleotide sequence shown in SEQ ID NO:1. The sequence of SEQ ID NO:1 corresponds to the human ITALY cDNA. This cDNA comprises sequences encoding the human ITALY protein (i.e., “the coding region”, from nucleotides 43-573), as well as 5′ untranslated sequences (nucleotides 1-42) and 3′ untranslated sequences (nucleotides 574-991). Alternatively, the nucleic acid molecule can comprise only the coding region of SEQ ID NO:1 (e.g., nucleotides 43-573, corresponding to SEQ ID NO:3).

In another preferred embodiment, an isolated nucleic acid molecule of the invention comprises the nucleotide sequence shown in SEQ ID NO:12. The sequence of SEQ ID NO:12 corresponds to the coding region of human Lor-2 cDNA. This cDNA comprises sequences encoding the human Lor-2 protein (i.e., “the coding region”, from nucleotides 143-2401 of SEQ ID NO:10).

In yet another embodiment, an isolated nucleic acid molecule of the invention comprises the nucleotide sequence shown in SEQ ID NO:10. The sequence of SEQ ID NO:10 corresponds to the coding and noncoding regions of human Lor-2 cDNA. This cDNA comprises sequences encoding the human Lor-2 protein (i.e., “the coding region”, from nucleotides 143-2401) and noncoding regions (i.e., from nucleotides 1-142 and from nucleotides 2402-2920).

In another preferred embodiment, an isolated nucleic acid molecule of the invention comprises the nucleotide sequence shown in SEQ ID NO:22. The sequence of SEQ ID NO:22 corresponds to the murine STRIFE1 cDNA. This cDNA comprises sequences encoding the murine STRIFE1 protein (i.e., “the coding region”, from nucleotides 107-751), as well as 5′ untranslated sequences (nucleotides 1 to 106) and 3′ untranslated sequences (nucleotides 752-981). Alternatively, the nucleic acid molecule can comprise only the coding region of SEQ ID NO:22 (e.g., nucleotides 107-751, corresponding to SEQ ID NO:24).

In another preferred embodiment, an isolated nucleic acid molecule of the invention comprises the nucleotide sequence shown in SEQ ID NO:26. The sequence of SEQ ID NO:5 corresponds to the murine STRIFE2 cDNA. This cDNA comprises sequences encoding the murine STRIFE2 protein (i.e., “the coding region”, from nucleotides 110-562), as well as 5′ untranslated sequences (nucleotides 1-109) and 3′ untranslated sequences (nucleotides 563-655). Alternatively, the nucleic acid molecule can comprise only the coding region of SEQ ID NO:26 (e.g., nucleotides 110-562, corresponding to SEQ ID NO:28).

In another preferred embodiment, an isolated nucleic acid molecule of the invention comprises the nucleotide sequence shown in SEQ ID NO:31, SEQ ID NO:33, SEQ ID NO:36, SEQ ID NO:38, or SEQ ID NO:40. The sequence of SEQ ID NO:31 corresponds to a human TRASH cDNA. This cDNA comprises sequences encoding a human TRASH protein (i.e., “the coding region”, from nucleotides 273 to 1025), as well as 5′ untranslated sequences (nucleotides 1 to 272) and 3′ untranslated sequences (nucleotides 1026 to 1346). Alternatively, the nucleic acid molecule can comprise only the coding region of SEQ ID NO:31 (e.g., nucleotides 273 to 1025, corresponding to nucleotides 1-753 of SEQ ID NO:33).

The human TRASH cDNA of SEQ ID NO:31 also contains additional in frame ATG codons at positions 324-326 and 408-410 of SEQ ID NO:31 encoding methionines at positions 18 and 46 of SEQ ID NO:32, respectively. Thus, in another embodiment, the nucleotide sequence of SEQ ID NO:31 comprises a coding region from nucleotides 324 to 1025 (SEQ ID NO:38), as well as 5′ untranslated sequences from nucleotides 1 to 323 and 3′ untranslated sequences from nucleotides 1026 to 1346. In yet another embodiment, the nucleotide sequence of SEQ ID NO:31 comprises a coding region from nucleotides 408 to 1025 (SEQ ID NO:40), as well as 5′ untranslated sequences from nucleotides 1 to 407 and 3′ untranslated sequences from nucleotides 1026 to 1346.

In another preferred embodiment, an isolated nucleic acid molecule of the invention comprises the nucleotide sequence shown in SEQ ID NO:45. The sequence of SEQ ID NO:45 corresponds to the human BDSF cDNA. This cDNA comprises sequences encoding the human BDSF protein (i.e., “the coding region”, from nucleotides 139-874), as well as 5′ untranslated sequences (nucleotides 1-138) and 3′ untranslated sequences (nucleotides 875-1119). Alternatively, the nucleic acid molecule can comprise only the coding region of SEQ ID NO:45 (e.g., nucleotides 139-874, corresponding to SEQ ID NO:47).

In another preferred embodiment, an isolated nucleic acid molecule of the invention comprises the nucleotide sequence shown in SEQ ID NO:50. The sequence of SEQ ID NO:50 corresponds to the murine BDSF cDNA. This cDNA comprises sequences encoding the murine BDSF protein (i.e., “the coding region”, from nucleotides 268-1023), as well as 5′ untranslated sequences (nucleotides 1-267) and 3′ untranslated sequences (nucleotides 1024-3196). Alternatively, the nucleic acid molecule can comprise only the coding region of SEQ ID NO:50 (e.g., nucleotides 268-1023, corresponding to SEQ ID NO:52).

In another preferred embodiment, an isolated nucleic acid molecule of the invention comprises the nucleotide sequence shown in SEQ ID NO:58. The sequence of SEQ ID NO:58 corresponds to the human LRSG-1 cDNA. This cDNA comprises sequences encoding the human LRSG-1 protein (i.e., “the coding region”, from nucleotides 244-1122), as well as 5′ untranslated sequences (nucleotides 1-243) and 3′ untranslated sequences (nucleotides 1123-2852). Alternatively, the nucleic acid molecule can comprise only the coding region of SEQ ID NO:58 (e.g., nucleotides 244-1122, corresponding to SEQ ID NO:60).

In another preferred embodiment, an isolated nucleic acid molecule of the invention comprises the nucleotide sequence shown in SEQ ID NO:67. The sequence of SEQ ID NO:67 corresponds to the murine LRSG-1 cDNA. This cDNA comprises sequences encoding the murine LRSG-1 protein (i.e., “the coding region”, from nucleotides 197-2215), as well as 5′ untranslated sequences (nucleotides 1-196) and 3′ untranslated sequences (nucleotides 2216-2815). Alternatively, the nucleic acid molecule can comprise only the coding region of SEQ ID NO:67 (e.g., nucleotides 197-2215, corresponding to SEQ ID NO:69).

In another preferred embodiment, an isolated nucleic acid molecule of the invention comprises the nucleotide sequence shown in SEQ ID NO:70. The sequence of SEQ ID NO:70 corresponds to the human STMST-1 cDNA. This cDNA comprises sequences encoding the human STMST-1 protein (i.e., “the coding region”, from nucleotides 404-1294), as well as 5′ untranslated sequences (nucleotides 1-403) and 3′ untranslated sequences (nucleotides 1295-2915). Alternatively, the nucleic acid molecule can comprise only the coding region of SEQ ID NO:70 (e.g., nucleotides 404-1294, corresponding to SEQ ID NO:72).

In another preferred embodiment, an isolated nucleic acid molecule of the invention comprises the nucleotide sequence shown in SEQ ID NO:73. The sequence of SEQ ID NO:73 corresponds to the human STMST-2 cDNA. This cDNA comprises sequences encoding the human STMST-2 protein (i.e., “the coding region”, from nucleotides 334-2160), as well as 5′ untranslated sequences (nucleotides 1-333) and 3′ untranslated sequences (nucleotides 2161-4166). Alternatively, the nucleic acid molecule can comprise only the coding region of SEQ ID NO:73 (e.g., nucleotides 334-2160, corresponding to SEQ ID NO:75).

In another preferred embodiment, an isolated nucleic acid molecule of the invention comprises a nucleic acid molecule which is a complement of the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, or a portion of any of these nucleotide sequences. A nucleic acid molecule which is complementary to the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, is one which is sufficiently complementary to the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, such that it can hybridize to the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, thereby forming a stable duplex.

In still another preferred embodiment, an isolated nucleic acid molecule of the present invention comprises a nucleotide sequence which is at least about 58%, 60%, 65%, 70%, 75%, 80%, 85%, 90%, 95% or more homologous to the entire length of the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the entire length of the nucleotide sequence of the DNA insert of the plasmid deposited with ATCC as Accession Number 98960, 98756, or 98695, or a portion of any of these nucleotide sequences.

Moreover, the nucleic acid molecule of the invention can comprise only a portion of the nucleic acid sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, for example a fragment which can be used as a probe or primer or a fragment encoding a biologically active portion of a protein of the invention. The nucleotide sequence determined from the cloning of the ITALY, Lor-2, STRIFE, TRASH, BDSF, LRSG, or STMST gene allows for the generation of probes and primers designed for use in identifying and/or cloning other ITALY, Lor-2, STRIFE, TRASH, BDSF, LRSG, or STMST family members, as well as ITALY, Lor-2, STRIFE, TRASH, BDSF, LRSG, or STMST homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, of an anti-sense sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, or of a naturally occurring allelic variant or mutant of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695. In an exemplary embodiment, a nucleic acid molecule of the present invention comprises a nucleotide sequence which is greater that 588, 600-650, 651-700, 701-750, or 751-800 nucleotides in length and hybridizes under stringent hybridization conditions to a nucleic acid molecule of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695.

Probes based on the nucleotide sequences of the invention can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a protein of the invention, such as by measuring a level of an ITALY, Lor-2, STRIFE, TRASH, BDSF, LRSG, or STMST-encoding nucleic acid in a sample of cells from a subject e.g., detecting ITALY, Lor-2, STRIFE, TRASH, BDSF, LRSG, or STMST mRNA levels or determining whether a genomic ITALY, Lor-2, STRIFE, TRASH, BDSF, LRSG, or STMST gene has been mutated or deleted.

A nucleic acid fragment encoding a “biologically active portion of a protein of the invention” can be prepared by isolating a portion of the nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, which encodes a polypeptide having an ITALY, Lor-2, STRIFE, TRASH, BDSF, LRSG, or STMST biological activity (the biological activities of the proteins of the invention are described herein), expressing the encoded portion of the protein of the invention (e.g., by recombinant expression in vitro) and assessing the activity of the encoded portion of the protein of the invention.

The invention further encompasses nucleic acid molecules that differ from the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, due to degeneracy of the genetic code and thus encode the same proteins of the invention as those encoded by the nucleotide sequence shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695. In another embodiment, an isolated nucleic acid molecule of the invention has a nucleotide sequence encoding a protein having an amino acid sequence shown in SEQ ID NO:2, 4, 11, 23, 27, 32, 37, 39, 41, 46, 49, 51, 54, 59, 68, 71, or 74.

In addition to the nucleotide sequences of the invention shown in SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, it will be appreciated by those skilled in the art that DNA sequence polymorphisms that lead to changes in the amino acid sequences of the proteins of the invention may exist within a population (e.g., the human population). Such genetic polymorphism in the ITALY, Lor-2, STRIFE, TRASH, BDSF, LRSG, or STMST genes may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. For example, the Lor-2 gene exhibits significant homology with a portion of human chromosome 2 near the D2S145 marker (e.g., having Accession Nos. AA191602 and R55706). Allelic variants of any of these genes can be identified by sequencing the corresponding chromosomal portion at the indication location in multiple individuals.

As used herein, the terms “gene” and “recombinant gene” refer to nucleic acid molecules comprising an open reading frame encoding a protein of the invention, preferably a mammalian ITALY, Lor-2, STRIFE, TRASH, BDSF, LRSG, or STMST protein. Such natural allelic variations can typically result in 1-5% variance in the nucleotide sequence of an ITALY, Lor-2, STRIFE, TRASH, BDSF, LRSG, or STMST gene. Any and all such nucleotide variations and resulting amino acid polymorphisms in ITALY, Lor-2, STRIFE, TRASH, BDSF, LRSG, or STMST genes that are the result of natural allelic variation and that do not alter the functional activity of an ITALY, Lor-2, STRIFE, TRASH, BDSF, LRSG, or STMST protein are intended to be within the scope of the invention.

Moreover, nucleic acid molecules encoding other ITALY family members (e.g., other IL-10 family members), other Lor-2 family members, other STRIFE family members, other TRASH family members, other BDSF family members, other LRSG family members (e.g., LRSG-2), or other STMST family members, and thus which have a nucleotide sequence which differs from the nucleic acid sequences of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695 are intended to be within the scope of the invention. For example, another ITALY, Lor-2, STRIFE, TRASH, BDSF, LRSG, or STMST cDNA can be identified based on the nucleotide sequence of human ITALY, Lor-2, STRIFE, TRASH, BDSF, LRSG, or STMST. Moreover, nucleic acid molecules encoding ITALY, Lor-2, STRIFE, TRASH, BDSF, LRSG, or STMST proteins from different species, and thus which have a nucleotide sequence which differs from the nucleic acid sequences of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695 are intended to be within the scope of the invention. For example, a mouse ITALY, Lor-2, STRIFE, TRASH, BDSF, LRSG, or STMST cDNA can be identified based on the nucleotide sequence of a human ITALY, Lor-2, TRASH, BDSF, LRSG, or STMST.

Nucleic acid molecules corresponding to natural allelic variants and homologues of the cDNAs of the invention can be isolated based on their homology to the ITALY, Lor-2, STRIFE, TRASH, BDSF, LRSG, or STMST nucleic acids disclosed herein using the cDNAs disclosed herein, or a portion thereof, as a hybridization probe according to standard hybridization techniques under stringent hybridization conditions. Nucleic acid sequence homology can be determined by employing the techniques taught herein.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695. In other embodiment, the nucleic acid is at least 30, 50, 100, 250, 500, 588, or 600 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the sequences of the invention that may exist in the population, the skilled artisan will further appreciate that changes can be introduced by mutation into the nucleotide sequences of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, thereby leading to changes in the amino acid sequence of the encoded proteins of the invention, without altering the functional ability of the proteins of the invention. For example, nucleotide substitutions leading to amino acid substitutions at “non-essential” amino acid residues can be made in the sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695. A “non-essential” amino acid residue is a residue that can be altered from the wild-type sequence of ITALY, Lor-2, STRIFE, TRASH, BDSF, LRSG, or STMST (e.g., the sequence of SEQ ID NO:2, 4, 11, 23, 27, 32, 37, 39, 41, 46, 49, 51, 54, 59, 68, 71, or 74) without altering the biological activity, whereas an “essential” amino acid residue is required for biological activity. For example, amino acid residues that are conserved among the ITALY proteins of the present invention are predicted to be particularly unamenable to alteration (e.g., the conserved cysteines involved in forming disulfide linkages). Furthermore, additional amino acid residues that are conserved between the ITALY proteins of the present invention and other members of the IL-10 protein families are not likely to be amenable to alteration. In another example, amino acid residues that are conserved among the Lor-2 proteins of the present invention are predicted to be particularly unamenable to alteration (e.g., amino acid residues conserved among the proteins aligned in FIGS. 7A-7B). Moreover, amino acid residues that are defined by the SRCR domains are particularly unamenable to alteration. Furthermore, additional amino acid residues that are conserved between the Lor-2 proteins of the present invention and other members of the lysyl oxidase superfamily or protein families containing LOX are not likely to be amenable to alteration. In another example, amino acid residues that are conserved among the STRIFE1 or STRIFE2 proteins of the present invention, are predicted to be particularly unamenable to alteration. Furthermore, amino acid residues that are conserved between STRIFE1 or STRIFE2 protein and other proteins having cysteine-rich domains are not likely to be amenable to alteration. In another example, amino acid residues that are conserved among the TRASH proteins of the present invention, are predicted to be particularly unamenable to alteration. Furthermore, amino acid residues that are conserved between TRASH protein and other proteins having TNFL-like domains are not likely to be amenable to alteration. In another example, amino acid residues that are conserved among the BDSF proteins of the present invention, are predicted to be particularly unamenable to alteration (e.g., the ten conserved cysteines involved in forming disulfide linkages or the conserved histidine, aspartate, or serine of the active enzymatic site). Moreover, amino acid residues that are defined by the Ig-like domain profile sequence are particularly unamenable to alteration. Furthermore, additional amino acid residues that are conserved between the BDSF proteins of the present invention and other BDSF family members. In another example, amino acid residues that are conserved among the LRSG proteins of the present invention, are predicted to be particularly unamenable to alteration (e.g., the ten conserved cysteines involved in forming disulfide linkages or the conserved histidine, aspartate, or serine of the active enzymatic site). Moreover, amino acid residues that are defined by the LRSG EGF-like domain and LRSG Fn type III-like domain signature motifs are particularly unamenable to alteration. Furthermore, additional amino acid residues that are conserved between the LRSG proteins of the present invention and other members of the LRR superfamily or protein families containing EGF-like or Fn type III-like domains are not likely to be amenable to alteration. In another example, amino acid residues that are conserved among the STMST proteins of the present invention, are predicted to be particularly unamenable to alteration. Moreover, amino acid residues that are defined by the 7 transmembrane signature profile and the spectrin α-chain, repeated domain signature profile are particularly unamenable to alteration. Furthermore, additional amino acid residues that are conserved between the STMST proteins of the present invention and members of the G protein coupled receptor protein family are not likely to be amenable to alteration (e.g., the conserved asn residue within the first TM domain, asn25 of SEQ ID NO:71 or SEQ ID NO:74; the conserved cys in the first extracellular loop, cys83 of SEQ ID NO:71 or SEQ ID NO:74; the conserved arg at the interface of the third TM domain and the first cytoplasmic loop, arg108 of SEQ ID NO:71 or SEQ ID NO:74; the conserved trp and pro in the fourth TM domain, trp135 and pro145 of SEQ ID NO:71 or SEQ ID NO:74; the conserved cys residue in the second extracellular domain, cys161 of SEQ ID NO:71 or SEQ ID NO:74; the conserved phe residue in the fifth TM domain, phe251 of SEQ ID NO:71 or SEQ ID NO:74; or the conserved pro in the seventh TM domain, pro294 of SEQ ID NO:74).

Accordingly, another aspect of the invention pertains to nucleic acid molecules encoding the proteins of the invention that contain changes in amino acid residues that are not essential for activity. Such ITALY, Lor-2, STRIFE, TRASH, BDSF, LRSG, and STMST proteins differ in amino acid sequence from SEQ ID NO:2, 4, 11, 23, 27, 32, 37, 39, 41, 46, 49, 51, 54, 59, 68, 71, or 74 yet retain biological activity. In one embodiment, the isolated nucleic acid molecule comprises a nucleotide sequence encoding a protein, wherein the protein comprises an amino acid sequence at least about 32%, 35%, 40%, 45%, 50%, 55%, 60%, 65%, 70%, 75%, 80%, 85%, 90%, 95% or more homologous to SEQ ID NO:2, 4, 11, 23, 27, 32, 37, 39, 41, 46, 49, 51, 54, 59, 68, 71, or 74.

An isolated nucleic acid molecule encoding an ITALY, Lor-2, STRIFE, TRASH, BDSF, LRSG, or STMST protein homologous to the protein of SEQ ID NO:2, 4, 11, 23, 27, 32, 37, 39, 41, 46, 49, 51, 54, 59, 68, 71, or 74 can be created by introducing one or more nucleotide substitutions, additions or deletions into the nucleotide sequence of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, such that one or more amino acid substitutions, additions or deletions are introduced into the encoded protein. Mutations can be introduced into SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an ITALY, Lor-2, STRIFE, TRASH, BDSF, LRSG, or STMST protein is preferably replaced with another amino acid residue from the same side chain family. Alternatively, in another embodiment, mutations can be introduced randomly along all or part of an ITALY, Lor-2, STRIFE, TRASH, BDSF, LRSG, or STMST coding sequence, such as by saturation mutagenesis, and the resultant mutants can be screened for ITALY, Lor-2, STRIFE, TRASH, BDSF, LRSG, or STMST biological activity to identify mutants that retain activity. Following mutagenesis of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695, the encoded protein can be expressed recombinantly and the activity of the protein can be determined.

In one embodiment, a mutant polypeptide that is a variant of a polypeptide of the invention can be assayed for: (1) the ability to form protein:protein interactions with a polypeptide of the invention; (2) the ability to bind a ligand of a polypeptide of the invention; (3) the ability to bind with a modulator or substrate of a polypeptide of the invention; (4) the ability to modulate a physiological activity of a polypeptide of the invention, such as one of those disclosed herein; or (5) the ability to catalyze a reaction catalyzed by a polypeptide of the invention. In another preferred embodiment, a mutant ITALY, Lor-2, STRIFE, TRASH, BDSF, LRSG, or STMST protein can be assayed for demonstration of the ITALY, Lor-2, STRIFE, TRASH, BDSF, LRSG, or STMST activities, as described herein.

In addition to the nucleic acid molecules encoding ITALY, Lor-2, STRIFE, TRASH, BDSF, LRSG, and STMST proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire ITALY, Lor-2, STRIFE, TRASH, BDSF, LRSG, or STMST coding strand, or to only a portion thereof. In one embodiment, an antisense nucleic acid molecule is antisense to a “coding region” of the coding strand of a nucleotide sequence encoding ITALY, Lor-2, STRIFE, TRASH, BDSF, LRSG, or STMST. The term “coding region” refers to the region of the nucleotide sequence comprising codons which are translated into amino acid residues (e.g., the coding region of human ITALY corresponds to SEQ ID NO:3; the coding region of human Lor-2 corresponds to SEQ ID NO:12; the coding region of mouse STRIFE1 corresponds to SEQ ID NO:24; the coding region of mouse STRIFE2 corresponds to SEQ ID NO:28; the coding region of human TRASH corresponds to SEQ ID NO:33, 38, or 40; the coding region of human BDSF-1 corresponds to SEQ ID NO:47; the coding region of mouse BDSF-1 corresponds to SEQ ID NO:52; the coding region of human LRSG-1 corresponds to SEQ ID NO:60; the coding region of mouse LRSG-1 corresponds to SEQ ID NO:69; the coding region of human STMST-1 corresponds to SEQ ID NO:72; and the coding region of human STMST-2 corresponds to SEQ ID NO:75). In another embodiment, the antisense nucleic acid molecule is antisense to a “noncoding region” of the coding strand of a nucleotide sequence encoding ITALY, Lor-2, STRIFE, TRASH, BDSF, LRSG, or STMST. The term “noncoding region” refers to 5′ and 3′ sequences which flank the coding region that are not translated into amino acids (i.e., also referred to as 5′ and 3′ untranslated regions).

Given the coding strand sequences encoding ITALY, Lor-2, STRIFE, TRASH, BDSF, LRSG, or STMST disclosed herein (e.g., SEQ ID NO:3), antisense nucleic acids of the invention can be designed according to the rules of Watson and Crick base pairing. The antisense nucleic acid molecule can be complementary to the entire coding region of ITALY, Lor-2, STRIFE, TRASH, BDSF, LRSG, or STMST mRNA, but more preferably is an oligonucleotide which is antisense to only a portion of the coding or noncoding region of ITALY, Lor-2, STRIFE, TRASH, BDSF, LRSG, or STMST mRNA. For example, the antisense oligonucleotide can be complementary to the region surrounding the translation start site of ITALY, Lor-2, STRIFE, TRASH, BDSF, LRSG, or STMST mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an ITALY, Lor-2, STRIFE, TRASH, BDSF, LRSG, or STMST protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave ITALY, Lor-2, STRIFE, TRASH, BDSF, LRSG, or STMST mRNA transcripts to thereby inhibit translation of ITALY, Lor-2, STRIFE, TRASH, BDSF, LRSG, or STMST mRNA. A ribozyme having specificity for an ITALY, Lor-2, STRIFE, TRASH, BDSF, LRSG, or STMST-encoding nucleic acid can be designed based upon the nucleotide sequence of an ITALY, Lor-2, STRIFE, TRASH, BDSF, LRSG, or STMST cDNA disclosed herein (i.e., SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of the DNA insert of the plasmid deposited with ATCC as Accession Number 98960, 98756, or 98695). For example, a derivative of a Tetrahymena L-19 IVS RNA can be constructed in which the nucleotide sequence of the active site is complementary to the nucleotide sequence to be cleaved in an ITALY, Lor-2, STRIFE, TRASH, BDSF, LRSG, or STMST-encoding mRNA. See, e.g., Cech et al. U.S. Pat. No. 4,987,071; and Cech et al. U.S. Pat. No. 5,116,742. Alternatively, ITALY, Lor-2, STRIFE, TRASH, BDSF, LRSG, or STMST mRNA can be used to select a catalytic RNA having a specific ribonuclease activity from a pool of RNA molecules. See, e.g., Bartel, D. and Szostak, J. W. (1993) Science 261:1411-1418.

Alternatively, ITALY, Lor-2, STRIFE, TRASH, BDSF, LRSG, or STMST gene expression can be inhibited by targeting nucleotide sequences complementary to the regulatory region of the ITALY, Lor-2, STRIFE, TRASH, BDSF, LRSG, or STMST (e.g., the ITALY, Lor-2, STRIFE, TRASH, BDSF, LRSG, or STMST promoter and/or enhancers) to form triple helical structures that prevent transcription of the ITALY, Lor-2, STRIFE, TRASH, BDSF, LRSG, or STMST gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioessays 14(12):807-15.

In yet another embodiment, the ITALY, Lor-2, STRIFE, TRASH, BDSF, LRSG, or STMST nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of ITALY, Lor-2, STRIFE, TRASH, BDSF, LRSG, or STMST nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of ITALY, Lor-2, STRIFE, TRASH, BDSF, LRSG, or STMST nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of ITALY, Lor-2, STRIFE, TRASH, BDSF, LRSG, or STMST can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of ITALY, Lor-2, STRIFE, TRASH, BDSF, LRSG, or STMST nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### II. Isolated ITALY, Lor-2, STRIFE, TRASH, BDSF, LRSG, and STMST Proteins and Anti-ITALY, Lor-2. STRIFE, TRASH, BDSF, LRSG, and STMST Antibodies

One aspect of the invention pertains to isolated ITALY, Lor-2, STRIFE, TRASH, BDSF, LRSG, and STMST proteins, and biologically active portions thereof, as well as polypeptide fragments suitable for use as immunogens to raise anti-ITALY, Lor-2, STRIFE, TRASH, BDSF, LRSG, and STMST antibodies. In one embodiment, native ITALY, Lor-2, STRIFE, TRASH, BDSF, LRSG, or STMST proteins can be isolated from cells or tissue sources by an appropriate purification scheme using standard protein purification techniques. In another embodiment, ITALY, Lor-2, STRIFE, TRASH, BDSF, LRSG, or STMST proteins are produced by recombinant DNA techniques. Alternative to recombinant expression, an ITALY, Lor-2, STRIFE, TRASH, BDSF, LRSG, or STMST protein or polypeptide can be synthesized chemically using standard peptide synthesis techniques.

An “isolated” or “purified” protein or biologically active portion thereof is substantially free of cellular material or other contaminating proteins from the cell or tissue source from which the ITALY, Lor-2, STRIFE, TRASH, BDSF, LRSG, or STMST protein is derived, or substantially free from chemical precursors or other chemicals when chemically synthesized. The language “substantially free of cellular material” includes preparations of ITALY, Lor-2, STRIFE, TRASH, BDSF, LRSG, or STMST protein in which the protein is separated from cellular components of the cells from which it is isolated or recombinantly produced. In one embodiment, the language “substantially free of cellular material” includes preparations of ITALY, Lor-2, STRIFE, TRASH, BDSF, LRSG, or STMST protein having less than about 30% (by dry weight) of non-ITALY, Lor-2, STRIFE, TRASH, BDSF, LRSG, or STMST protein (also referred to herein as a “contaminating protein”), more preferably less than about 20% of non-ITALY, Lor-2, STRIFE, TRASH, BDSF, LRSG, or STMST protein, still more preferably less than about 10% of non-ITALY, Lor-2, STRIFE, TRASH, BDSF, LRSG, or STMST protein, and most preferably less than about 5% non-ITALY, Lor-2, STRIFE, TRASH, BDSF, LRSG, or STMST protein. When the ITALY, Lor-2, STRIFE, TRASH, BDSF, LRSG, or STMST protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of ITALY, Lor-2, STRIFE, TRASH, BDSF, LRSG, or STMST protein in which the protein is separated from chemical precursors or other chemicals which are involved in the synthesis of the protein. In one embodiment, the language “substantially free of chemical precursors or other chemicals” includes preparations of ITALY, Lor-2, STRIFE, TRASH, BDSF, LRSG, or STMST protein having less than about 30% (by dry weight) of chemical precursors or non-ITALY, Lor-2, STRIFE, TRASH, BDSF, LRSG, or STMST chemicals, more preferably less than about 20% chemical precursors or non-ITALY, Lor-2, STRIFE, TRASH, BDSF, LRSG, or STMST chemicals, still more preferably less than about 10% chemical precursors or non-ITALY, Lor-2, STRIFE, TRASH, BDSF, LRSG, or STMST chemicals, and most preferably less than about 5% chemical precursors or non-ITALY, Lor-2, STRIFE, TRASH, BDSF, LRSG, or STMST chemicals.

Biologically active portions of an ITALY, Lor-2, STRIFE, TRASH, BDSF, LRSG, or STMST protein include peptides comprising amino acid sequences sufficiently homologous to or derived from the amino acid sequence of the ITALY, Lor-2, STRIFE, TRASH, BDSF, LRSG, or STMST protein, e.g., the amino acid sequence shown in SEQ ID NO:2, 4, 11, 23, 27, 32, 37, 39, 41, 46, 49, 51, 54, 59, 68, 71, or 74, which include fewer amino acids than the full length ITALY, Lor-2, STRIFE, TRASH, BDSF, LRSG, or STMST proteins, and exhibit at least one activity of an ITALY, Lor-2, STRIFE, TRASH, BDSF, LRSG, or STMST protein. Typically, biologically active portions comprise a domain or motif with at least one activity of the ITALY, Lor-2, STRIFE, TRASH, BDSF, LRSG, or STMST protein. A biologically active portion of an ITALY, Lor-2, STRIFE, TRASH, BDSF, LRSG, or STMST protein can be a polypeptide which is, for example, 10, 25, 50, 100 or more amino acids in length.

In one embodiment, a biologically active portion of an ITALY protein comprises at least a disulfide forming cysteine residue. In another embodiment, a biologically active portion of an ITALY protein comprises at least an α-helical structure. In another embodiment a biologically active portion of an ITALY protein comprises at least a disulfide forming cysteine residue and an α-helical structure.

In another embodiment, a biologically active portion of a Lor-2 protein comprises a signal sequence and/or is secreted. In another embodiment, a biologically active portion of a Lor-2 protein lacks a signal sequence and/or is intracellular.

In another embodiment, a biologically active portion of a STRIFE1 or STRIFE2 protein comprises at least a cysteine-rich domain. In another embodiment, a biologically active portion of a STRIFE1 or STRIFE2 protein comprises at least a cysteine-rich domain, wherein the cysteine-domain includes at least one module. In yet another embodiment, a biologically active portion of a STRIFE1 or STRIFE2 protein comprises at least a signal sequence. In yet a further embodiment, a biologically active portion of a STRIFE1 or STRIFE2 protein comprises at least a cysteine-rich domain and a signal sequence.

In an alternative embodiment, a biologically active portion of a STRIFE1 or STRIFE2 protein comprises a STRIFE1 or STRIFE2 amino acid sequence lacking a signal sequence. In another alternative embodiment, a biologically active portion of a STRIFE1 or STRIFE2 protein comprises a STRIFE1 or STRIFE2 amino acid sequence lacking a cysteine-rich domain.

In another embodiment, a biologically active portion of a TRASH protein comprises at least a TNF signature motif. In yet another embodiment, a biologically active portion of a TRASH protein comprises at least a TNF-like N-terminal signal transmembrane anchor for a type II membrane protein. In another embodiment, a biologically active portion of a TRASH protein further comprises two cysteine residues which may be disulfide linked. In another embodiment, a biologically active portion of a TRASH protein further comprises two putative N-linked glycosylation sites.

In another embodiment, a biologically active portion of a BDSF protein comprises an Ig-like domain. In another embodiment, a biologically active portion of a BDSF protein comprises at least an Ig-like domain and a signal sequence.

In another embodiment, a biologically active portion of a LRSG protein comprises at least an Fn type III-like domain. In another embodiment, a biologically active portion of an LRSG protein comprises at least an EGF-like domain. In another embodiment, a biologically active portion of a LRSG protein comprises at least a leucine-rich region. In another embodiment, a biologically active portion of a LRSG protein comprises at least one leucine-rich repeat. In another embodiment a biologically active portion of a LRSG protein comprises at least an Fn type III-like domain and an EGF-like domain. In another embodiment, a biologically active portion of a LRSG protein comprises at least a Fn type III-like domain, an EGF-like domain and a transmembrane domain. In another embodiment, a biologically active portion of a LRSG protein comprises at least a leucine rich region, an Fn type III-like domain, an EGF-like domain, and a transmembrane domain.

In another embodiment, a biologically active portion of an STMST protein comprises at least a transmembrane domain. In another embodiment, a biologically active portion of an STMST protein comprises at least one 7 transmembrane receptor profile. In another embodiment, a biologically active portion of an STMST protein comprises at least a spectrin α-chain, repeated domain profile. In another embodiment a biologically active portion of an STMST protein comprises at least a 7 transmembrane receptor profile and a spectrin α-chain profile.

It is to be understood that a preferred biologically active portion of an ITALY, Lor-2, STRIFE, TRASH, BDSF, LRSG, or STMST protein of the present invention may contain at least one of the above-identified structural domains. A more preferred biologically active portion of an ITALY, Lor-2, STRIFE, TRASH, BDSF, LRSG, or STMST protein may contain at least two of the above-identified structural domains. Moreover, other biologically active portions, in which other regions of the protein are deleted, can be prepared by recombinant techniques and evaluated for one or more of the functional activities of a native ITALY, Lor-2, STRIFE, TRASH, BDSF, LRSG, or STMST protein.

In a preferred embodiment, the ITALY, Lor-2, STRIFE, TRASH, BDSF, LRSG, or STMST protein has an amino acid sequence shown in SEQ ID NO:2, 4, 11, 23, 27, 32, 37, 39, 41, 46, 49, 51, 54, 59, 68, 71, or 74. In other embodiments, the ITALY, Lor-2, STRIFE, TRASH, BDSF, LRSG, or STMST protein is substantially homologous to SEQ ID NO:2, 4, 11, 23, 27, 32, 37, 39, 41, 46, 49, 51, 54, 59, 68, 71, or 74, and retains the functional activity of the protein of SEQ ID NO:2, 4, 11, 23, 27, 32, 37, 39, 41, 46, 49, 51, 54, 59, 68, 71, or 74, yet differs in amino acid sequence due to natural allelic variation or mutagenesis, as described in detail in subsection I above. Accordingly, in another embodiment, the ITALY, Lor-2, STRIFE, TRASH, BDSF, LRSG, or STMST protein is a protein which comprises an amino acid sequence at least about 32%, 35%, 40%, 45%, 50%, 55%, 60%, 65%, 70%, 75%, 80%, 85%, 90%, 95% or more homologous to SEQ ID NO:2, 4, 11, 23, 27, 32, 37, 39, 41, 46, 49, 51, 54, 59, 68, 71, or 74.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the ITALY amino acid sequence of SEQ ID NO:2 having 177 amino acid residues, at least 80, preferably at least 100, more preferably at least 120, even more preferably at least 140, and even more preferably at least 150, 160 or 170 amino acid residues are aligned; when aligning a second sequence to the Lor-2 amino acid sequence of SEQ ID NO:11 having 753 amino acid residues, at least 226, preferably at least 301, more preferably at least 377, even more preferably at least 452, and even more preferably at least 527, 602 or 678 amino acid residues are aligned; when aligning a second sequence to the STRIFE1 amino acid sequence of SEQ ID NO:23 having 214 amino acid residues, at least 64, preferably at least 86, more preferably at least 107, even more preferably at least 128, and even more preferably at least 150, 172 or 193 amino acid residues are aligned; when aligning a second sequence to the STRIFE2 amino acid sequence of SEQ ID NO:27 having 150 amino acid residues, at least 45, preferably at least 60, more preferably at least 75, even more preferably at least 90, and even more preferably at least 105, 120 or 135 amino acid residues are aligned; when aligning a second sequence to the TRASH amino acid sequence of SEQ ID NO:32 having 250 amino acid residues, at least 75, preferably at least 100, more preferably at least 125, even more preferably at least 150, and even more preferably at least 175, 200 or 225 amino acid residues are aligned; when aligning a second sequence to the BDSF-1 amino acid sequence of SEQ ID NO:46 having 244 amino acid residues, at least 73, preferably at least 98, more preferably at least 122, even more preferably at least 146, and even more preferably at least 171, 195 or 220 amino acid residues are aligned; when aligning a second sequence to the BDSF-1 amino acid sequence of SEQ ID NO:51 having 251 amino acid residues, at least 75, preferably at least 100, more preferably at least 126, even more preferably at least 151, and even more preferably at least 176, 201 or 226 amino acid residues are aligned; when aligning a second sequence to the LRSG-1 amino acid sequence of SEQ ID NO:59 having 673 amino acid residues, at least 202, preferably at least 269, more preferably at least 337, even more preferably at least 404, and even more preferably at least 471, 538 or 606 amino acid residues are aligned; when aligning a second sequence to the LRSG-1 amino acid sequence of SEQ ID NO:68 having 673 amino acid residues, at least 202, preferably at least 269, more preferably at least 337, even more preferably at least 404, and even more preferably at least 471, 538 or 606 amino acid residues are aligned; when aligning a second sequence to the STMST-1 amino acid sequence of SEQ ID NO:71 having 297 amino acid residues, at least 89, preferably at least 119, more preferably at least 149, even more preferably at least 178, and even more preferably at least 208, 238 or 267 amino acid residues are aligned; and when aligning a second sequence to the STMST-2 amino acid sequence of SEQ ID NO:74 having 609 amino acid residues, at least 183, preferably at least 244, more preferably at least 305, even more preferably at least 365, and even more preferably at least 426, 487 or 548 amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to ITALY, Lor-2, STRIFE, TRASH, BDSF, LRSG, or STMST nucleic acid molecules of the invention. BLAST protein searches can be performed with the XBLAST program, score=50, wordlength=3 to obtain amino acid sequences homologous to ITALY, Lor-2, STRIFE, TRASH, BDSF, LRSG, or STMST protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See the website for the National Center for Biotechnology Information. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The invention also provides ITALY, Lor-2, STRIFE, TRASH, BDSF, LRSG, or STMST chimeric or fusion proteins. As used herein, an ITALY, Lor-2, STRIFE, TRASH, BDSF, LRSG, or STMST “chimeric protein” or “fusion protein” comprises an ITALY, Lor-2, STRIFE, TRASH, BDSF, LRSG, or STMST polypeptide operatively linked to a non-ITALY, Lor-2, STRIFE, TRASH, BDSF, LRSG, or STMST polypeptide. An “ITALY, Lor-2, STRIFE, TRASH, BDSF, LRSG, or STMST polypeptide” refers to a polypeptide having an amino acid sequence corresponding to ITALY, Lor-2, STRIFE, TRASH, BDSF, LRSG, or STMST, whereas a “non-ITALY, Lor-2, STRIFE, TRASH, BDSF, LRSG, or STMST polypeptide” refers to a polypeptide having an amino acid sequence corresponding to a protein which is not substantially homologous to the ITALY, Lor-2, STRIFE, TRASH, BDSF, LRSG, or STMST protein, e.g., a protein which is different from the ITALY, Lor-2, STRIFE, TRASH, BDSF, LRSG, or STMST protein and which is derived from the same or a different organism. Within an ITALY, Lor-2, STRIFE, TRASH, BDSF, LRSG, or STMST fusion protein the ITALY, Lor-2, STRIFE, TRASH, BDSF, LRSG, or STMST polypeptide can correspond to all or a portion of an ITALY, Lor-2, STRIFE, TRASH, BDSF, LRSG, or STMST protein. In a preferred embodiment, an ITALY, Lor-2, STRIFE, TRASH, BDSF, LRSG, or STMST fusion protein comprises at least one biologically active portion of an ITALY, Lor-2, STRIFE, TRASH, BDSF, LRSG, or STMST protein. In another preferred embodiment, an ITALY, Lor-2, STRIFE, TRASH, BDSF, LRSG, or STMST fusion protein comprises at least two biologically active portions of an ITALY, Lor-2, STRIFE, TRASH, BDSF, LRSG, or STMST protein. Within the fusion protein, the term “operatively linked” is intended to indicate that the ITALY, Lor-2, STRIFE, TRASH, BDSF, LRSG, or STMST polypeptide and the non-ITALY, Lor-2, STRIFE, TRASH, BDSF, LRSG, or STMST polypeptide are fused in-frame to each other. The non-ITALY, Lor-2, STRIFE, TRASH, BDSF, LRSG, or STMST polypeptide can be fused to the N-terminus or C-terminus of the ITALY, Lor-2, STRIFE, TRASH, BDSF, LRSG, or STMST polypeptide.

For example, in one embodiment, the fusion protein is a GST-ITALY, Lor-2, STRIFE, TRASH, BDSF, LRSG, or STMST fusion protein in which the ITALY, Lor-2, STRIFE, TRASH, BDSF, LRSG, or STMST sequences are fused to the C-terminus of the GST sequences. Such fusion proteins can facilitate the purification of recombinant ITALY, Lor-2, STRIFE, TRASH, BDSF, LRSG, or STMST.

In another embodiment, the fusion protein is an ITALY, Lor-2, STRIFE, TRASH, BDSF, LRSG, or STMST protein containing a heterologous signal sequence at its N-terminus. For example, the native ITALY signal sequence (i.e., about amino acids 1 to 24 of SEQ ID NO:2), Lor-2 signal sequence (i.e., about amino acids 1 to 25 of SEQ ID NO:11), STRIFE1 or STRIFE2 signal sequence (i.e., about amino acids 1-29 of SEQ ID NO:23 or SEQ ID NO:27), TRASH TNF-like N-terminal signal transmembrane anchor for a type II membrane protein sequence (i.e., about amino acids 1 to 44 of SEQ ID NO:32), BDSF signal sequence (i.e., about amino acids 1 to 25 of SEQ ID NO:46 or about amino acids 1 to 24 of SEQ ID NO:51), LRSG signal sequence (i.e., about amino acids 1 to 23 of SEQ ID NO:59), or STMST signal sequence can be removed and replaced with a signal sequence from another protein. In certain host cells (e.g., mammalian host cells), expression and/or secretion of ITALY, Lor-2, STRIFE, TRASH, BDSF, LRSG, or STMST can be increased through use of a heterologous signal sequence.

The ITALY, Lor-2, STRIFE, TRASH, BDSF, LRSG, or STMST fusion proteins of the invention can be incorporated into pharmaceutical compositions and administered to a subject in vivo. The ITALY, Lor-2, STRIFE, TRASH, BDSF, LRSG, or STMST fusion proteins can be used to affect the bioavailability of an ITALY, Lor-2, STRIFE, TRASH, BDSF, LRSG, or STMST substrate. Use of ITALY fusion proteins may be useful therapeutically for the treatment of immune or inflammatory disorders, e.g., Rheumatoid Arthritis, Systemic Lupus Erythematosus, Myasthenia Gravis, Grave's Disease, Sjogren Syndrome, Polymyositis and Dermatomyositis, Psoriasis, Pemphigus Vulgaris, Bullous Pemphigoid, Inflammatory Bowel Disease, Kawasaki Disease, Asthma, or Graft v. Host Disease; or proliferative disorders, e.g., cancer such as melanoma, prostate cancer, cervical cancer, breast cancer, colon cancer, or sarcoma. Use of Lor-2 fusion proteins may be useful therapeutically for the treatment of cardiovascular disorders (e.g., congestive heart failure). Use of STRIFE1 or STRIFE2 fusion proteins may be useful therapeutically for the treatment of TNF-associated disorders, e.g., inflammatory, immune, or neoplastic disorders. Use of TRASH fusion proteins may be useful therapeutically, for example, in regulation of the cellular immune response, regulation of inflammation, or regulation of hematopoiesis. Use of BDSF fusion proteins may be useful therapeutically for the treatment of proliferative disorders (e.g., prostate cancer). Use of LRSG fusion proteins may be useful therapeutically for the treatment of proliferative disorders (e.g., prostate cancer). Use of STMST fusion proteins may be useful therapeutically for the treatment of respiratory disorders (e.g., asthma).

Moreover, the ITALY, Lor-2, STRIFE, TRASH, BDSF, LRSG, or STMST-fusion proteins of the invention can be used as immunogens to produce anti-ITALY, Lor-2, STRIFE, TRASH, BDSF, LRSG, or STMST antibodies in a subject, to purify ITALY, Lor-2, STRIFE, TRASH, BDSF, LRSG, or STMST ligands and in screening assays to identify molecules which inhibit the interaction of ITALY, Lor-2, STRIFE, TRASH, BDSF, LRSG, or STMST with an ITALY, Lor-2, STRIFE, TRASH, BDSF, LRSG, or STMST substrate.

Preferably, an ITALY, Lor-2, STRIFE, TRASH, BDSF, LRSG, or STMST chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An ITALY, Lor-2, STRIFE, TRASH, BDSF, LRSG, or STMST-encoding nucleic acid can be cloned into such an expression vector such that the fusion moiety is linked in-frame to the ITALY, Lor-2, STRIFE, TRASH, BDSF, LRSG, or STMST protein.

The present invention also pertains to variants of the ITALY, Lor-2, STRIFE, TRASH, BDSF, LRSG, or STMST proteins which function as either ITALY, Lor-2, STRIFE, TRASH, BDSF, LRSG, or STMST agonists (mimetics) or as ITALY, Lor-2, STRIFE, TRASH, BDSF, LRSG, or STMST antagonists. Variants of the ITALY, Lor-2, STRIFE, TRASH, BDSF, LRSG, or STMST proteins can be generated by mutagenesis, e.g., discrete point mutation or truncation of an ITALY, Lor-2, STRIFE, TRASH, BDSF, LRSG, or STMST protein. An agonist of the ITALY, Lor-2, STRIFE, TRASH, BDSF, LRSG, or STMST proteins can retain substantially the same, or a subset, of the biological activities of the naturally occurring form of an ITALY, Lor-2, STRIFE, TRASH, BDSF, LRSG, or STMST protein. An antagonist of an ITALY, Lor-2, STRIFE, TRASH, BDSF, LRSG, or STMST protein can inhibit one or more of the activities of the naturally occurring form of the ITALY, Lor-2, STRIFE, TRASH, BDSF, LRSG, or STMST protein by, for example, competitively modulating an immune system activity of an ITALY, Lor-2, STRIFE, TRASH, BDSF, LRSG, or STMST protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the ITALY, Lor-2, STRIFE, TRASH, BDSF, LRSG, or STMST protein.

In one embodiment, variants of an ITALY, Lor-2, STRIFE, TRASH, BDSF, LRSG, or STMST protein which function as either ITALY, Lor-2, STRIFE, TRASH, BDSF, LRSG, or STMST agonists (mimetics) or as ITALY, Lor-2, STRIFE, TRASH, BDSF, LRSG, or STMST antagonists can be identified by screening combinatorial libraries of mutants, e.g., truncation mutants, of an ITALY, Lor-2, STRIFE, TRASH, BDSF, LRSG, or STMST protein for ITALY, Lor-2, STRIFE, TRASH, BDSF, LRSG, or STMST protein agonist or antagonist activity. In one embodiment, a variegated library of ITALY, Lor-2, STRIFE, TRASH, BDSF, LRSG, or STMST variants is generated by combinatorial mutagenesis at the nucleic acid level and is encoded by a variegated gene library. A variegated library of ITALY, Lor-2, STRIFE, TRASH, BDSF, LRSG, or STMST variants can be produced by, for example, enzymatically ligating a mixture of synthetic oligonucleotides into gene sequences such that a degenerate set of potential ITALY, Lor-2, STRIFE, TRASH, BDSF, LRSG, or STMST sequences is expressible as individual polypeptides, or alternatively, as a set of larger fusion proteins (e.g., for phage display) containing the set of ITALY, Lor-2, STRIFE, TRASH, BDSF, LRSG, or STMST sequences therein. There are a variety of methods which can be used to produce libraries of potential ITALY, Lor-2, STRIFE, TRASH, BDSF, LRSG, or STMST variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ITALY, Lor-2, STRIFE, TRASH, BDSF, LRSG, or STMST sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of an ITALY, Lor-2, STRIFE, TRASH, BDSF, LRSG, or STMST protein coding sequence can be used to generate a variegated population of ITALY, Lor-2, STRIFE, TRASH, BDSF, LRSG, or STMST fragments for screening and subsequent selection of variants of an ITALY, Lor-2, STRIFE, TRASH, BDSF, LRSG, or STMST protein. In one embodiment, a library of coding sequence fragments can be generated by treating a double stranded PCR fragment of an ITALY, Lor-2, STRIFE, TRASH, BDSF, LRSG, or STMST coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the ITALY, Lor-2, STRIFE, TRASH, BDSF, LRSG, or STMST protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ITALY, Lor-2, STRIFE, TRASH, BDSF, LRSG, or STMST proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify ITALY, Lor-2, STRIFE, TRASH, BDSF, LRSG, or STMST variants (Arkin and Youvan (1992) Proc. Natl. Acad. Sci. USA 89:7811-7815; Delagrave et al. (1993) Protein Eng. 6(3):327-331).

In one embodiment, cell based assays can be exploited to analyze a variegated ITALY, Lor-2, STRIFE, TRASH, BDSF, LRSG, or STMST library. For example, a library of expression vectors can be transfected into a cell line which ordinarily synthesizes and secretes ITALY, Lor-2, STRIFE, TRASH, BDSF, LRSG, or STMST. The transfected cells are then cultured such that ITALY, Lor-2, STRIFE, TRASH, BDSF, LRSG, or STMST and a particular mutant ITALY, Lor-2, STRIFE, TRASH, BDSF, LRSG, or STMST are secreted and the effect of expression of the mutant on ITALY, Lor-2, STRIFE, TRASH, BDSF, LRSG, or STMST activity in cell supernatants can be detected, e.g., by any of a number of enzymatic assays. Plasmid DNA can then be recovered from the cells which score for inhibition, or alternatively, potentiation of ITALY, Lor-2, STRIFE, TRASH, BDSF, LRSG, or STMST activity, and the individual clones further characterized.

An isolated ITALY, Lor-2, STRIFE, TRASH, BDSF, LRSG, or STMST protein, or a portion or fragment thereof, can be used as an immunogen to generate antibodies that bind ITALY, Lor-2, STRIFE, TRASH, BDSF, LRSG, or STMST using standard techniques for polyclonal and monoclonal antibody preparation. A full-length ITALY, Lor-2, STRIFE, TRASH, BDSF, LRSG, or STMST protein can be used or, alternatively, the invention provides antigenic peptide fragments of ITALY, Lor-2, STRIFE, TRASH, BDSF, LRSG, or STMST for use as immunogens. The antigenic peptide of ITALY, Lor-2, STRIFE, TRASH, BDSF, LRSG, or STMST comprises at least 8 amino acid residues of the amino acid sequence shown in SEQ ID NO:2, 4, 11, 23, 27, 32, 37, 39, 41, 46, 49, 51, 54, 59, 68, 71, or 74 and encompasses an epitope of ITALY, Lor-2, STRIFE, TRASH, BDSF, LRSG, or STMST such that an antibody raised against the peptide forms a specific immune complex with ITALY, Lor-2, STRIFE, TRASH, BDSF, LRSG, or STMST. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of ITALY, Lor-2, STRIFE, TRASH, BDSF, LRSG, or STMST that are located on the surface of the protein, e.g., hydrophilic regions.

An ITALY, Lor-2, STRIFE, TRASH, BDSF, LRSG, or STMST immunogen typically is used to prepare antibodies by immunizing a suitable subject, (e.g., rabbit, goat, mouse or other mammal) with the immunogen. An appropriate immunogenic preparation can contain, for example, recombinantly expressed ITALY, Lor-2, STRIFE, TRASH, BDSF, LRSG, or STMST protein or a chemically synthesized ITALY, Lor-2, STRIFE, TRASH, BDSF, LRSG, or STMST polypeptide. The preparation can further include an adjuvant, such as Freund's complete or incomplete adjuvant, or similar immunostimulatory agent. Immunization of a suitable subject with an immunogenic ITALY, Lor-2, STRIFE, TRASH, BDSF, LRSG, or STMST preparation induces a polyclonal anti-ITALY, Lor-2, STRIFE, TRASH, BDSF, LRSG, or STMST antibody response.

Accordingly, another aspect of the invention pertains to anti-ITALY, Lor-2, STRIFE, TRASH, BDSF, LRSG, and STMST antibodies. The term “antibody” as used herein refers to immunoglobulin molecules and immunologically active portions of immunoglobulin molecules, i.e., molecules that contain an antigen binding site which specifically binds (immunoreacts with) an antigen, such as ITALY, Lor-2, STRIFE, TRASH, BDSF, LRSG, or STMST. Examples of immunologically active portions of immunoglobulin molecules include F(ab) and F(ab′)² fragments which can be generated by treating the antibody with an enzyme such as pepsin or papain, respectively. The invention provides polyclonal and monoclonal antibodies that bind ITALY, Lor-2, STRIFE, TRASH, BDSF, LRSG, or STMST. The term “monoclonal antibody” or “monoclonal antibody composition”, as used herein, refers to a population of antibody molecules that contain only one species of an antigen binding site capable of immunoreacting with a particular epitope of ITALY, Lor-2, STRIFE, TRASH, BDSF, LRSG, or STMST. A monoclonal antibody composition thus typically displays a single binding affinity for a particular ITALY, Lor-2, STRIFE, TRASH, BDSF, LRSG, or STMST protein with which it immunoreacts.

Polyclonal anti-ITALY, Lor-2, STRIFE, TRASH, BDSF, LRSG, or STMST antibodies can be prepared as described above by immunizing a suitable subject with an ITALY, Lor-2, STRIFE, TRASH, BDSF, LRSG, or STMST immunogen. The anti-ITALY, Lor-2, STRIFE, TRASH, BDSF, LRSG, or STMST antibody titer in the immunized subject can be monitored over time by standard techniques, such as with an enzyme linked immunosorbent assay (ELISA) using immobilized ITALY, Lor-2, STRIFE, TRASH, BDSF, LRSG, or STMST. If desired, the antibody molecules directed against ITALY, Lor-2, STRIFE, TRASH, BDSF, LRSG, or STMST can be isolated from the mammal (e.g., from the blood) and further purified by well known techniques, such as protein A chromatography to obtain the IgG fraction. At an appropriate time after immunization, e.g., when the anti-ITALY, Lor-2, STRIFE, TRASH, BDSF, LRSG, or STMST antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with an ITALY, Lor-2, STRIFE, TRASH, BDSF, LRSG, or STMST immunogen as described above, and the culture supernatants of the resulting hybridoma cells are screened to identify a hybridoma producing a monoclonal antibody that binds ITALY, Lor-2, STRIFE, TRASH, BDSF, LRSG, or STMST.

Any of the many well known protocols used for fusing lymphocytes and immortalized cell lines can be applied for the purpose of generating an anti-ITALY, Lor-2, STRIFE, TRASH, BDSF, LRSG, or STMST monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind ITALY, Lor-2, STRIFE, TRASH, BDSF, LRSG, or STMST, e.g., using a standard ELISA assay.

Alternative to preparing monoclonal antibody-secreting hybridomas, a monoclonal anti-ITALY, Lor-2, STRIFE, TRASH, BDSF, LRSG, or STMST antibody can be identified and isolated by screening a recombinant combinatorial immunoglobulin library (e.g., an antibody phage display library) with ITALY, Lor-2, STRIFE, TRASH, BDSF, LRSG, or STMST to thereby isolate immunoglobulin library members that bind ITALY, Lor-2, STRIFE, TRASH, BDSF, LRSG, or STMST. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. Nature (1990) 348:552-554.

Additionally, recombinant anti-ITALY, Lor-2, STRIFE, TRASH, BDSF, LRSG, or STMST antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Fremont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Biotechnology (NY) 12:899-903).

An anti-ITALY, Lor-2, STRIFE, TRASH, BDSF, LRSG, or STMST antibody (e.g., monoclonal antibody) can be used to isolate ITALY, Lor-2, STRIFE, TRASH, BDSF, LRSG, or STMST by standard techniques, such as affinity chromatography or immunoprecipitation. An anti-ITALY, Lor-2, STRIFE, TRASH, BDSF, LRSG, or STMST antibody can facilitate the purification of natural ITALY, Lor-2, STRIFE, TRASH, BDSF, LRSG, or STMST from cells and of recombinantly produced ITALY, Lor-2, STRIFE, TRASH, BDSF, LRSG, or STMST expressed in host cells. Moreover, an anti-ITALY, Lor-2, STRIFE, TRASH, BDSF, LRSG, or STMST antibody can be used to detect ITALY, Lor-2, STRIFE, TRASH, BDSF, LRSG, or STMST protein (e.g., in a cellular lysate or cell supernatant) in order to evaluate the abundance and pattern of expression of the ITALY, Lor-2, STRIFE, TRASH, BDSF, LRSG, or STMST protein. Anti-ITALY, Lor-2, STRIFE, TRASH, BDSF, LRSG, or STMST antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

An antibody (or fragment thereof) can be conjugated to a therapeutic moiety such as a cytotoxin, a therapeutic agent, or a radioactive agent (e.g., a radioactive metal ion). Cytotoxins and cytotoxic agents include any agent that is detrimental to cells. Examples of such agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin {formerly designated daunomycin} and doxorubicin), antibiotics (e.g., dactinomycin {formerly designated actinomycin}, bleomycin, mithramycin, and anthramycin), and anti-mitotic agents (e.g., vincristine and vinblastine).

Conjugated antibodies of the invention can be used for modifying a given biological response, the drug moiety not being limited to classical chemical therapeutic agents. For example, the drug moiety can be a protein or polypeptide possessing a desired biological activity. Such proteins include, for example, toxins such as abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Techniques for conjugating a therapeutic moiety to an antibody are well known (see, e.g., Amon et al. (1985) “Monoclonal Antibodies for Immunotargeting of Drugs in Cancer Therapy”, in Monoclonal Antibodies and Cancer Therapy, Reisfeld et al., Eds., Alan R. Liss, Inc. pp. 243-256; Hellstrom et al. (1987) “Antibodies for Drug Delivery”, in Controlled Drug Delivery, 2nd ed., Robinson et al., Eds., Marcel Dekker, Inc., pp. 623-653; Thorpe (1985) “Antibody Carriers of Cytotoxic Agents in Cancer Therapy: A Review”, in Monoclonal Antibodies '84: Biological and Clinical Applications, Pinchera et al., Eds., pp. 475-506; “Analysis, Results, and Future Prospective of the Therapeutic Use of Radiolabeled Antibody in Cancer Therapy”, in Monoclonal Antibodies for Cancer Detection and Therapy, Baldwin et al., Eds., Academic Press, pp. 303-316, (1985); and Thorpe et al. (1982) Immunol. Rev. 62:119-158). Alternatively, an antibody can be conjugated to a second antibody to form an antibody heteroconjugate as described by Segal in U.S. Pat. No. 4,676,980.

### III. Recombinant Expression Vectors and Host Cells

Another aspect of the invention pertains to vectors, preferably expression vectors, containing a nucleic acid encoding an ITALY, Lor-2, STRIFE, TRASH, BDSF, LRSG, or STMST protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., ITALY, Lor-2, STRIFE, TRASH, BDSF, LRSG, or STMST proteins, mutant forms of ITALY, Lor-2, STRIFE, TRASH, BDSF, LRSG, or STMST proteins, fusion proteins, and the like).

The recombinant expression vectors of the invention can be designed for expression of ITALY, Lor-2, STRIFE, TRASH, BDSF, LRSG, or STMST proteins in prokaryotic or eukaryotic cells. For example, ITALY, Lor-2, STRIFE, TRASH, BDSF, LRSG, or STMST proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in ITALY, Lor-2, STRIFE, TRASH, BDSF, LRSG, or STMST activity assays, (e.g., direct assays or competitive assays described in detail below), or to generate antibodies specific for ITALY, Lor-2, STRIFE, TRASH, BDSF, LRSG, or STMST proteins, for example. In a preferred embodiment, an ITALY, Lor-2, STRIFE, TRASH, BDSF, LRSG, or STMST fusion protein expressed in a retroviral expression vector of the present invention can be utilized to infect bone marrow cells which are subsequently transplanted into irradiated recipients. The pathology of the subject recipient is then examined after sufficient time has passed (e.g., six (6) weeks).

Examples of suitable inducible non-fusion E. coli expression vectors include pTrc (Amann et al., (1988) Gene 69:301-315) and pET 11d (Studier et al., Gene Expression Technology Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the ITALY, Lor-2, STRIFE, TRASH, BDSF, LRSG, or STMST expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, ITALY, Lor-2, STRIFE, TRASH, BDSF, LRSG, or STMST proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to ITALY, Lor-2, STRIFE, TRASH, BDSF, LRSG, or STMST mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an ITALY, Lor-2, STRIFE, TRASH, BDSF, LRSG, or STMST protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an ITALY, Lor-2, STRIFE, TRASH, BDSF, LRSG, or STMST protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an ITALY, Lor-2, STRIFE, TRASH, BDSF, LRSG, or STMST protein. Accordingly, the invention further provides methods for producing an ITALY, Lor-2, STRIFE, TRASH, BDSF, LRSG, or STMST protein using the host cells of the invention. In one embodiment, the method comprises culturing the host cell of invention (into which a recombinant expression vector encoding an ITALY, Lor-2, STRIFE, TRASH, BDSF, LRSG, or STMST protein has been introduced) in a suitable medium such that an ITALY, Lor-2, STRIFE, TRASH, BDSF, LRSG, or STMST protein is produced. In another embodiment, the method further comprises isolating an ITALY, Lor-2, STRIFE, TRASH, BDSF, LRSG, or STMST protein from the medium or the host cell.

The host cells of the invention can also be used to produce non-human transgenic animals. For example, in one embodiment, a host cell of the invention is a fertilized oocyte or an embryonic stem cell into which ITALY, Lor-2, STRIFE, TRASH, BDSF, LRSG, or STMST-coding sequences have been introduced. Such host cells can then be used to create non-human transgenic animals in which exogenous ITALY, Lor-2, STRIFE, TRASH, BDSF, LRSG, or STMST sequences have been introduced into their genome or homologous recombinant animals in which endogenous ITALY, Lor-2, STRIFE, TRASH, BDSF, LRSG, or STMST sequences have been altered. Such animals are useful for studying the function and/or activity of an ITALY, Lor-2, STRIFE, TRASH, BDSF, LRSG, or STMST and for identifying and/or evaluating modulators of ITALY, Lor-2, STRIFE, TRASH, BDSF, LRSG, or STMST activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous ITALY, Lor-2, STRIFE, TRASH, BDSF, LRSG, or STMST gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an ITALY, Lor-2, STRIFE, TRASH, BDSF, LRSG, or STMST-encoding nucleic acid into the male pronuclei of a fertilized oocyte, e.g., by microinjection, retroviral infection, and allowing the oocyte to develop in a pseudopregnant female foster animal. The ITALY, Lor-2, STRIFE, TRASH, BDSF, LRSG, or STMST cDNA sequence of SEQ ID NO:1, 10, 22, 26, 31, 45, 59, 58, 67, 70, or 73 can be introduced as a transgene into the genome of a non-human animal. Alternatively, a nonhuman homologue of a human ITALY, Lor-2, STRIFE, TRASH, BDSF, LRSG, or STMST gene, such as a mouse or rat ITALY, Lor-2, STRIFE, TRASH, BDSF, LRSG, or STMST gene, can be used as a transgene. Alternatively, an ITALY, Lor-2, STRIFE, TRASH, BDSF, LRSG, or STMST gene homologue, such as another IL-10 family member, ITALY family member, STRIFE family member, TRASH family member, BDSF family member, LRSG family member, or STMST family member, can be isolated based on hybridization to the ITALY, Lor-2, STRIFE, TRASH, BDSF, LRSG, or STMST cDNA sequences of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the DNA insert of the plasmid deposited with ATCC as Accession Number 98960, 98756, or 98695 (described further in subsection I above) and used as a transgene. Intronic sequences and polyadenylation signals can also be included in the transgene to increase the efficiency of expression of the transgene. A tissue-specific regulatory sequence(s) can be operably linked to an ITALY, Lor-2, STRIFE, TRASH, BDSF, LRSG, or STMST transgene to direct expression of an ITALY, Lor-2, STRIFE, TRASH, BDSF, LRSG, or STMST protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of an ITALY, Lor-2, STRIFE, TRASH, BDSF, LRSG, or STMST transgene in its genome and/or expression of ITALY, Lor-2, STRIFE, TRASH, BDSF, LRSG, or STMST mRNA in tissues or cells of the animals. A transgenic founder animal can then be used to breed additional animals carrying the transgene. Moreover, transgenic animals carrying a transgene encoding an ITALY, Lor-2, STRIFE, TRASH, BDSF, LRSG, or STMST protein can further be bred to other transgenic animals carrying other transgenes.

To create a homologous recombinant animal, a vector is prepared which contains at least a portion of an ITALY, Lor-2, STRIFE, TRASH, BDSF, LRSG, or STMST gene into which a deletion, addition or substitution has been introduced to thereby alter, e.g., functionally disrupt, the ITALY, Lor-2, STRIFE, TRASH, BDSF, LRSG, or STMST gene. The ITALY, Lor-2, STRIFE, TRASH, BDSF, LRSG, or STMST gene can be a human gene (e.g., the cDNA of SEQ ID NO:1, 3, 10, 12, 31, 33, 36, 38, 40, 45, 47, 48, 58, 60, 70, 72, 73, or 75), but more preferably, is a non-human homologue of a human ITALY, Lor-2, STRIFE, TRASH, BDSF, LRSG, or STMST gene (e.g., the cDNA of SEQ ID NO:22, 24, 25, 26, 28, 29, 50, 52, 53, 67, or 69, or a cDNA isolated by stringent hybridization with the nucleotide sequence of SEQ ID NO:1, 3, 10, 12, 31, 33, 36, 38, 40, 45, 47, 48, 58, 60, 70, 72, 73, or 75). For example, a mouse ITALY, Lor-2, STRIFE, TRASH, BDSF, LRSG, or STMST gene can be used to construct a homologous recombination vector suitable for altering an endogenous ITALY, Lor-2, STRIFE, TRASH, BDSF, LRSG, or STMST gene in the mouse genome. In a preferred embodiment, the vector is designed such that, upon homologous recombination, the endogenous ITALY, Lor-2, STRIFE, TRASH, BDSF, LRSG, or STMST gene is functionally disrupted (i.e., no longer encodes a functional protein; also referred to as a “knock out” vector). Alternatively, the vector can be designed such that, upon homologous recombination, the endogenous ITALY, Lor-2, STRIFE, TRASH, BDSF, LRSG, or STMST gene is mutated or otherwise altered but still encodes functional protein (e.g., the upstream regulatory region can be altered to thereby alter the expression of the endogenous ITALY, Lor-2, STRIFE, TRASH, BDSF, LRSG, or STMST protein). In the homologous recombination vector, the altered portion of the ITALY, Lor-2, STRIFE, TRASH, BDSF, LRSG, or STMST gene is flanked at its 5′ and 3′ ends by additional nucleic acid sequence of the ITALY, Lor-2, STRIFE, TRASH, BDSF, LRSG, or STMST gene to allow for homologous recombination to occur between the exogenous ITALY, Lor-2, STRIFE, TRASH, BDSF, LRSG, or STMST gene carried by the vector and an endogenous ITALY, Lor-2, STRIFE, TRASH, BDSF, LRSG, or STMST gene in an embryonic stem cell. The additional flanking ITALY, Lor-2, STRIFE, TRASH, BDSF, LRSG, or STMST nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced ITALY, Lor-2, STRIFE, TRASH, BDSF, LRSG, or STMST gene has homologously recombined with the endogenous ITALY, Lor-2, STRIFE, TRASH, BDSF, LRSG, or STMST gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-humans animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

### IV. Pharmaceutical Compositions

The ITALY, Lor-2, STRIFE, TRASH, BDSF, LRSG, or STMST nucleic acid molecules, ITALY, Lor-2, STRIFE, TRASH, BDSF, LRSG, or STMST proteins, and anti-ITALY, Lor-2, STRIFE, TRASH, BDSF, LRSG, or STMST antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The agent which modulates expression or activity can, for example, be a small molecule. For example, such small molecules include peptides, peptidomimetics, amino acids, amino acid analogs, polynucleotides, polynucleotide analogs, nucleotides, nucleotide analogs, organic or inorganic compounds (I.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents and protein or polypeptide agents depends upon a number of factors within the ken of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Examples of doses of a small molecule include milligram or microgram amounts per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Examples of doses of a protein or polypeptide include gram, milligram or microgram amounts per kilogram of subject or sample weight (e.g., about 1 microgram per kilogram to about 5 grams per kilogram, about 100 micrograms per kilogram to about 500 milligrams per kilogram, or about 1 milligram per kilogram to about 50 milligrams per kilogram). For antibodies, examples of dosages are from about 0.1 milligram per kilogram to 100 milligrams per kilogram of body weight (generally 10 milligrams per kilogram to 20 milligrams per kilogram). If the antibody is to act in the brain, a dosage of 50 milligrams per kilogram to 100 milligrams per kilogram is usually appropriate. It is furthermore understood that appropriate doses of one of these agents depend upon the potency of the agent with respect to the expression or activity to be modulated. Such appropriate doses can be determined using the assays described herein. When one or more of these agents is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an ITALY, Lor-2, STRIFE, TRASH, BDSF, LRSG, or STMST protein or anti-ITALY, Lor-2, STRIFE, TRASH, BDSF, LRSG, or STMST antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### V. Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

As described herein, an ITALY protein of the invention has one or more of the following activities: (i) it can activate an ITALY-dependent signal transduction pathway; (ii) it can modulate secretion of a proinflammatory cytokine e.g., IL-1α, IL-1β, IL-6, or TNF-α; (iii) it can modulate secretion of a chemokine, e.g., IL-8 or macrophage inflammatory protein; (iv) it can modulate surface expression of a cellular adhesion molecule e.g., ICAM1; (v) it can modulate T cell proliferation and differentiation; (vi) it can modulate B cell proliferation and differentiation; (vii) it can modulate immune activity of a cell, e.g., a T cell; and (viii) it can modulate inflammatory activity of a cell, e.g., a T cell, and can thus be used in, for example, (i) activation of an ITALY-dependent signal transduction pathway; (ii) modulation of secretion of a proinflammatory cytokine e.g., IL-1α, IL-1β, IL-6, or TNF-α; (iii) modulation of secretion of a chemokine, e.g., IL-8 or macrophage inflammatory protein; (iv) modulation of surface expression of MHC molecules, e.g., MHC class II molecules, or cellular adhesion molecules, e.g., ICAM1; (v) modulation of inflammatory activity of a cell, e.g., a T cell; (vi) modulation of immune activity of a cell, e.g., a T cell (e.g., T cell proliferation, IL-2 secretion, or IL-5 secretion) or a B cell (e.g., B cell proliferation, differentiation, or antibody production); and (vii) modulation of proliferation and/or differentiation of a cell, e.g., a B cell, a T cell, a central nervous system cell, a prostate cell, a cervical cell, a breast cell, a colon cell, or an epithelial cell.

As described herein, a Lor-2 protein of the invention has one or more of the following activities: (i) interaction of a Lor-2 protein with a Lor-2 target molecule; (ii) interaction of a Lor-2 protein with a Lor-2 target molecule, wherein the Lor-2 target is a ligand; (iii) interaction of a Lor-2 protein with a Lor-2 target molecule, wherein the Lor-2 target is an extracellular matrix component (e.g., collagen or elastin); and (iv) modification of a Lor-2 target molecule (e.g., posttranslational modification).

Further as described herein, a Lor-2 protein of the invention has one or more of the above activities and can thus be used in, for example: (1) crosslinking an extracellular matrix component; (2) regulating bone resorption; (3) regulating copper metabolism; (4) modulating maturation and/or stabilization of extracellular matrix components; (5) regulating cellular signaling; (6) regulating cellular adhesion; (7) regulating cardiac cellular processes; and (8) modulating a Lor-2-related disorder as defined herein.

As described herein, a STRIFE1 protein of the invention has one or more of the following activities: (i) interaction of a STRIFE1 protein on the cell surface with a second non-STRIFE1 protein molecule on the surface of the same cell; (ii) interaction of a STRIFE1 protein on the cell surface with a second non-STRIFE1 protein molecule on the surface of a different cell; (iii) complex formation between a membrane-bound STRIFE1 protein and a cytokine, e.g., TNF; (iv) interaction of a STRIFE1 protein with an intracellular protein including SH2 domain-containing proteins or cytoskeletal proteins; (v) formation of a homogeneous multimeric signaling complex with like STRIFE1 proteins; and (vi) formation of a heterogeneous multimeric signaling complex with other TNFR superfamily proteins. As described herein, STRIFE2 protein of the invention has one or more of the following activities: (i) interaction of a STRIFE2 protein with a membrane-bound STRIFE2 receptor; (ii) interaction of a STRIFE2 protein with a soluble form of a STRIFE2 receptor; (iii) interaction of a STRIFE2 protein with an intracellular protein via a membrane-bound STRIFE2 receptor; (iv) complex formation between a soluble STRIFE2 protein and a second soluble STRIFE2 binding partner; (v) complex formation between a soluble STRIFE2 protein and a second soluble STRIFE2 binding partner, wherein the STRIFE2 binding partner is a non-STRIFE2 protein molecule; and (vi) complex formation between a soluble STRIFE2 protein and a second soluble STRIFE2 binding partner, wherein the STRIFE2 binding partner is a second STRIFE2 protein molecule. The STRIFE1 and STRIFE2 proteins of the invention can thus be used in, for example, (1) modulation of cellular signal transduction, either in vitro or in vivo; (2) regulation of gene transcription in a cell involved in development or differentiation, either in vitro or in vivo; (3) regulation of gene transcription in a cell involved in development or differentiation, wherein at least one gene encodes a differentiation-specific protein; (4) regulation of gene transcription in a cell involved in development or differentiation, wherein at least one gene encodes a second secreted protein; (5) regulation of gene transcription in a cell involved in development or differentiation, wherein at least one gene encodes a signal transduction molecule; and (6) regulation of cellular proliferation, either in vitro or in vivo.

As described herein, a TRASH protein of the invention has one or more of the following activities: (i) activation of a TRASH-dependent signal transduction pathway; (ii) cytolysis of certain tumor cell lines; (iii) modulation of secretion of inflammatory mediators/cytokines; (iv) modulation of the development or differentiation of a TRASH-expressing cell; (v) modulation of the development or differentiation of a non-TRASH-expressing cell; or (vi) modulation of host resistance to infectious agents.

As described herein, a BDSF protein of the invention has one or more of the following activities: (i) interaction of a BDSF protein in the extracellular milieu with a non-BDSF protein molecule on the surface of the same cell which secreted the BDSF protein molecule; (ii) interaction of a BDSF protein in the extracellular milieu with a non-BDSF protein molecule on the surface of a different cell from that which secreted the BDSF protein molecule; (iii) complex formation between a BDSF protein and a BDSF receptor; (iv) complex formation between a BDSF protein and non-BDSF receptor; and (v) interaction of a BDSF protein with a second protein in the extracellular milieu, and can thus be used in, for example, (1) modulation of cellular signal transduction, either in vitro or in vivo; (2) modulation of protein:protein interaction, either in vitro or in vivo; (3) regulation of cellular proliferation; or (4) regulation of cellular differentiation.

As described herein, a LRSG protein of the invention has one or more of the following activities: (i) interaction of a LRSG protein with a LRSG target molecule; (ii) interaction of a LRSG protein with a LRSG target molecule, wherein the LRSG target is an extracellular matrix protein; (iii) interaction of a LRSG protein with a LRSG target molecule, wherein the LRSG target is an intracellular signaling molecule; and (iv) interaction of a LRSG protein with a LRSG target molecule, wherein the LRSG target is a second molecule on the cell surface which interacts with an intracellular signaling molecule, and can thus be used in, for example, (1) modulation of cellular signal transduction, either in vitro or in vivo; (2) modulation of protein:protein interaction, either in vitro or in vivo; (3) regulation of cellular proliferation; or (4) regulation of cellular differentiation.

As described herein, an STMST protein of the invention has one or more of the following activities: (i) interaction of an STMST protein with soluble STMST ligand; (ii) interaction of an STMST protein with a membrane-bound non-STMST protein; (iii) interaction of an STMST protein with an intracellular protein (e.g., an intracellular enzyme or signal transduction molecule); and (iv) indirect interaction of an STMST protein with an intracellular protein (e.g., a downstream signal transduction molecule, and can thus be used, for example, in (1) modulation of cellular signal transduction, either in vitro or in vivo; (2) regulation of gene transcription in a cell expressing an STMST protein; (3) regulation of gene transcription in a cell expressing an STMST protein, wherein said cell is involved inflammation; (4) regulation of cellular proliferation; (5) regulation of cellular differentiation; (6) regulation of development; (7) regulation of cell death; (8) regulation of inflammation; and (9) regulation of respiratory cell function.

In another embodiment, an STMST modulator is useful for (1) modulating bone homeostasis (e.g., stimulation of bone homeostasis) and/or modulation of bone formation (e.g., stimulation of bone mass and/or inhibition of bone loss); (2) regulating, preventing and/or treating bone-related and/or bone resorption disorders including, but not limited to osteoporosis, Paget's disease, osteoarthritis, degenerative arthritis, osteogenesis imperfecta, fibrous dysplasia, hypophosphatasia, bone sarcoma, myeloma bone disorder (e.g., osteolytic bone lesions) and hypercalcemia; (3) management of bone fragility (e.g., decrease bone fragility); and (4) prevention and/or treatment of bone pain, bone deformities, and/or bone fractures.

In another embodiment, a STMST modulator is useful for: (1) modulation (e.g., repression or stimulation) of brain anabolic circuits or pathways; (2) modulation (e.g., repression or stimulation of brain catabolic pathways; (3) modulation of food intake and/or feeding behavior (e.g., stimulation of or inhibition/suppression of food intake and/or feeding behavior); (4) modulation of energy expenditure (e.g., suppression or stimulation of energy expenditure); (5) regulation of energy homeostasis; (6) regulation of body fat mass; (7) regulation of body temperature; (8) regulation of the sleep-wake cycle; (9) regulation of memory and/or behavior; (10) control of thirst; and (11) regulation of autonomic nervous system function.

The isolated nucleic acid molecules of the invention can be used, for example, to express ITALY, Lor-2, STRIFE, TRASH, BDSF, LRSG, or STMST protein (e.g., via a recombinant expression vector in a host cell in gene therapy applications), to detect ITALY, Lor-2, STRIFE, TRASH, BDSF, LRSG, or STMST mRNA (e.g., in a biological sample) or a genetic alteration in an ITALY, Lor-2, STRIFE, TRASH, BDSF, LRSG, or STMST gene, and to modulate ITALY, Lor-2, STRIFE, TRASH, BDSF, LRSG, or STMST activity, as described further below. The ITALY, Lor-2, STRIFE, TRASH, BDSF, LRSG, or STMST proteins can be used to treat disorders characterized by insufficient or excessive production of an ITALY, Lor-2, STRIFE, TRASH, BDSF, LRSG, or STMST substrate or production of ITALY, Lor-2, STRIFE, TRASH, BDSF, LRSG, or STMST inhibitors. In addition, the ITALY, Lor-2, STRIFE, TRASH, BDSF, LRSG, or STMST proteins can be used to screen for naturally occurring ITALY, Lor-2, STRIFE, TRASH, BDSF, LRSG, or STMST substrates, to screen for drugs or compounds which modulate ITALY, Lor-2, STRIFE, TRASH, BDSF, LRSG, or STMST activity, as well as to treat disorders characterized by insufficient or excessive production of ITALY, Lor-2, STRIFE, TRASH, BDSF, LRSG, or STMST protein or production of ITALY, Lor-2, STRIFE, TRASH, BDSF, LRSG, or STMST protein forms which have decreased or aberrant activity compared to ITALY, Lor-2, STRIFE, TRASH, BDSF, LRSG, or STMST wild type protein (e.g., immune system or inflammatory disorders, e.g., Rheumatoid Arthritis (Bucht A. et al. (1996) Clin. Exp. Immunol. 103:357-67 and Walmsley M. et al. (1996) Arthritis and Rheumatism 39:495-503), Systemic Lupus Erythematosus (Houssian F. A. et al, (1995) Lupus 4:393-5), Myasthenia Gravis (Zhang G. X. et al. (1997) Muscle Nerve 20:543-51), Grave's Disease (Kallmann B. A. et al, 1997) IDDM 46:237-43), Sjogren Syndrome (Ohyama Y. et al. (1996) Arthritis Rheumatism 39:1376-84), Sepsis, Polymyositis and Dermatomyositis (Hagiwara E. et al. (1996) Clin. Exp. Rheum. 14:485-91), Psoriasis (Michel G. et al. (1997) Inflammation Res. 46:32-4), Pemphigus Vulgaris (Wucherpfenig K. W. et al, (1995) Proc. Natl. Acad. Sci. 92:11935-9), Bullous Pemphigoid (Schmidt E. et al. (1996) Arch. Dermatol. Res. 288:353-7), Inflammatory Bowel Disease (Berg D. J. et al. (1996) J. Clin. Invest. 98:1010-44), Kawasaki Disease (Hira J. et al. (1997) Int. Arch. Allergy Immunol. 112:152-6), Asthma (Kawano Y. et al. (1995) Clin. Exp. Immunol. 102:389-94), and Graft v. Host Disease, e.g., in bone marrow transplantation; proliferative disorders, e.g., cancer such as melanoma, prostate cancer, cervical cancer, breast cancer, colon cancer, or sarcoma; or differentiative or developmental disorders). Moreover, soluble forms of the ITALY, Lor-2, STRIFE, TRASH, BDSF, LRSG, or STMST protein can be used to bind other membrane-bound cytokine receptors and influence bioavailability of such a receptor's cognate ligand. Moreover, the anti-ITALY, Lor-2, STRIFE, TRASH, BDSF, LRSG, or STMST antibodies of the invention can be used to detect and isolate ITALY, Lor-2, STRIFE, TRASH, BDSF, LRSG, or STMST proteins, regulate the bioavailability of ITALY, Lor-2, STRIFE, TRASH, BDSF, LRSG, or STMST proteins, and modulate ITALY, Lor-2, STRIFE, TRASH, BDSF, LRSG, or STMST activity.

It will therefore be appreciated that the ITALY, Lor-2, STRIFE, TRASH, BDSF, LRSG, or STMST polypeptides, nucleic acids, and modulators thereof can be used to modulate the function, morphology, proliferation and/or differentiation of cells in the tissues in which it is expressed, as described herein. Such molecules can thus be used to treat disorders associated with abnormal or aberrant metabolism or function of cells in the tissues in which it is expressed.

For example, when ITALY, Lor-2, STRIFE, TRASH, BDSF, LRSG, or STMST polypeptides and nucleic acids are expressed in the pancreas, the polypeptides, nucleic acids, and modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

In another example, when ITALY, Lor-2, STRIFE, TRASH, BDSF, LRSG, or STMST polypeptides and nucleic acids are expressed in the heart, the polypeptides, nucleic acids, and modulators thereof can be used to treat heart disorders, e.g., ischemic heart disease, atherosclerosis, hypertension, angina pectoris, Hypertrophic Cardiomyopathy, and congenital heart disease.

In still another example, the ITALY, Lor-2, STRIFE, TRASH, BDSF, LRSG, or STMST polypeptides, nucleic acids, and modulators thereof may be used to modulate the pituitary gland, which secretes such hormones as thyroid stimulating hormone (TSH), follicle stimulating hormone (FSH), adrenocotropic hormone (ACTH), and others. It controls the activity of many other endocrine glands (thyroid, ovaries, adrenal, etc.). Pituitary related disorders include, among others, acromegaly, Cushing's syndrome, craniopharyngiomas, Empty Sella syndrome, hypogonadism, hypopituitarism, and hypophysitis, in addition to disorders of the endocrine glands the pituitary controls.

In another example, the ITALY, Lor-2, STRIFE, TRASH, BDSF, LRSG, or STMST polypeptides, nucleic acids, and modulators thereof can be used to treat disorders of the adrenal cortex, such as hypoadrenalism (e.g., primary chronic or acute adrenocortical insufficiency, and secondary adrenocortical insufficiency), hyperadrenalism (Cushing's syndrome, primary hyperaldosteronism, adrenal virilism, and adrenal hyperplasia), or neoplasia (e.g., adrenal adenoma and cortical carcinoma).

In another example, the ITALY, Lor-2, STRIFE, TRASH, BDSF, LRSG, or STMST polypeptides, nucleic acids, and modulators thereof can be used to treat disorders of the adrenal medulla, such as neoplasms (e.g., pheochromocytomas, neuroblastomas, and ganglioneuromas).

In another example, the ITALY, Lor-2, STRIFE, TRASH, BDSF, LRSG, or STMST polypeptides, nucleic acids, and modulators thereof can be used to treat disorders of the thyroid gland, such as hyperthyroidism (e.g., diffuse toxic hyperplasia, toxic multinodular goiter, toxic adenoma, and acute or subacute thyroiditis), hypothyroidism (e.g., cretinism and myxedema), thyroiditis (e.g., Hashimoto's thyroiditis, subacute granulomatous thyroiditis, subacute lymphocytic thyroiditis, Riedel's thyroiditis), Graves' disease, goiter (e.g., simple diffuse goiter and multinodular goiter), or tumors (e.g., adenoma, papillary carcinoma, follicular carcinoma, medullary carcinoma, undifferentiated malignant carcinoma, Hodgkin's disease, and non-Hodgkin's lymphoma).

In another example, the ITALY, Lor-2, STRIFE, TRASH, BDSF, LRSG, or STMST polypeptides, nucleic acids, and modulators thereof can be used to treat gastric disorders, such as congenital anomalies (e.g., diaphragmatic hernias, pyloric stenosis, gastric diverticula, and gastric dilatation), gastritis (e.g., acute mucosal inflammation, chronic fundal gastritis, chronic antral gastritis, hypertrophic gastritis, granulomatous gastritis, eosinophilic gastritis), ulcerations (e.g., peptic ulcers, gastric ulcers, and duodenal ulcers), or tumors (e.g., benign polyps, malignant carcinoma, argentaffinomas, carcinoids, gastrointestinal lymphomas, carcomas, and metastatic carcinoma).

In another example, the ITALY, Lor-2, STRIFE, TRASH, BDSF, LRSG, or STMST polypeptides, nucleic acids, and modulators thereof can be used to treat placental disorders, such as toxemia of pregnancy (e.g., preeclampsia and eclampsia), placentitis, or spontaneous abortion.

In another example, the ITALY, Lor-2, STRIFE, TRASH, BDSF, LRSG, or STMST polypeptides, nucleic acids, and modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis, Goodpasture's syndrome, idiopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another example, the ITALY, Lor-2, STRIFE, TRASH, BDSF, LRSG, or STMST polypeptides, nucleic acids, and modulators thereof can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

In another example, the ITALY, Lor-2, STRIFE, TRASH, BDSF, LRSG, or STMST polypeptides, nucleic acids, and modulators thereof can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cardiomyopathy).

In another example, the ITALY, Lor-2, STRIFE, TRASH, BDSF, LRSG, or STMST polypeptides, nucleic acids, and modulators thereof can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis), hepatitis (e.g., chronic active hepatitis, acute viral hepatitis, and toxic and drug-induced hepatitis), cirrhosis (e.g., alcoholic cirrhosis, biliary cirrhosis, and hemochromatosis), or malignant tumors (e.g., primary carcinoma, hepatoma, hepatoblastoma, liver cysts, and angiosarcoma).

In another example, the ITALY, Lor-2, STRIFE, TRASH, BDSF, LRSG, or STMST polypeptides, nucleic acids, and modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

In another example, the ITALY, Lor-2, STRIFE, TRASH, BDSF, LRSG, or STMST polypeptides, nucleic acids, and modulators thereof can be used to treat testicular disorders, such as unilateral testicular enlargement (e.g., nontuberculous, granulomatous orchitis); inflammatory diseases resulting in testicular dysfunction (e.g., gonorrhea and mumps); cryptorchidism; sperm cell disorders (e.g., immotile cilia syndrome and germinal cell aplasia); acquired testicular defects (e.g., viral orchitis); and tumors (e.g., germ cell tumors, interstitial cell tumors, androblastoma, testicular lymphoma and adenomatoid tumors).

In another example, the ITALY, Lor-2, STRIFE, TRASH, BDSF, LRSG, or STMST polypeptides, nucleic acids, and modulators thereof can be used to treat uterine disorders, e.g., hyperplasia of the endometrium, uterine cancers (e.g., uterine leiomyomoma, uterine cellular leiomyoma, leiomyosarcoma of the uterus, malignant mixed mullerian Tumor of uterus, uterine Sarcoma), and dysfunctional uterine bleeding (DUB).

In another example, ITALY, Lor-2, STRIFE, TRASH, BDSF, LRSG, or STMST polypeptides, nucleic acids, and modulators thereof can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

In another example, ITALY, Lor-2, STRIFE, TRASH, BDSF, LRSG, or STMST nucleic acids, proteins, and modulators thereof can be used to modulate or treat disorders that include hepatitis B as well as brain and CNS related disorders. Such brain and CNS related disorders include but are not limited to bacterial and viral meningitis, Alzheimers Disease, cerebral toxoplasmosis, Parkinson's disease, multiple sclerosis, brain cancers (e.g., metastatic carcinoma of the brain, glioblastoma, lymphoma, astrocytoma, acoustic neuroma), hydrocephalus, and encephalitis.

In another example, ITALY, Lor-2, STRIFE, TRASH, BDSF, LRSG, or STMST nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. ITALY, Lor-2, STRIFE, TRASH, BDSF, LRSG, or STMST nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus ITALY, Lor-2, STRIFE, TRASH, BDSF, LRSG, or STMST nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfment of bacteria and viruses in the bloodstream.

In another example, ITALY, Lor-2, STRIFE, TRASH, BDSF, LRSG, or STMST nucleic acids, proteins, and modulators thereof can be used to modulate the proliferation, differentiation, and/or function of cells that form bone matrix, e.g., osteoblasts and osteoclasts, and can be used to modulate the formation of bone matrix. Thus ITALY, Lor-2, STRIFE, TRASH, BDSF, LRSG, or STMST nucleic acids, proteins, and modulators thereof can be used to treat cartilage and bone associated diseases and disorders, and can play a role in bone growth, formation, and remodeling. Examples of cartilage and bone associated diseases and disorders include e.g., bone cancer, achondroplasia, myeloma, fibrous dysplasia, scoliosis, osteoarthritis, osteosarcoma, and osteoporosis.

In another example, ITALY, Lor-2, STRIFE, TRASH, BDSF, LRSG, or STMST nucleic acids, proteins, and modulators thereof can be used to modulate the proliferation, differentiation, and/or function of cells that appear in the bone marrow, e.g., stem cells (e.g., hematopoietic stem cells), and blood cells, e.g., erythrocytes, platelets, and leukocytes. Thus ITALY, Lor-2, STRIFE, TRASH, BDSF, LRSG, or STMST nucleic acids, proteins, and modulators thereof can be used to treat bone marrow, blood, and hematopoietic associated diseases and disorders, e.g., acute myeloid leukemia, hemophilia, leukemia, anemia (e.g., sickle cell anemia), and thalassemia.

In another example, ITALY, Lor-2, STRIFE, TRASH, BDSF, LRSG, or STMST nucleic acids, proteins, and modulators thereof can be used to modulate the proliferation, differentiation, and/or function of bone and cartilage cells, e.g., chondrocytes and osteoblasts, and to treat bone and/or cartilage associated diseases or disorders. Examples of bone and/or cartilage diseases and disorders include bone and/or cartilage injury due to for example, trauma (e.g., bone breakage, cartilage tearing), degeneration (e.g., osteoporosis), degeneration of joints, e.g., arthritis, e.g., osteoarthritis, and bone wearing.

In another example, ITALY, Lor-2, STRIFE, TRASH, BDSF, LRSG, or STMST polypeptides, nucleic acids, and modulators thereof can be used to treat esophageal and other digestive system related disorders, e.g., dysphagia (e.g., oropharyngeal dysphagia, esophageal dysphagia), pyrosis, achalasia, diffuse esophageal spasm, nutcracker esophagus, and gastroesophageal reflux disease.

In another example, ITALY, Lor-2, STRIFE, TRASH, BDSF, LRSG, or STMST polypeptides, nucleic acids, and modulators thereof can be used to treat eye disorders, e.g., Retinitis Pigmentosa, Cataract, Color Blindness, Conjunctivitis, Dry Eyes, Glaucoma, Keratoconus, Macular Degeneration, Microphthalmia and Anophthalmia, Myopia, Nystagmus, Retinitis Pigmentosa, and Trachoma.

In another example, ITALY, Lor-2, STRIFE, TRASH, BDSF, LRSG, or STMST polypeptides, nucleic acids, and modulators thereof can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

In another example, ITALY, Lor-2, STRIFE, TRASH, BDSF, LRSG, or STMST polypeptides, nucleic acids, and modulators thereof can be used to treat ovarian disorders, such as ovarian endometriosis, non-neoplastic cysts (e.g., follicular and luteal cysts and polycystic ovaries) and tumors (e.g., tumors of surface epithelium, germ cell tumors, ovarian fibroma, sex cord-stromal tumors, and ovarian cancers (e.g., metastatic carcinomas, and ovarian teratoma).

In another example, ITALY, Lor-2, STRIFE, TRASH, BDSF, LRSG, or STMST polypeptides, nucleic acids, and modulators thereof can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

In another example, ITALY, Lor-2, STRIFE, TRASH, BDSF, LRSG, or STMST polypeptides, nucleic acids, and modulators thereof can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., colitis (e.g., idiopathic ulcerative colitis, pseudomembranous colitis), and lymphopathia venereum), Crohn's disease, and tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas).

In another example, ITALY, Lor-2, STRIFE, TRASH, BDSF, LRSG, or STMST polypeptides, nucleic acids, and modulators thereof can be used to treat leukocytic disorders, such as leukopenias (e.g., neutropenia, monocytopenia, lymphopenia, and granulocytopenia), leukocytosis (e.g., granulocytosis, lymphocytosis, eosinophilia, monocytosis, acute and chronic lymphadenitis), malignant lymphomas (e.g., Non-Hodgkin's lymphomas, Hodgkin's lymphomas, leukemias, agnogenic myeloid metaplasia, multiple myeloma, plasmacytoma, Waldenstrom's macroglobulinemia, heavy-chain disease, monoclonal gammopathy, histiocytoses, eosinophilic granuloma, and angioimmunoblastic lymphadenopathy).

In another example, STMST polypeptides, nucleic acids, and modulators thereof can be used in treating, for example, hypothalamic dysfunction and/or disorders, body weight disorders (e.g., anorexia, obesity and/or hyperphagia), eating disorders (e.g., anorexia nervosa and/or bulimia nervosa), cachexia, AIDS-related wasting, and cancer-related wasting. As used herein, the term “hypothalamic dysfunction” includes a mis-regulated or aberrantly regulated function or activity attributed to the hypothalamus in an animal (e.g., in a human), for example, a mis-regulated or aberrantly regulated hypothalamic activity, as described herein. As used herein, the term “hypothalamic disorder” includes a disease or disorder characterized by at least one phenotypic manifestation (e.g., a clinically detectable manifestation or symptom) of a hypothalamic dysfunction, as defined herein. Modulation of an STMST activity as described above may be included as part of a multi-drug regime that targets multiple sites within the weight regulatory system, temperature regulatory system, sleep-wake cycle control system, memory and/or behavior regulatory systems, thirst regulatory system and/or autonomic nervous system.

In another example, STMST polypeptides, nucleic acids, and modulators thereof can be used in treating disorders related to vascular tone, for example, cardiovascular disorders such as hypertension, arteriosclerosis, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, atrial fibrilation, Jervell syndrome, Lange-Nielsen syndrome, long-QT syndrome, congestive heart failure, sinus node dysfunction, angina, heart failure, atrial fibrillation, atrial flutter, dilated cardiomyopathy, idiopathic cardiomyopathy, myocardial infarction, coronary artery disease, coronary artery spasm, arrhythmia, atherosclerosis, transplant atherosclerosis, varicose veins, migraine headaches, cluster headaches, vascular disease, diabetic vascular disease, pulmonary vascular disease, peripheral vascular disease, renovascular hypertension, intravascular tumor, pulmonary vasculitis, vascular tone disorders in pregnancy, pulmonary capillaritis, peripheral arterial disease, idiopathic hypereosiniphilic syndrome, aortic aneurysm, respiratory disease, vasospasm, systemic sclerosis, preeclampsia, graft vessel disease, cardiac allograft vasculopathy, vascular ischemic injury, familial amyloidotic polyneuropathy, acute atherosis, cardiovascular disease, Kawasaki disease, ischemic syndromes, chronic heart failure, and fibrosis.

In another example, STMST polypeptides, nucleic acids, and modulators thereof can be used in treating disorders related to angiogenesis, for example, diabetic retinopathy, neovascularization, psoriasis, endometriosis, Grave's disease, ischemic disease, chronic inflammatory diseases, macular degeneration, neovascular glaucoma, retinal fibroplasia, uveitis, eye diseases associated with choroidal neovascularization and iris neovascularization, hereditary hemorrhagic telangiectasia type 1, fibrodysplasia ossificans progressiva, idiopathic pulmonary fibrosis, and Klippel-Trenaunay-Weber syndrome.

A. Screening Assays:

The invention provides a method (also referred to herein as a “screening assay”) for identifying modulators, i.e., candidate or test compounds or agents (e.g., peptides, peptidomimetics, small molecules or other drugs) which bind to ITALY, Lor-2, STRIFE, TRASH, BDSF, LRSG, or STMST proteins, have a stimulatory or inhibitory effect on, for example, ITALY, Lor-2, STRIFE, TRASH, BDSF, LRSG, or STMST expression or ITALY, Lor-2, STRIFE, TRASH, BDSF, LRSG, or STMST activity, or have a stimulatory or inhibitory effect on, for example, the expression or activity of ITALY, Lor-2, STRIFE, TRASH, BDSF, LRSG, or STMST substrate.

In one embodiment, the invention provides assays for screening candidate or test compounds which are substrates of an ITALY, Lor-2, STRIFE, TRASH, BDSF, LRSG, or STMST protein or polypeptide or biologically active portion thereof. In another embodiment, the invention provides assays for screening candidate or test compounds which bind to or modulate the activity of an ITALY, Lor-2, STRIFE, TRASH, BDSF, LRSG, or STMST protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses an ITALY, Lor-2, STRIFE, TRASH, BDSF, LRSG, or STMST protein or biologically active portion thereof is contacted with a test compound and the ability of the test compound to modulate ITALY, Lor-2, STRIFE, TRASH, BDSF, LRSG, or STMST activity determined. Determining the ability of the test compound to modulate ITALY, Lor-2, STRIFE, TRASH, BDSF, LRSG, or STMST activity can be accomplished by monitoring the bioactivity of an ITALY, Lor-2, STRIFE, TRASH, BDSF, LRSG, or STMST substrate (e.g., a growth factor or other bioactive peptide). The cell, for example, can be of mammalian origin or a yeast cell. Determining the ability of the test compound to modulate ITALY, Lor-2, STRIFE, TRASH, BDSF, LRSG, or STMST activity can be accomplished, for example, by coupling the ITALY, Lor-2, STRIFE, TRASH, BDSF, LRSG, or STMST substrate with a radioisotope or enzymatic label such that binding of the ITALY, Lor-2, STRIFE, TRASH, BDSF, LRSG, or STMST substrate to its cognate receptor can be determined by detecting the labeled ITALY, Lor-2, STRIFE, TRASH, BDSF, LRSG, or STMST substrate in a complex. For example, compounds (e.g., ITALY, Lor-2, STRIFE, TRASH, BDSF, LRSG, or STMST substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an ITALY, Lor-2, STRIFE, TRASH, BDSF, LRSG, or STMST substrate) to interact with its cognate receptor without the labeling of any of the interactants. For example, a microphysiometer can be used to detect the interaction of a compound with its cognate receptor without the labeling of either the compound or the receptor. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In a preferred embodiment, the assay comprises contacting a cell which expresses an ITALY, Lor-2, STRIFE, TRASH, BDSF, LRSG, or STMST protein or biologically active portion thereof, with an ITALY, Lor-2, STRIFE, TRASH, BDSF, LRSG, or STMST substrate to form an assay mixture, contacting the assay mixture with a test compound, and determining the ability of the test compound to modulate the activity of the ITALY, Lor-2, STRIFE, TRASH, BDSF, LRSG, or STMST protein or biologically active portion thereof, wherein determining the ability of the test compound to modulate the activity of the ITALY, Lor-2, STRIFE, TRASH, BDSF, LRSG, or STMST protein or biologically active portion thereof, comprises determining the ability of the test compound to modulate a biological activity of the ITALY, Lor-2, STRIFE, TRASH, BDSF, LRSG, or STMST expressing cell (e.g., determining the ability of the test compound to modulate proliferation or differentiation of the cell).

In another preferred embodiment, the assay comprises contacting a cell which is responsive to an ITALY, Lor-2, STRIFE, TRASH, BDSF, LRSG, or STMST protein or biologically active portion thereof, with an ITALY, Lor-2, STRIFE, TRASH, BDSF, LRSG, or STMST protein or biologically-active portion thereof, to form an assay mixture, contacting the assay mixture with a test compound, and determining the ability of the test compound to modulate the activity of the ITALY, Lor-2, STRIFE, TRASH, BDSF, LRSG, or STMST protein or biologically active portion thereof, wherein determining the ability of the test compound to modulate the activity of the ITALY, Lor-2, STRIFE, TRASH, BDSF, LRSG, or STMST protein or biologically active portion thereof comprises determining the ability of the test compound to modulate a biological activity of the ITALY, Lor-2, STRIFE, TRASH, BDSF, LRSG, or STMST-responsive cell (e.g., determining the ability of the test compound to modulate proliferation or differentiation of the cell).

In another embodiment, an assay is a cell-based assay comprising contacting a cell expressing an ITALY, Lor-2, STRIFE, TRASH, BDSF, LRSG, or STMST target molecule (e.g., an ITALY, Lor-2, STRIFE, TRASH, BDSF, LRSG, or STMST substrate) with a test compound and determining the ability of the test compound to modulate (e.g. stimulate or inhibit) the activity of the ITALY, Lor-2, STRIFE, TRASH, BDSF, LRSG, or STMST target molecule. Determining the ability of the test compound to modulate the activity of an ITALY, Lor-2, STRIFE, TRASH, BDSF, LRSG, or STMST target molecule can be accomplished, for example, by determining the ability of the ITALY, Lor-2, STRIFE, TRASH, BDSF, LRSG, or STMST protein to bind to or interact with the ITALY, Lor-2, STRIFE, TRASH, BDSF, LRSG, or STMST target molecule.

Determining the ability of the ITALY, Lor-2, STRIFE, TRASH, BDSF, LRSG, or STMST protein to bind to or interact with an ITALY, Lor-2, STRIFE, TRASH, BDSF, LRSG, or STMST target molecule can be accomplished by one of the methods described above for determining direct binding. In a preferred embodiment, determining the ability of the ITALY, Lor-2, STRIFE, TRASH, BDSF, LRSG, or STMST protein to bind to or interact with an ITALY, Lor-2, STRIFE, TRASH, BDSF, LRSG, or STMST target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response, for example, cellular proliferation or metastasis.

In yet another embodiment, an assay of the present invention is a cell-free assay in which an ITALY, Lor-2, STRIFE, TRASH, BDSF, LRSG, or STMST protein or biologically active portion thereof is contacted with a test compound and the ability of the test compound to bind to the ITALY, Lor-2, STRIFE, TRASH, BDSF, LRSG, or STMST protein or biologically active portion thereof is determined. Binding of the test compound to the ITALY, Lor-2, STRIFE, TRASH, BDSF, LRSG, or STMST protein can be determined either directly or indirectly as described above. In a preferred embodiment, the assay includes contacting the ITALY, Lor-2, STRIFE, TRASH, BDSF, LRSG, or STMST protein or biologically active portion thereof with a known compound which binds ITALY, Lor-2, STRIFE, TRASH, BDSF, LRSG, or STMST to form an assay mixture, contacting the assay mixture with a test compound, and determining the ability of the test compound to interact with an ITALY, Lor-2, STRIFE, TRASH, BDSF, LRSG, or STMST protein, wherein determining the ability of the test compound to interact with an ITALY, Lor-2, STRIFE, TRASH, BDSF, LRSG, or STMST protein comprises determining the ability of the test compound to preferentially bind to ITALY, Lor-2, STRIFE, TRASH, BDSF, LRSG, or STMST or biologically active portion thereof as compared to the known compound.

In another embodiment, the assay is a cell-free assay in which an ITALY, Lor-2, STRIFE, TRASH, BDSF, LRSG, or STMST protein or biologically active portion thereof is contacted with a test compound and the ability of the test compound to modulate (e.g., stimulate or inhibit) the activity of the ITALY, Lor-2, STRIFE, TRASH, BDSF, LRSG, or STMST protein or biologically active portion thereof is determined. Determining the ability of the test compound to modulate the activity of an ITALY, Lor-2, STRIFE, TRASH, BDSF, LRSG, or STMST protein can be accomplished, for example, by determining the ability of the ITALY, Lor-2, STRIFE, TRASH, BDSF, LRSG, or STMST protein to bind to an ITALY, Lor-2, STRIFE, TRASH, BDSF, LRSG, or STMST target molecule by one of the methods described above for determining direct binding. Determining the ability of the ITALY, Lor-2, STRIFE, TRASH, BDSF, LRSG, or STMST protein to bind to an ITALY, Lor-2, STRIFE, TRASH, BDSF, LRSG, or STMST target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an ITALY, Lor-2, STRIFE, TRASH, BDSF, LRSG, or STMST protein can be accomplished by determining the ability of the ITALY, Lor-2, STRIFE, TRASH, BDSF, LRSG, or STMST protein to further modulate the activity of a downstream effector (e.g., a growth factor mediated signal transduction pathway component) of an ITALY, Lor-2, STRIFE, TRASH, BDSF, LRSG, or STMST target molecule (e.g., a growth factor). For example, the activity of the effector molecule on an appropriate target can be determined or the binding of the effector to an appropriate target can be determined as previously described.

In yet another embodiment, the cell-free assay involves contacting an ITALY, Lor-2, STRIFE, TRASH, BDSF, LRSG, or STMST protein or biologically active portion thereof with a known compound which binds the ITALY, Lor-2, STRIFE, TRASH, BDSF, LRSG, or STMST protein to form an assay mixture, contacting the assay mixture with a test compound, and determining the ability of the test compound to interact with the ITALY, Lor-2, STRIFE, TRASH, BDSF, LRSG, or STMST protein, wherein determining the ability of the test compound to interact with the ITALY, Lor-2, STRIFE, TRASH, BDSF, LRSG, or STMST protein comprises determining the ability of the ITALY, Lor-2, STRIFE, TRASH, BDSF, LRSG, or STMST protein to preferentially bind to or modulate the activity of an ITALY, Lor-2, STRIFE, TRASH, BDSF, LRSG, or STMST target molecule.

The cell-free assays of the present invention are amenable to use of both soluble and/or membrane-bound forms of isolated proteins (e.g. ITALY, Lor-2, STRIFE, TRASH, BDSF, LRSG, or STMST proteins or biologically active portions thereof or receptors to which ITALY, Lor-2, STRIFE, TRASH, BDSF, LRSG, or STMST targets bind). In the case of cell-free assays in which a membrane-bound form an isolated protein is used (e.g., a cell surface receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either ITALY, Lor-2, STRIFE, TRASH, BDSF, LRSG, or STMST or its target molecule to facilitate separation of complexed from uncomplexed forms of one or both of the proteins, as well as to accommodate automation of the assay. Binding of a test compound to an ITALY, Lor-2, STRIFE, TRASH, BDSF, LRSG, or STMST protein, or interaction of an ITALY, Lor-2, STRIFE, TRASH, BDSF, LRSG, or STMST protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/ITALY, Lor-2, STRIFE, TRASH, BDSF, LRSG, or STMST fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or ITALY, Lor-2, STRIFE, TRASH, BDSF, LRSG, or STMST protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of ITALY, Lor-2, STRIFE, TRASH, BDSF, LRSG, or STMST binding or activity determined using standard techniques.

Other techniques for immobilizing proteins on matrices can also be used in the screening assays of the invention. For example, either an ITALY, Lor-2, STRIFE, TRASH, BDSF, LRSG, or STMST protein or an ITALY, Lor-2, STRIFE, TRASH, BDSF, LRSG, or STMST target molecule can be immobilized utilizing conjugation of biotin and streptavidin. Biotinylated ITALY, Lor-2, STRIFE, TRASH, BDSF, LRSG, or STMST protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with ITALY, Lor-2, STRIFE, TRASH, BDSF, LRSG, or STMST protein or target molecules but which do not interfere with binding of the ITALY, Lor-2, STRIFE, TRASH, BDSF, LRSG, or STMST protein to its target molecule can be derivatized to the wells of the plate, and unbound target or ITALY, Lor-2, STRIFE, TRASH, BDSF, LRSG, or STMST protein trapped in the wells by antibody conjugation. Methods for detecting such complexes, in addition to those described above for the GST-immobilized complexes, include immunodetection of complexes using antibodies reactive with the ITALY, Lor-2, STRIFE, TRASH, BDSF, LRSG, or STMST protein or target molecule, as well as enzyme-linked assays which rely on detecting an enzymatic activity associated with the ITALY, Lor-2, STRIFE, TRASH, BDSF, LRSG, or STMST protein or target molecule.

In another embodiment, modulators of ITALY, Lor-2, STRIFE, TRASH, BDSF, LRSG, or STMST expression are identified in a method wherein a cell is contacted with a candidate compound and the expression of ITALY, Lor-2, STRIFE, TRASH, BDSF, LRSG, or STMST mRNA or protein in the cell is determined. The level of expression of ITALY, Lor-2, STRIFE, TRASH, BDSF, LRSG, or STMST mRNA or protein in the presence of the candidate compound is compared to the level of expression of ITALY, Lor-2, STRIFE, TRASH, BDSF, LRSG, or STMST mRNA or protein in the absence of the candidate compound. The candidate compound can then be identified as a modulator of ITALY, Lor-2, STRIFE, TRASH, BDSF, LRSG, or STMST expression based on this comparison. For example, when expression of ITALY, Lor-2, STRIFE, TRASH, BDSF, LRSG, or STMST mRNA or protein is greater (statistically significantly greater) in the presence of the candidate compound than in its absence, the candidate compound is identified as a stimulator of ITALY, Lor-2, STRIFE, TRASH, BDSF, LRSG, or STMST mRNA or protein expression. Alternatively, when expression of ITALY, Lor-2, STRIFE, TRASH, BDSF, LRSG, or STMST mRNA or protein is less (statistically significantly less) in the presence of the candidate compound than in its absence, the candidate compound is identified as an inhibitor of ITALY, Lor-2, STRIFE, TRASH, BDSF, LRSG, or STMST mRNA or protein expression. The level of ITALY, Lor-2, STRIFE, TRASH, BDSF, LRSG, or STMST mRNA or protein expression in the cells can be determined by methods described herein for detecting ITALY, Lor-2, STRIFE, TRASH, BDSF, LRSG, or STMST mRNA or protein.

In yet another aspect of the invention, the ITALY, Lor-2, STRIFE, TRASH, BDSF, LRSG, or STMST proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with ITALY, Lor-2, STRIFE, TRASH, BDSF, LRSG, or STMST (“ITALY, Lor-2, STRIFE, TRASH, BDSF, LRSG, or STMST-binding proteins” or “ITALY, Lor-2, STRIFE, TRASH, BDSF, LRSG, or STMST-bp”) and are involved in ITALY, Lor-2, STRIFE, TRASH, BDSF, LRSG, or STMST activity. Such ITALY, Lor-2, STRIFE, TRASH, BDSF, LRSG, or STMST-binding proteins are also likely to be involved in the propagation of signals by the ITALY, Lor-2, STRIFE, TRASH, BDSF, LRSG, or STMST proteins or ITALY, Lor-2, STRIFE, TRASH, BDSF, LRSG, or STMST targets as, for example, downstream elements of an ITALY, Lor-2, STRIFE, TRASH, BDSF, LRSG, or STMST-mediated signaling pathway. Alternatively, such ITALY, Lor-2, STRIFE, TRASH, BDSF, LRSG, or STMST-binding proteins are likely to be ITALY, Lor-2, STRIFE, TRASH, BDSF, LRSG, or STMST inhibitors.

The two-hybrid system is based on the modular nature of most transcription factors, which consist of separable DNA-binding and activation domains. Briefly, the assay utilizes two different DNA constructs. In one construct, the gene that codes for an ITALY, Lor-2, STRIFE, TRASH, BDSF, LRSG, or STMST protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an ITALY, Lor-2, STRIFE, TRASH, BDSF, LRSG, or STMST-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the ITALY, Lor-2, STRIFE, TRASH, BDSF, LRSG, or STMST protein.

This invention further pertains to novel agents identified by the above-described screening assays. Accordingly, it is within the scope of this invention to further use an agent identified as described herein in an appropriate animal model. For example, an agent identified as described herein (e.g., an ITALY, Lor-2, STRIFE, TRASH, BDSF, LRSG, or STMST modulating agent, an antisense ITALY, Lor-2, STRIFE, TRASH, BDSF, LRSG, or STMST nucleic acid molecule, an ITALY, Lor-2, STRIFE, TRASH, BDSF, LRSG, or STMST-specific antibody, or an ITALY, Lor-2, STRIFE, TRASH, BDSF, LRSG, or STMST-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the ITALY, Lor-2, STRIFE, TRASH, BDSF, LRSG, or STMST nucleotide sequences, described herein, can be used to map the location of the ITALY, Lor-2, STRIFE, TRASH, BDSF, LRSG, or STMST genes on a chromosome. The mapping of the ITALY, Lor-2, STRIFE, TRASH, BDSF, LRSG, or STMST sequences to chromosomes is an important first step in correlating these sequences with genes associated with disease. For example, the Lor-2 gene maps to human chromosome 2 between WI-5987 (13.9cR) and GCT1B4 (16.7cR), and the LRSG-1 gene was found to map to human chromosome 16 between markers WI-7742 and WI-3061 (46-52cM).

Briefly, ITALY, Lor-2, STRIFE, TRASH, BDSF, LRSG, or STMST genes can be mapped to chromosomes by preparing PCR primers (preferably 15-25 bp in length) from the ITALY, Lor-2, STRIFE, TRASH, BDSF, LRSG, or STMST nucleotide sequences. Computer analysis of the ITALY, Lor-2, STRIFE, TRASH, BDSF, LRSG, or STMST sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the ITALY, Lor-2, STRIFE, TRASH, BDSF, LRSG, or STMST sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the ITALY, Lor-2, STRIFE, TRASH, BDSF, LRSG, or STMST nucleotide sequences to design oligonucleotide primers, sublocalization can be achieved with panels of fragments from specific chromosomes. Other mapping strategies which can similarly be used to map a 9o, 1p, or 1v sequence to its chromosome include in situ hybridization (described in Fan, Y. et al. (1990) Proc. Natl. Acad. Sci. USA, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature, 325:783-787.

Moreover, differences in the DNA sequences between individuals affected and unaffected with a disease associated with the ITALY, Lor-2, STRIFE, TRASH, BDSF, LRSG, or STMST gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The ITALY, Lor-2, STRIFE, TRASH, BDSF, LRSG, and STMST sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the ITALY, Lor-2, STRIFE, TRASH, BDSF, LRSG, or STMST nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The ITALY, Lor-2, STRIFE, TRASH, BDSF, LRSG, and STMST nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO:1, 10, 22, 26, 31, 45, 50, 58, 67, 70, or 73, can comfortably provide positive individual identification with a panel of perhaps 10 to 1,000 primers which each yield a noncoding amplified sequence of 100 bases. If predicted coding sequences, such as those in SEQ ID NO:3, 12, 24, 28, 33, 38, 40, 47, 52, 60, 69, 72, or 75 are used, a more appropriate number of primers for positive individual identification would be 500-2,000.

If a panel of reagents from ITALY, Lor-2, STRIFE, TRASH, BDSF, LRSG, or STMST nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of Partial ITALY, Lor-2, STRIFE, TRASH, BDSF, LRSG, and STMST Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO:1, 10, 22, 26, 31, 45, 50, 58, 67, 70, or 73 are particularly appropriate for this use as greater numbers of polymorphisms occur in the noncoding regions, making it easier to differentiate individuals using this technique. Examples of polynucleotide reagents include the ITALY, Lor-2, STRIFE, TRASH, BDSF, LRSG, or STMST nucleotide sequences or portions thereof, e.g., fragments derived from the noncoding regions of SEQ ID NO:1, 10, 22, 26, 31, 45, 50, 58, 67, 70, or 73, having a length of at least 20 bases, preferably at least 30 bases.

The ITALY, Lor-2, STRIFE, TRASH, BDSF, LRSG, or STMST nucleotide sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such ITALY, Lor-2, STRIFE, TRASH, BDSF, LRSG, or STMST probes can be used to identify tissue by species and/or by organ type.

In a similar fashion, these reagents, e.g., ITALY, Lor-2, STRIFE, TRASH, BDSF, LRSG, or STMST primers or probes can be used to screen tissue culture for contamination (i.e. screen for the presence of a mixture of different types of cells in a culture).

C. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining ITALY, Lor-2, STRIFE, TRASH, BDSF, LRSG, or STMST protein and/or nucleic acid expression as well as ITALY, Lor-2, STRIFE, TRASH, BDSF, LRSG, or STMST activity, in the context of a biological sample (e.g., blood, serum, cells, tissue) to thereby determine whether an individual is afflicted with a disease or disorder, or is at risk of developing a disorder, associated with aberrant ITALY, Lor-2, STRIFE, TRASH, BDSF, LRSG, or STMST expression or activity. The invention also provides for prognostic (or predictive) assays for determining whether an individual is at risk of developing a disorder associated with ITALY, Lor-2, STRIFE, TRASH, BDSF, LRSG, or STMST protein, nucleic acid expression or activity. For example, mutations in an ITALY, Lor-2, STRIFE, TRASH, BDSF, LRSG, or STMST gene can be assayed in a biological sample. Such assays can be used for prognostic or predictive purpose to thereby prophylactically treat an individual prior to the onset of a disorder characterized by or associated with ITALY, Lor-2, STRIFE, TRASH, BDSF, LRSG, or STMST protein, nucleic acid expression or activity.

Another aspect of the invention pertains to monitoring the influence of agents (e.g., drugs, compounds) on the expression or activity of ITALY, Lor-2, STRIFE, TRASH, BDSF, LRSG, or STMST in clinical trials.

These and other agents are described in further detail in the following sections.

1. Diagnostic Assays

An exemplary method for detecting the presence or absence of ITALY, Lor-2, STRIFE, TRASH, BDSF, LRSG, or STMST protein or nucleic acid in a biological sample involves obtaining a biological sample from a test subject and contacting the biological sample with a compound or an agent capable of detecting ITALY, Lor-2, STRIFE, TRASH, BDSF, LRSG, or STMST protein or nucleic acid (e.g., mRNA, genomic DNA) that encodes ITALY, Lor-2, STRIFE, TRASH, BDSF, LRSG, or STMST protein such that the presence of ITALY, Lor-2, STRIFE, TRASH, BDSF, LRSG, or STMST protein or nucleic acid is detected in the biological sample. A preferred agent for detecting ITALY, Lor-2, STRIFE, TRASH, BDSF, LRSG, or STMST mRNA or genomic DNA is a labeled nucleic acid probe capable of hybridizing to ITALY, Lor-2, STRIFE, TRASH, BDSF, LRSG, or STMST mRNA or genomic DNA. The nucleic acid probe can be, for example, a full-length ITALY, Lor-2, STRIFE, TRASH, BDSF, LRSG, or STMST nucleic acid, such as the nucleic acid of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the DNA insert of the plasmid deposited with ATCC as Accession Number 98960, 98756, or 98695, or a portion thereof, such as an oligonucleotide of at least 15, 30, 50, 100, 250 or 500 nucleotides in length and sufficient to specifically hybridize under stringent conditions to ITALY, Lor-2, STRIFE, TRASH, BDSF, LRSG, or STMST mRNA or genomic DNA. Other suitable probes for use in the diagnostic assays of the invention are described herein.

A preferred agent for detecting ITALY, Lor-2, STRIFE, TRASH, BDSF, LRSG, or STMST protein is an antibody capable of binding to ITALY, Lor-2, STRIFE, TRASH, BDSF, LRSG, or STMST protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect ITALY, Lor-2, STRIFE, TRASH, BDSF, LRSG, or STMST mRNA, protein, or genomic DNA in a biological sample in vitro as well as in vivo. For example, in vitro techniques for detection of ITALY, Lor-2, STRIFE, TRASH, BDSF, LRSG, or STMST mRNA include Northern hybridizations and in situ hybridizations. In vitro techniques for detection of ITALY, Lor-2, STRIFE, TRASH, BDSF, LRSG, or STMST protein include enzyme linked immunosorbent assays (ELISAs), Western blots, immunoprecipitations and immunofluorescence. In vitro techniques for detection of ITALY, Lor-2, STRIFE, TRASH, BDSF, LRSG, or STMST genomic DNA include Southern hybridizations. Furthermore, in vivo techniques for detection of ITALY, Lor-2, STRIFE, TRASH, BDSF, LRSG, or STMST protein include introducing into a subject a labeled anti-ITALY, Lor-2, STRIFE, TRASH, BDSF, LRSG, or STMST antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting ITALY, Lor-2, STRIFE, TRASH, BDSF, LRSG, or STMST protein, mRNA, or genomic DNA, such that the presence of ITALY, Lor-2, STRIFE, TRASH, BDSF, LRSG, or STMST protein, mRNA or genomic DNA is detected in the biological sample, and comparing the presence of ITALY, Lor-2, STRIFE, TRASH, BDSF, LRSG, or STMST protein, mRNA or genomic DNA in the control sample with the presence of ITALY, Lor-2, STRIFE, TRASH, BDSF, LRSG, or STMST protein, mRNA or genomic DNA in the test sample.

The invention also encompasses kits for detecting the presence of ITALY, Lor-2, STRIFE, TRASH, BDSF, LRSG, or STMST in a biological sample. For example, the kit can comprise a labeled compound or agent capable of detecting ITALY, Lor-2, STRIFE, TRASH, BDSF, LRSG, or STMST protein or mRNA in a biological sample; means for determining the amount of ITALY, Lor-2, STRIFE, TRASH, BDSF, LRSG, or STMST in the sample; and means for comparing the amount of ITALY, Lor-2, STRIFE, TRASH, BDSF, LRSG, or STMST in the sample with a standard. The compound or agent can be packaged in a suitable container. The kit can further comprise instructions for using the kit to detect ITALY, Lor-2, STRIFE, TRASH, BDSF, LRSG, or STMST protein or nucleic acid.

2. Prognostic Assays

The diagnostic methods described herein can furthermore be utilized to identify subjects having or at risk of developing a disease or disorder associated with aberrant ITALY, Lor-2, STRIFE, TRASH, BDSF, LRSG, or STMST expression or activity. For example, the assays described herein, such as the preceding diagnostic assays or the following assays, can be utilized to identify a subject having or at risk of developing a disorder associated with ITALY, Lor-2, STRIFE, TRASH, BDSF, LRSG, or STMST protein, nucleic acid expression or activity such as prostate cancer. Alternatively, the prognostic assays can be utilized to identify a subject having or at risk for developing prostate cancer. Thus, the present invention provides a method for identifying a disease or disorder associated with aberrant ITALY, Lor-2, STRIFE, TRASH, BDSF, LRSG, or STMST expression or activity in which a test sample is obtained from a subject and ITALY, Lor-2, STRIFE, TRASH, BDSF, LRSG, or STMST protein or nucleic acid (e.g., mRNA, genomic DNA) is detected, wherein the presence of ITALY, Lor-2, STRIFE, TRASH, BDSF, LRSG, or STMST protein or nucleic acid is diagnostic for a subject having or at risk of developing a disease or disorder associated with aberrant ITALY, Lor-2, STRIFE, TRASH, BDSF, LRSG, or STMST expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant ITALY, Lor-2, STRIFE, TRASH, BDSF, LRSG, or STMST expression or activity. For example, such methods can be used to determine whether a subject can be effectively treated with an agent for prostate cancer. Thus, the present invention provides methods for determining whether a subject can be effectively treated with an agent for a disorder associated with aberrant ITALY, Lor-2, STRIFE, TRASH, BDSF, LRSG, or STMST expression or activity in which a test sample is obtained and ITALY, Lor-2, STRIFE, TRASH, BDSF, LRSG, or STMST protein or nucleic acid expression or activity is detected (e.g., wherein the abundance of ITALY, Lor-2, STRIFE, TRASH, BDSF, LRSG, or STMST protein or nucleic acid expression or activity is diagnostic for a subject that can be administered the agent to treat a disorder associated with aberrant ITALY, Lor-2, STRIFE, TRASH, BDSF, LRSG, or STMST expression or activity.)

The methods of the invention can also be used to detect genetic alterations in an ITALY, Lor-2, STRIFE, TRASH, BDSF, LRSG, or STMST gene, thereby determining if a subject with the altered gene is at risk for a disorder characterized by an aberrant proliferative response. In preferred embodiments, the methods include detecting, in a sample of cells from the subject, the presence or absence of a genetic alteration characterized by at least one of an alteration affecting the integrity of a gene encoding an ITALY, Lor-2, STRIFE, TRASH, BDSF, LRSG, or STMST protein, or the mis-expression of the ITALY, Lor-2, STRIFE, TRASH, BDSF, LRSG, or STMST gene. For example, such genetic alterations can be detected by ascertaining the existence of at least one of 1) a deletion of one or more nucleotides from an ITALY, Lor-2, STRIFE, TRASH, BDSF, LRSG, or STMST gene; 2) an addition of one or more nucleotides to an ITALY, Lor-2, STRIFE, TRASH, BDSF, LRSG, or STMST gene; 3) a substitution of one or more nucleotides of an ITALY, Lor-2, STRIFE, TRASH, BDSF, LRSG, or STMST gene, 4) a chromosomal rearrangement of an ITALY, Lor-2, STRIFE, TRASH, BDSF, LRSG, or STMST gene; 5) an alteration in the level of a messenger RNA transcript of an ITALY, Lor-2, STRIFE, TRASH, BDSF, LRSG, or STMST gene, 6) aberrant modification of an ITALY, Lor-2, STRIFE, TRASH, BDSF, LRSG, or STMST gene, such as of the methylation pattern of the genomic DNA, 7) the presence of a non-wild type splicing pattern of a messenger RNA transcript of an ITALY, Lor-2, STRIFE, TRASH, BDSF, LRSG, or STMST gene, 8) a non-wild type level of an ITALY, Lor-2, STRIFE, TRASH, BDSF, LRSG, or STMST-protein, 9) allelic loss of an ITALY, Lor-2, STRIFE, TRASH, BDSF, LRSG, or STMST gene, and 10) inappropriate post-translational modification of an ITALY, Lor-2, STRIFE, TRASH, BDSF, LRSG, or STMST-protein. As described herein, there are a large number of assay techniques known in the art which can be used for detecting alterations in an ITALY, Lor-2, STRIFE, TRASH, BDSF, LRSG, or STMST gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the ITALY, Lor-2, STRIFE, TRASH, BDSF, LRSG, or STMST-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to an ITALY, Lor-2, STRIFE, TRASH, BDSF, LRSG, or STMST gene under conditions such that hybridization and amplification of the ITALY, Lor-2, STRIFE, TRASH, BDSF, LRSG, or STMST gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an ITALY, Lor-2, STRIFE, TRASH, BDSF, LRSG, or STMST gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in ITALY, Lor-2, STRIFE, TRASH, BDSF, LRSG, or STMST can be identified by hybridizing a sample and control nucleic acids, e.g., DNA or RNA, to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, genetic mutations in ITALY, Lor-2, STRIFE, TRASH, BDSF, LRSG, or STMST can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ITALY, Lor-2, STRIFE, TRASH, BDSF, LRSG, or STMST gene and detect mutations by comparing the sequence of the sample ITALY, Lor-2, STRIFE, TRASH, BDSF, LRSG, or STMST with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the ITALY, Lor-2, STRIFE, TRASH, BDSF, LRSG, or STMST gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type ITALY, Lor-2, STRIFE, TRASH, BDSF, LRSG, or STMST sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in ITALY, Lor-2, STRIFE, TRASH, BDSF, LRSG, or STMST cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an ITALY, Lor-2, STRIFE, TRASH, BDSF, LRSG, or STMST sequence, e.g., a wild-type ITALY, Lor-2, STRIFE, TRASH, BDSF, LRSG, or STMST sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in ITALY, Lor-2, STRIFE, TRASH, BDSF, LRSG, or STMST genes. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat. Res. 285:125-144; and Hayashi (1992) Genet. Anal. Tech. Appl. 9:73-79). Single-stranded DNA fragments of sample and control ITALY, Lor-2, STRIFE, TRASH, BDSF, LRSG, or STMST nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an ITALY, Lor-2, STRIFE, TRASH, BDSF, LRSG, or STMST gene.

Furthermore, any cell type or tissue in which ITALY, Lor-2, STRIFE, TRASH, BDSF, LRSG, or STMST is expressed may be utilized in the prognostic assays described herein.

3. Monitoring of Effects During Clinical Trials

Monitoring the influence of agents (e.g., drugs, compounds) on the expression or activity of an ITALY, Lor-2, STRIFE, TRASH, BDSF, LRSG, or STMST protein (e.g., modulation of angiogenesis or of an inflammatory response) an be applied not only in basic drug screening, but also in clinical trials. For example, the effectiveness of an agent determined by a screening assay as described herein to increase ITALY, Lor-2, STRIFE, TRASH, BDSF, LRSG, or STMST gene expression, protein levels, or upregulate ITALY, Lor-2, STRIFE, TRASH, BDSF, LRSG, or STMST activity, can be monitored in clinical trials of subjects exhibiting decreased ITALY, Lor-2, STRIFE, TRASH, BDSF, LRSG, or STMST gene expression, protein levels, or down-regulated ITALY, Lor-2, STRIFE, TRASH, BDSF, LRSG, or STMST activity. Alternatively, the effectiveness of an agent determined by a screening assay to decrease ITALY, Lor-2, STRIFE, TRASH, BDSF, LRSG, or STMST gene expression, protein levels, or downregulate ITALY, Lor-2, STRIFE, TRASH, BDSF, LRSG, or STMST activity, can be monitored in clinical trials of subjects exhibiting increased ITALY, Lor-2, STRIFE, TRASH, BDSF, LRSG, or STMST gene expression, protein levels, or upregulated ITALY, Lor-2, STRIFE, TRASH, BDSF, LRSG, or STMST activity. In such clinical trials, the expression or activity of an ITALY, Lor-2, STRIFE, TRASH, BDSF, LRSG, or STMST gene, and preferably, other genes that have been implicated in, for example, a proliferative disorder can be used as a “read out” or markers of the phenotype of a particular cell.

For example, and not by way of limitation, genes, including ITALY, Lor-2, STRIFE, TRASH, BDSF, LRSG, or STMST, that are modulated in cells by treatment with an agent (e.g., compound, drug or small molecule) which modulates ITALY, Lor-2, STRIFE, TRASH, BDSF, LRSG, or STMST activity (e.g., identified in a screening assay as described herein) can be identified. Thus, to study the effect of agents on proliferative disorders, for example, in a clinical trial, cells can be isolated and RNA prepared and analyzed for the levels of expression of ITALY, Lor-2, STRIFE, TRASH, BDSF, LRSG, or STMST and other genes implicated in the proliferative disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of ITALY, Lor-2, STRIFE, TRASH, BDSF, LRSG, or STMST or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of an ITALY, Lor-2, STRIFE, TRASH, BDSF, LRSG, or STMST protein, mRNA, or genomic DNA in the preadministration sample; (iii) obtaining one or more post-administration samples from the subject; (iv) detecting the level of expression or activity of the ITALY, Lor-2, STRIFE, TRASH, BDSF, LRSG, or STMST protein, mRNA, or genomic DNA in the post-administration samples; (v) comparing the level of expression or activity of the ITALY, Lor-2, STRIFE, TRASH, BDSF, LRSG, or STMST protein, mRNA, or genomic DNA in the pre-administration sample with the ITALY, Lor-2, STRIFE, TRASH, BDSF, LRSG, or STMST protein, mRNA, or genomic DNA in the post administration sample or samples; and (vi) altering the administration of the agent to the subject accordingly. For example, increased administration of the agent may be desirable to increase the expression or activity of ITALY, Lor-2, STRIFE, TRASH, BDSF, LRSG, or STMST to higher levels than detected, i.e., to increase the effectiveness of the agent. Alternatively, decreased administration of the agent may be desirable to decrease expression or activity of ITALY, Lor-2, STRIFE, TRASH, BDSF, LRSG, or STMST to lower levels than detected, i.e. to decrease the effectiveness of the agent. According to such an embodiment, ITALY, Lor-2, STRIFE, TRASH, BDSF, LRSG, or STMST expression or activity may be used as an indicator of the effectiveness of an agent, even in the absence of an observable phenotypic response.

C. Methods of Treatment:

The present invention provides for both prophylactic and therapeutic methods of treating a subject at risk of (or susceptible to) a disorder or having a disorder associated with aberrant ITALY, Lor-2, STRIFE, TRASH, BDSF, LRSG, or STMST expression or activity. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the ITALY, Lor-2, STRIFE, TRASH, BDSF, LRSG, or STMST molecules of the present invention or ITALY, Lor-2, STRIFE, TRASH, BDSF, LRSG, or STMST modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant ITALY, Lor-2, STRIFE, TRASH, BDSF, LRSG, or STMST expression or activity, by administering to the subject an ITALY, Lor-2, STRIFE, TRASH, BDSF, LRSG, or STMST or an agent which modulates ITALY, Lor-2, STRIFE, TRASH, BDSF, LRSG, or STMST expression or at least one ITALY, Lor-2, STRIFE, TRASH, BDSF, LRSG, or STMST activity. Subjects at risk for a disease which is caused or contributed to by aberrant ITALY, Lor-2, STRIFE, TRASH, BDSF, LRSG, or STMST expression or activity can be identified by, for example, any or a combination of diagnostic or prognostic assays as described herein. Administration of a prophylactic agent can occur prior to the manifestation of symptoms characteristic of the ITALY, Lor-2, STRIFE, TRASH, BDSF, LRSG, or STMST aberrancy, such that a disease or disorder is prevented or, alternatively, delayed in its progression. Depending on the type of ITALY, Lor-2, STRIFE, TRASH, BDSF, LRSG, or STMST aberrancy, for example, an ITALY, Lor-2, STRIFE, TRASH, BDSF, LRSG, or STMST, ITALY, Lor-2, STRIFE, TRASH, BDSF, LRSG, or STMST agonist or ITALY, Lor-2, STRIFE, TRASH, BDSF, LRSG, or STMST antagonist agent can be used for treating the subject. The appropriate agent can be determined based on screening assays described herein.

2. Therapeutic Methods

Another aspect of the invention pertains to methods of modulating ITALY, Lor-2, STRIFE, TRASH, BDSF, LRSG, or STMST expression or activity for therapeutic purposes. Accordingly, in an exemplary embodiment, the modulatory method of the invention involves contacting a cell with an ITALY, Lor-2, STRIFE, TRASH, BDSF, LRSG, or STMST or agent that modulates one or more of the activities of ITALY, Lor-2, STRIFE, TRASH, BDSF, LRSG, or STMST protein activity associated with the cell. An agent that modulates ITALY, Lor-2, STRIFE, TRASH, BDSF, LRSG, or STMST protein activity can be an agent as described herein, such as a nucleic acid or a protein, a naturally-occurring target molecule of an ITALY, Lor-2, STRIFE, TRASH, BDSF, LRSG, or STMST protein (e.g., an ITALY, Lor-2, STRIFE, TRASH, BDSF, LRSG, or STMST substrate), an ITALY, Lor-2, STRIFE, TRASH, BDSF, LRSG, or STMST antibody, an ITALY, Lor-2, STRIFE, TRASH, BDSF, LRSG, or STMST agonist or antagonist, a peptidomimetic of an ITALY, Lor-2, STRIFE, TRASH, BDSF, LRSG, or STMST agonist or antagonist, or other small molecule. In one embodiment, the agent stimulates one or more ITALY, Lor-2, STRIFE, TRASH, BDSF, LRSG, or STMST activities. Examples of such stimulatory agents include active ITALY, Lor-2, STRIFE, TRASH, BDSF, LRSG, or STMST protein and a nucleic acid molecule encoding ITALY, Lor-2, STRIFE, TRASH, BDSF, LRSG, or STMST that has been introduced into the cell. In another embodiment, the agent inhibits one or more ITALY, Lor-2, STRIFE, TRASH, BDSF, LRSG, or STMST activities. Examples of such inhibitory agents include antisense ITALY, Lor-2, STRIFE, TRASH, BDSF, LRSG, or STMST nucleic acid molecules, anti-ITALY, Lor-2, STRIFE, TRASH, BDSF, LRSG, or STMST antibodies, and ITALY, Lor-2, STRIFE, TRASH, BDSF, LRSG, or STMST inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of an ITALY, Lor-2, STRIFE, TRASH, BDSF, LRSG, or STMST protein or nucleic acid molecule. In one embodiment, the method involves administering an agent (e.g., an agent identified by a screening assay described herein), or combination of agents that modulates (e.g., upregulates or downregulates) ITALY, Lor-2, STRIFE, TRASH, BDSF, LRSG, or STMST expression or activity. In another embodiment, the method involves administering an ITALY, Lor-2, STRIFE, TRASH, BDSF, LRSG, or STMST protein or nucleic acid molecule as therapy to compensate for reduced or aberrant ITALY, Lor-2, STRIFE, TRASH, BDSF, LRSG, or STMST expression or activity.

Stimulation of ITALY, Lor-2, STRIFE, TRASH, BDSF, LRSG, or STMST activity is desirable in situations in which ITALY, Lor-2, STRIFE, TRASH, BDSF, LRSG, or STMST is abnormally downregulated and/or in which increased ITALY, Lor-2, STRIFE, TRASH, BDSF, LRSG, or STMST activity is likely to have a beneficial effect. For example, stimulation of ITALY, Lor-2, STRIFE, TRASH, BDSF, LRSG, or STMST activity is desirable in situations in which an ITALY, Lor-2, STRIFE, TRASH, BDSF, LRSG, or STMST is downregulated and/or in which increased ITALY, Lor-2, STRIFE, TRASH, BDSF, LRSG, or STMST activity is likely to have a beneficial effect. Likewise, inhibition of ITALY, Lor-2, STRIFE, TRASH, BDSF, LRSG, or STMST activity is desirable in situations in which ITALY, Lor-2, STRIFE, TRASH, BDSF, LRSG, or STMST is abnormally upregulated and/or in which decreased ITALY, Lor-2, STRIFE, TRASH, BDSF, LRSG, or STMST activity is likely to have a beneficial effect.

3. Pharmacogenomics

The ITALY, Lor-2, STRIFE, TRASH, BDSF, LRSG, or STMST molecules of the present invention, as well as agents, or modulators which have a stimulatory or inhibitory effect on ITALY, Lor-2, STRIFE, TRASH, BDSF, LRSG, or STMST activity (e.g., ITALY, Lor-2, STRIFE, TRASH, BDSF, LRSG, or STMST gene expression) as identified by a screening assay described herein can be administered to individuals to treat (prophylactically or therapeutically) disorders (e.g., immune or inflammatory disorders, e.g., Rheumatoid Arthritis, Systemic Lupus Erythematosus, Myasthenia Gravis, Grave's Disease, Sjogren Syndrome, Sepsis, Polymyositis and Dermatomyositis, Psoriasis, Pemphigus Vulgaris, Bullous Pemphigoid, Inflammatory Bowel Disease, Kawasaki Disease, Asthma, or Graft v. Host Disease, e.g., bone marrow transplantation; or proliferative disorders, e.g., cancer such as melanoma, prostate cancer, cervical cancer, breast cancer, colon cancer, or sarcoma; TNF-associated disorders; or differentiative or developmental disorders) associated with aberrant ITALY, Lor-2, STRIFE, TRASH, BDSF, LRSG, or STMST activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an ITALY, Lor-2, STRIFE, TRASH, BDSF, LRSG, or STMST molecule or ITALY, Lor-2, STRIFE, TRASH, BDSF, LRSG, or STMST modulator as well as tailoring the dosage and/or therapeutic regimen of treatment with an ITALY, Lor-2, STRIFE, TRASH, BDSF, LRSG, or STMST molecule or ITALY, Lor-2, STRIFE, TRASH, BDSF, LRSG, or STMST modulator.

Pharmacogenomics deals with clinically significant hereditary variations in the response to drugs due to altered drug disposition and abnormal action in affected persons. See e.g., Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., an ITALY, Lor-2, STRIFE, TRASH, BDSF, LRSG, or STMST protein or ITALY, Lor-2, STRIFE, TRASH, BDSF, LRSG, or STMST receptor of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a drug (e.g., an ITALY, Lor-2, STRIFE, TRASH, BDSF, LRSG, or STMST molecule or ITALY, Lor-2, STRIFE, TRASH, BDSF, LRSG, or STMST modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an ITALY, Lor-2, STRIFE, TRASH, BDSF, LRSG, or STMST molecule or ITALY, Lor-2, STRIFE, TRASH, BDSF, LRSG, or STMST modulator, such as a modulator identified by one of the exemplary screening assays described herein.

This invention is further illustrated by the following examples which should not be construed as limiting. The contents of all references, patents and published patent applications cited throughout this application are incorporated herein by reference.

### EXAMPLES

### Example 1

### Identification and Characterization of Human ITALY cDNA

In this example, the identification and characterization of the gene encoding human ITALY (also referred to as “TANGO 116”) is described.

Isolation of the Human ITALY cDNA

The invention is based, at least in part, on the discovery of a human gene encoding a novel protein, referred to herein as ITALY. In order to identify novel IL-10 family members, the Cytokine Cluster Program was used. This program is based on the observation that some genes were generated by gene duplication during evolution and the duplicated genes can be situated in close proximity to each other. Briefly, using a pair of human IL-10 primers for PCR, a positive BAC clone was identified from a BAC library (Research Genetics, Huntsville Ala.). The DNA from this BAC was extracted and made into a random-sheared genomic library. 4000 clones from this BAC were selected for sequencing. The resulting genomic sequences were then assembled into contigs and used to search proprietary and public data bases.

One genomic contig was found to match with four dbest (EST database) sequences (the forward and reverse sequences from two identical clones from human pregnant uterus: AA151652/AA151733 and AA151656/AA151736). Using one dbest clone (IMAGE ID# 503280) as a probe, a full-length cDNA clone was obtained by screening a PHA-stimulated PBL cDNA library (Clontech, Palo Alto, Calif.).

The sequence of the entire clone was determined and found to contain an open reading frame of 177 amino acids termed “Interleukin Ten Associated Locus Yang” or ITALY. Signal peptide algorithms predict that ITALY contains a signal peptide (amino acids 1-24 of SEQ ID NO:2). Cleavage of the putative signal peptide would result in the secretion of a 153 amino acid protein with a predicted molecular weight of 18 kilodaltons (kD).

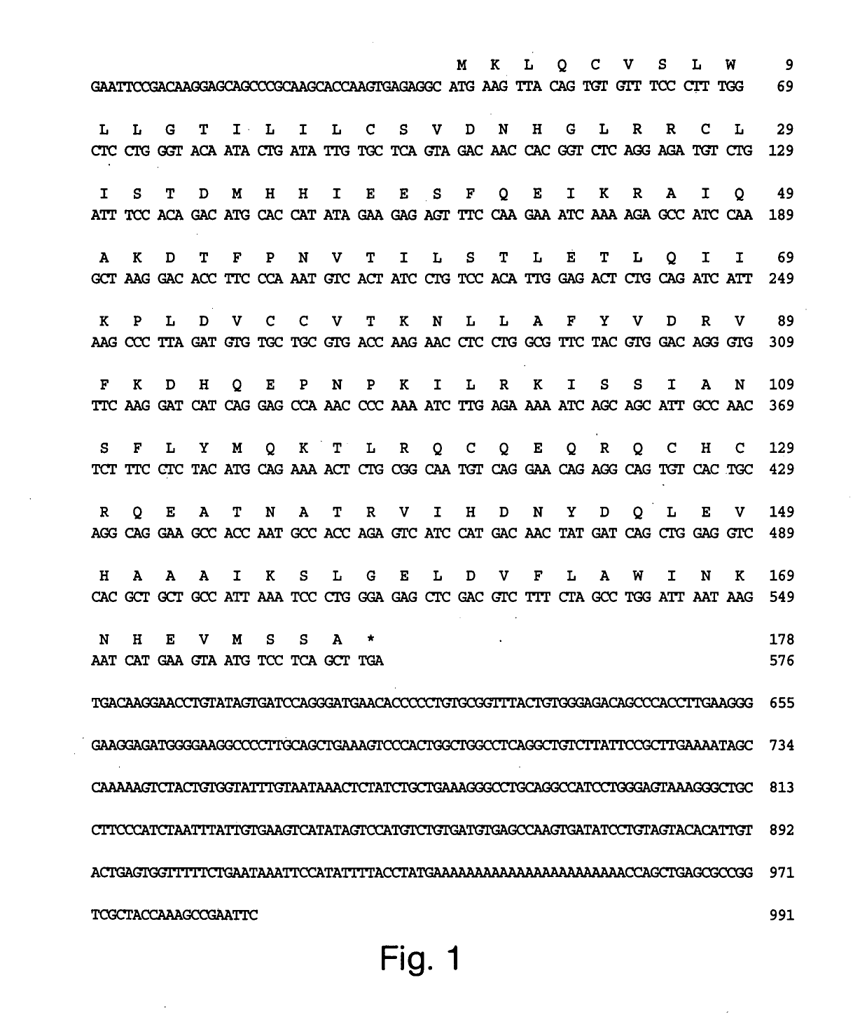

The nucleotide sequence encoding the human ITALY protein is shown in FIG. 1 and is set forth as SEQ ID NO:1. The full length protein encoded by this nucleic acid comprises about 177 amino acids and has the amino acid sequence shown in FIG. 1 and set forth as SEQ ID NO:2. The coding region (open reading frame) of SEQ ID NO:1 is set forth as SEQ ID NO:3. Clone fthP116a, comprising the entire coding region of human ITALY was deposited with the American Type Culture Collection (ATCC®), Rockville, Md., on Mar. 11, 1998, and assigned Accession No. 98960.

### Analysis of Human ITALY

A BLAST search (Altschul et al. (1990) J. Mol. Biol. 215:403) of the nucleotide and protein sequences of human ITALY revealed that ITALY is similar to the following proteins: human MDA-7 (Accession No. Q13007; SEQ ID NO:9), mouse MDA-7 (SEQ ID NO:8; from a proprietary database), human IL-10 (Accession No. P22301; SEQ ID NO:5), mouse IL-10 (Accession No. P18893; SEQ ID NO:6), and viral IL-10 (Accession No. P03180; SEQ ID N07). Each of these proteins is a member of the IL-10 family and contains both disulfide forming cysteine residues and an α-helical structure, although ITALY shares no greater than 32% identity with any of these cytokines. In particular, ITALY is 32% identical to human MDA-7 (Accession Number Q13007) (over nucleotides 157-561) and 22% identical to cat IL-10 (Accession Number P55029) (over nucleotides 214-543), at the amino acid level. An alignment of human ITALY and the above-described proteins is presented in FIG. 2.

Tissue Distribution of ITALY mRNA

This Example describes the tissue distribution of ITALY mRNA, as determined by Northern blot hybridization and PCR.

Northern blot hybridizations with the various RNA samples were performed under standard conditions and washed under stringent conditions, i.e., 0.2×SSC at 65° C. A DNA probe corresponding to the coding region of ITALY (SEQ ID NO:3) was used. The DNA was radioactively labeled with ³²P-dCTP using the Prime-It kit (Stratagene, La Jolla, Calif.) according to the instructions of the supplier. Filters containing human mRNA (MultiTissue Northern I and MultiTissue Northern II from Clontech, Palo Alto, Calif.) were probed in ExpressHyb hybridization solution (Clontech) and washed at high stringency according to manufacturer's recommendations.

ITALY message was only detected by PCR from activated peripheral blood monocytes cDNA library (Clontech). No expression of ITALY was detected from any normal human tissue by northern analysis. The human tissues tested were: heart, brain, placenta, lung, liver, skeletal muscle, kidney, pancreas, spleen, lymph node, thymus, peripheral blood leukocyte, bone marrow, fetal liver, adrenal medulla, thyroid, adrenal cortex, testis, small intestine, stomach, prostate, uterus, and colon. However, a 2 Kb ITALY message was detected in brain tumors from unidentified cancer patients.

### Example 2

### Identification and Characterization of Human Lor-2 cDNA

In this example, the identification and characterization of the gene encoding human Lor-2 (i.e., Lysyl Oxidase Related-2, also referred to as Myocardium Secreted Protein-18 or “MSP-18”) is described.

Isolation of the Human Lor-2 cDNA

The invention is based, at least in part, on the discovery of the human gene encoding Lor-2. Human Lor-2 was isolated from a cDNA library which was prepared from tissue obtained from subjects suffering from congestive heart failure. Briefly, a cardiac tissue sample was obtained from a biopsy of a 42 year old woman suffering from congestive heart failure. mRNA was isolated from the cardiac tissue and a cDNA library was prepared therefrom using art-known methods (described in, for example, Molecular Cloning A Laboratory Manual, 2nd Ed., ed. by Sambrook, Fritsch and Maniatis (Cold Spring Harbor Laboratory Press: 1989). Using a program which identifies the presence of signal peptides (Nielsen, H. et al. (1997) Protein Eng. 10:1-6) a positive clone was isolated.

The sequence of the positive clone was determined and found to contain an open reading frame. The nucleotide sequence encoding the human Lor-2 protein comprises about 2920 nucleic acids, and has the nucleotide sequence shown in FIGS. 3A-3B and set forth as SEQ ID NO:10. The protein encoded by this nucleic acid comprises about 753 amino acids, and has the amino acid sequence shown in FIGS. 4A-4C and set forth as SEQ ID NO:11.

### Analysis of Human Lor-2

A BLAST search (Altschul et al. (1990) J. Mol. Biol. 215:403) of the nucleotide and protein sequences of human Lor-2 revealed that Lor-2 is similar to the following protein molecules: a human lysyl oxidase-related protein (Accession No. U89942; SEQ ID NO:16) having approximately 56.9% identity over amino acids 33-752 of Lor-2 (SEQ ID NO:11); and a second murine lysyl-oxidase related protein (Accession No. AF053368; SEQ ID NO:17) having approximately 92.6% identity over amino acids 1-753, e.g., over the entire length) of Lor-2 (SEQ ID NO:11). (Identities were calculated using the LALIGN algorithm of Huang and Miller (1991) Adv. Appl. Math. 12:373-381).

The Lor-2 protein is predicted to have a signal peptide from amino acid residues 1-25 of SEQ ID NO:11. Accordingly, a mature Lor-2 protein is predicted to include amino acid residues 26-753 of SEQ ID NO:11. Lor-2 is also predicted to have 5 N-glycosylation sites, 8 protein kinase phosphorylation (“PKC”) sites, 14 casein kinase II phosphorylation sites, 19 N-myristoylation sites, and 1 amidation site. Predicted N-glycosylation sites are found, for example, from about amino acid 111-114, 266-269, 390-393, 481-484, and 625-628 of SEQ ID NO:11. Predicted PKC phosphorylation sites are found, for example, from about amino acid 97-99, 104-106, 221-223, 268-270, 352-354, 510-512, 564-566, and 649-651 of SEQ ID NO:11. Predicted casein kinase II phosphorylation sites are found, for example, from about amino acid 31-34, 68-71, 115-118, 120-123, 135-138, 330-333, 352-355, 377-380, 392-395, 411-414, 424-427, 493-496, 527-530, and 617-620 of SEQ ID NO:11. Predicted N-myristoylation sites are found, for example, from about amino acids 13-18, 116-121, 130-135, 273-278, 312-317, 359-364, 378-383, 403-408, 443-448, 451-456, 463-468, 470-475, 489-494, 506-511, 515-520, 521-526, 626-631, 661-666, and 746-751 of SEQ ID NO:11. A predicted amidation site is found, for example, from amino acid 117-180 of SEQ ID NO:11.

Moreover, Lor-2 has a 4 scavenger receptor cysteine-rich domains from amino acid residues 51-145, 183-282, 310-407, and 420-525 of SEQ ID NO:11. The third scavenger receptor cysteine-rich domain includes a speract receptor repeated domain signature from amino acid residues 312-349 of SEQ ID NO:11. Lor-2 further has a lysyl oxidase domain from residues 330-732 of SEQ ID NO:11. (See, for example, FIGS. 7A-7B). Within the lysyl oxidase domain of Lor-2, there exists a fragment having significant homology to the lysyl oxidase putative copper-binding region, termed the “copper-binding talon”. A ProSite consensus pattern describing the copper-binding talon is as follows: W-E-W-H-S-C-H-Q-H-Y-H (SEQ ID NO:18) (see also PROSITE documentation PDOC00716 and Krebs and Krawetz (1993) Biochem. Biophys. Acta 1202:7-12). Amino acid residues 601-701 of human Lor-2 (SEQ ID NO:11) have ˜73% identity with this consensus sequence (8/11 residues) including each of the four conserved histidines, three of which are believed to be copper ligands residing within an octahedral coordination complex of lysyl oxidase.

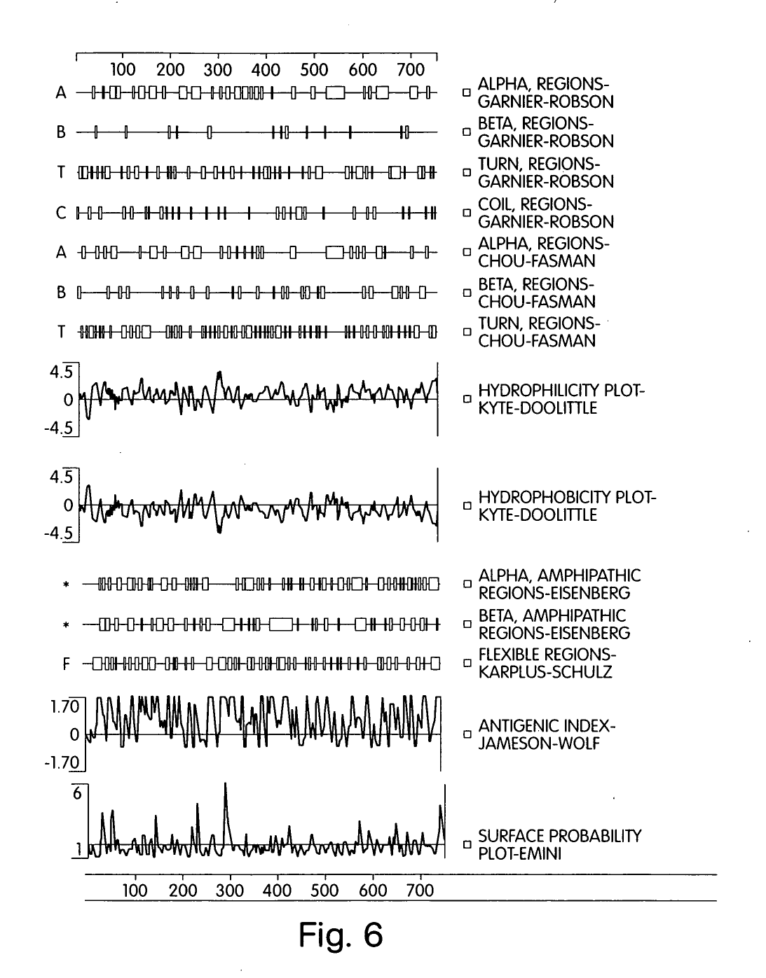

Analysis of primary and secondary protein structures, as shown in FIG. 6, was performed as follows: alpha, beta turn and coil regions, Garnier-Robson algorithm (Garnier et al. (1978) J. Mol. Biol. 120:97); alpha, beta, and turn regions, Chou-Fasman algorithm (Chou and Fasman (1978) Adv. Enzymol. Mol. 47:45-148); hydrophilicity and hydrophobicity plots, Kyte-Doolittle algorithm (Kyte and Doolittle (1982) J. Mol. Biol. 157:105-132); alpha amphipathic and beta amphipathic regions, Eisenberg algorithm (Eisenberg et al. (1982) Nature 299:371-374); flexible regions, Karplus-Schulz algorithm (Karplus and Schulz (1985) Naturwissens-Chafen 72:212-213); antigenic index, Jameson-Wolf algorithm (Jameson and Wolf (1988) CABIOS 4:121-136); surface probability plot, Emini algorithm (Emini et al. (1985) J Virol 55:836-839).

### Prediction of the Chromosomal Location of Lor-2-Electronic Mapping

To predict the chromosomal location of Lor-2, the Lor-2 nucleotide sequence of SEQ ID NO:10 was used to query, using the BLASTN program (Altschul S. F. et al. (1990) J. Mol. Biol. 215: 403-410) with a word length of 12 and using the BLOSUM62 scoring matrix, a database of human nucleotide sequences originating from nucleotide molecules (e.g., EST sequences, STS sequences and the like) that have been mapped to the human genome. Nucleotide sequences which had been previously mapped to human chromosome 2 near the D2S145 marker (e.g., having Accession Nos. AA191602 and R55706) were found to have high sequence identity to portions of the Lor-2 nucleotide sequence (3′ UTR sequence) indicating that Lor-2 maps to the same chromosomal location. Moreover, it is predicted that allelic variants of Lor-2 will map the same chromosomal location and species orthologs of Lor-2 will map to loci syntenic with the human Lor-2 locus.

### Confirmation and Analysis of the Chromosomal Location of Lor-2-PCR Mapping

The hLor-2 gene was mapped to human chromosome 2 (i.e., 2 μl 1-p13), which is syntenic to mouse chromosome 6, by PCR typing of the Genebridge (G4) radiation hybrid panel (Research Genetics, Inc., Huntsville, Ala.). Typing of the DNA and comparison to radiation hybrid map data at the Whitehead Institute Center for Genome Research (WICGR) tightly linked the hLor-2 gene to a region on human chromosome 2 between WI-5987 (13.9cR) and GCT1B4 (16.7cR).

The huLor-2 primers used in the PCR mapping studies were: forward—GCTTACCAAGAAACCCATGTCAGC (SEQ ID NO:20) and reverse—GGCAGTTAGTCAGGTGCTGC (SEQ ID NO:21). The radiation hybrid mapping studies were performed as follows: PCR reactions of radiation hybrid panels, GeneBridge 4 (Research Genetics, Inc., Huntsville, Ala.) were assembled in duplicate using an automated PCR assembly program on a TECAN Genesis. Each reaction consisted of: 5 μl DNA template (10 ng/μl), 1.5 μl 10×PCR buffer, 1.2 μl dNTPs (2.5 mM), 1.1 μl forward primer (6.6 μM) 1.15 μl reverse primer (6.6 μM), and 5 μl 1:75 platinum Taq. The reactions were thermocycled on a Perkin-Elmer 9600 for 95° C. 10 minutes (for the platinum Taq), [95° C. 40 sec, 52° C. 40 sec, 72° C., 50 sec] 35×, 72° C., 5 minutes, 4° C. hold. Resulting PCR products were run out on a 2% agarose gel and visualized on a UV light box.

The positive hybrids for the Genebridge 4 panel were submitted to the Whitehead Genome Center for placement in relation to a framework map.

Human Lor-2 mapped in close proximity to known genes including actin, gamma 2, smooth muscle, enteric (“ACTG2”), nucleolysin TIA1, semaphorin W (“SEMAW”), dysferlin (“DYSF”), docking protein 1 (“DOK1), glutamine-fructose-6-phosphate transaminase 1 (“GFPT”), the KIAA0331 gene, deoxyguanosine kinase (“DGUOK”), the TSC501 gene, eukaryotic translation initiation factor 3, subunit 10 (“EIF3S1”), tachykinin receptor I (“TACR1”), tissue-type plasminogen activator (“PLAT”) and dual specificity phosphatase 11 (“DUSP11”). Nearby disease mutations and/or loci include Alstrom syndrome (“ALMS1”), an autosomal recessively inherited syndrome characterized by retinal degeneration, obesity, diabetes mellitus, neurogenous deafness, hepatic dysfunction, and in some cases, late onset cardiomyopathy (see e.g., Alstrom et al. (1959) Acta Psychiat. Neurol. Scand. 34 (suppl. 129): 1-35; Alter and Moshang (1993) Am. J. Dis. Child. 147:97-99; Awazu et al. (1997) Am. J. Med. Genet. 69:13-16; Aynaci et al. (1995) (Letter) Clin. Genet. 48:164-166; Charles et al. (1990) J. Med. Genet. 27:590-592; Cohen and Kisch (1994) Israel J. Med. Sci. 30:234-236; Collin et al. (1997) Hum. Molec. Genet. 6:213-219; Collin et al. (1999) (Letter) Clin. Genet. 55:61-62; Connolly et al. (1991) Am. J. Med. Genet. 40:421-424; Goldstein and Fialkow (1973) Medicine 52:53-71; Macari et al. (1998) Hum. Genet. 103:658-661; Marshall et al. (1997) Am. J. Med. Genet. 73:150-161; Michaud et al. (1996) J. Pediat. 128:225-229; Millay et al. (1986) Am. J. Ophthal. 102:482-490; Rudiger et al. (1985) Hum. Genet. 69:76-78; Russell-Eggitt et al. (1998) Opthalmology 105:1274-1280; Tremblay et al. (1993) Am. J. Ophthal. 115:657-665; Warren et al. (1987) Am. Heart J. 114:1522-1524 and Weinstein et al. (1969) New Eng. J. Med. 281:969-977), orofacial cleft 2 (“OFC2”) (see e.g., Carinci et al. (1995) (Letter) Am. J. Hum. Genet. 56:337-339; Pezzetti et al. (1998) Genomics 50:299-305 and Scapoli et al. (1997) Genomics 43:216-220) and Parkinson's disease 3 (see e.g., Di Rocco et al. (1996) Adv. Neurol. 69:3-11 and Gasser et al. (1998) Nature Genet. 18:262-265). Additional information regarding Alstrom syndrome, orofacial cleft 2 and Parkinson disease 3 can be found collected under Accession Nos. 203800, 602966 and 602404, respectively, in the Online Mendelian Inheritance in Man (“OMIM™”) database, the contents of which are incorporated herein by reference.

Moreover, the syntenic location on mouse chromosome 6 is near ovarian teratoma susceptibility 1 (“Ots-1”), disruption of corticosterone in adrenal cortex cells (“Cor”), brain protein 1 (“Brp1”), lymphocyte antigen 36 (“Ly36”), major liver protein 1 (“Lvp1”), cerebellar deficient folia (“cdf”), motor neuron degeneration 2 (“mnd2”), truncate (“tc”) and faded (“fe”). Of particular interest are the Lor-2 neighbors Ots-1 and Cor, both of which a postulated to play a role in tumor susceptibility. The Ots-1 locus was identified by linkage analysis of female LT/Sv mice, a strain characterized by its abnormally high incidence of spontaneous ovarian teratomas, which are extremely rare for other mouse strains. Ots-1 was identified as the single major locus that increases the frequency of teratomas in a semidominant manner (Lee et al. (1997) Cancer Res. 57:590-593. Likewise, the cor locus was identified as being associated with a phenotype of the AJ mouse strain (a strain susceptible to many neoplasms and infectious agents, presumably due to a deficiency in the prophylactic activities of endogenous glucocorticoids (e.g., adrenalcortical corticosterone (“CS”)) (Thaete et al. (1990) Proc. Soc. Exp. Biol. Med. 194:97-102). Accordingly, at least two loci in the near vicinity of mouse Lor-2 on chromosome 6 are associated with tumor susceptibility. Additional information regarding the Ots-1 and Cor loci can be found collected under Accession Nos. MGI:85864 and MGI:58993, respectively, in the Mouse Genomics Informatics database (available online), the contents of which are incorporated herein by reference. Likewise, information regarding the cdf locus, the mnd2 locus and the mouse Lor-2 gene (i.e., the mouse ortholog of human Lor-2) can be found collected under Accession Nos. MGI: 86274, MGI:97039 and MGI: 1337004, respectively.

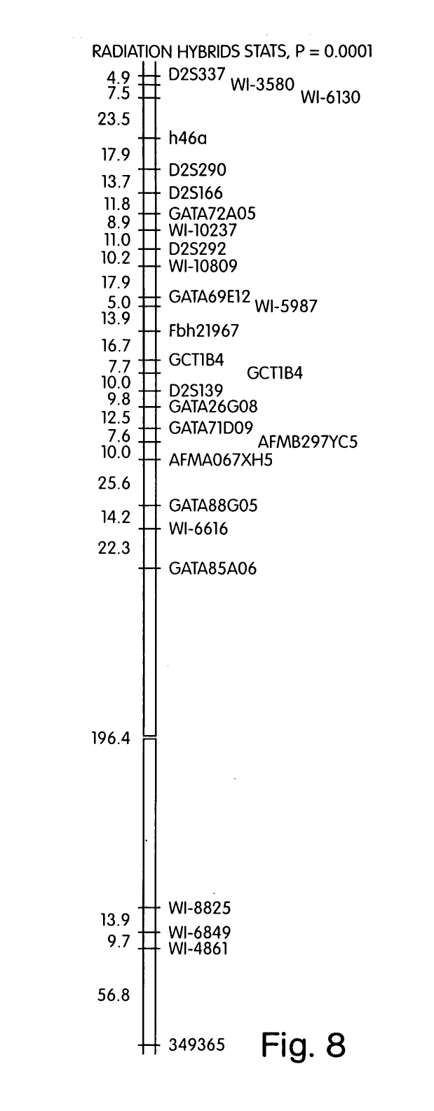

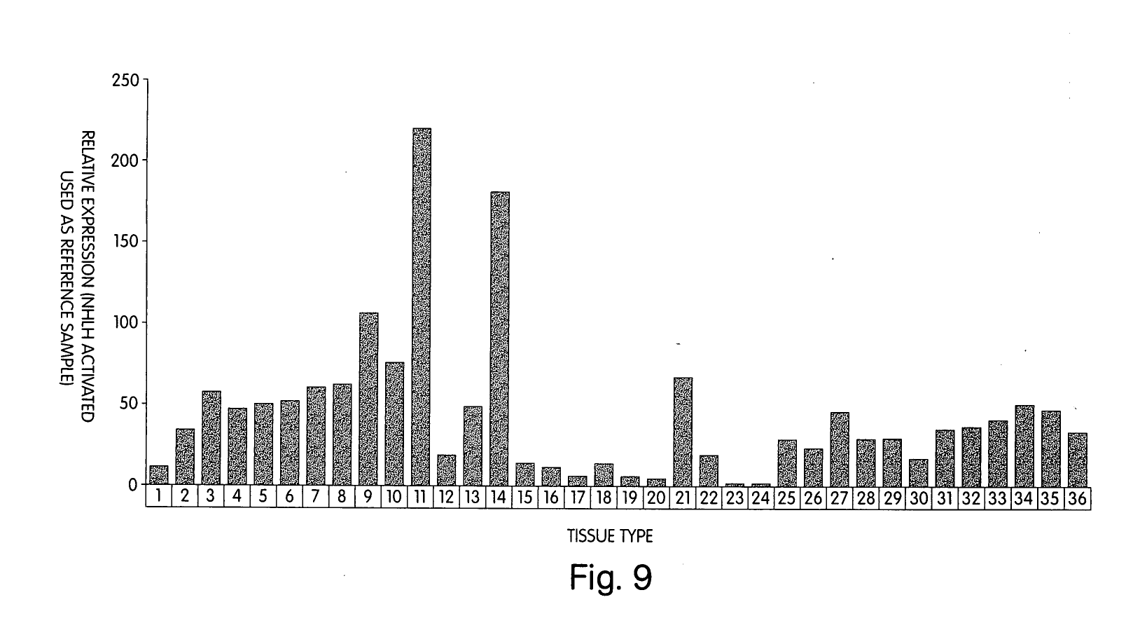

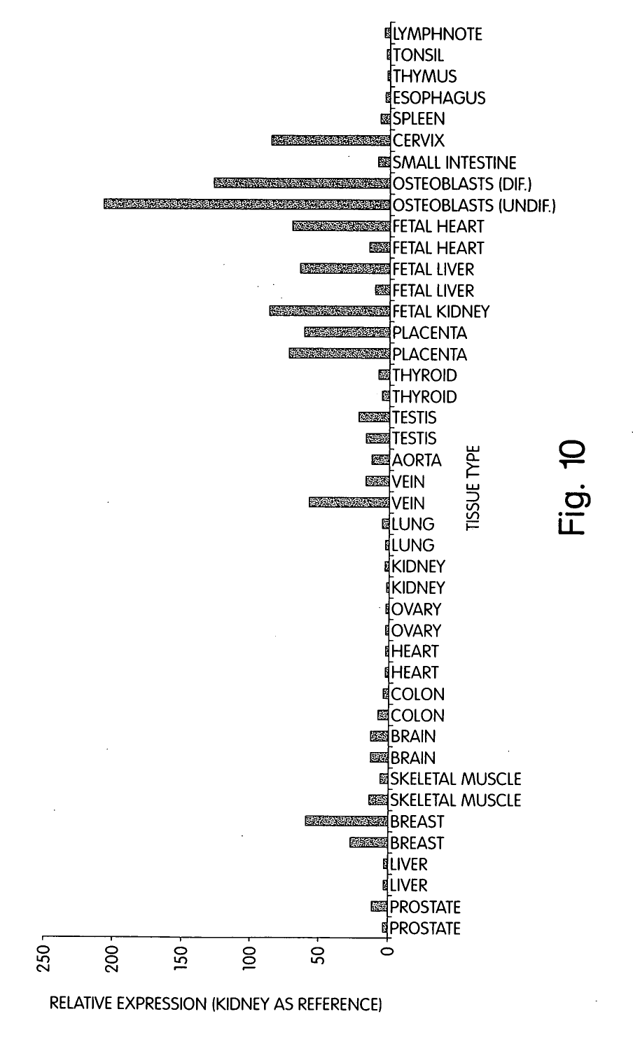

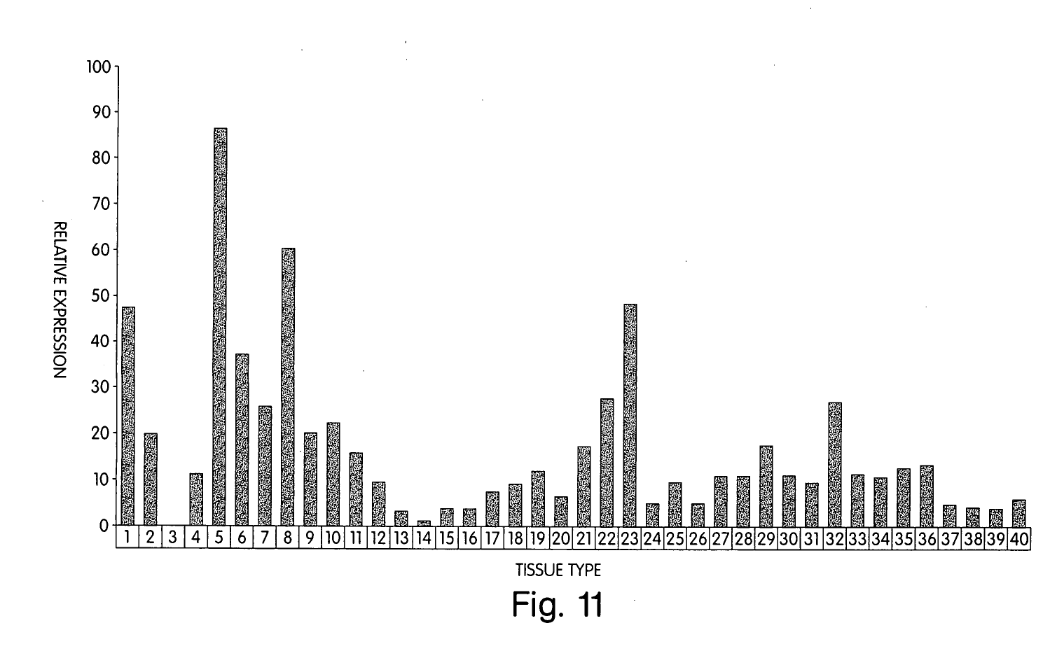

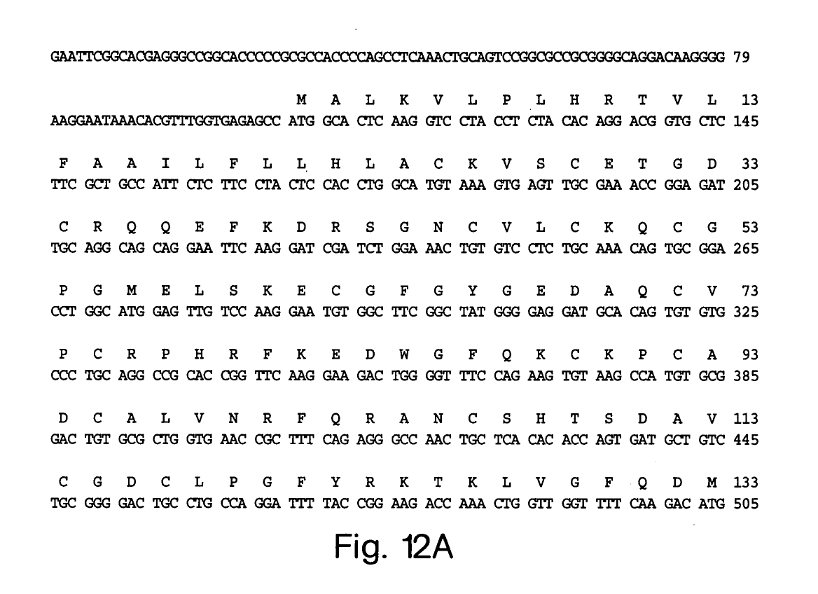

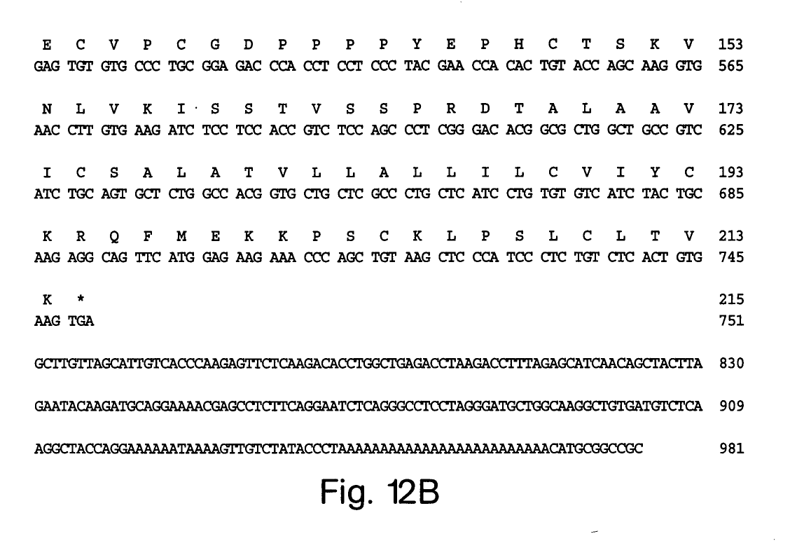

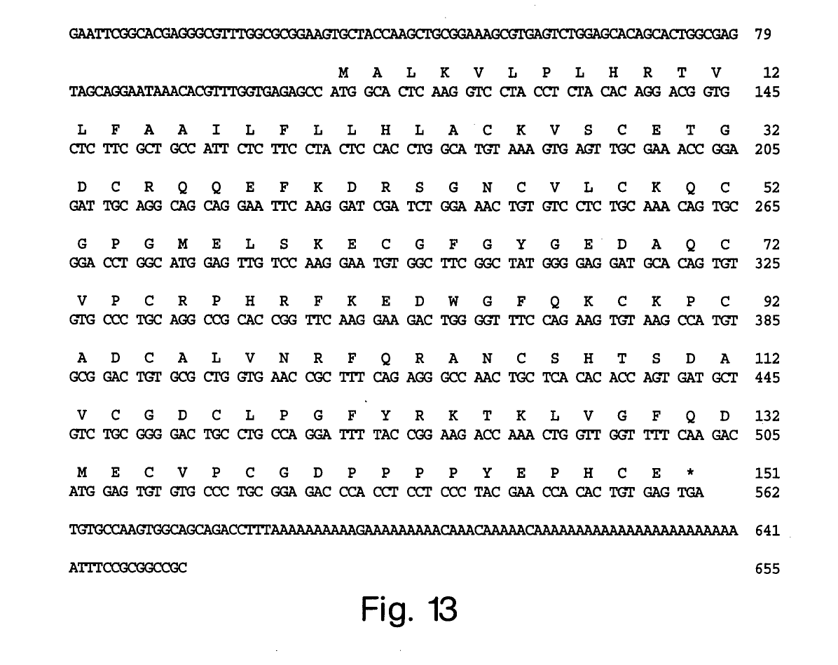

Additional markers (e.g., EST markers, STS markers and the like) are set forth in FIG. 8, as are the relative distances between markers.

Tissue Distribution of Lor-2 mRNA

Standard molecular biology methods (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989) were used to construct cDNA libraries in plasmid vectors from multiple human tissues. Individual cDNA clones from each library were isolated and sequenced and their nucleotide sequences were input into a database. The Lor-2 nucleotide sequence of SEQ ID NO:10 was used to query the tissue-specific library cDNA clone nucleotide sequence database using the BLASTN program (Altschul S. F. et al, (1990) J. Mol. Biol. 215: 403-410) with a word length of 12 and using the BLOSUM62 scoring matrix. Nucleotide sequences identical to portions of the Lor-2 nucleotide sequence of SEQ ID NO:10 were found in cDNA libraries originating from human endothelial cells, lymph node, bone, heart, neuron, and testes. Lor-2 nucleic acid sequences, fragments thereof, proteins encoded by these sequences, and fragments thereof as well as modulators of Lor-2 gene or protein activity may be useful for diagnosing or treating diseases that involve the tissues in which the Lor-2 mRNA is expressed. Likewise, when a similar analysis was performed using the Lor-2 sequence of SEQ ID NO:10 to query publicly available nucleotide sequence databases (e.g., DBEST databases) using BLAST, sequences having high homology to the 3′ untranslated region of human Lor-2 were identified in a Soares placenta normalized library and in Soares testis, B-cell and lung normalized libraries.

Northern blot hybridization with RNA samples was next performed under standard conditions and washed under stringent conditions, i.e., 0.2×SSC at 65° C. A DNA probe was radioactively labeled with ³²P-dCTP using the Prime-It kit (Stratagene, La Jolla, Calif.) according to the instructions of the supplier. Filters containing various tissue and cell line mRNAs were probed in ExpressHyb hybridization solution (Clontech) and washed at high stringency according to manufacturer's recommendations.

On a human mRNA blot containing mRNA from heart, brain, placenta, lung, liver, skeletal muscle, kidney, and pancreas, Lor-2 transcript (˜3.0 kb) was detected in all tissues tested but was most strongly detected in heart and placenta. Moreover, Lor-2 mRNA was strongly expressed in the G361 melanoma cell line and in the SW480 adenocarcinoma colon cell lines (as compared to expression in the HL60, HeLa53, K562, Molty, Raji, and SW480 cell lines (SW480 cell line expressing a 2.4 kb transcript). Transcripts of Skb and 2 kb were also detected evidencing possible splice variants of Lor-2.

Testing of a larger panel of human tissues revealed the following expression levels. Expression levels were normalized to beta 2 expression.

TABLE I
hu Lor-2 Expression in Normal Tissues

| | huLor-2 | Beta 2 | Relative |
|---|---|---|---|
| Tissue Source | Expression | Expression | Expression* |
| Lymph Node (MPI 79) | 30.550 | 18.170 | 10.78 |
| Lymph Node (NDR 173) | 29.930 | 19.190 | 33.59 |
| Heart (PIT 272) | 26.145 | 18.170 | 57.06 |
| Heart (PIT 273) | 29.375 | 19.110 | 46.85 |
| Lung (MPI 131) | 29.650 | 19.480 | 50.04 |
| Lung (NDR 185) | 27.165 | 17.050 | 51.96 |
| Kidney (MPI 58) | 30.695 | 20.790 | 60.13 |
| Spleen (MPI 360) | 27.005 | 17.150 | 62.25 |
| SK Muscle (MPI 38) | 29.480 | 20.400 | 106.15 |
| Fetal Liver (MPI 425) | 30.065 | 20.520 | 75.85 |
| Fetal Liver (MPI 133) | 31.570 | 23.550 | 221.32 |
| Tonsil (MPI 37) | 29.480 | 17.890 | 18.64 |
| Colon (MPI 383) | 30.045 | 19.830 | 48.50 |
| Brain (MPI 422) | 30.525 | 22.220 | 181.65 |
| Liver (MPI 75) | 32.935 | 20.940 | 14.07 |
| Liver (MPI 365) | 31.060 | 18.770 | 11.35 |
| Liver (MPI 339) | 33.985 | 20.740 | 5.92 |
| Liver (MPI 154) | 32.000 | 19.970 | 13.74 |
| Liver (NDR 206) | 33.750 | 20.370 | 5.41 |
| Liver (PIT 260) | 32.705 | 18.970 | 4.23 |
| CD14 | 26.945 | 17.190 | 66.49 |
| Granulocytes | 30.825 | 19.240 | 18.77 |
| NHLH (resting) | 36.595 | 19.920 | 1.10 |
| NHLH (activated) | 35.570 | 19.760 | 1.00 |
| Liver Fibrosis (MPI 447) | 29.320 | 18.300 | 27.67 |
| Liver Fibrosis (NDR 190) | 36.495 | 24.180 | 22.55 |
| Liver Fibrosis (NDR 191) | 30.105 | 19.770 | 44.63 |
| Liver Fibrosis (NDR 192) | 33.415 | 22.410 | 27.95 |
| Liver Fibrosis (NDR 193) | 30.795 | 19.830 | 28.74 |
| Liver Fibrosis (NDR 204) | 33.360 | 21.580 | 16.34 |
| Liver Fibrosis (NDR 126) | 31.900 | 21.180 | 34.18 |
| Liver Fibrosis (NDR 113) | 29.175 | 18.510 | 36.51 |
| Liver Fibrosis (NDR 79) | 30.870 | 20.390 | 40.22 |
| Liver Fibrosis (NDR 112) | 31.955 | 21.770 | 49.52 |
| Liver Fibrosis (NDR 225) | 30.645 | 20.350 | 45.89 |
| Liver Fibrosis (NDR 141) | 33.045 | 22.250 | 32.45 |

*NHLH activated used as reference sample

Next, Lor-2 expression levels were measured in a variety of tissue and cell samples using the Taqman™ procedure. The Taqman™ procedure is a quantitative, real-time PCR-based approach to detecting mRNA. The RT-PCR reaction exploits the 5′ nuclease activity of AmpliTaq Gold™ DNA Polymerase to cleave a TaqMan™ probe during PCR. Briefly, cDNA is generated from the samples of interest and serves as the starting materials for PCR amplification. In addition to the 5′ and 3′ gene-specific primers, a gene-specific oligonucleotide probe (complementary to the region being amplified) is included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product.

TABLE II
hu Lor-2 3′ UTR Expression in Normal Human Tissues

| Tissue Source | Relative Expression* |
|---|---|
| Prostate | 2.5 |
| Prostate | 10.9 |
| Liver | 2.4 |
| Liver | 2.5 |
| Breast | 26.7 |
| Breast | 59.3 |
| Skeletal Muscle | 13.4 |
| Skeletal Muscle | 5.5 |
| Brain | 12.6 |
| Brain | 12.7 |
| Colon | 7.2 |
| Colon | 3.4 |
| Heart | 1.8 |
| Heart | 1.8 |
| Ovary | 1.8 |
| Ovary | 1.4 |
| Kidney | 1.0 |
| Kidney | 2.3 |
| Lung | 1.8 |
| Lung | 4.2 |
| Vein | 57.5 |
| Vein | 16.1 |
| Aorta | 11.8 |
| Testis | 16.4 |
| Testis | 21.7 |
| Thyroid | 4.4 |
| Thyroid | 7.2 |
| Placenta | 73.3 |
| Placenta | 61.8 |
| Fetal Kidney | 87.7 |
| Fetal Liver | 10.0 |
| Fetal Liver | 64.7 |
| Fetal Heart | 14.4 |
| Fetal Heart | 70.8 |
| Osteoblasts (undif.) | 207.9 |
| Osteoblasts (dif.) | 128.0 |
| Small Intestine | 7.9 |
| Cervix | 86.5 |
| Spleen | 6.3 |
| Esophagus | 2.4 |
| Thymus | 1.4 |
| Tonsil | 1.7 |
| Lymph node | 3.1 |

*Kidney used as reference sample

The highest expression was observed in osteoblasts, cervix, kidney and placenta on the normal human tissue panel tested.

### Example 3

### Expression of Lor-2 mRNA in Clinical Tumor Samples and in Xenograft Cell Lines

In this example, RT-PCR was used to detect the presence of Lor-2 mRNA in various tumor and metastatic tissue samples as compared to normal tissue samples. RT-PCR was also used to detect the presence of Lor-2 mRNA in various xenograft cell lines. In breast tissue, Lor-2 mRNA was detected in 0/1 normal tissue samples as compared to 3/4 tumor clinical samples after 30 cycles of PCR. In xenograft cell lines isolated from breast tissue, Lor-2 mRNA was detected in 1/1 normal and 3/3 xenograft cell lines (cell lines MCF7, ZR75 and T47D). In lung tissue, Lor-2 mRNA was detected in 0/2 normal tissue samples as compared to 2/8 tumor tissue samples. In xenograft cell lines isolated from lung tissue, Lor-2 mRNA was detected in 0/5 xenograft cell lines after 30 cycles of PCR. In a second experiment performed with lung tissue, Lor-2 mRNA was detected in 2/2 normal and 8/8 tumor tissue samples, as well as in 5/5 xenograft cell lines (cell lines A549, H69, H125, H322 and H460) after 35 cycles of PCR. In colon tissue, Lor-2 mRNA was detected in 2/2 normal, 5/5 tumor and 5/5 metastatic samples, as well as in 7/7 xenograft cell lines (cell lines HCT116, HCT15, HT29, SW620, SW480, DLD1 and KM12) after 35 cycles of PCR. In liver tissue, LOR-2 mRNA was detected in 2/2 normal samples after 35 cycles of PCR. These data reveal that there exists a correlation between tumors and Lor-2 expression, at least in breast and lung tissues.

To further investigate this finding, Lor-2 mRNA levels were measured by quantitative PCR using the Taqman™ procedure as described above. The procedure was carried out on cDNA generated from various carcinoma samples and compared to normal counterpart tissue samples. In 5/7 breast carcinomas, a 2-86 fold upregulation of Lor-2 was observed as compared to 2/4 normal breast tissue samples. Likewise, in 4/7 lung carcinomas, a 2-17 fold upregulation was observed as compared to 3/4 normal lung tissue samples. The relative levels of Lor-2 mRNA detected in various normal, tumor and metastases samples are set forth in Table III.

TABLE III
hu Lor-2 Expression - Taqman Analysis of Oncology Panel

| Tissue Source | Relative Expression |
|---|---|
| Breast N | 46.85 |
| Breast N | 18.96 |
| Breast N | 1.00 |
| Breast N | 11.75 |
| Breast T | 86.52 |
| Breast T | 37.27 |
| Breast T | 25.72 |
| Breast T | 60.76 |
| Breast T | 19.84 |
| Breast T | 22.24 |
| Breast T | 16.26 |
| Lung N | 9.32 |
| Lung N | 3.34 |
| Lung N | 1.65 |
| Lung N | 3.84 |
| Lung T | 4.26 |
| Lung T | 7.39 |
| Lung T | 9.13 |
| Lung T | 12.08 |
| Lung T | 6.48 |
| Lung T | 17.27 |
| Lung T | 28.15 |
| Colon N | 48.50 |
| Colon N | 4.94 |
| Colon N | 10.09 |
| Colon N | 4.94 |
| Colon T | 10.78 |
| Colon T | 10.89 |
| Colon T | 17.39 |
| Colon T | 10.82 |
| Colon T | 9.09 |
| Colon T | 26.63 |
| Liver Met | 10.93 |
| Liver Met | 10.30 |
| Liver Met | 12.25 |
| Liver Met | 12.91 |
| Liver N | 4.30 |
| Liver N | 3.69 |
| Liver N | 3.48 |
| Liver N | 5.41 |

These data reveal a significant upregulation of Lor-2 mRNA in at least breast and lung carcinomas. Moreover, there was a significant upregulation of Lor-2 expression in metastatic as compared to normal liver samples. Given that the mRNA for Lor-2 is expressed in a variety of tumors, with significant upregulation in carcinoma samples in comparison to normal samples, it is believed that inhibition of Lor-2 activity may inhibit tumor progression by affecting the adhesive properties of the tumor cells to surrounding tissues.

### Example 4

### Identification of Murine STRIFE1 and STRIFE2 cDNA

In this example, the isolation and characterization of the cDNA encoding murine STRIFE1 and STRIFE2 is described. STRIFE is a mouse gene which encodes a protein belonging to the TNFR family. Two splice forms have been identified, one that is predicted to be membrane bound (STRIFE1) and one that is secreted (STRIFE2).

STRIFE was identified as a TNFR homologue by a computer-based search of the public EST databases. More specifically, the murine STRIFE1 and STRIFE2 cDNA were identified by searching against a copy of the GenBank nucleotide database using the BLASTN™ program (BLASTN 1.3 MP: Altschul et al., J. Mol. Bio. 215:403, 1990). Numerous clones that consisted mostly of 3′ reads and some that were 5′ reads within the 3′ untranslated region were found by this search. The sequences were analyzed against a non-redundant protein database with the BLASTX™ program, which translates a nucleic acid sequence in all six frames and compares it against available protein databases (BLASTX 1.3 MP:Altschul et al., supra). This protein database is a combination of the Swiss-Prot, PIR, and NCBI GenPept protein databases. Two clones (Accession Numbers AA036247 and AA003356) were obtained from the IMAGE consortium, and fully sequenced. The additional sequencing of AA036247 (T 127a; STRIFE1) extended the original EST by 623 nucleotides (see SEQ ID NO:22) and the further sequencing of AA003356 (T127b; STRIFE2) extended the original EST by 254 nucleotides (see SEQ ID NO:26).

A BLASTN™ search of the EST database revealed the following ESTs having significant homology to clone Accession Number AA036247:

| | | Base Pairs | % | |
|---|---|---|---|---|
| EST Database hits | Species | Covered | Identity | Coding? |
| Accession # AA495217 | zebrafish | 602-711 | 82 | yes |

A BLASTN™ search of the EST and nucleotide database revealed the following ESTs and nucleotides having significant homology to clone Accession Number AA003356:

| | | Base Pairs | % | |
|---|---|---|---|---|
| EST Database hits | Species | Covered | Identity | Coding? |
| Accession # AA686080 | rat | 297-367 | 64 | yes |
| Accession # AA209382 | human | 150-210 | 67 | yes |
| Accession # AA409240 | mouse | 284-319 | 80 | yes |
| Accession # N91779 | mouse | 519-489 | 83 | yes |

### Example 5

### Tissue Expression of the STRIFE1 and STRIFE2 Gene

Human I and mouse multiple tissue northern (MTN) blots (Clontech, Palo Alto, Calif.) containing 2 μg of poly A+ RNA per lane were probed with a 750 bp EcoRI/NotI fragment of the mouse STRIFE1 cDNA. The filters were prehybridized in 10 ml of Express Hyb hybridization solution (Clontech, Palo Alto, Calif.) at 68° C. for 1 hour, after which 100 ng of ³²P labeled probe was added. The probe was generated using the Stratagene Prime-It kit, Catalog Number 300392 (Clontech, Palo Alto, Calif.). Hybridization was allowed to proceed at 68° C. for approximately 2 hours. The filters were washed in a 0.05% SDS/2×SSC solution for 15 minutes at room temperature and then twice with a 0.1% SDS/0.1×SSC solution for 20 minutes at 50° C. and then exposed to autoradiography film overnight at −80° C. with one screen. The mouse tissues tested included: heart, brain, spleen, lung, liver, skeletal muscle, kidney, and testis. The human tissues tested included: heart, placenta, lung, liver, skeletal muscle, kidney, and pancreas.

There was a strong hybridization to both mouse and human heart, brain, and lung indicating that the approximately 4.4 kb STRIFE1 and STRIFE2 gene transcript is expressed in these tissues.

### Example 6

### Characterization of the Murine STRIFE1 and STRIFE2 Proteins

STRIFE1 is approximately 981 nucleotides in length and has an open reading frame of 645 nucleotides that is predicted to encode a protein of 214 amino acids. STRIFE2 is approximately 655 nucleotides long with an open reading frame of 453 nucleotides predicted to encode a protein of 150 amino acids. Both clones have been subcloned into a variety of expression vectors including those for retroviral delivery and for expression in bacterial, yeast and mammalian cells.

BlastX searching of the protein database confirms the homology of this clone to various members of the TNFR family. The extracellular domains of STRIFE1 and STRIFE2 are approximately 40% identical to OX40. Importantly, a number of cysteine residues within the extracellular domains of STRIFE1 and STRIFE2 match the cysteine-rich domain signature of the TNFR/NGFR family (Prosite Accession PDOC00561). The program SignalP (Nielsen et al, 1997) predicts a 30 amino acid signal peptide at the very N-terminus of both STRIFE1 and STRIFE2 (i.e., residues 1-29 of SEQ ID NOs:22 and 6). The predicted molecular weight for STRIFE1 is approximately 23.55 kDa with the signal peptide and 20.34 kDa without the signal peptide which is presumed to be cleaved in the mature protein. There are no obvious motifs in the small intracellular domain of STRIFE1. STRIFE2 is predicted to be 16.72 kDa with the signal peptide and 13.51 kDa without the signal peptide.

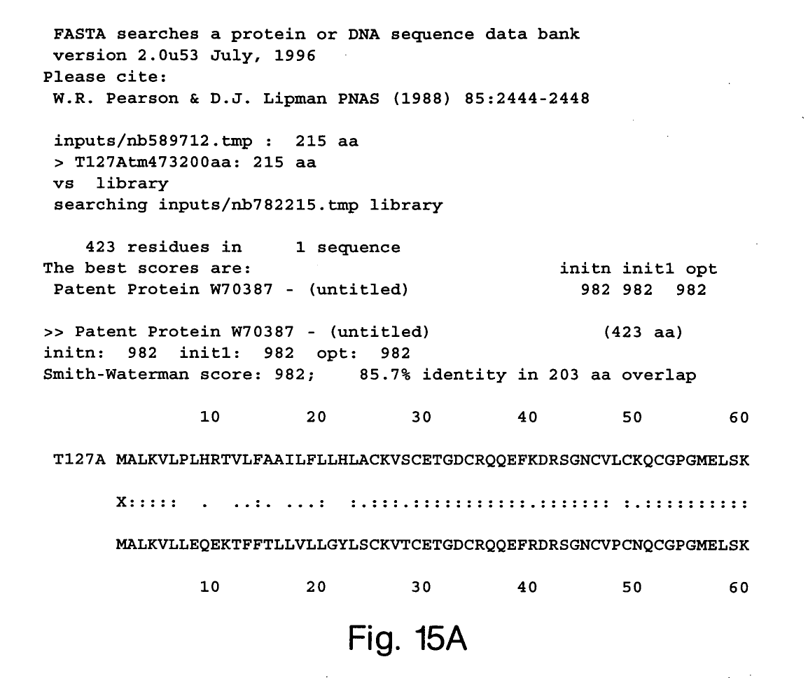

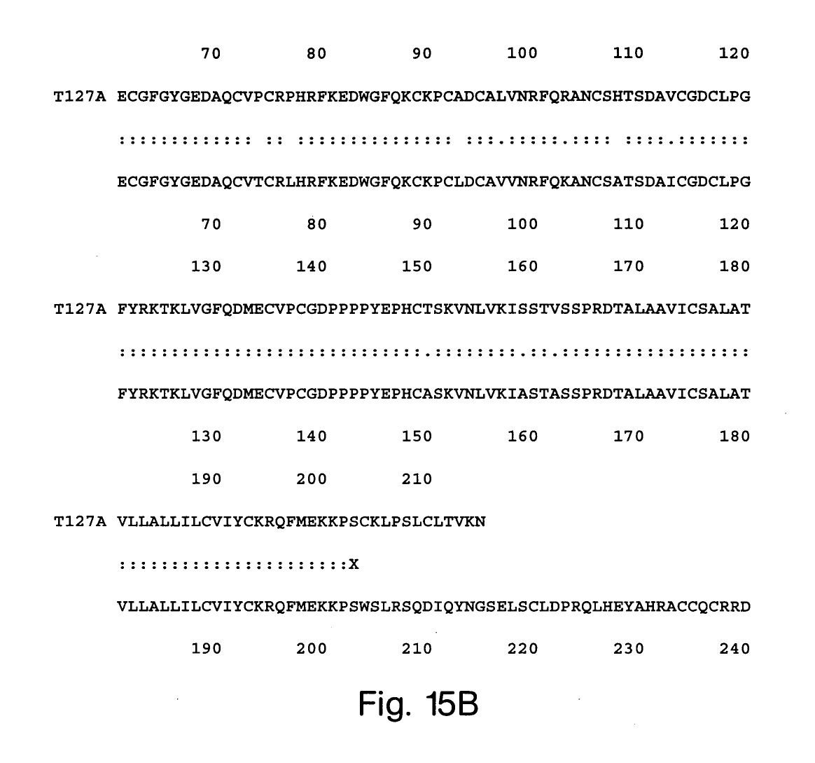

A FASTA search (described in Pearson W. R. & Lipman D. J. (1988) Proc. Natl. Acad. Sci. USA 85:2444-2448, score matrix: PAM120) using the STRIFE1 protein sequence as a query, indicates that STRIFE1 is 85.7% identical to the human OAF065 receptor (Accession number W70387; described in PCT application number WO 98/38304, published on Sep. 3, 1998) over amino acid residues 1-203. The results from this search are shown in FIGS. 15A-15B.

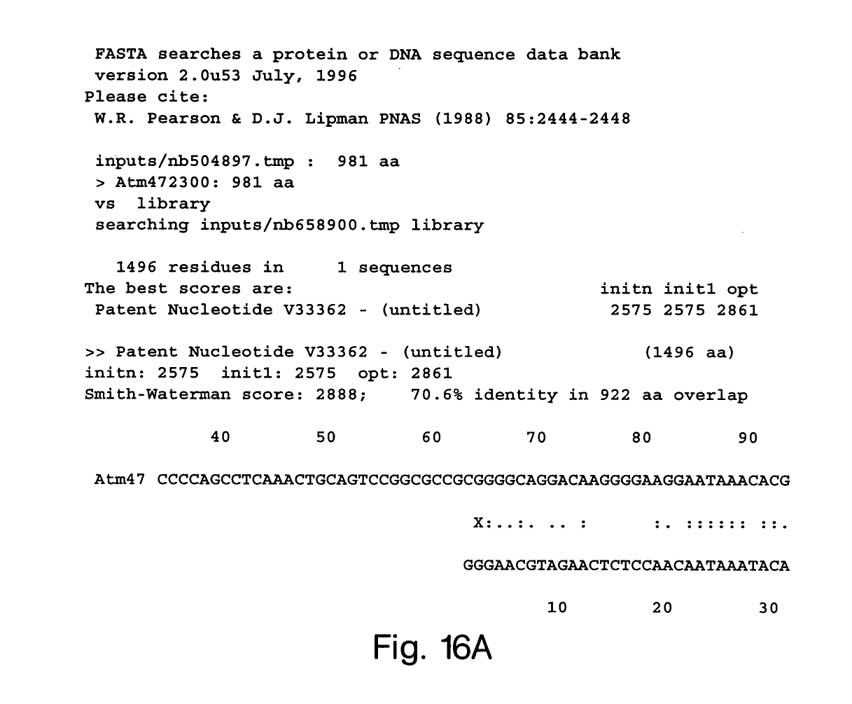

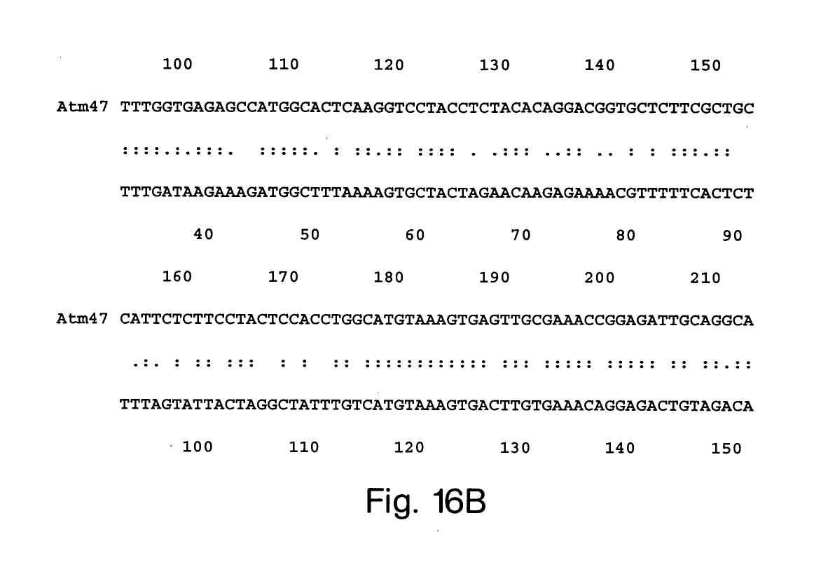

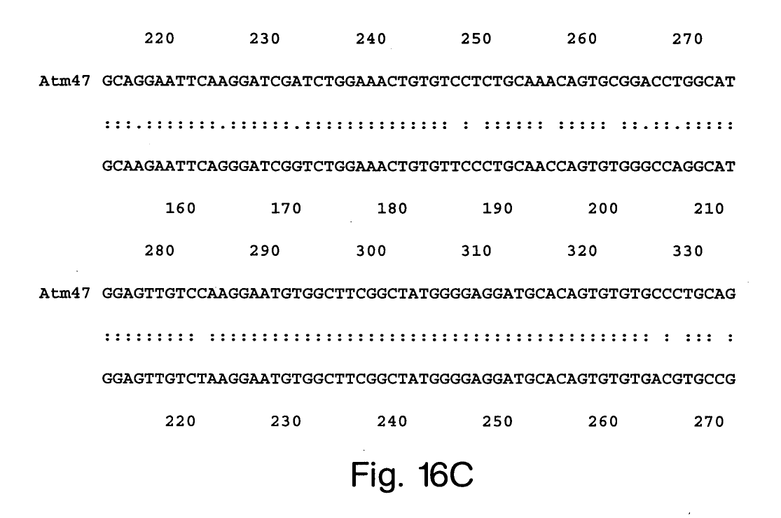

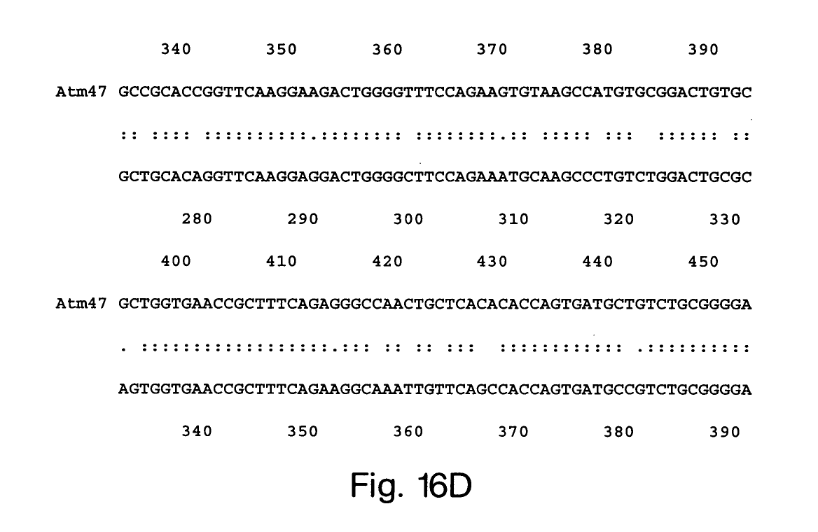

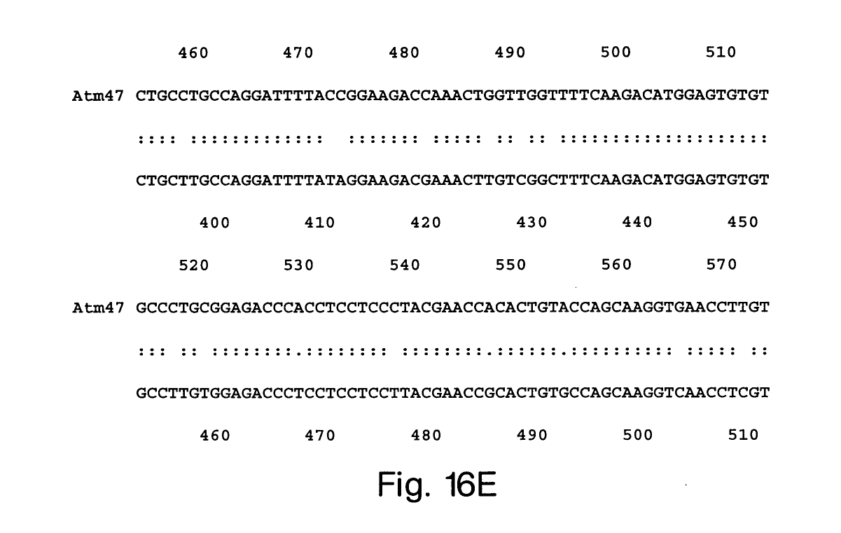

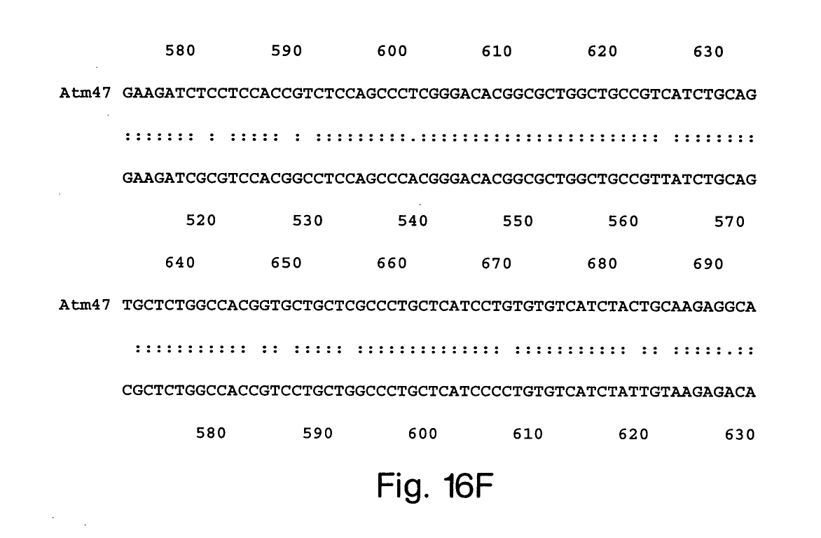

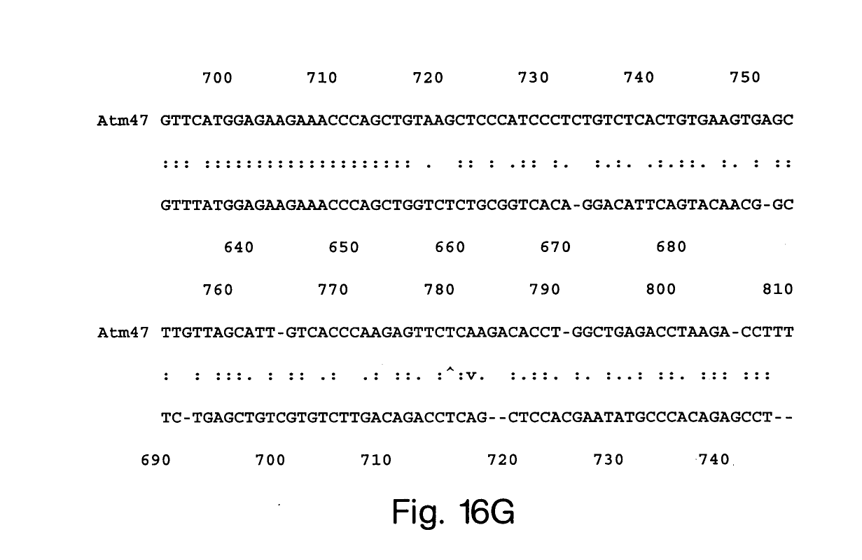

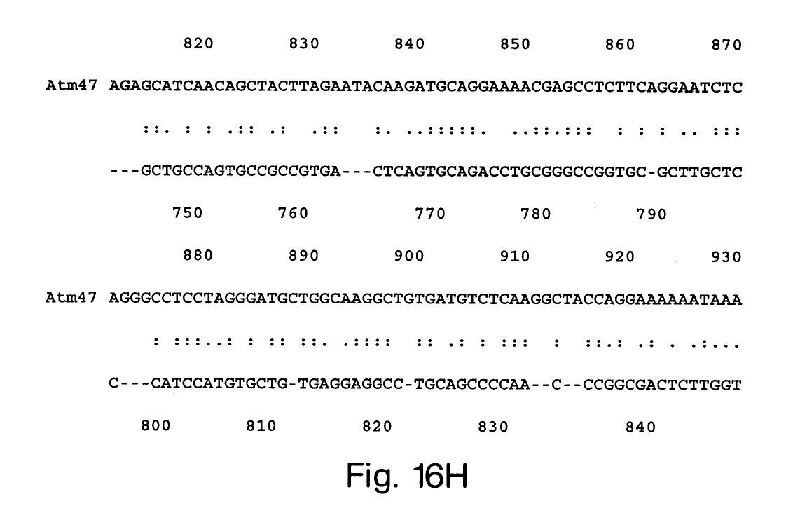

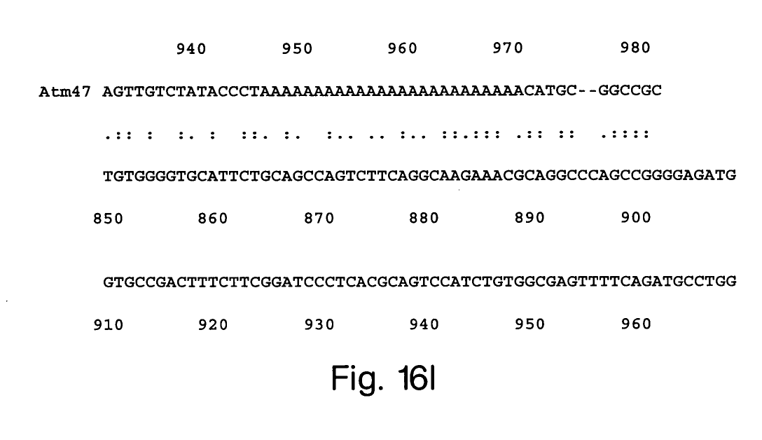

A FASTA search (described in Pearson W. R. & Lipman D. J. (1988) Proc. Natl. Acad. Sci. USA 85:2444-2448, score matrix: PAM 120) using the STRIFE1 nucleotide sequence as a query, indicates that STRIFE1 is 70.6% identical to the nucleic acid molecule encoding the human OAF065 receptor (Accession number V33362; described in PCT application number WO 98/38304, published on Sep. 3, 1998) over nucleotide residues 65-981. The results from this search are shown in FIGS. 16A-161.

### Structure of the STRIFE1 and STRIFE2 Family Proteins

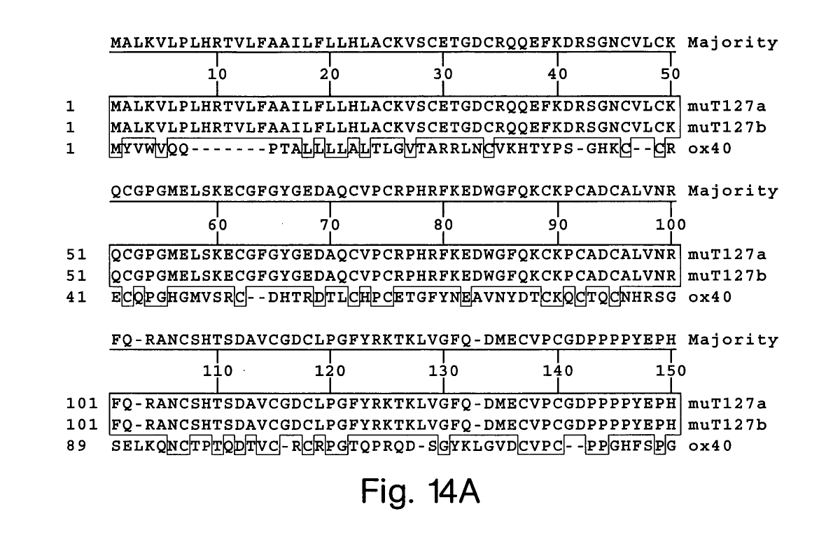

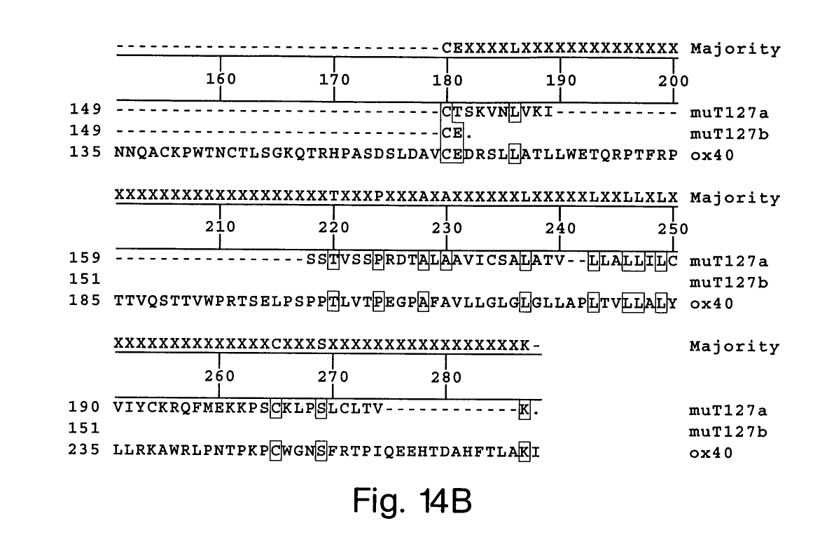

An alignment of the amino acid sequences of murine STRIFE1, STRIFE2, and murine OX40 (Accession Number P47741; SEQ ID NO:30) is shown in FIGS. 14A-14B. Amino acid residues which are conserved between murine STRIFE1 and STRIFE2 family members are highlighted. The percent identity was calculated using the alignment generated using MegAlign™ sequence alignment software. The initial pairwise alignment step was performed using a Wilbur-Lipmann algorithm with a K-tuple of 2, a GAP penalty of 5, a window of 4, and diagonals saved set to =4. The multiple alignment step was performed using the Clustal algorithm with a PAM 250 residue weight Table, a GAP penalty of 10, and a GAP length penalty of 10.

### Example 7

### Isolation and Characterization of Human TRASH cDNAs

In this example, the isolation of the gene encoding human TRASH (also referred to as “TANGO 118”) is described.

A human TRASH cDNA was identified by searching against a copy of the GenBank nucleotide database using the BLASTN™ program (BLASTN 1.3 MP: Altschul et al., J. Mol. Bio. 215:403, 1990). Numerous clones that consisted mostly of 3′ reads and some that were 5′ reads within the 3′ untranslated region were found by this search. The sequences were analyzed against a non-redundant protein database with the BLASTX™ program, which translates a nucleic acid sequence in all six frames and compares it against available protein databases (BLASTX 1.3 MP: Altschul et al., supra). This protein database is a combination of the Swiss-Prot, PIR, and NCBI GenPept protein databases. One clone was obtained from the IMAGE consortium, and fully sequenced. The additional sequencing of this clone extended the original EST by 865 nucleotides further 5′. The cDNA for this clone is approximately 1346 nucleotides in length and has an open reading frame of 753 nucleotides that is predicted to encode a protein of 250 amino acids.

The original first pass sequence of the clone showed homology to human TNF-α using the BLASTX™ program. The nucleotide sequence and predicted amino acid sequence are shown in FIG. 17 (corresponding to SEQ ID NO:31 and SEQ ID NO:32, respectively). The human TRASH protein (corresponding to amino acids 1-250 of the predicted amino acid sequence, SEQ ID NO:32) shows 21.0% identity to the human TNF-α protein (SEQ ID NO:43) and 24.6% identity to the human Tweak protein (SEQ ID NO:44) (see FIG. 18). The percent identity was calculated using the alignment generated using MegAlign™ sequence alignment software. The initial pairwise alignment step was performed using a Wilbur-Lipmann algorithm with a K-tuple of 1, a GAP penalty of 3, a window of 5, and diagonals saved set to =4. The multiple alignment step was performed using the Clustal algorithm with a PAM 250 residue weight Table, a GAP penalty of 10, and a GAP length penalty of 10.

This human TRASH protein contains a TNF signature motif, a TNF-like N-terminal signal transmembrane anchor for a type II membrane protein (corresponding to amino acids 1-44 of the predicted amino acid sequence, SEQ ID NO:32), two cysteine residues which may be disulfide linked (corresponding to amino acids 196 and 211 of the predicted amino acid sequence, SEQ ID NO:32), and two putative N-lined glycosylation sites (corresponding to amino acids 124 and 237 of the predicted amino acid sequence, SEQ ID NO:32). This human TRASH protein also contains three potential initiating ATG codons that would result in polypeptides of 250aa (SEQ ID NO:32), 233aa (SEQ ID NO:39), or 205aa (SEQ ID NO:41) encoding methionines at positions 1, 18, and 46 of SEQ ID NO:32, respectively. The predicted molecular weight for the 250aa TRASH is approximately 27.4 kDa.

A BLASTN™ search of the EST database revealed the following ESTs having significant homology to clone Accession # AA481-449:

| | | Base Pairs | % | |
|---|---|---|---|---|
| EST Database hits | Species | Covered | Identity | Coding? |
| Accession # AA405973 | human | 730-1224 | 97 | yes |
| Accession # AA293679 | human | 1318-884 | 100 | yes |
| Accession # AA394070 | human | 1318-891 | 100 | yes |
| Accession # AA443577 | human | 939-515 | 99 | yes |

### Example 8

### Distribution of TRASH mRNA in Human Tissues

### Northern Blot Analysis

The expression of TRASH was analyzed using Northern blot hybridization. For analysis of human TRASH, the 1.3 kb insert of AA4481449 was used as a probe. The probe DNA was radioactively labeled with ³²P-dCTP using the Prime-It Kit™ (Stratagene, La Jolla, Calif.) according to the instructions of the supplier. Filters containing mRNA (human MTNI and MTNII and murine embryo MTN from Clontech, Palo Alto, Calif.;) were probed in ExpressHyb™ hybridization solution (Clontech) and washed at high stringency according to manufacturer's recommendations.

Expression was found predominantly in the peripheral blood leukocytes where a message of approximately 1.5 kb transcript was observed. In addition, a 1.5 kb transcript was observed in the spleen and lymph nodes and lung. A slightly larger message, approximately 1.7 kb was observed in the colon, spleen, and kidney.

### Example 9

### Identification and Characterization of Human and Murine BDSF cDNA

In this example, the identification and characterization of the genes encoding human BDSF-1 (also referred to as “TANGO 122” and “hT122”) and murine BDSF-1 (also referred to as “mT122”) are described.

Isolation of the Human BDSF cDNA

The invention is based, at least in part, on the discovery of a human gene encoding a novel brain-derived signaling factor protein, referred to herein as BDSF. The methodology used to isolate the human BDSF-1 gene takes advantage of the fact that molecules such as BDSF-1 have an amino terminal signal sequence which directs certain secreted and membrane-bound proteins through the cellular secretory apparatus.

The human BDSF-1 mRNA was identified by screening of a human fetal brain cDNA library. This library was prepared using mRNA purchased from Clontech, Palo Alto (Cat. no, 6573-1). A signal trap cDNA library was prepared by ligating random primed double stranded cDNA into the expression vector, ptrAP1, resulting in fusions of cDNAs to the reporter, alkaline phosphatase (AP). DNAs from individual clones from this library were prepared by standard techniques and transfected into human embryonic kidney fibroblasts (293T cells). After 48 hours, cell supernatants were collected and assayed for AP activity. Clones giving rise to detectable AP activity in the supernatants of transfected cells were analyzed further by DNA sequencing and the novel clones subjected to further DNA sequencing.

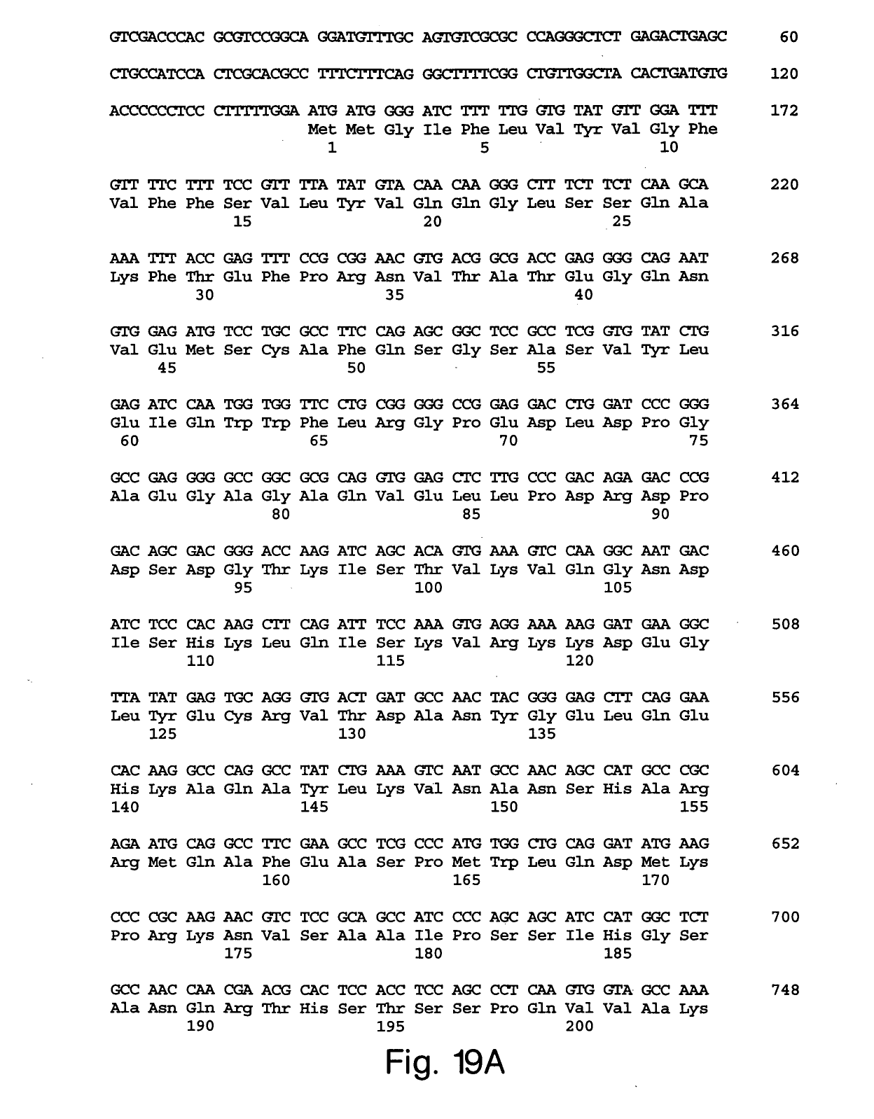

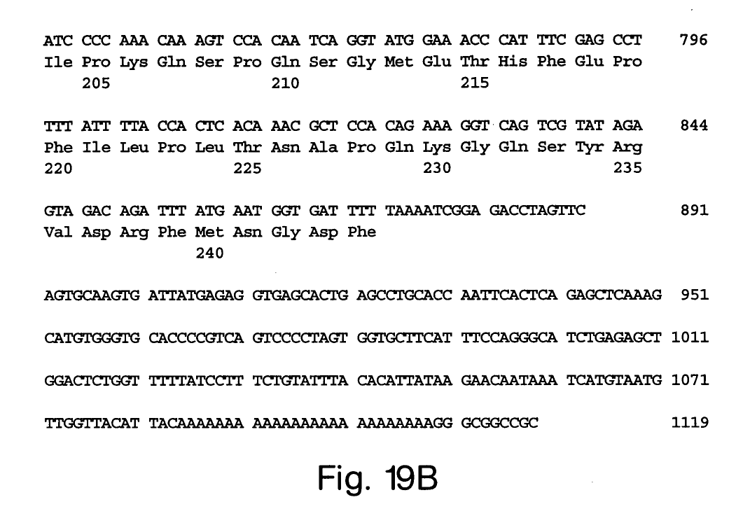

The nucleotide sequence encoding the human BDSF-1 protein is shown in FIGS. 19A-19B and is set forth as SEQ ID NO:45. The full length protein encoded by this nucleic acid is comprised of about 244 amino acids and has the amino acid sequence shown in FIGS. 19A-19B and set forth as SEQ ID NO:46. The coding portion (open reading frame) of SEQ ID NO:45 is set forth as SEQ ID NO:47. Notable features of the human BDSF-1 protein include a signal peptide (about amino acids 1-25 of SEQ ID NO:46), an Ig-like domain (about amino acids 41-129 of SEQ ID NO:46) and two conserved cysteine residues (about amino acids 48 and 127 of SEQ ID NO:46). A clone, comprising the entire coding region of human BDSF-1 has been deposited with the American Type Culture Collection (ATCC), Rockville, Md., on May 15, 1998 as Accession No. 98756.

Isolation of the Murine BDSF cDNA

The murine BDSF-1 gene was identified from a murine choroid plexus cDNA library. More specifically, a murine choroid plexus cDNA library was plated out and colonies picked into 96 well plates. The colonies were cultured, plasmids were prepared from each well, and several of the inserts were sequenced. The nucleotide sequences were compared against the human BDSF-1 nucleotide sequence. Upon review of the results from this sequence comparison, a murine BDSF-1 gene was obtained.

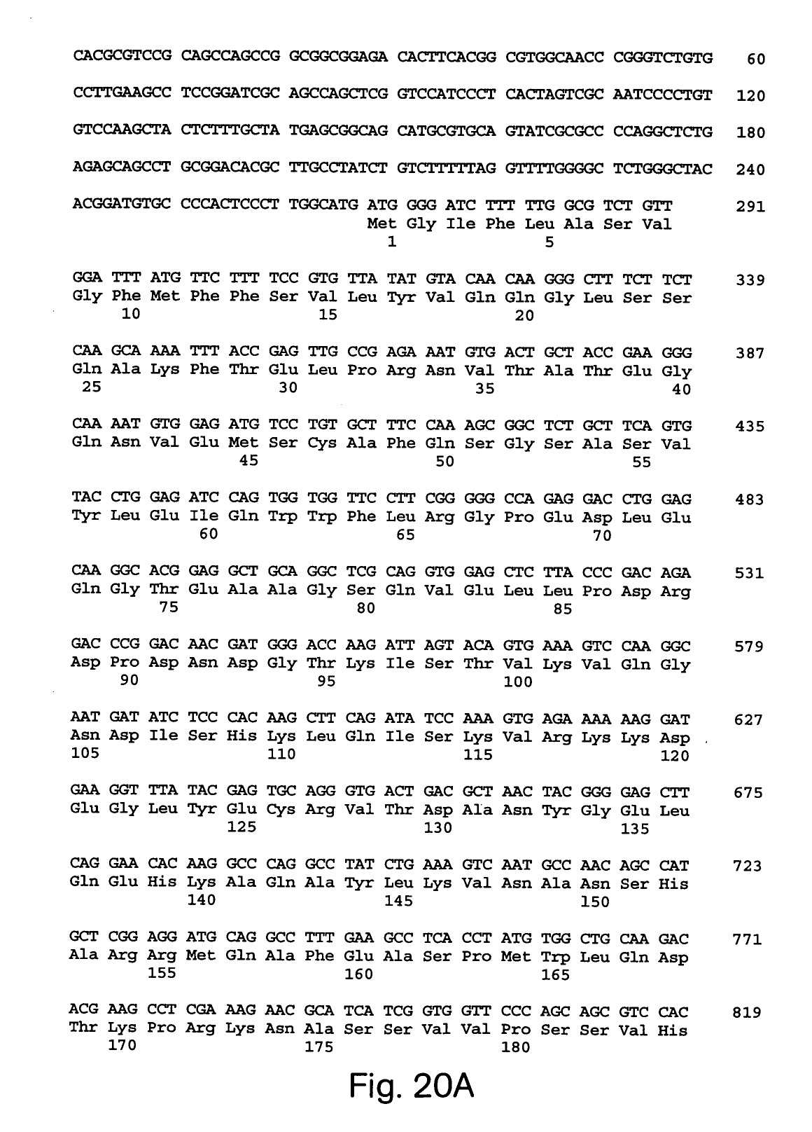

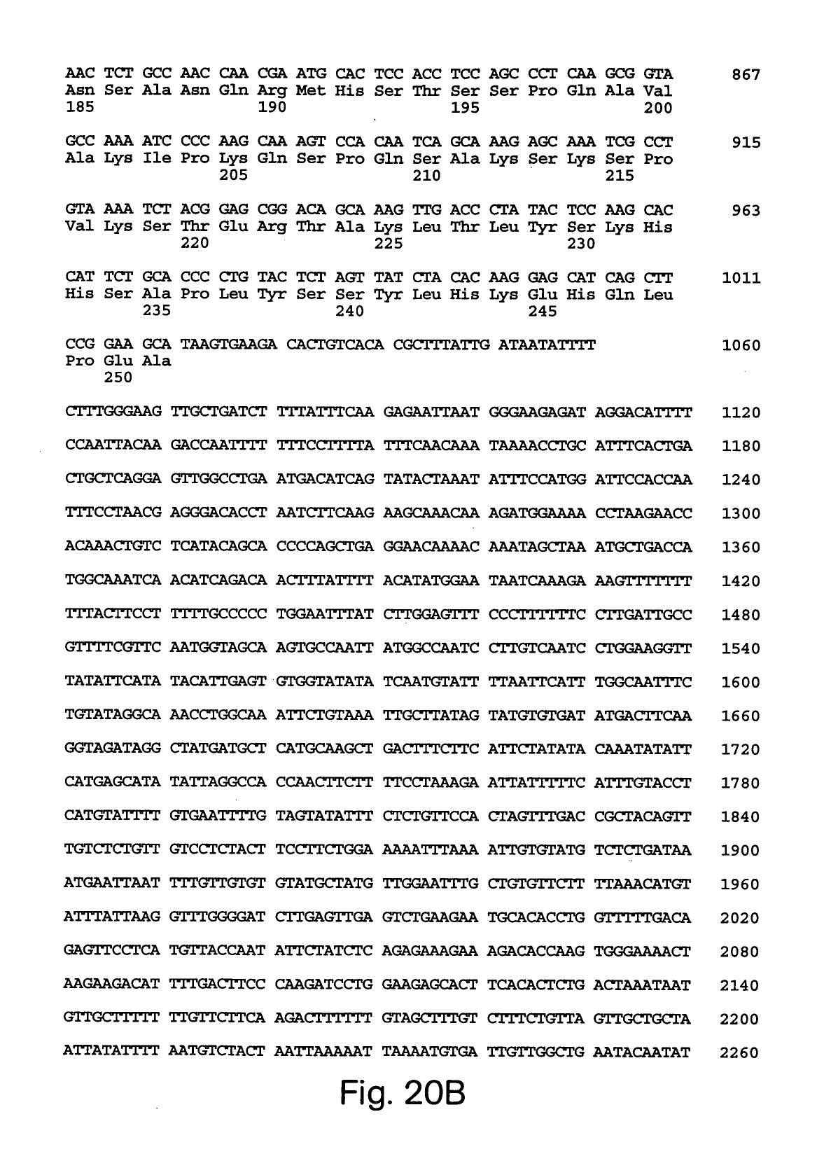

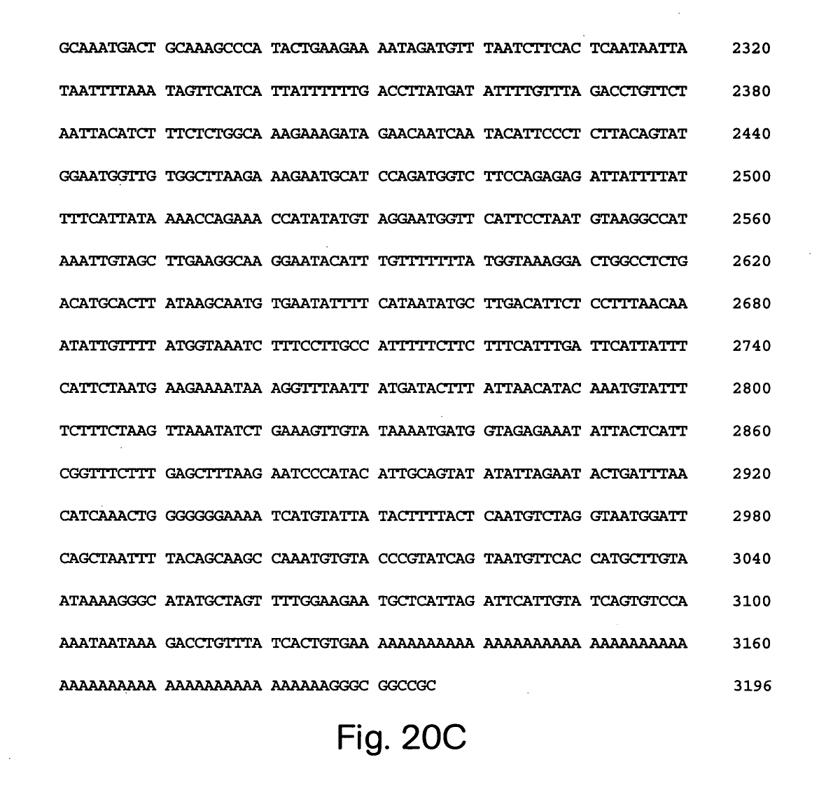

The nucleotide sequence encoding the murine BDSF-1 protein is shown in FIGS. 20A-20C and is set forth as SEQ ID NO:50. The full length protein encoded by this nucleic acid is comprised of about 251 amino acids and has the amino acid sequence shown in FIGS. 20A-20C and set forth as SEQ ID NO:51. The coding portion (open reading frame) of SEQ ID NO:50 is set forth as SEQ ID NO:52. Notable features of the murine BDSF-1 protein include a signal peptide (about amino acids 1-24 of SEQ ID NO:51), an Ig-like domain (about amino acids 40-128 of SEQ ID NO:51) and two conserved cysteine residues (about amino acids 47 and 126 of SEQ ID NO:51).

### Analysis of Human and Murine BDSF

A BLAST search (Altschul et al. (1990) J. Mol. Biol. 215:403) of the nucleotide and protein sequences of human BDSF and murine BDSF has revealed that BDSF does not display significant homology to other proteins except for distant similarity to other Ig-like domain containing proteins. An alignment of human BDSF and murine BDSF, however, indicated that these proteins are 77.4% identical overall and 90.5% identical over the first 211 amino acids. The alignment of human BDSF-1 and murine BDSF-1 is presented in FIG. 21. The alignment of human BDSF-1 and murine BDSF-1 was performed using the ALIGN program. When utilizing the ALIGN program for comparing amino acid sequences, a PAM120 weight residue table, a gap length penalty of 12, and a gap penalty of 4 was used.

A search using the amino acid sequence of SEQ ID NO:46 was performed against the HMM database resulting in the identification of an Ig-like domain in the amino acid sequence of SEQ ID NO:46 and a score of 22.43 against the Ig family HMM Accession PF00047. The results of the search are set forth below:

| | | |
|---|---|---|
| Score: 22.43 | SEQ ID NO: 46: aa41-129 | HMM: aa1-47 (SEQ ID NO: 57) |

| | |
|---|---|
| | GqsVTLTCmVs.fhPpdYt.IwWY.rNgqpi.................. |
| 41 | GQNVEMSCAFQSGSASVYLEIQWWFLRGPEDLDPGAEGAGAQVELLPDR |
| | .....................tLtInsWqyEDsGtYwCmV |
| 90 | DPDSDGTKISTVKVQGNDISHKLQISKVRKKDEGLYECRV |

In another example, a search was performed using the amino acid sequence of SEQ ID NO:51 against the HMM database resulting in the identification of an Ig-like domain in the amino acid sequence of SEQ ID NO:51 and a score of 22.43 against the Ig family HMM Accession PF00047. The results of the search are set forth below.

| | | |
|---|---|---|
| Score: 22.43 | SEQ ID NO: 51 aa40-128 | HMM: aa1-47 (SEQ ID NO: 57) |

| | |
|---|---|
| | GqsVTLTCmVs.fhPpdYt.IwWY.rNgqpi.................. |
| 40 | GQNVEMSCAFQSGSASVYLEIQWWFLRGPEDLEQGTEAAGSQVELLPDR |
| | .....................tLtInsWqyEDsGtYwCmV |
| 89 | DPDNDGTKISTVKVGGNDISHKLQISKVRKKDEGLYECRV |

Accordingly, in one embodiment of the invention, a BDSF protein is a human BDSF-1 protein having an Ig-like domain at about amino acids 41-129 of SEQ ID NO:46. Such an Ig-like domain has the amino acid sequence of SEQ ID NO:55.

### Expression of Human BDSF

The expression of human BDSF was analyzed using Northern blot hybridization and a probe specific for human BDSF. The DNA was radioactively labeled with ³²P dCTP using the Prime-It-kit (Stratagene, La Jolla, Calif.) according to the instructions of the supplier. Filters containing human mRNA (Multi-Tissue Northern I, Multi-Tissue Northern II and Multi-Tissue Northern III from Clontech, Palo Alto, Calif.) were probed in ExpressHyb hybridization solution (Clontech) and washed at high stringency according to manufacturer's recommendations. In addition, filters containing human brain mRNA (Brain-Subregion Blot from Clontech) were also probed for human BDSF-1 expression.

Results of Northern blot hybridization indicate that human BDSF is expressed as an approximately 5.0 kilobase transcript in all tissues (spleen, lymph node, thymus, peripheral blood leukocytes, bone marrow, stomach, thyroid, spinal cord, trachea, adrenal, testis, small intestine, heart, placenta, lung, liver, kidney and pancreas). BDSF mRNA expression was also observed in human fetal liver.

The highest level of human BDSF expression was found in the adult brain where the pattern of transcripts was also different than for the other tissues. In the brain, major transcripts of 2.6 kb, 3.2 kb and 6.5 kb were observed. This pattern of BDSF mRNA transcripts in the brain is found in the sub-regions of the brain including amygdala, caudate nucleus, hippocampus, substania nigra, sub-thalamate nucleus and thalamus, but not in corpus callosum.

### Example 10

### Identification and Characterization of Human LRSG-1 cDNA

In this example, the identification and characterization of the gene encoding human LRSG-1 (also referred to as human “TANGO 124”) is described.

Isolation of the Human LRSG-1 cDNA

The invention is based, at least in part, on the discovery of a human gene encoding a novel leucine-rich repeat containing protein, referred to herein as LRSG-1. Human astrocytes (obtained from Clonetics Corporation; San Diego, Calif.) were expanded in culture with Astrocyte Growth Media (AGM; Clonetics) according to the recommendations of the supplier. When the cells reached ˜80-90% confluence, they were stimulated with 200 units/ml Interleukin 1-Beta (Boehringer Mannheim) and cyclohexamide (CHI; 40 micrograms/ml) for 4 hours. Total RNA was isolated using the RNeasy Midi Kit (Qiagen; Chatsworth, Calif.), and the poly A+ fraction was further purified using Oligotex beads (Qiagen).

Three micrograms of poly A+ RNA were used to synthesize a cDNA library using the Superscript cDNA Synthesis kit (Gibco BRL; Gaithersburg, Md.). Complementary DNA was directionally cloned into the expression plasmid pMET7 using the SalI and NotI sites in the polylinker to construct a plasmid library. Transformants were picked and grown up for single-pass sequencing. Additionally, astrocyte cDNA was ligated into the SalI/NotI sites of the ZipLox vector (Gibco BRL) for construction of a lambda phage cDNA library. A clone (jthxe016d10) that encoded a protein with limited homology to decorin, insulin-like growth factor binding protein and biglycan was identified. Full sequencing of the clone demonstrated that it contained an ˜2.8 kb insert with a single large open reading frame predicted to encode a 673 amino acid transmembrane protein.

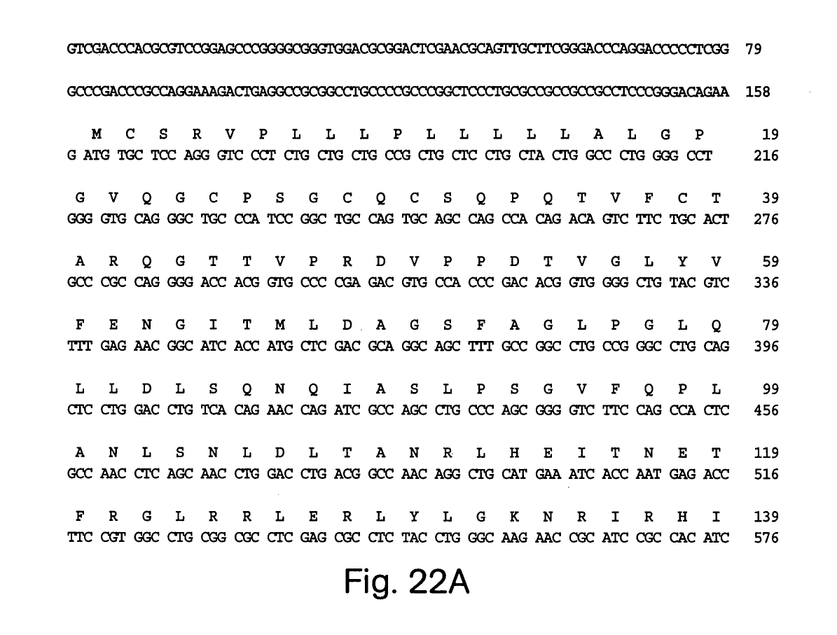

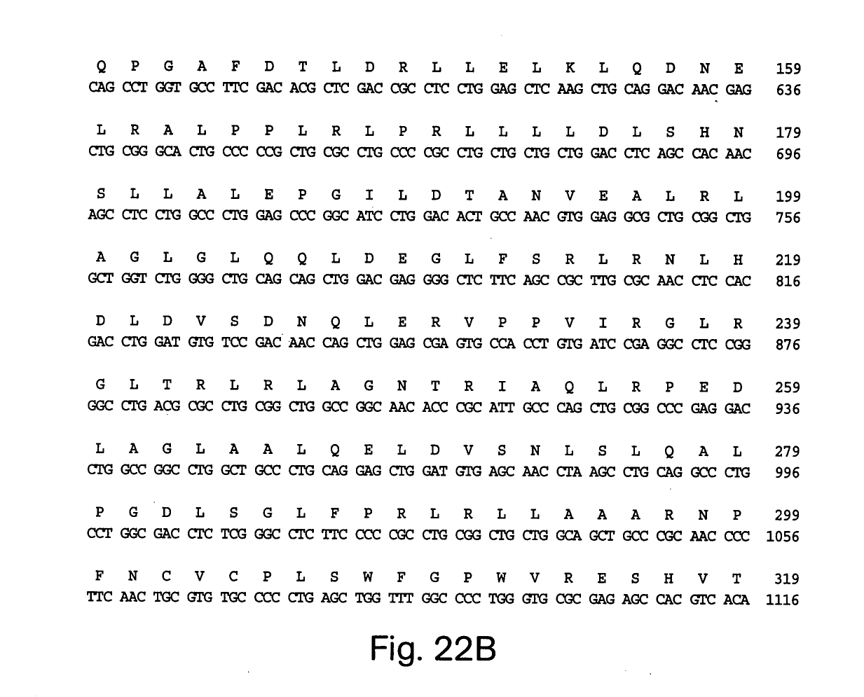

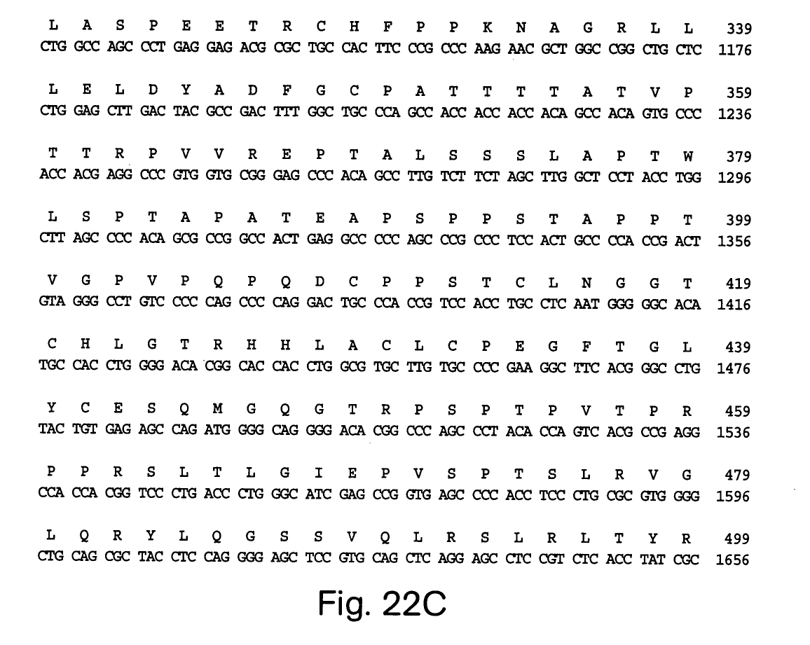

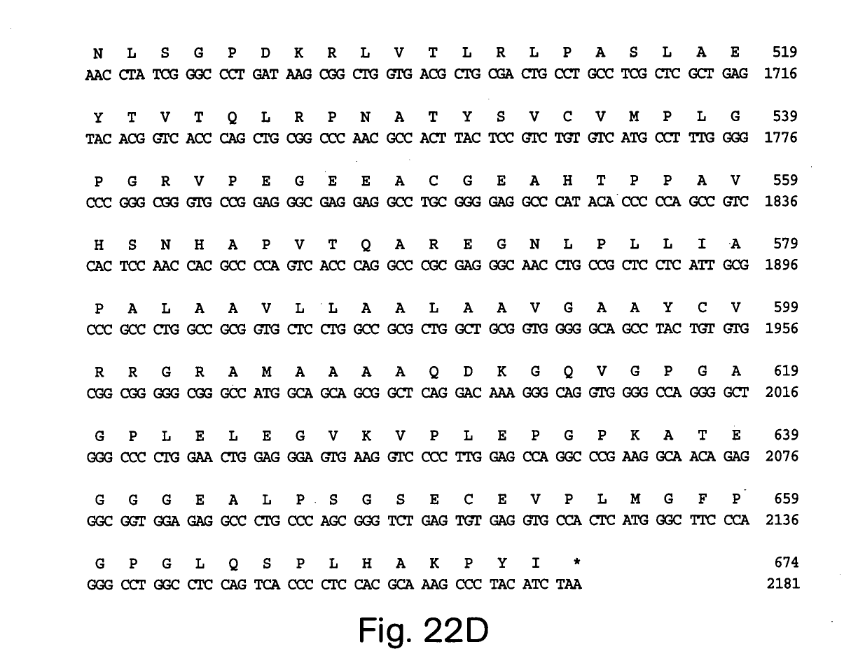

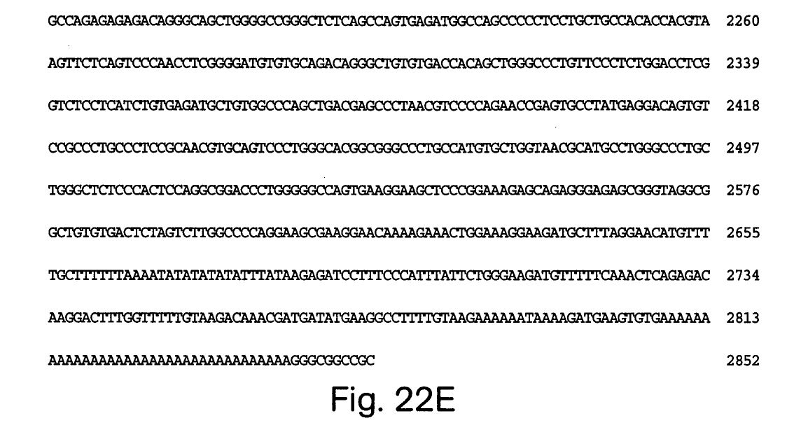

The nucleotide sequence encoding the human LRSG-1 protein is shown in FIGS. 22A-22E and is set forth as SEQ ID NO:58. The full length protein encoded by this nucleic acid is comprised of about 673 amino acids and has the amino acid sequence shown in FIGS. 22A-22E and set forth as SEQ ID NO:59. The coding portion (open reading frame) of SEQ ID NO:58 is set forth as SEQ ID NO:60. Clone jthxe016d10, comprising the entire coding region of human LRSG-1 has been deposited with the American Type Culture Collection (ATCC), Manassas, Va. on Mar. 12, 1998 as accession Number 98695.

Notable features of the human LRSG-1 protein include a signal peptide (about amino acids 1-23 of SEQ ID NO:59), a transmembrane domain (about amino acids 576-599 of SEQ ID NO:59) an EGF-like domain (about amino acids 409-441) and a fibronectin type III-like domain (about amino acids 460-535 of SEQ ID NO:59). Th human LRSG-1 protein further includes a leucine-rich region (about amino acids 77-309 of SEQ ID NO:59) which includes at least 7 leucine-rich repeats (about amino acids 77-309, 101-123, 125-147, 149-171, 217-238, 240-263, and 289-309 of SEQ ID NO:59).

### Analysis of Human LRSG-1

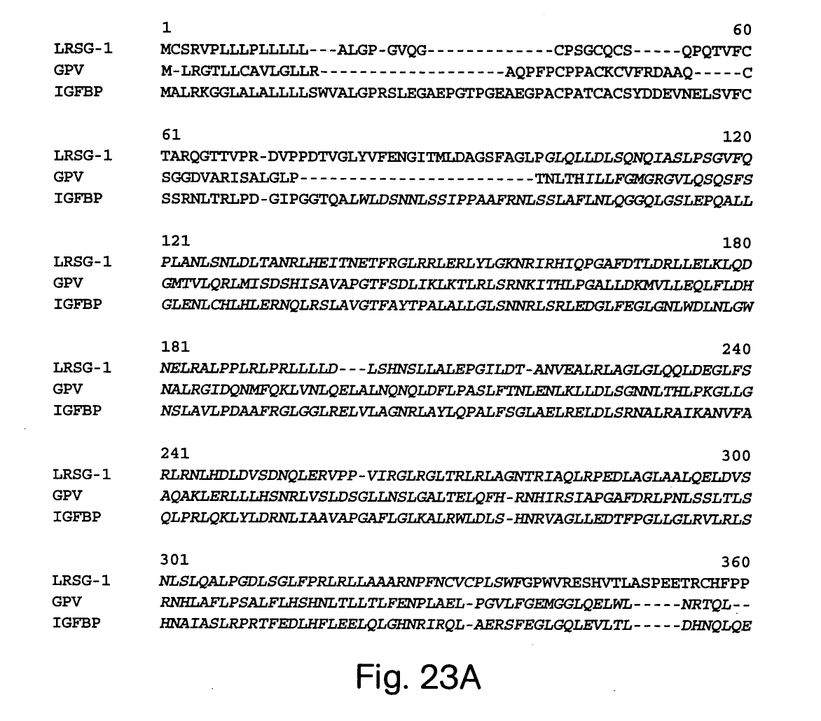

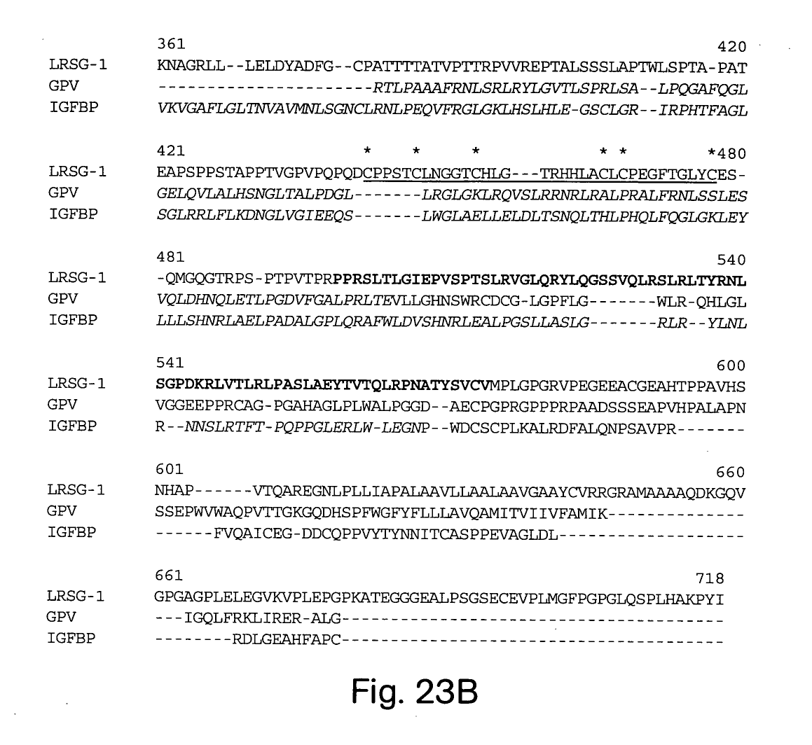

A BLAST search (Altschul et al. (1990) J. Mol. Biol. 215:403) of the nucleotide and protein sequences of human LRSG-1 has revealed that LRSG-1 has structural similarities with both platelet glycoprotein V precursor (GPV) (SwissProt Accession No. P40197; SEQ ID NO:61) and insulin-like growth factor binding protein complex acid labile chain precursor (ALS) (SwissProt Accession No. 002833; SEQ ID NO:62). Each of these proteins is a leucine-rich repeat containing protein although LRSG-1 shares no greater than 30% identity with any of these LRR-containing proteins. An alignment of human LRSG-1 and the above-described proteins is presented in FIGS. 23A-23B.

Human LSRG-1 was analyzed for the presence of an FN type III-like domain. An alignment with an FN type III-like consensus sequence is shown below for amino acid residues 460-535 of SEQ ID NO:59:

| | |
|---|---|
| FN type III-like consensus | PsPPrNLrvtdITpTSItVSWtPPe..gNgpItgYr |
| | P+ L +++++PTS++V ++ + +++ R |
| LRSG-1 | PPRSLTLGIEPVSPTSLRVGLQRYLQGSSVQLRSLR |
| FN type III-like consensus | IqYRWpvNdne..WnEfnVPrttnsYTItnLrPGTeYeFRV |
| | ++YR + +++ +++++P + +YT+T LRP+ +Y++ V |
| LRSG-1 | LTYR-NLSGPDKRLVTLRLPASLAEYTVTQLRPNATYSVCV |

### Expression of LRSG-1

The expression of LRSG-1 was analyzed using Northern blot hybridization. A 579 base pair (bp) DNA fragment from the N-terminal portion of the coding region was generated using PCR for use as a probe. The DNA was radioactively labeled with ³²P dCTP using the Prime-It-kit (Stratagene, La Jolla, Calif.) according to the instructions of the supplier. Filters containing human mRNA (Multi-Tissue Northern I and Multi-Tissue Northern II from Clontech, Palo Alto, Calif.) were probed in ExpressHyb hybridization solution (Clontech) and washed at high stringency according to manufacturer's recommendations.

Results of Northern blot hybridization indicate that LRSG-1 is expressed as an approximately 3.0 kilobase transcript in all tissues (spleen, thymus, prostate, testes, ovary, small intestine, colon, heart, brain, placenta, lung, liver, skeletal muscle, kidney and pancreas) with the exception of peripheral blood leukocytes. The highest levels of LRSG-1 were found in placenta, kidney and testis.

### Example 11

### Identification and Characterization of Murine LRSG-1 cDNA

In this example, the identification and characterization of the gene encoding murine LRSG-1 (also referred to as murine “TANGO 124”) is described.

Using sequence information from the cloning of human LRSG-1, a murine homologue was identified.

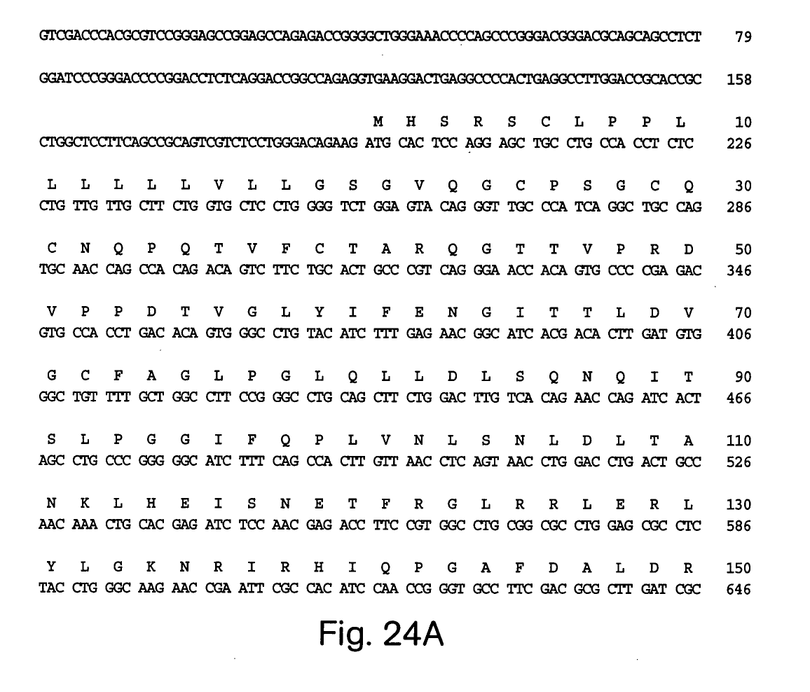

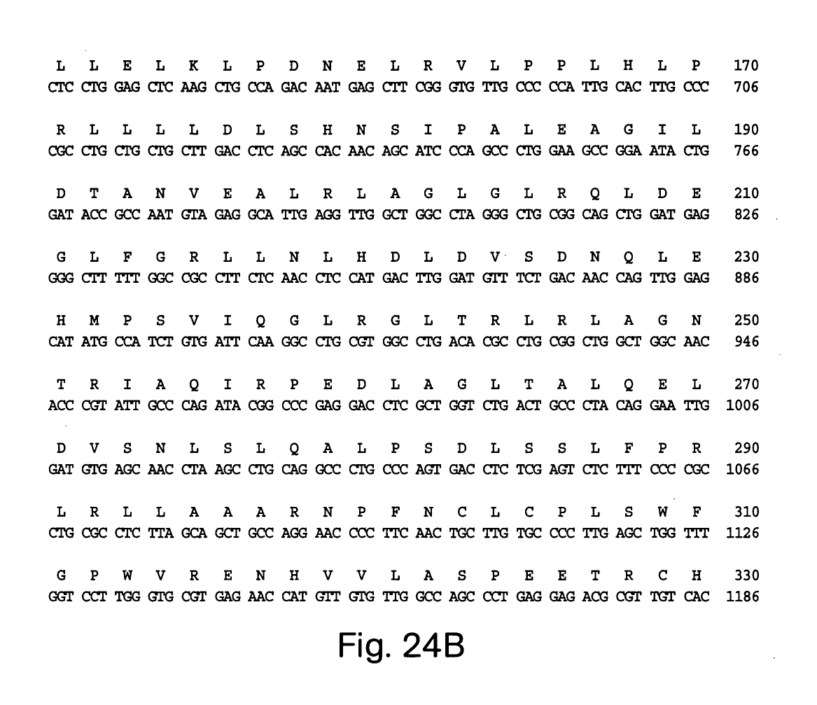

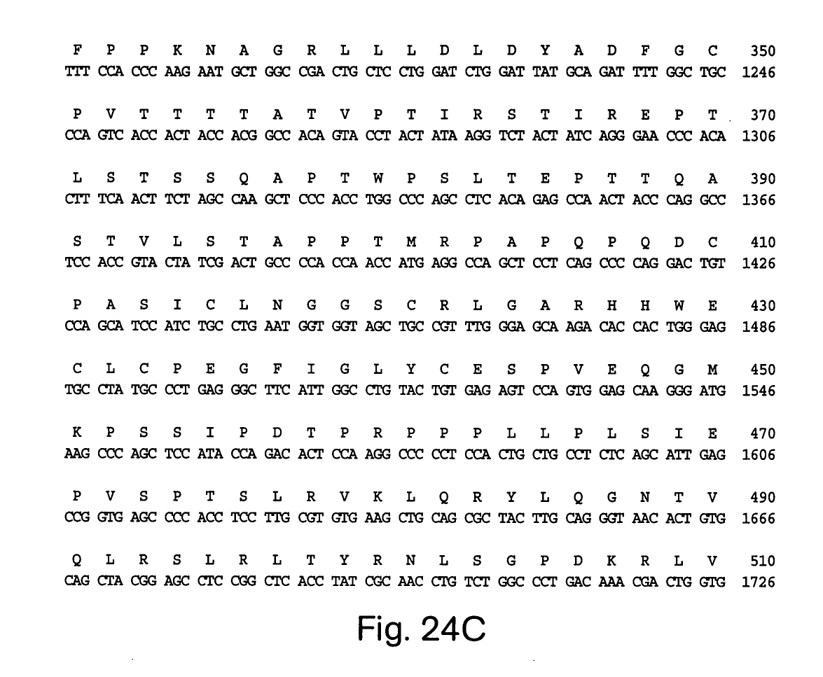

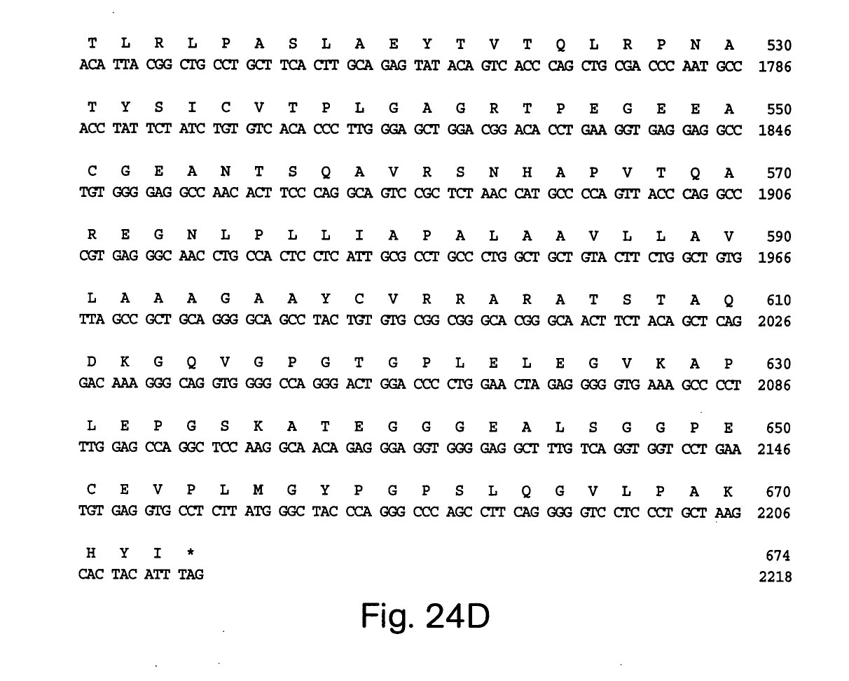

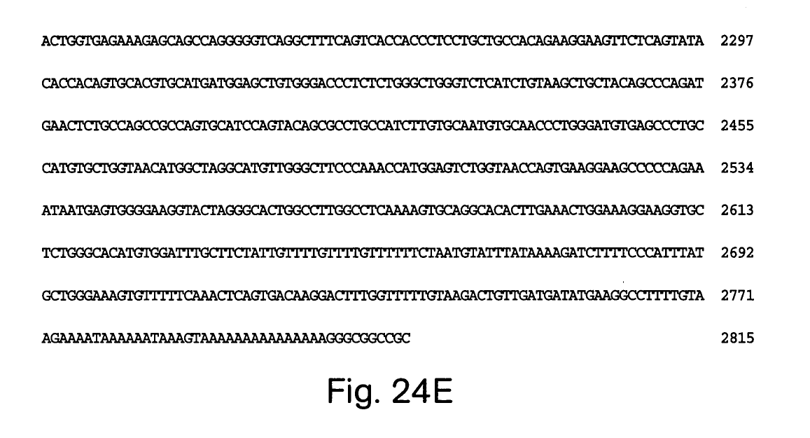

The nucleotide sequence encoding the murine LRSG-1 protein is shown in FIGS. 24A-24E and is set forth as SEQ ID NO:67. The full length protein encoded by this nucleic acid is comprised of about 673 amino acids and has the amino acid sequence shown in FIGS. 24A-24E and set forth as SEQ ID NO:68. The coding portion (open reading frame) of SEQ ID NO:67 is set forth as SEQ ID NO:69.

Notable features of the murine LRSG-1 protein include a signal peptide (about amino acids 1-24 of SEQ ID NO:68), a transmembrane domain (about amino acids 577-600 of SEQ ID NO:68) an EGF-like domain (about amino acids 410-442) and a fibronectin type III-like domain (about amino acids 461-636 of SEQ ID NO:68). The murine LRSG-1 protein further includes a leucine-rich region (about amino acids 78-310 of SEQ ID NO:68) which includes at least 7 leucine-rich repeats (about amino acids 78-310, 102-124, 126-148, 150-172, 218-238, 241-264, and 290-310 of SEQ ID NO:68). An Fn type III-like domain was identified at about residues 461-536 of SEQ ID NO:68

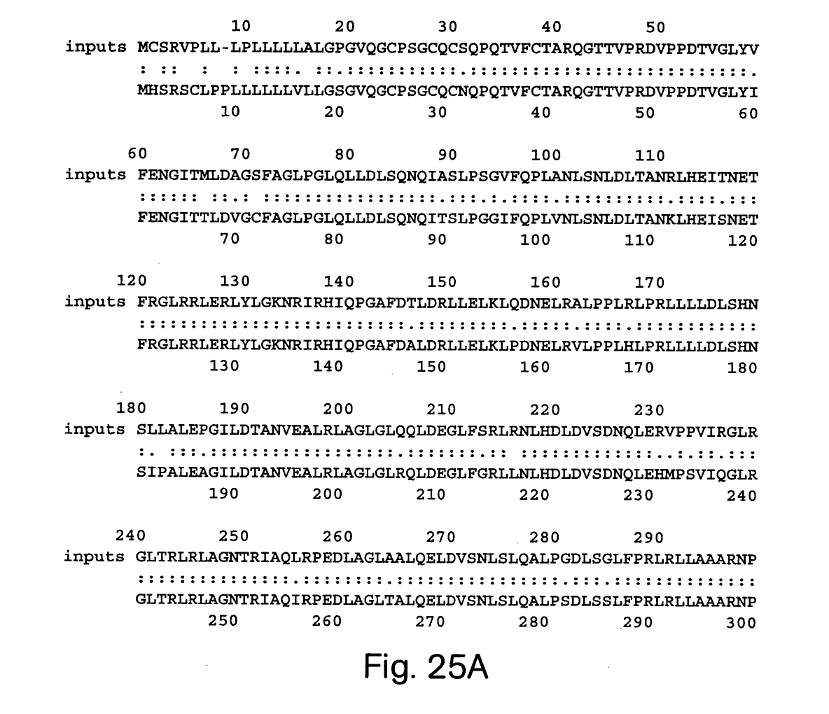

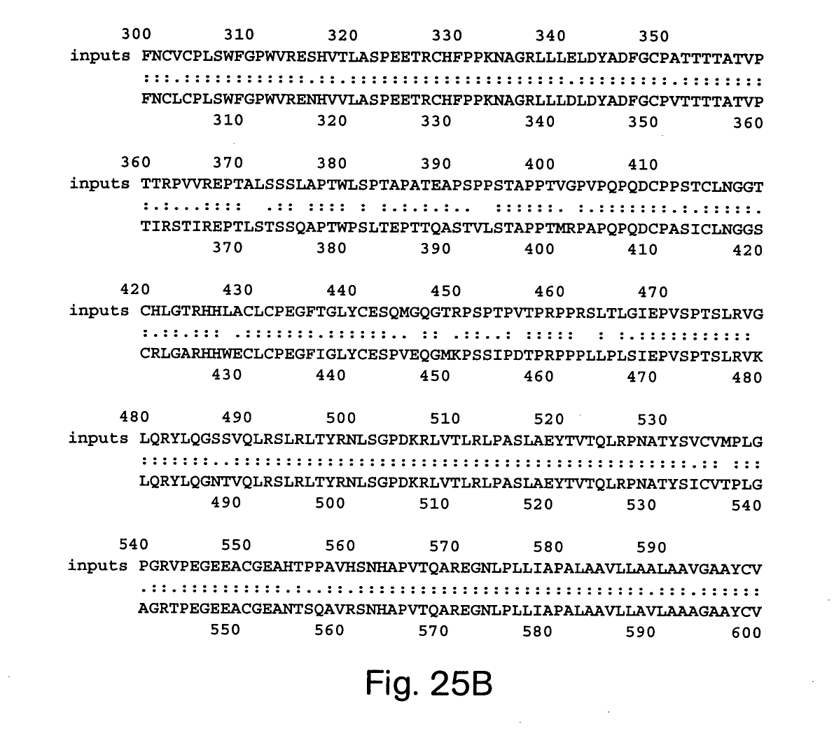

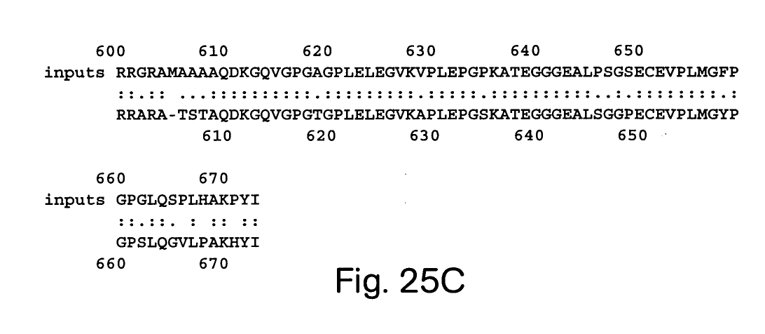

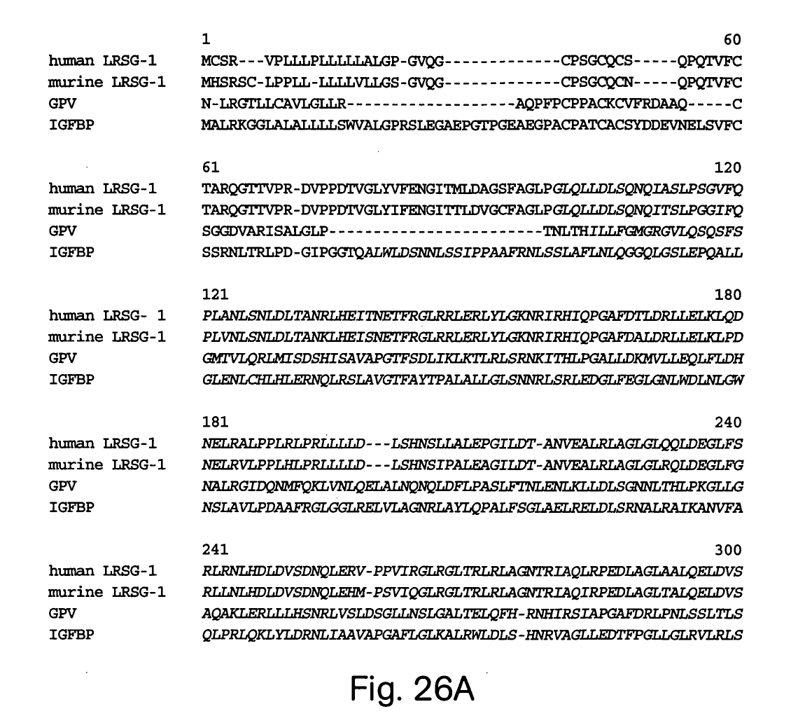

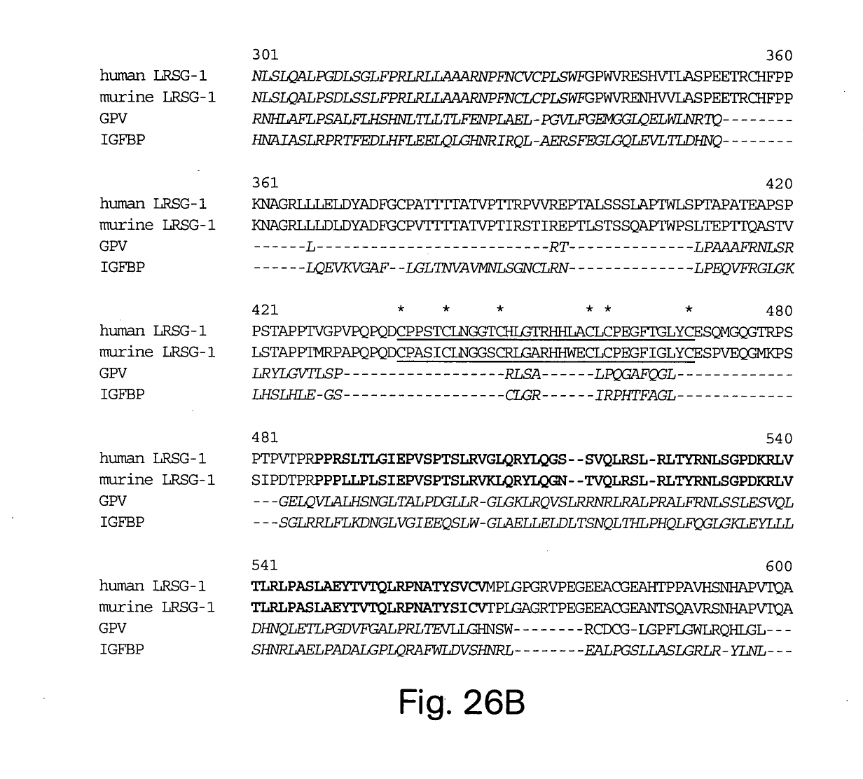

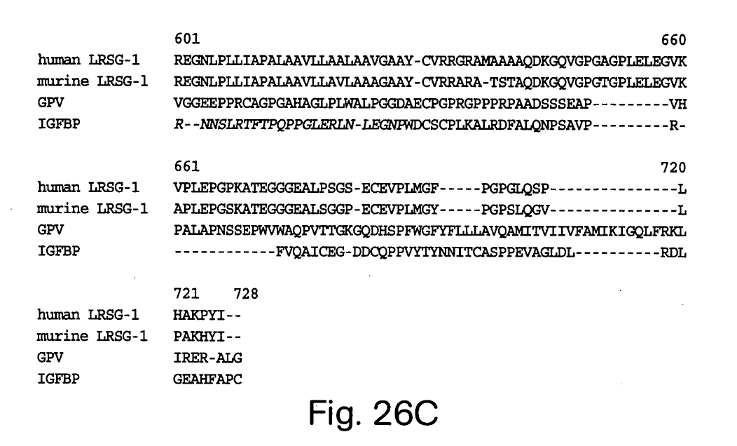

FIGS. 25A-25C set forth a pairwise alignment of the amino acid sequences of human and murine LRSG-1. The alignment was generated using the ALIGN algorithm, version 2, which is part the GCG software package. ALIGN (Myers and Miller, CABIOS (1989)) calculates a global alignment of two sequences and is part the GCG software package. The alignment was generated using a PAM120 scoring matrix and gap penalties of −12/−4. Murine LRSG-1 is 83.2% identical to human LRSG-1 (global alignment score: 2910).

### Example 12

### Chromosomal Mapping of LRSG-1 cDNA

PCR primers were designed based of the coding sequence of human LRSG-1 and used to generate probes for chromosomal mapping. LRSG-1 was found to map to chromosome 16 between markers WI-7742 and WI-3061 (46-52cM).

### Example 13

### Identification and Characterization of STMST-1 cDNAs

In this example, the identification and characterization of the genes encoding human STMST-1 and STMST-2 (also referred to as “TANGO123a” and “TANGO 123c”, respectively) is described.

Isolation of the Human STMST cDNAs

In order to identify novel secreted and/or membrane-bound proteins, a program termed ‘signal sequence trapping’ was utilized to analyze the sequences of several cDNAs of a cDNA library derived from bronchial epithelial cells which had been stimulated with the cytokine, TNFα. This analysis identified a partial human clone having an insert of approximately 231 kb containing a protein-encoding sequence of approximately 225 nucleotides capable of encoding approximately 75 amino acids of STMST (e.g., the start met through residue 74 of, for example, SEQ ID NO:71). This cDNA was used to re-screen the library. Two full-length cDNA clones were isolated. Sequencing of these clones revealed the nucleotide sequences of human STMST-1 and STMST-2.

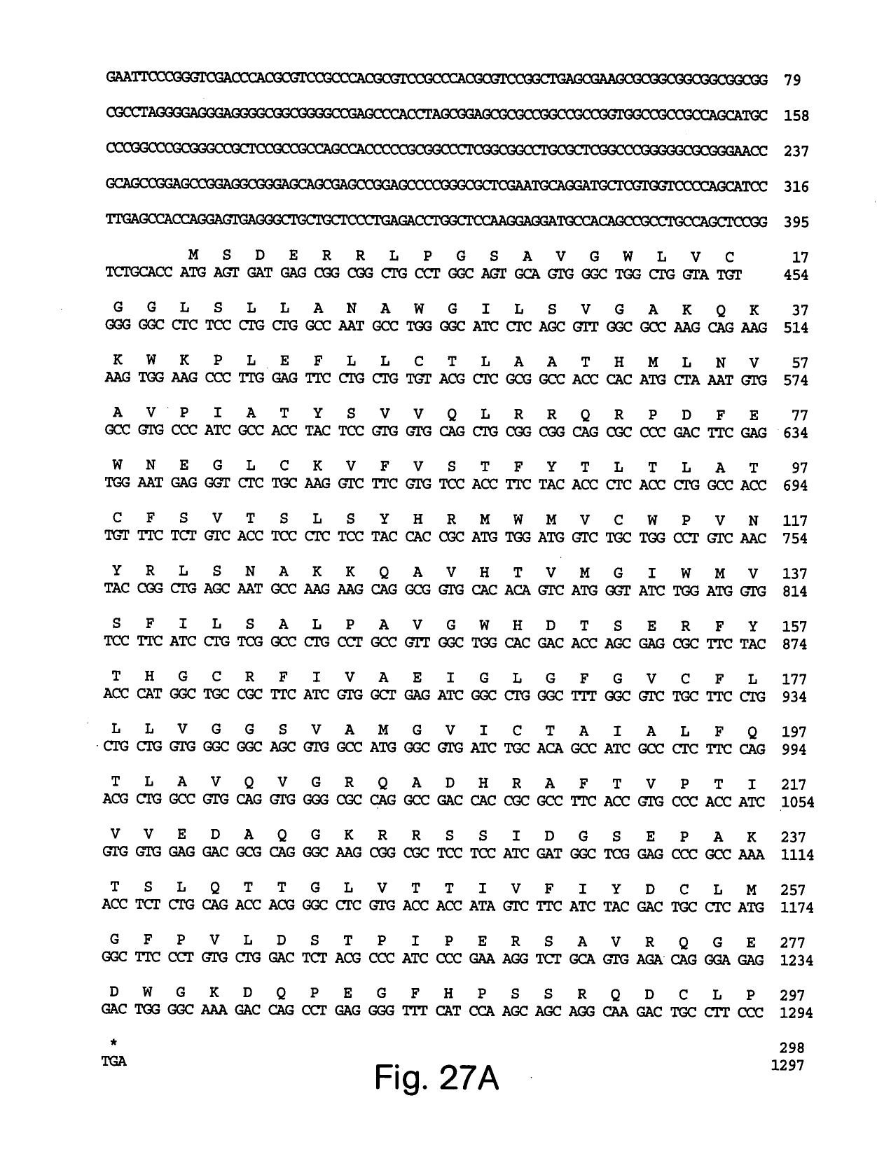

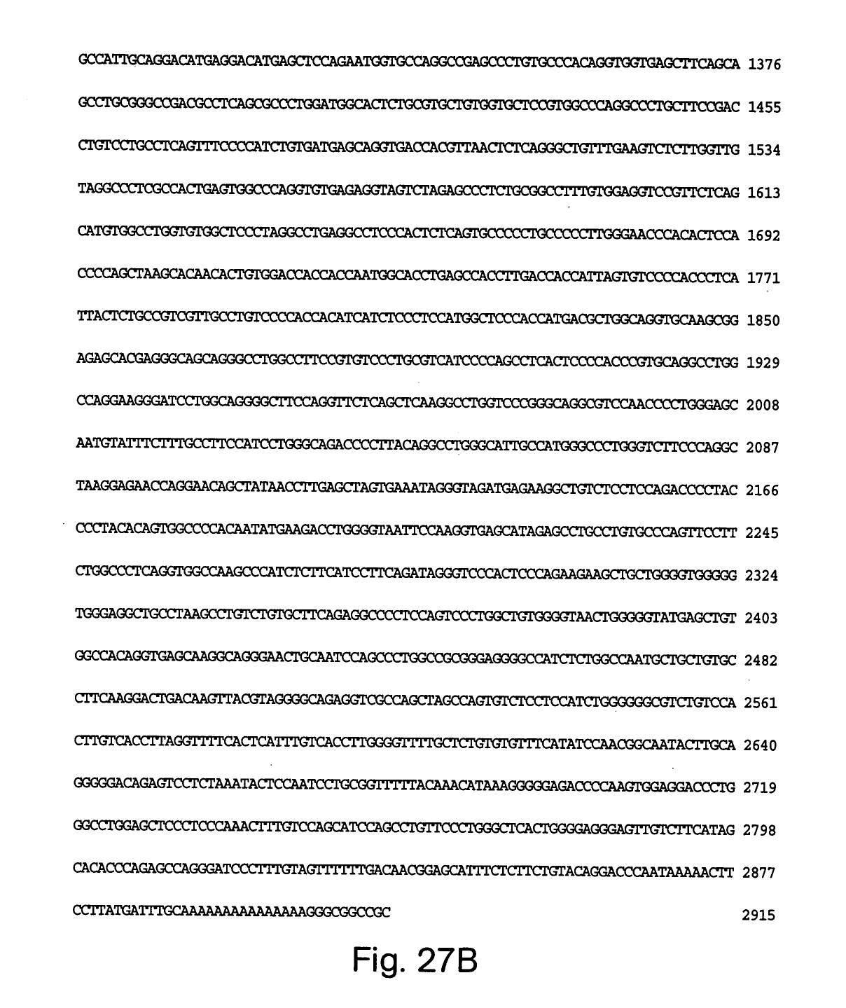

The nucleotide sequence encoding the human STMST-1 protein is shown in FIGS. 27A-27B and is set forth as SEQ ID NO:70. The full length protein encoded by this nucleic acid is comprised of about 297 amino acids and has the amino acid sequence shown in FIGS. 27A-27B and set forth as SEQ ID NO:71. The coding portion (open reading frame) of SEQ ID NO:70 is set forth as SEQ ID NO:72.

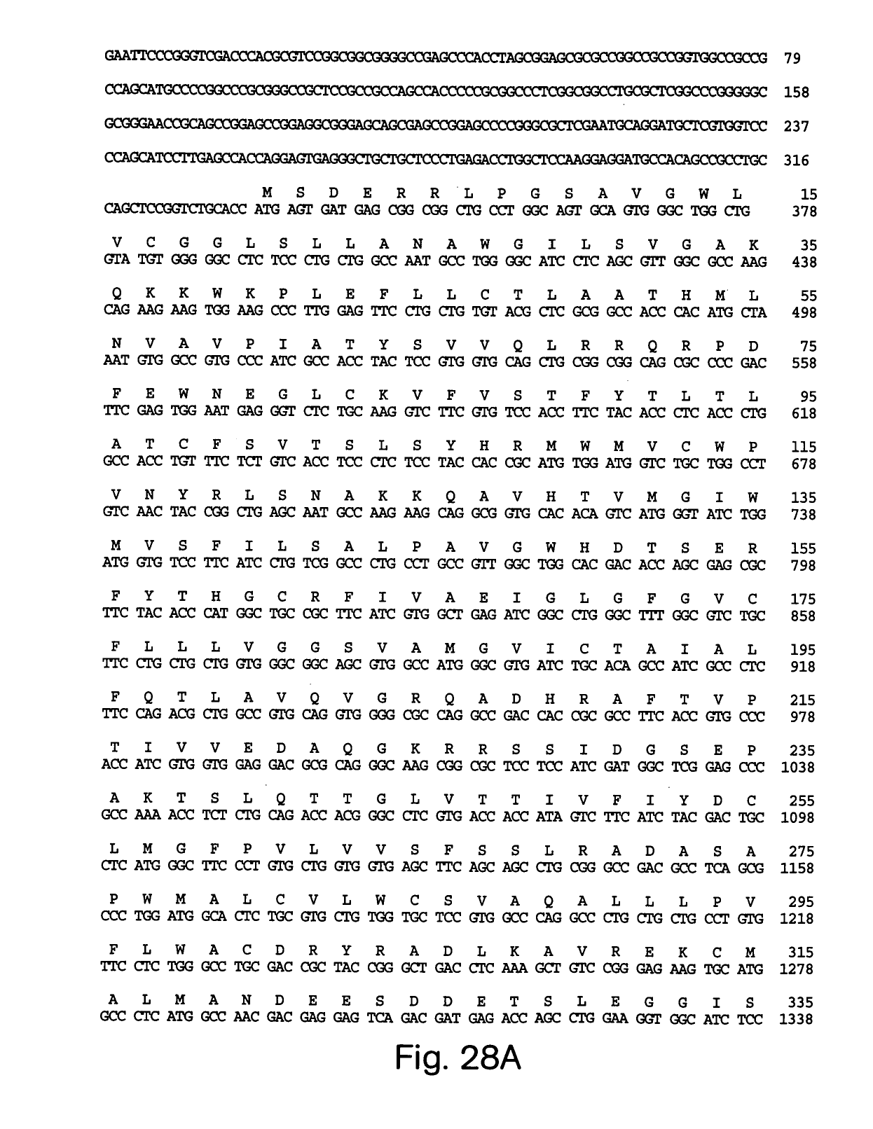

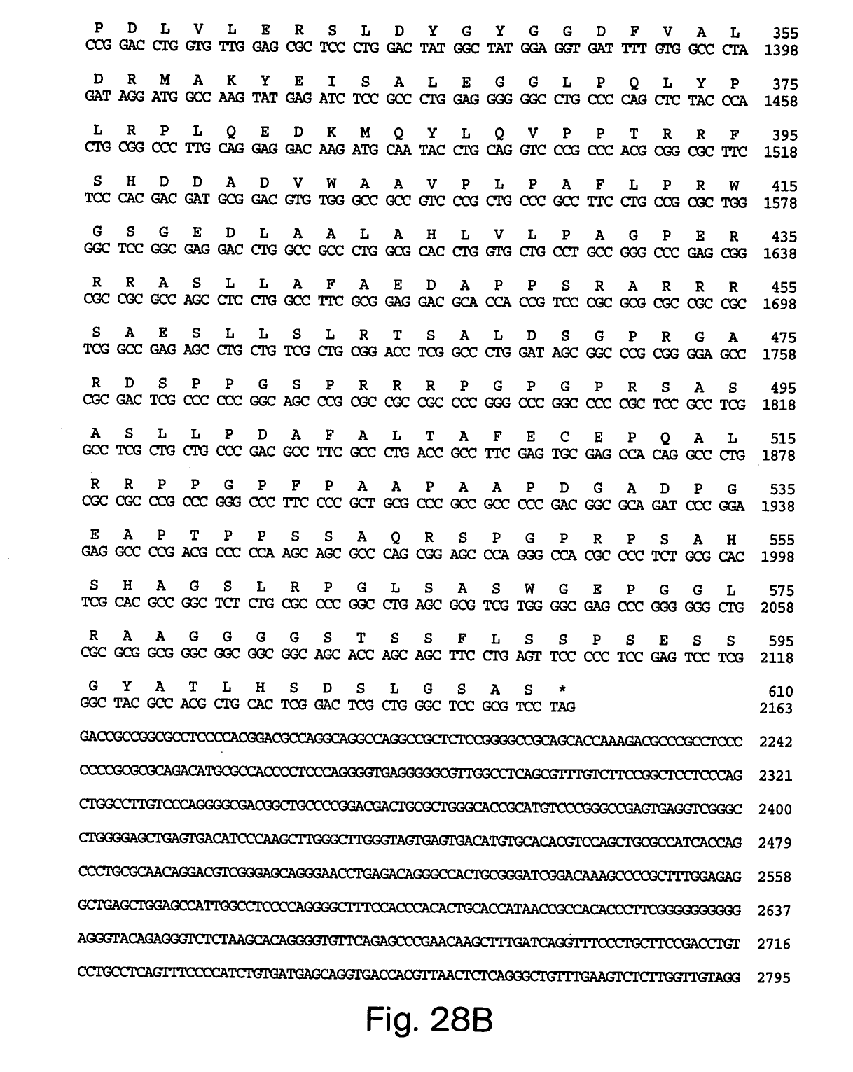

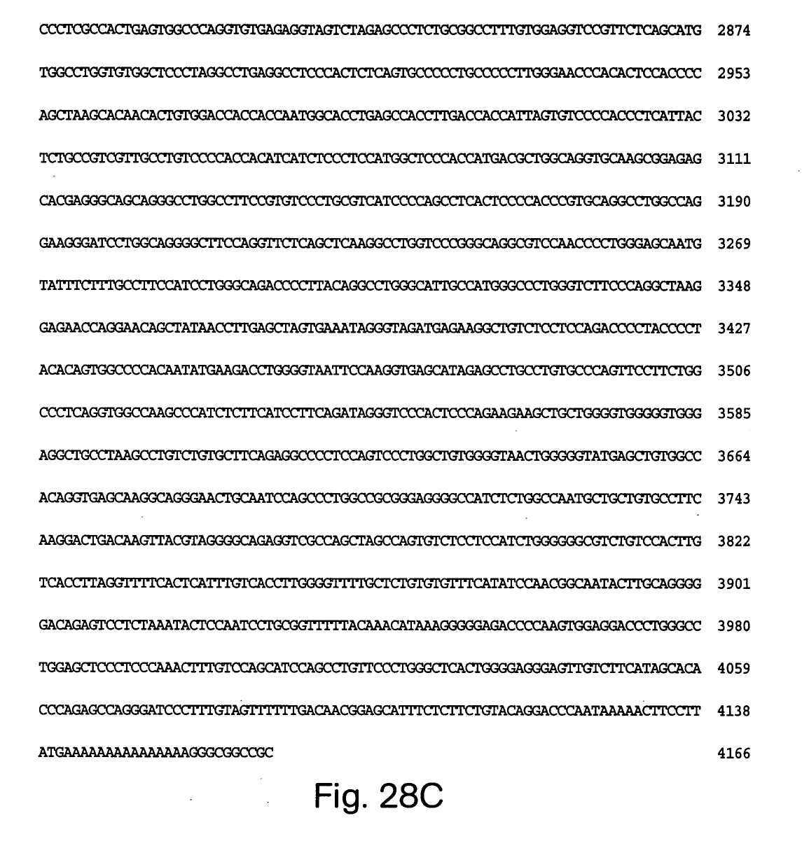

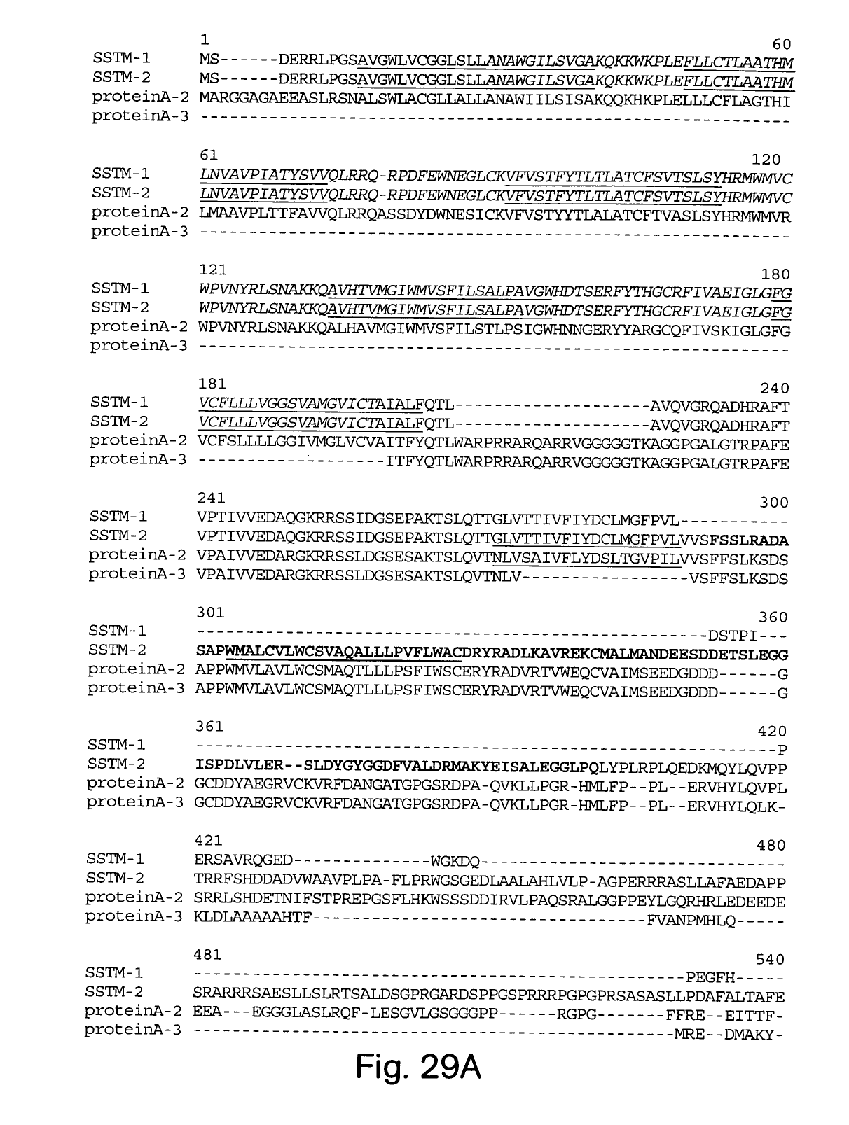

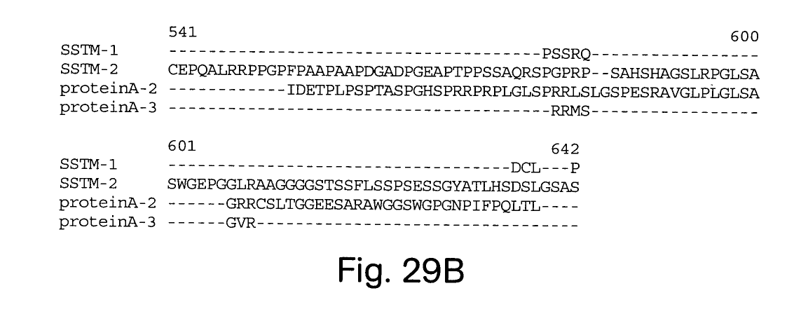

The nucleotide sequence encoding the human STMST-2 protein is shown in FIGS. 28A-28C and is set forth as SEQ ID NO:73. The full length protein encoded by this nucleic acid is comprised of about 609 amino acids and has the amino acid sequence shown in FIGS. 28A-28C and set forth as SEQ ID NO:74. The coding portion (open reading frame) of SEQ ID NO:73 is set forth as SEQ ID NO:75.

### Analysis of Human STMST-1 and STMST-2

Examination of the cDNA sequences depicted in FIGS. 27A-27B and 28A-28C showed that they were likely encoded by alternatively spliced mRNAs derived from the same gene. Thus, the amino acid sequence of STMST-1 diverges from that of STMST-2 at about amino acid residue 263 of SEQ ID NO:71 or SEQ ID NO:74. The amino acid sequence of STMST-1 lacks the extensive cytoplasmic domain of STMST-2.

A BLAST search (Altschul et al. (1990) J. Mol. Biol. 215:403) of the nucleotide sequence of human STMST-2 has revealed that STMST-2 is significantly similar to a protein identified as protein A-2 (human A-2, Accession No. U47928; murine A-2, Accession No. AC002393) which were sequenced as part of the sequencing of human chromosome 12p13 and mouse chromosome 6, respectively. The human A-2 protein appears to be one of a family of alternatively-spliced gene products which further includes protein A-1 (Accession No. U47925) as well as A-3 (Accession No. U47929). The A-2 proteins, like the STMST proteins of the present invention, include many features indicative of the G protein-coupled receptor family of proteins.

For instance, the STMSTs of the present invention contain conserved cysteines found in the first 2 extracellular loops (prior to the third and fifth transmembrane domains) of most GPCRs (cys 83 and cys 161 of SEQ ID NO:71 or SEQ ID NO:74). A highly conserved asparagine residue in the first transmembrane domain is present (asn25 in SEQ ID NO:71 or SEQ ID NO:74). Transmembrane domain two of the STMST proteins contains a highly conserved leucine (leu49 of SEQ ID NO:71 or SEQ ID NO:74). The two cysteine residues are believed to form a disulfide bond that stabilizes the functional protein structure. A highly conserved tryptophan and proline in the fourth transmembrane domain of the STMST proteins is present (trp135 and pro 145 of SEQ ID NO:71 or SEQ ID NO:74). The third cytoplasmic loop contains 49 amino acid residues and is thus the longest cytoplasmic loop of the three, characteristic of G protein coupled receptors. Moreover, a highly conserved proline in the sixth transmembrane domain is present (pro260 of SEQ ID NO:71 and SEQ ID NO:74). The proline residues in the fourth, fifth, sixth, and seventh transmembrane domains are thought to introduce kinks in the alpha-helices and may be important in the formation of the ligand binding pocket. Furthermore, the conserved (in the second cytoplasmic loop) HRM motif found in almost all Rhodopsin family GPCRs is present in the STMST proteins of the instant invention (his107, arg108, met109 of SEQ ID NO:71 or SEQ ID NO:74). (The arginine of the HRM sequence is thought to be the most important amino acid in GPCRs and is invariant). Moreover, an almost invariant proline is present in the seventh transmembrane domain of STMST-2 (pro294 of SEQ ID NO:74).

As such, the STMST family of proteins, like the A-2 family of proteins, are referred to herein as G protein-coupled receptor-like proteins.

STMST-1 is also predicted to contain the following sites: cAMP and cGMP-dependent protein kinase phosphorylation site at amino acid residues 225-228 (KRRS; SEQ ID NO:90); Protein kinase C phosphorylation sites at residues 153-155 (SER) and at residues 290-292 (SSR); Casein kinase II phosphorylation sites at residues 228-231 (SSID; SEQ ID NO:91) and at residues 291-294 (SRQD; SEQ ID NO:92); N-myristoylation sites at residues 9-14 (GSAVGW; SEQ ID NO:93), residues 169-174 (GLGFGV; SEQ ID NO:94), residues 181-186 (GGSVAM; SEQ ID NO:95), residues 187-192 (GVICTA; SEQ ID NO:96), residues 232-237 (GSEPAK; SEQ ID NO:97), and at residues 244-249 (GLVTTI; SEQ ID NO:98); Amidation site at residues 223-226 (QGKR; SEQ ID NO:99).

Likewise, STMST is predicted to contain the following sites: cAMP- and cGMP-dependent protein kinase phosphorylation sites at amino acid residues 225-228 (KRRS; SEQ ID NO:100), residues 393-396 (RRFS; SEQ ID NO:101), residues 436-439 (RRAS; SEQ ID NO:102), and at residues 453-456 (RRRS; SEQ ID NO:103); Protein kinase C phosphorylation sites at residues 253-255 (SER), residues 268-270 (SLR), residues 392-394 (TRR), residues 462-464 (SLR), residues 482-484 (SPR), and at residues 560-562 (SLR); Casein kinase II phosphorylation sites at residues 228-231 (SSID; SEQ ID NO:104), residues 324-327 (SDDE; SEQ ID NO:105), residues 328-331 (TSLE; SEQ ID NO:106), residues 364-367 (SALE; SEQ ID NO:107), residues 396-399 (SHDD; SEQ ID NO:108), residues 417-420 (SGED; SEQ ID NO:109), residues 466-469 (SALD; SEQ ID NO:110), residues 506-509 (TAFE; SEQ ID NO:111), residues 568-571 (SWGE; SEQ ID NO:112), and at residues 590-593 (SPSE; SEQ ID NO:113); Tyrosine kinase phosphorylation site at residues 342-348 (RSLDYGY; SEQ ID NO:114); N-myristoylation sites at residues 9-14 (GSAVGW; SEQ ID NO:115), residues 169-174 (GLGFGV; SEQ ID NO:116), residues 181-186 (GGSVAM; SEQ ID NO:117), residues 187-192 (GVICTA; SEQ ID NO:118), residues 232-237 (GSEPAK; SEQ ID NO:119), residues 244-249 (GLVTTI; SEQ ID NO:120), residues 531-536 (GADPGE; SEQ ID NO:121), residues 564-569 (GLSASW; SEQ ID NO:122), residues 573-578 (GGLRAA; SEQ ID NO:123), and at residues 579-584 (GGGGST; SEQ ID NO:124); Amidation site at residues 223-226 (QGKR; SEQ ID NO:125).

The following table depicts an alignment of the transmembrane domain of 5 GPCRs. The conserved residues described herein are indicated by asterisks. An alignment of the transmembrane domains of 44 representative GPCRs can be found online.

| ALIGNMENT OF: | | | | |
|---|---|---|---|---|
| thrombin | (6.) | human | P25116 | (SEQ ID NO: 80) |
| rhodopsin | (19.) | human | P08100 | (SEQ ID NO: 81) |
| m1ACh | (21.) | rat | P08482 | (SEQ ID NO: 82) |
| IL-8RA | (30.) | human | P25024 | (SEQ ID NO: 83) |
| octopamine | (40.) | Drosophila melanogaster | P22270 | (SEQ ID NO: 84) |
| TM1 | | | | |
| | | * | | |
| 6. | 102 | TLFVPSVYTGVFVVSLPLNIMAIVVFILKMK | 132 | (SEQ ID NO: 137) |
| 19. | 37 | FSMLAAYMFLLIVLGFPINFLTLYVTVQHKK | 67 | (SEQ ID NO: 138) |
| 21. | 25 | VAFIGITTGLLSLATVTGNLLVLISFKVNTE | 55 | (SEQ ID NO: 139) |
| 30. | 39 | KYVVIIAYALVFLLSLLGNSLVMLVILYSRV | 69 | (SEQ ID NO: 140) |
| 40. | 109 | ALLTALVLSVIIVLTIIGNILVILSVFTYKP | 139 | (SEQ ID NO: 141) |
| | | \| | | |
| | | 1111111111111111111111111111111 | | |
| | | 3333333344444444445555555555666 | | |
| | | 2345678901234567890123456789012 | | |
| TM2 | | | | |
| | | * * | | |
| 6. | 138 | VVYMLHLATADVLFVSVLPFKISYYFSG | 165 | (SEQ ID NO: 142) |
| 19. | 73 | NYILLNLAVADLFMVLGGFTSTLYTSLH | 100 | (SEQ ID NO: 143) |
| 21. | 61 | NYFLLSLACADLIIGTFSMNLYTTYLLM | 88 | (SEQ ID NO: 144) |
| 30. | 75 | DVYLLNLALADLLFALTLPIWAASKVNG | 102 | (SEQ ID NO: 145) |
| 40. | 145 | NFFIVSLAVADLTVALLVLPFNVAYSIL | 172 | (SEQ ID NO: 146) |
| | | \| | | |
| | | 2222222222222222222222222222 | | |
| | | 4444444444555555555566666666 | | |
| | | 0123456789012345678901234567 | | |
| TM3 | | | | |
| | | * | | |
| 6. | 176 | RFVTAAFYCNMYASILLMTVISIDR | 200 | (SEQ ID NO: 147) |
| 19. | 111 | NLEGFFATLGGEIALWSLVVLAIER | 135 | (SEQ ID NO: 148) |
| 21. | 99 | DLWLALDYVASNASVMNLLLISFDR | 123 | (SEQ ID NO: 149) |
| 30. | 111 | KVVSLLKEVNFYSGILLLACISVDR | 135 | (SEQ ID NO: 150) |
| 40. | 183 | KLWLTCDVLCCTSSILNLCAIALDR | 207 | (SEQ ID NO: 151) |
| | | \| | | |
| | | 3333333333333333333333333 | | |
| | | 2222333333333344444444445 | | |
| | | 6789012345678901234567890 | | |
| TM4 | | | | |
| | | * * | | |
| 6. | 215 | TLGRASFTCLAIWALAIAGVVPLVLKE | 241 | (SEQ ID NO: 152) |
| 19. | 149 | GENHAIMGVAFTWVMALACAAPPLAGW | 175 | (SEQ ID NO: 153) |
| 21. | 138 | TPRRAALMIGLAWLVSFVLWAPAILFW | 164 | (SEQ ID NO: 154) |
| 30. | 149 | KRHLVKFVCLGCWGLSMNLSLPFFLFR | 175 | (SEQ ID NO: 155) |
| 40. | 222 | TVGRVLLLISGVWLLSLLISSPPLIGW | 248 | (SEQ ID NO: 156) |
| | | \| | | |
| | | 444444444444444444444444444 | | |
| | | 334444444444555555555566666 | | |
| | | 890123456789012345678901234 | | |
| TM5 | | | | |
| | | * * * | | |
| 6. | 268 | AYYFSAFSAVFFFVPLIISTVCYVSIIRC | 296 | (SEQ ID NO: 157) |
| 19. | 201 | ESFVIYMFVVHFTIPMIIIFFCYGQLVFT | 229 | (SEQ ID NO: 158) |
| 21. | 186 | PIITFGTAMAAFYLPVTVMCTLYWRIYRE | 214 | (SEQ ID NO: 159) |
| 30. | 200 | MVLRILPHTFGFIVPLFVMLFCYGFTLRT | 228 | (SEQ ID NO: 160) |
| 40. | 267 | RGYVIYSSLGSFFIPLAIMTIVYIEIFVA | 295 | (SEQ ID NO: 161) |
| | | \| | | |
| | | 55555555555555555555555555555 | | |
| | | 33334444444444555555555566666 | | |
| | | 67890123456789012345678901234 | | |
| TM6 | | | | |
| | | * * * | | |
| 6. | 313 | FLSAAVFCIFIICFGPTNVLLIAHYSFL | 340 | (SEQ ID NO: 162) |
| 19. | 252 | RMVIIMVIAFLICWVPYASVAFYIFTHQ | 279 | (SEQ ID NO: 163) |
| 21. | 365 | RTLSAILLAFILTWTPYNIMVLVSTFCK | 397 | (SEQ ID NO: 164) |
| 30. | 242 | RVIFAVVLIFLLCWLPYNLVLLADTLMR | 269 | (SEQ ID NO: 165) |
| 40. | 529 | RTLGIIMGVFVICWLPFFLMYVILPFCQ | 556 | (SEQ ID NO: 166) |
| | | \| | | |
| | | 6666666666666666666666666666 | | |
| | | 3333344444444445555555556666 | | |
| | | 5678901234567890123456789012 | | |
| TM7 | | | | |
| | | ** * | | |
| 6. | 347 | EAAYFAYLLCVCVSSISSCIDPLIYYYASSECQ | 379 | (SEQ ID NO: 167) |
| 19. | 282 | NFGPIFMTIPAFFAKSAAIYNPVIYIMMNKQFR | 314 | (SEQ ID NO: 168) |
| 21. | 394 | CVPETLWELGYWLCYVNSTVNPMCYALCNKAFR | 426 | (SEQ ID NO: 169) |
| 30. | 281 | NNIGRALDATEILGFLHSCLNPIIYAFIGQNFR | 313 | (SEQ ID NO: 170) |
| 40. | 559 | CPTNKFKNFITWLGYINSGLNPVIYTIFNLDYR | 591 | (SEQ ID NO: 171) |
| | | \| | | |
| | | 777777777777777777777777777777777 | | |
| | | 233333333334444444444555555555566 | | |
| | | 901234567890123456789012345678901 | | |

A search using the amino acid sequence of SEQ ID NO:71 was performed against the HMM database resulting in the identification of a 7 TM receptor profile in the amino acid sequence of SEQ ID NO:71. The results of the search are set forth below.

Score: 44.14 SEQ ID NO: 71 aa24-191
HMM: aa1-174 (SEQ ID NO: 172)

| | GNiLVIWvIcRyRRMRTPMNYFIvNLAvADLLFslft.MPFWMv |
|---|---|
| | YyvMq |
| 24 | ANAWGILSVGAKQKKWKPLEFLLCTLAATHMLN-VAVPIATYSV |
| | VQLRR |
| | gRWpFGdfMCrIWmYFDYMNYASIFfLTcISIDRYLWAICHPMr |
| | YmR |
| 72 | QRPDFEWNEGLCKVFVSTFYTLTLATCFSVTSLSYHRMWMVCWP |
| | VNYRL |
| | WMTpRHRAWvMIiiIWvMSFlISMPPFLMFrWstyrDEneWNmT |
| | WCmIyD |
| 121 | SNAKK-QAVHTVMGIWMVSFILSALPA-VG-W-HDTSERFYTHG- |
| | CRFIV |
| | WPewMWrWYvILmtiimgFYIPMiIMlF |
| 166 | AEIGLGFGVCFLLLV-GGSVA-MGVICT |

A search was performed against the HMM database resulting in the identification of a spectrin alpha chain profile in the amino acid sequence of SEQ ID NO:71. The results of the search are set forth below.

Score: 8.78 SEQ ID NO: 71 aa266-372
HMM: aa1-106 (SEQ ID NO: 174)

| | IqeRMnElndRWerLkelMeqRRQMLedSmrlQQFfRDmDEeEs |
|---|---|
| | WInEK |
| 266 | FSSLRADASAPWMALCVLWCSVAQALLLPVFLWACDRYRADLKA |
| | VREKC |
| | EqilnSDDYGkDLtsVQnLlkKHQaFEaDIaAHE.dRIqalnef |
| 315 | MALMANDEESDDETSLEGGISPDLVLERSLDYGYGGDFVALDRM |
| | aqqLIqenHYasEe |
| 365 | AKYEISALEGGLPQ |

Tissue Distribution of STMST-1 mRNA

This Example describes the tissue distribution of STMST mRNA, as determined by Northern blot hybridization.

Northern blot hybridizations with the various RNA samples were performed (Clontech Multi-tissue Northern I and human fetal tissue northern) under standard conditions and washed under stringent conditions. A 4.5 Kb mRNA transcript was detected in heart, brain, placenta, lung, liver, skeletal muscle, fetal brain, fetal lung, and fetal kidney. Expression was highest in fetal brain.

Northern blot hybridization of poly A+ mRNA samples were also performed (Human Clontech poly A+ northern). A ˜4.5 mRNA transcript was expressed in the following tissues at relative levels of heart>brain>placenta>liver>kidney.

### Example 14

### Characterization of STMST Expression by RT-PCR

In this example, STMST expression levels were measured in a variety of tissue and cell samples using the Taqman™ procedure. The Taqman™ procedure is a quantitative, real-time PCR-based approach to detecting mRNA. The RT-PCR reaction exploits the 5′ nuclease activity of AmpliTaq Gold™ DNA Polymerase to cleave a TaqMan™ probe during PCR. Briefly, cDNA is generated from the samples of interest and serves as the starting materials for PCR amplification. In addition to the 5′ and 3′ gene-specific primers, a gene-specific oligonucleotide probe (complementary to the region being amplified) is included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. Table I sets forth the relative expression of STMST mRNA in a variety of tissues.

TABLE II
STMST Expression in Human Tissues

| Tissue Source | Relative Expression |
|---|---|
| aorta/normal | 0.39 |
| fetal heart/normal | 14.94 |
| heart/normal | 5.70 |
| heart/CHF | 3.23 |
| vein/normal | 0.22 |
| SMC/aortic | 4.38 |
| nerve/normal | 1.83 |
| spinal cord/normal | 0.28 |
| brain cortex/normal | 25.30 |
| brain hypothalamus/normal | 20.47 |
| glioblastoma | 0.75 |
| breast/normal | 1.41 |
| breast/tumor | 1.40 |
| ovary/normal | 0.89 |
| ovary/tumor | 1.25 |
| pancreas/normal | 1.67 |
| prostate/normal | 1.87 |
| prostate/tumor | 2.12 |
| colon/normal | 0.31 |
| colon/tumor | 2.71 |
| colon/BD | 0.49 |
| kidney/normal | 1.12 |
| liver/normal | 0.99 |
| liver/fibrosis | 1.14 |
| fetal liver/normal | 0.38 |
| lung/normal | 1.30 |
| lung/COPD | 1.20 |
| spleen/normal | 0.64 |
| tonsil/normal | 0.03 |
| lymph node/normal | 0.18 |
| thymus/normal | 1.52 |
| epithelial cells/prostate | 50 |
| endothelial cells/aortic | 1.06 |
| skeletal muscle/normal | 0.33 |
| fibroblasts/dermal | 35.28 |
| skin/normal | 5.43 |
| adipose/normal | 0.10 |
| osteoblasts/primary | 13.37 |
| osteoblast/undif. | 0.75 |
| osteoblasts/diff. | 0.75 |
| osteoclasts | 0.29 |

The highest expression was observed in epithelial cells, brain (cortex/hypothalamus), osteoblasts and dermal fibroblasts in the human tissue panel tested.

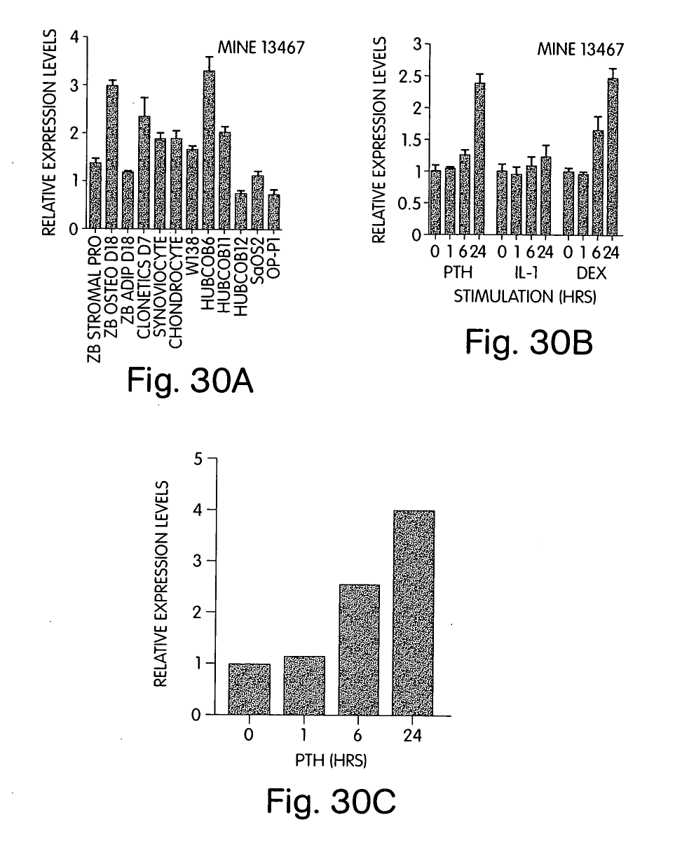

To further investigate the high expression in primary osteoblasts, STMST expression levels were measured by quantitative PCR using the Taqman™ procedure as described above. The relative levels of STMST expression in various cell lines is depicted in FIG. 30A. The data demonstrate that at least three-fold STMST expression is seen in the ZB Osteo D18 cell line as well as in HUBCOB6 cells. Expression was also significantly increased in Clonetics D7 cells (i.e., differentiated osteoblasts). The data presented in FIG. 30B depict relative STMST expression levels in primary osteoblasts treated for 0, 1, 6 or 24 hours with either parathyroid hormone (PTH), interleukin-1 (IL-1) or dexamethasone (DEX). As clearly demonstrated by the data in FIG. 30B, expression of STMST is upregulated in primary human osteoblasts stimulated for 24 hours with PTH. Transcriptional profiling analysis of a cDNA array (FIG. 30C) confirms that expression in primary human osteoblasts is inducible by treatment of cells with parathyroid hormone (PTH). These data suggest that STMST and/or STMST agonism may mimic PTH anabolic effects on bone.

### Example 15

### Characterization of STMST Expression in Osteogenic Cells by Northern Blot Analysis and In Situ Analysis

Northern blot hybridization of poly A+ from the following samples was performed under standard hybridization and wash conditions: human bone (total mRNA), human bone (poly A+ RNA), HuBCOB6 (primary osteoblasts), HuBCOB11 (primary osteoblasts), huBCOB12 (primary osteoblasts), U2OS (osteoblast cell line), human spleen control (total mRNA) and human skeletal muscle (total mRNA). STMST transcript was also detected in human spleen mRNA.

In situ analysis was performed according to standard methodologies on tissue sections of human fetal bone. To generate a sense probe the following primers were used; forward primer: AGATGCCACCTTCCAGGCT (SEQ ID NO:85) and reverse primer: GGAGAAGTGCATGGCCCTC (SEQ ID NO:86) resulting in a sense probe having the following sequence: TCTCATCGTCTGACTCCTCGTCGTTGG (SEQ ID NO:87). Sense STMST probe hybridized particularly to osteoblasts within human fetal bone sections, consistent with coexpression of STMST with PTH-R positive osteoblasts. Importantly, this pattern of expression was very similar to the expression pattern for that of PTH-R, a good pre-osteoblast/osteoblast lineage marker.

### Example 16

### STMST-2 mRNA In Situ Hybridization

This Example describes the characterization of brain-specific STMST expression.

The distribution of STMST-2 mRNA in mouse brain was examined as follows. Mouse brain was frozen with powdered dry ice, and cryostat sections were cut at 10 μm thickness through hypothalamus region, mounted on superfrost plus sides (VWR) and stored at −80° until needed.

Prior to analysis, mouse brain sections were air dried for 20 minutes and then incubated with ice cold 4% PFA (paraformaldehyde)/1×PBS for 10 minutes. The slides were then washed with 1×PBS twice (5 minutes each time), incubated with 0.25% acetic anhydride/1 M triethanolamine for 10 minutes, washed with PBS for 5 minutes and dehydrated with 70%, 80%, 95% and 100% ethanol (1 minute each). Sections were incubated with chloroform for 5 minutes, rehydrated with 100% and 95% ethanol, then air dried. Hybridizations were performed using the following ³⁵S-radiolabeled (5×10⁷ cpm/ml) cRNA probes: 5‘GGCGGTGCACACAGTTAT’3 (SEQ ID NO:88) and 5′ AGAGAGCGCTCCAAATACCAT3′ (SEQ ID NO:89) in the presence of 50% formamide, 10% dextran sulfate, 1×Denhardt's solution, 600 mm NaCl, 10 mm DTT, 0.25% SDS and 100 μg/ml RNase A in TNE at 37° C. for 30 minutes, washed in TNE for 10 minutes, incubated once in 2×SSC at 600 for 1 hour, once in 0.2×SSC at 60° for 1 hour, 0.2×SSC at 65° for 1 hour and dehydrated with 50%, 70%, 80%, 95% and 100% ethanol. Localization of mRNA transcripts was detected by dipping slides in Kodak NBT-2 photoemulsion and exposing for 14 days at 4° C., followed by development with Kodak Dektol developer. Slides were counterstained with haemotoxylin and eosin and photographed. Controls for the in situ hybridization experiments included the use of a sense probe which showed no signal above background levels.

This analysis revealed that STMST-2 mRNA is expressed within the arcuate nucleus and the ventromedial nucleus of the hypothalamus, both of which are implicated in control of feeding behavior, as described herein.

### Example 17

### Regulation of STMST Expression During Tube Formation of Endothelial Cells

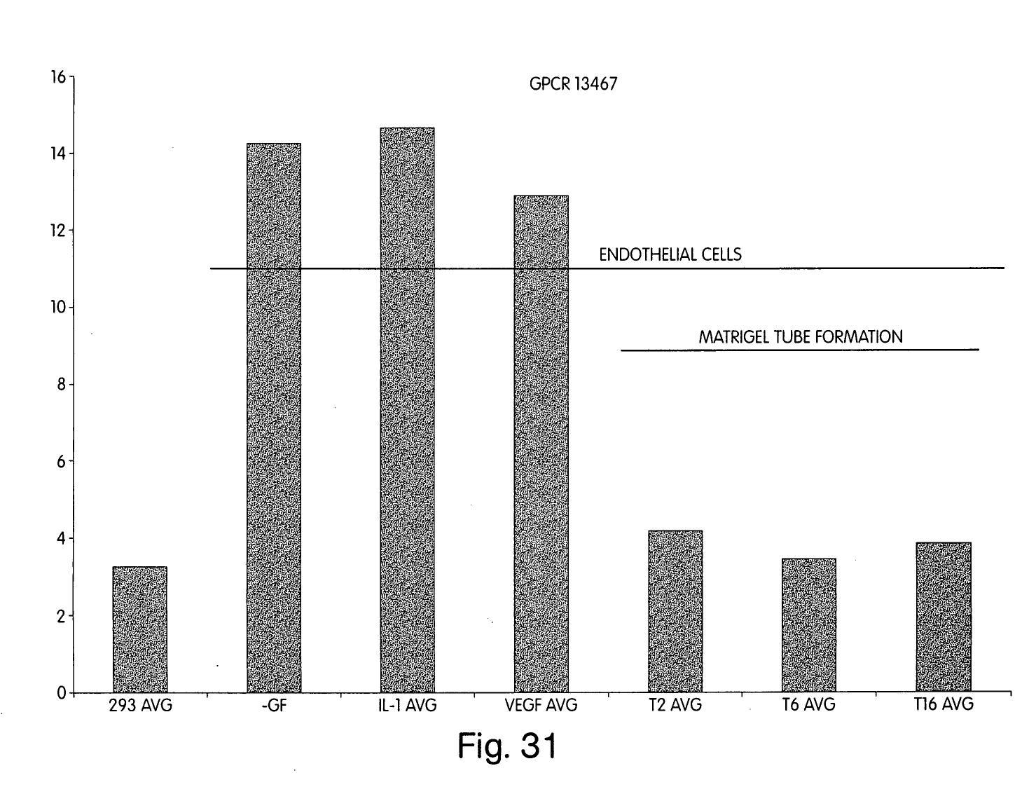

This example describes the characterization of STMST expression during tube formation of endothelial cells. FIG. 31 shows relative mRNA expression levels of STMST, as determined by microarray hybridization. Human umbilical vein endothelial cells (HUVECs) were grown under a variety of conditions. On tissue culture plastic, expression of STMST is higher in endothelial cells than in non-endothelial 293 cells. When endothelial cells are cultured on Matrigel and form vascular tube-like structures, STMST expression is markedly down regulated. This result suggests that expression of STMST may inhibit vascular tube formation, so it is down-regulated to allow vascular tube formation to proceed. This process is believed to be similar to angiogenesis. In addition to its relevance to angiogenesis, the data also suggest roles in atherosclerosis and the control of vascular tone, as endothelial cell phenotype plays an important role in both of these processes. At least two genes with established relevance to atherosclerosis and control of vascular tone, cyclo-oxygenase-2 and endothelin-1, are regulated in this model.

### Example 18

### Expression of Recombinant ITALY, Lor-2, STRIFE, TRASH, BDSF, LRSG, and STMST Protein in Bacterial Cells

In this example, ITALY, Lor-2, STRIFE, TRASH, BDSF, LRSG, or STMST is expressed as a recombinant glutathione-S-transferase (GST) fusion polypeptide in E. coli and the fusion polypeptide is isolated and characterized. Specifically, ITALY, Lor-2, STRIFE, TRASH, BDSF, LRSG, or STMST is fused to GST and this fusion polypeptide is expressed in E. coli, e.g., strain PEB 199. As the human ITALY, Lor-2, STRIFE, TRASH, BDSF, LRSG, or STMST protein is predicted to be approximately 18 kDa, and GST is predicted to be 26 kDa, the fusion polypeptide is predicted to be approximately 44 kDa, in molecular weight. Expression of the GST-ITALY, Lor-2, STRIFE, TRASH, BDSF, LRSG, or STMST fusion protein in PEB 199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion polypeptide is determined.

### Example 19

### Expression of Recombinant ITALY, Lor-2, STRIFE, TRASH, BDSF, LRSG, and STMST PROTEIN in COS Cells

To express the ITALY, Lor-2, STRIFE, TRASH, BDSF, LRSG, or STMST gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire ITALY, Lor-2, STRIFE, TRASH, BDSF, LRSG, or STMST protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the ITALY, Lor-2, STRIFE, TRASH, BDSF, LRSG, or STMST DNA sequence is amplified by PCR using two primers. The 5′ primer contains the restriction site of interest followed by approximately twenty nucleotides of the ITALY, Lor-2, STRIFE, TRASH, BDSF, LRSG, or STMST coding sequence starting from the initiation codon; the 3′ end sequence contains complementary sequences to the other restriction site of interest, a translation stop codon, the HA tag or FLAG tag and the last 20 nucleotides of the ITALY, Lor-2, STRIFE, TRASH, BDSF, LRSG, or STMST coding sequence. The PCR amplified fragment and the pCDNA/Amp vector are digested with the appropriate restriction enzymes and the vector is dephosphorylated using the CIAP enzyme (New England Biolabs, Beverly, Mass.). Preferably the two restriction sites chosen are different so that the ITALY, Lor-2, STRIFE, TRASH, BDSF, LRSG, or STMST gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the ITALY, Lor-2, STRIFE, TRASH, BDSF, LRSG, or STMST-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. The expression of the ITALY, Lor-2, STRIFE, TRASH, BDSF, LRSG, or STMST polypeptide is detected by radiolabeling (³⁵S-methionine or ³⁵S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the ITALY, Lor-2, STRIFE, TRASH, BDSF, LRSG, or STMST coding sequence is cloned directly into the polylinker of the pCDNA/Amp vector using the appropriate restriction sites. The resulting plasmid is transfected into COS cells in the manner described above, and the expression of the ITALY, Lor-2, STRIFE, TRASH, BDSF, LRSG, or STMST polypeptide is detected by radiolabeling and immunoprecipitation using an ITALY, Lor-2, STRIFE, TRASH, BDSF, LRSG, or STMST specific monoclonal antibody.

### Example 20

### Retroviral Delivery of ITALY, Lor-2, STRIFE, TRASH, BDSF, LRSG, and STMST into Mice

The entire open reading frame of ITALY, LOR-2, STRIFE, TRASH, BDSF, LRSG, or STMST is subcloned into the retroviral vector MSCVneo, described in Hawley et al. (1994) Gene Therapy 1:136-138. Cells (293Ebna, Invitrogen) are then transiently transfected with the ITALY, LOR-2, STRIFE, TRASH, BDSF, LRSG, or STMST construct and with constructs containing viral regulatory elements, to produce high titre retrovirus containing the ITALY, LOR-2, STRIFE, TRASH, BDSF, LRSG, or STMST gene. This virus is then used to transfect mice. These mice are then tested for any gross pathology and for changes in their immune response using standard assays.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule having a nucleotide sequence which is at least 90% identical to the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof;
b) a nucleic acid molecule comprising at least 15 nucleotide residues and having a nucleotide sequence identical to at least 15 consecutive nucleotide residues of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof;
c) a nucleic acid molecule which encodes a polypeptide comprising the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695;
d) a nucleic acid molecule which encodes a fragment of a polypeptide comprising the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695, wherein the fragment comprises at least 10 consecutive amino acid residues of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695;
e) a nucleic acid molecule which encodes a fragment of a polypeptide comprising the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695, wherein the fragment comprises consecutive amino acid residues corresponding to at least half of the full length of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695; and
f) a nucleic acid molecule which encodes a naturally occurring allelic variant of a polypeptide comprising the amino acid sequence of any of SEQ ID NO:2, 4, 11, 23, 27, 32, 37, 39, 41, 46, 49, 51, 54, 59, 68, 71, or 74, wherein the nucleic acid molecule hybridizes with a nucleic acid molecule consisting of the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof under stringent conditions.

2. The isolated nucleic acid molecule of claim 1, which is selected from the group consisting of:
a) a nucleic acid having the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof; and
b) a nucleic acid molecule which encodes a polypeptide having the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695;
b) a naturally occurring allelic variant of a polypeptide comprising the amino acid sequence of any of SEQ ID NO:2, 4, 11, 23, 27, 32, 37, 39, 41, 46, 49, 51, 54, 59, 68, 71, or 74, wherein the polypeptide is encoded by a nucleic acid molecule which hybridizes with a nucleic acid molecule consisting of the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof.

9. The isolated polypeptide of claim 8 having the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695;
b) a polypeptide comprising a fragment of the amino acid sequence of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695, wherein the fragment comprises at least 10 contiguous amino acids of any of SEQ ID NO:2, 4, 11, 23, 27, 32, 37, 39, 41, 46, 49, 51, 54, 59, 68, 71, or 74, or the amino acid sequence encoded by the nucleotide sequence of any of the clones deposited as ATCC Accession number 98960, 98756, or 98695; and
c) a naturally occurring allelic variant of a polypeptide comprising the amino acid sequence of any of SEQ ID NO:2, 4, 11, 23, 27, 32, 37, 39, 41, 46, 49, 51, 54, 59, 68, 71, or 74, or a complement thereof, wherein the polypeptide is encoded by a nucleic acid molecule which hybridizes with a nucleic acid molecule consisting of the nucleotide sequence of any of SEQ ID NO:1, 3, 10, 12, 22, 24, 25, 26, 28, 29, 31, 33, 36, 38, 40, 45, 47, 48, 50, 52, 53, 58, 60, 67, 69, 70, 72, 73, or 75, or the nucleotide sequence of any of the clones deposited as ATCC Accession number 98960, 98756, or 98695, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay;
c) detection of binding using an assay for an activity characteristic of the polypeptide.

21. A method for modulating the activity of a polypeptide of claim 8 comprising contacting a polypeptide or a cell expressing a polypeptide of claim 8 with a compound which binds with the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.
